(19) United States
(12) Patent Application Publication
(10) Pub. No.: US 2025/0340646 A1
(43) Pub. Date: Nov. 6, 2025

(54) ANTI-PD-L1 MONOCLONAL ANTIBODIES AND FUSION PROTEINS WITH INTERLEUKIN-15, INTERLEUKIN-15 RECEPTOR ALPHA, OR INTERLEUKIN-2

(71) Applicant: Elpis Biopharmaceuticals, Lexington, MA (US)

(72) Inventors: Kehao Zhao, Lexington, MA (US); Yan Chen, Lexington, MA (US); Samuel Clement Hassan, Lexington, MA (US); Jenna Nguyen, Lexington, MA (US); Ning Jiang, Lexington, MA (US)

(21) Appl. No.: 18/262,122

(22) PCT Filed: Jan. 21, 2022
(86) PCT No.: PCT/US2022/013417

§ 371 Date: Jul. 19, 2023

Related U.S. Application Data
(60) Provisional application No. 63/140,749, filed on Jan. 22, 2021.

(51) Int. Cl. C07K 16/28; A61K 39/00; A61P 35/00; A61P 37/04; C07K 14/54; C07K 14/55; C07K 14/715

(52) U.S. Cl. CPC C07K 16/2827; A61P 35/00; A61P 37/04; C07K 14/5443; C07K 14/55; C07K 14/7155; A61K 2039/505; C07K 2317/52; C07K 2317/55; C07K 2317/565; C07K 2317/622; C07K 2317/76; C07K 2317/92; C07K 2319/00

(57) ABSTRACT

The instant disclosure provides proteins with antibody heavy chain variable domains and light chain variable domains that can be paired to form antigen-binding sites that specifically bind to PD-L1. In certain embodiments, the proteins or antigen-binding sites form antibody or bispecific antibody, such as, for example, PD-L1/IL-2Rβ bispecific antibody. Also provided are pharmaceutical compositions comprising such proteins, therapeutic methods for using such proteins, and pharmaceutical compositions thereof, including for the treatment of cancer.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 300096_402WO_SEQUENCE_LISTING.txt. The text file is 220 KB, was created on Jan. 20, 2021, and is being submitted electronically via EFS-Web.

### BACKGROUND

PD-L1 (programmed death-ligand 1), also known as CD274 or B7 homolog 1 (B7-H1) is a type 1 transmembrane protein that inhibits TCR-mediated activation of IL-2 production and T cell proliferation when PD-L1 binds to PD-1 on T cells. Anti-PD-L1 antibodies have been used as therapeutics for the treatment of cancer. However, there remains a need for biotherapeutic agents to more effectively modulate tumor growth.

### BRIEF SUMMARY

The present disclosure provides proteins with antibody heavy chain variable domains and light chain variable domains that can be paired to form antigen-binding sites that specifically bind to PD-L1. The proteins or antigen-binding sites, of the present disclosure, may form an antibody or bifunctional antibody, such as, for example, PD-L1/IL-2Rβ bifunctional antibody. The proteins or antigen-binding sites of the present disclosure may be used to treat or prevent cancerous or infectious conditions and disorders.

In some embodiments, disclosed herein is an antigen-binding site that binds PD-L1, comprising: a heavy chain variable domain (VH) comprising complementarity-determining region 1 (CDR1) sequence of SEQ ID NO: 3, 11, 19, 33, 52, or 63; complementarity-determining region 2 (CDR2) sequence of SEQ ID NO: 4, 12, 20, 34, 41, 53, or 64; and complementarity-determining region 3 (CDR3) sequence of SEQ ID NO: 5, 13, 21, 27, 35, 46, 54, 65, 71, 74, 77, 80, 83, 86, or 89; and a light chain variable domain (VL) comprising CDR1 sequence of SEQ ID NO: 6, 14, 22, 28, 36, 47, 55, or 66; CDR2 sequence of SEQ ID NO: 7, 15, 23, 29, 37, 42, 48, 56, 60, or 67; and CDR3 sequences of SEQ ID NO: 8, 16, 24, 30, 38, 43, 49, 57, or 68.

In some embodiments, the antigen binding site is present in a single-chain fragment variable fragment (scFv), an antigen-binding fragment (Fab), an antibody, or similar antigen binding protein.

In some embodiments, disclosed herein is a bifunctional protein, comprising:
(a) an antigen-binding site that binds PD-L1, comprising:
   (i) a heavy chain variable domain (VH) comprising complementarity-determining region 1 (CDR1) of SEQ ID NO: 3, 11, 19, 33, 52, or 63; complementarity-determining region 2 (CDR2) of SEQ ID NO: 4, 12, 20, 34, 41, 53, or 64; and complementarity-determining region 3 (CDR3) of SEQ ID NO: 55, 13, 21, 27, 35, 46, 54, 65, 71, 74, 77, 80, 83, 86, or 89; and
   (ii) a light chain variable domain (VL) comprising CDR1 sequence of SEQ ID NO: 6, 14, 22, 28, 36, 47, 55, or 66; CDR2 sequence of SEQ ID NO: 7, 15, 23, 29, 37, 42, 48, 56, 60, or 67; and CDR3 sequences of SEQ ID NO: 8, 16, 24, 30, 38, 43, 49, 57, or 68; and
(b) an interleukin-15 (IL-15) polypeptide, an interleukin-15 receptor alpha (IL-15Rα) polypeptide, a wild-type interleukin-2 (IL-2) polypeptide, or an engineered IL-2 polypeptide, or a functional fragment or variant thereof.

In some embodiments, the present disclosure provides a method of treating a disease in a subject in need thereof, the method comprising administering to the subject a therapeutically effective amount an antigen-binding site, a protein or antibody, a bifunctional protein or antibody, or a pharmaceutical composition thereof. In certain aspects, the disease is cancer.

These and other aspects of the present disclosure will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows ELISA binding of identified scFvs to PD-L1.

FIGS. 2A and 2B show FACS analysis of identified scFvs binding to K562 cells engineered to express high levels of cell surface human PD-L1.

FIGS. 3A and 3B show PD-1 and PD-L1 competition results for identified scFvs in HTRF.

FIG. 4 shows results of a Jurkat cell NFAT reporter assay for identified scFvs.

FIG. 5 shows ELISA binding of affinity matured scFvs to PD-L1.

FIG. 6 shows FACS analysis of affinity matured scFvs binding to K562 cells engineered to express high levels of cell surface human PD-L1.

FIG. 7 shows PD-1 and PD-L1 competition results for affinity matured scFvs in HTRF.

FIG. 8 shows the Jurkat cell NFAT reporter assay for affinity matured scFvs.

FIG. 9 shows antibodies, bifunctional antibodies, and alternative molecular formats. Bifunctional formats include those that bind to PD-L1 and comprise an IL-2 cytokine or bind to PD-L1 and comprise a IL-15 cytokine, respectively.

FIG. 10 shows FACS analysis of anti-PD-L1 IgG antibody binding to K562 cells engineered to express high levels of cell surface human PD-L1.

FIG. 11 shows the Jurkat cell NFAT reporter assay for anti-PD-L1/IL-15 fusion antibodies.

FIGS. 12A-12D shows the p-STAT5 activation results of anti-PD-L1/IL-15 fusion antibodies. FIG. 12A shows p-STAT5 activation in CD4+FoxP3− T cells. FIG. 12B shows p-STAT5 activation in NK cells. FIG. 12C shows p-STAT5 activation in CD8+ T cells. FIG. 12D shows p-STAT5 activation in T regulatory cells.

FIGS. 13A and 13B show ELISA binding of IL-2-Fc clones to IL-2 receptors. FIG. 13A shows ELISA binding of IL-2-Fc clones to IL-2Rα receptor. FIG. 13B shows ELISA binding of IL-2-Fc to IL-2Rβ receptor.

FIGS. 14A-14H show p-STAT5 activation assays by IL-2-Fc clones performed on cells from human PBMCs of two separate donors. FIG. 14A shows p-STAT5 activation in CD4+ T cells from donor 126. FIG. 14B shows p-STAT5 activation in CD8+ T cells from donor 126. FIG. 14C shows p-STAT5 activation in NK cells from donor 126. FIG. 14D shows p-STAT5 activation in T regulatory cells from donor 126. FIG. 14E shows p-STAT5 activation in CD4+ T cells from donor 359. FIG. 14F shows p-STAT5 activation in CD8+ T cells from donor 359. FIG. 14G shows p-STAT5 activation in NK cells from donor 359. FIG. 14H shows p-STAT5 activation in T regulatory cells from donor 359.

FIGS. 15A-15C show ELISA binding of IL-2/anti-PD-L1 scFv Fc variants to IL-2 receptors (IL-2Rα or IL-2Rβ) and PD-L1. FIG. 15A shows ELISA binding of IL-2/anti-PD-L1 scFv Fc variants to IL-2Rα. FIG. 15B shows ELISA binding of IL-2/anti-PD-L1 scFv Fc variants to IL-2Rβ. FIG. 15C shows ELISA binding of IL-2/anti-PD-L1 scFv Fc variants to PD-L1.

FIG. 16 shows the Jurkat cell NFAT reporter assay for PD-1 and PD-L1 interaction blocking activity of IL-2/anti-PD-L1 scFv Fc variants.

FIGS. 17A-17H show p-STAT5 activation of CD8+ T cells, NK cells, and T regulatory cells from human PBMCs of two separate donors, by IL-2/anti-PD-L1 scFv Fc variants. FIG. 17A shows p-STAT5 profiling curve for CD4+ cells from donor 359. FIG. 17B shows p-STAT5 profiling curve for CD8+ cells from donor 359. FIG. 17C shows p-STAT5 profiling curve for NK cells from donor 359. FIG. 17D shows p-STAT5 profiling curve for T regulatory cells from donor 359. FIG. 17E shows p-STAT5 profiling curve for CD4+ cells from donor 126. FIG. 17F shows p-STAT5 profiling curve for CD8+ cells from donor 126. FIG. 17G shows p-STAT5 profiling curve for NK cells from donor 126. FIG. 17H shows p-STAT5 profiling curve for T regulatory cells from donor 126.

FIGS. 18A-18C show ELISA binding of IL-2/anti-PD-L1 Fab variants to IL-2 receptors (IL-2Rα or IL-2Rβ) and PD-L1. FIG. 18A shows ELISA binding of IL-2/anti-PD-L1 Fab variants to IL-2Rα. FIG. 18B shows ELISA binding of IL-2/anti-PD-L1 Fab variants to IL-2Rβ. FIG. 18C shows ELISA binding of IL-2/anti-PD-L1 Fab variants to PD-L1.

FIG. 19 shows the Jurkat cell NFAT reporter assay for PD-1 and PD-L1 interaction blocking activity of IL-2/anti-PD-L1 Fab variants.

FIG. 20 shows sensorgram of an IL-2/anti-PD-L1 Fab bifunctional variant simultaneously binding to PD-L1 and IL-2 receptor (IL-2Rα or IL-2Rβ).

FIGS. 21A-21H show p-STAT5 activation of CD8+ T cells, NK cells, and T regulatory cells from human PBMCs of two separate donors, by IL-2/anti-PD-L1 Fab variants. FIG. 21A shows p-STAT5 profiling curve for CD4+ cells from donor 857. FIG. 21B shows p-STAT5 profiling curve for CD8+ cells from donor 857. FIG. 21C shows p-STAT5 profiling curve for NK cells from donor 857. FIG. 21D shows p-STAT5 profiling curve for T regulatory cells from donor 857. FIG. 21E shows p-STAT5 profiling curve for CD4+ cells from donor 359. FIG. 21F shows p-STAT5 profiling curve for CD8+ cells from donor 359. FIG. 21G shows p-STAT5 profiling curve for NK cells from donor 359. FIG. 21H shows p-STAT5 profiling curve for T regulatory cells from donor 359.

FIGS. 22A-22D shows p-STAT5 activation of CD4+ Tcells, CD8+ T cells, NK cells, and T regulatory cells in mouse splenocytes, by IL-2/anti-PD-L1 Fab variants. FIG. 22A shows p-STAT5 activation of CD4+ T cells. FIG. 22B shows p-STAT5 activation of CD8+ T cells. FIG. 22C shows p-STAT5 activation of NK cells. FIG. 22D shows p-STAT5 activation of T regulatory cells.

FIG. 23 shows tumor growth inhibition curves plotted from inhibition data in humanized mouse MB-231 model after treatment with an engineered anti-PD-L1 mAb (EP205/EP206) Q3D or Atezolizumab for 20 days.

FIGS. 24A-24B show levels of human TNFα (FIG. 24A) and IFNγ (FIG. 24B) in plasma from humanized mouse MB-231 model after treatment with anti-PD-L1 mAb (EP205/EP206) Q3D or Atezolizumab for 20 days.

FIGS. 25A-25D show blood immune cell profiling of MC38 tumor bearing mice after treatment with IL-2/anti-PD-L1 Fab variants for 11 days. FIG. 25A shows profiling of CD4+ FOXP3− T cells (control). FIG. 25B shows profiling of CD8+ T cells. FIG. 25C shows profiling of NK cells. FIG. 25D shows profiling of T regulatory cells.

FIGS. 26A-26D show splenocyte immune cell profiling of MC38 tumor bearing mice after treatment with IL-2/anti-PD-L1 Fab variants for 11 days. FIG. 26A shows profiling of CD4+ FOXP3− T cells (control). FIG. 26B shows profiling of CD8+ T cells. FIG. 26C shows profiling of NK cells. FIG. 26D shows profiling of T regulatory cells.

FIG. 27 shows tumor growth inhibition curves plotted from inhibition data of tumor volume over time obtained from inhibition data in B16F10-PD-L1 tumor in hPD1 transgenic mice that were treated with IL-2/anti-PD-L1 Fab variants or Atezolizumab for 20 days.

FIG. 28 shows tumor growth inhibition curves plotted from inhibition data of tumor volume over time obtained from MB-231 tumor NCG mice model that were treated with IL-2/anti-PD-L1 Fab variants or Atezolizumab for 20 days.

FIG. 29A-29B shows expression levels of PD-1 in T cells following treatment of human PBMC by anti-PD-L1-Fab/IL2 fusion proteins. FIG. 29A shows expression of PD-1 in CD8+ T cells after 5-days of treatment with EP290/EP325/EP205 or EP415/EP325/EP205. FIG. 29B shows expression of PD-1 in T regulatory cells after 5-days of treatment with EP290/EP325/EP205 or EP415/EP325/EP205.

FIG. 30A-30B shows binding of anti-PD-L1-Fab/IL2 fusion proteins to IL2Rβγ. FIG. 30A shows expression levels of CD25, CD122, and PD-L1 in the HEK Blue IL2 cell line. FIG. 30B shows binding of EP290/EP325/EP205, EP415/EP325/EP205 to IL2R in the presence or absence of an anti-CD25 antibody.

FIG. 31A-31B shows tumor localization of fluorescently labelled EP415/EP325/EP205 in vivo. FIG. 31A shows a representative image of C57BL/6N and B6N Albino tumor bearing mice 24 hours after fluorescently labelled EP415/EP325/EP205 injection. FIG. 31B shows time dependent enrichment of EP415/EP325/EP205 to MC-38-hPD-L1 and MC-38 tumor sites.

FIG. 32A-32E shows tumor volume over time in humanized NCG mice and quantifies immune cells present at the tumor site. FIG. 32A shows a tumor growth inhibition curve of EP415/EP325/EP205 against anti-PD1/PD-L1 responsive cancer cell MDA-MB-231 in the humanized NCG mice. FIG. 32B shows the level of CD4+ T cells in the tumor site. FIG. 32C shows the level of CD8+ T cells in the tumor site. FIG. 32D shows the level of NK cells in the tumor site. FIG. 32E shows the level of TReg cells in the tumor site.

FIGS. 33A-33B show the results of experiments performed in murine models of Cold tumors. FIG. 33A shows the tumor growth inhibition curve of EP415/EP325/EP205 compared to vehicle control and anti-PD-L1 EP205/EP206 against COLO205 tumors in the humanized mice. FIG. 33B shows the corresponding body weight change of the mice in FIG. 33A.

FIG. 34A-34F shows the results of experiments performed in murine models of an anti-PD1 antibody resistant tumor. FIG. 34A shows the tumor growth inhibition curve of EP415/EP325/EP205 against H1975 cancer cell tumors in the humanized mice. FIG. 34B shows the corresponding body weight change of the mice in FIG. 34B. FIG. 34C shows the number of hCD45 expressing cells per mm³ of tumor. FIG. 34D shows the ratio of CD8+ T cells to Treg cells. FIG. 34E shows the number of CD8+ T cells per mm³ of tumor. FIG. 34F shows the ratio of NK cells to T regulatory cells.

FIG. 35A-35C shows results of experiments performed in a cynomolgus monkey model. FIG. 35A shows the concentration of EP415/EP325/EP205 detected in monkey plasma by ELISA and plotted against time. FIG. 35B shows the percentage of immune cell populations in monkey blood after dosing with 0.1 mg/kg EP415/EP325/EP205. FIG. 35C shows the percentage of immune cell populations in monkey blood after dosing with 0.5 mg/kg EP415/EP325/EP205.

### DETAILED DESCRIPTION

Anti-PD-L1 antibody has been a promising immunotherapy, but there remains a need for utilizing biotherapeutic agents to more effectively modulate tumor. Immunotherapy using cytokines, such as IL-2 and IL-15, has been shown effective in cancer treatment. Thus, antibodies targeting PD-L1 may prove to be a useful immunomodulation when further targeting IL-2 and IL-15. Provided herein are new anti-PD-L1 antibodies, scFv, and Fab polypeptides. In addition, provided herein are bifunctional fusion proteins comprising (a) anti-PD-L1 antibodies, scFv, or Fab polypeptides, and (b) (i) IL-15, IL-15Rα, or both, or (ii) IL-2 or engineered variants thereof.

The instant disclosure provides antigen-binding sites that bind human PD-L1. These antigen-binding sites can bind various epitopes in an extracellular domain of PD-L1. Proteins and protein conjugates containing such antigen-binding sites, such as, antibodies, bifunctional antibodies, antibody-drug conjugates, immunocytokines, and bispecific T-cell engagers, as well as immune effector cells (e.g., T cells) expressing a protein containing such an antigen-binding site (e.g., a chimeric antigen receptor (CAR)), are useful for treating diseases, such as cancer, associated with PD-L1. The instant disclosure also provides pharmaceutical compositions comprising such proteins, protein conjugates, immune effector cells, as well as therapeutic methods for using such proteins, protein conjugates, immune effector cells, and pharmaceutical compositions, including, but not limited to, cancer treatment. Various aspects of the antigen-binding sites described in the instant disclosure are described in the sections below; however, aspects of the antigen-binding sites described in a particular section of the instant disclosure are not to be deemed limiting to any specific sections.

### Definitions

Prior to setting forth this disclosure in more detail, it may be helpful to an understanding thereof to provide definitions of certain terms to be used herein. Additional definitions are set forth throughout this disclosure.

Unless otherwise defined herein, scientific and technical terms shall have the meanings that are commonly understood by those of ordinary skill in the art. Further, unless otherwise required by context, singular terms shall include pluralities and plural terms shall include the singular.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated components. The use of the alternative (e.g., “or”) should be understood to mean either one, both, or any combination thereof of the alternatives. As used herein, the terms “include,” “have,” and “comprise” are used synonymously, which terms and variants thereof are intended to be construed as non-limiting.

“Optional” or “optionally” means that the subsequently described element, component, event, or circumstance may or may not occur, and that the description includes instances in which the element, component, event, or circumstance occurs and instances in which they do not.

In addition, it should be understood that the individual constructs, or groups of constructs, derived from the various combinations of the structures and subunits described herein, are disclosed by the present application to the same extent as if each construct or group of constructs was set forth individually. Thus, selection of particular structures or particular subunits is within the scope of the present disclosure.

The term “consisting essentially of” is not equivalent to “comprising” and refers to the specified materials or steps of a claim, or to those that do not materially affect the basic characteristics of a claimed subject matter. For example, a protein domain, region, or module (e.g., a binding domain, hinge region, or linker) or a protein (which may have one or more domains, regions, or modules) “consists essentially of” a particular amino acid sequence when the amino acid sequence of a domain, region, module, or protein includes extensions, deletions, mutations, or a combination thereof (e.g., amino acids at the amino- or carboxy-terminus or between domains) that, in combination, contribute to at most 20% (e.g., at most 15%, 10%, 8%, 6%, 5%, 4%, 3%, 2% or 1%) of the length of a domain, region, module, or protein and do not substantially affect (i.e., do not reduce the activity by more than 50%, such as no more than 40%, 30%, 25%, 20%, 15%, 10%, 5%, or 1%) the activity of the domain(s), region(s), module(s), or protein (e.g., the target binding affinity of a binding protein).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

As used herein, “protein” or “polypeptide” refers to a polymer of amino acid residues. Proteins apply to naturally occurring amino acid polymers, as well as to amino acid polymers in which one or more amino acid residue is an artificial chemical mimetic of a corresponding naturally occurring amino acid and non-naturally occurring amino acid polymers. Variants of proteins, peptides, and polypeptides of this disclosure are also contemplated. In certain embodiments, variant proteins, peptides, and polypeptides comprise or consist of an amino acid sequence that is at least 70%, 75%, 80%, 85%, 90%, 91%, 92%, 93%, 94%, 95%, 96%, 97%, 98%, 99%, or 99.9% identical to an amino acid sequence of a defined or reference amino acid sequence as described herein.

As used herein, “nucleic acid molecule” or “polynucleotide” or “polynucleic acid” refers to a polymeric compound including covalently linked nucleotides, which can be made up of natural subunits (e.g., purine or pyrimidine bases) or non-natural subunits (e.g., morpholine ring). Purine bases include adenine, guanine, hypoxanthine, and xanthine, and pyrimidine bases include uracil, thymine, and cytosine. Nucleic acid molecules include polyribonucleic acid (RNA), which includes mRNA, microRNA, siRNA, viral genomic RNA, and synthetic RNA, and polydeoxyribonucleic acid (DNA), which includes cDNA, genomic DNA, and synthetic DNA, either of which may be single or double stranded. If single-stranded, the nucleic acid molecule may be the coding strand or non-coding (anti-sense) strand. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of nucleic acid molecules of this disclosure are also contemplated. Variant nucleic acid molecules are at least 70%, 75%, 80%, 85%, 90%, and are preferably 95%, 96%, 97%, 98%, 99%, or 99.9% identical a nucleic acid molecule of a defined or reference polynucleotide as described herein, or that hybridize to a polynucleotide under stringent hybridization conditions of 0.015M sodium chloride, 0.0015M sodium citrate at about 65-68° C. or 0.015M sodium chloride, 0.0015M sodium citrate, and 50% formamide at about 42° C. Nucleic acid molecule variants retain the capacity to encode a binding domain thereof having a functionality described herein, such as binding a target molecule.

As used herein, “percent sequence identity” refers to a relationship between two or more sequences, as determined by comparing the sequences. Preferred methods to determine sequence identity are designed to give the best match between the sequences being compared. For example, the sequences are aligned for optimal comparison purposes (e.g., gaps can be introduced in one or both of a first and a second amino acid or nucleic acid sequence for optimal alignment). Further, non-homologous sequences may be disregarded for comparison purposes. The percent sequence identity referenced herein is calculated over the length of the reference sequence, unless indicated otherwise. Methods to determine sequence identity and similarity can be found in publicly available computer programs. Sequence alignments and percent identity calculations may be performed using a BLAST program (e.g., BLAST 2.0, BLASTP, BLASTN, or BLASTX). The mathematical algorithm used in the BLAST programs can be found in Altschul et al., Nucleic Acids Res. 25:3389-3402, 1997. Within the context of this disclosure, it will be understood that where sequence analysis software is used for analysis, the results of the analysis are based on the “default values” of the program referenced. “Default values” mean any set of values or parameters which originally load with the software when first initialized.

The term “isolated” means that the material is removed from its original environment (e.g., the natural environment if it is naturally occurring). For example, a naturally occurring nucleic acid or polypeptide present in a living animal is not isolated, but the same nucleic acid or polypeptide, separated from some or all of the co-existing materials in the natural system, is isolated. Such nucleic acid could be part of a vector and/or such nucleic acid or polypeptide could be part of a composition (e.g., a cell lysate), and still be isolated in that such vector or composition is not part of the natural environment for the nucleic acid or polypeptide.

A “functional variant” refers to a polypeptide or polynucleotide that is structurally similar or substantially structurally similar to a parent or reference compound of this disclosure, but differs slightly in composition (e.g., one base, atom or functional group is different, added, or removed), such that the polypeptide or encoded polypeptide is capable of performing at least one function of the parent polypeptide with at least 50% efficiency, preferably at least 55%, 60%, 70%, 75%, 80%, 85%, 90%, 95%, 96%, 97%, 98%, 99%, 99.9%, or 100% level of activity of the parent polypeptide. In other words, a functional variant of a polypeptide or encoded polypeptide of this disclosure has “similar binding,” “similar affinity” or “similar activity” when the functional variant displays no more than a 50% reduction in performance in a selected assay as compared to the parent or reference polypeptide, such as an assay for measuring binding affinity (e.g., Biacore® or tetramer staining measuring an association (Ka) or a dissociation (KD) constant).

As used herein, a “functional portion” or “functional fragment” refers to a polypeptide or polynucleotide that comprises only a domain, portion or fragment of a parent or reference compound, and the polypeptide or encoded polypeptide retains at least 50% activity associated with the domain, portion or fragment of the parent or reference compound, preferably at least 55%, 60%, 70%, 75%, 80%, 85%, 90%, 95%, 96%, 97%, 98%, 99%, 99.9%, or 100% level of activity of the parent polypeptide, or provides a biological benefit (e.g., effector function). A “functional portion” or “functional fragment” of a polypeptide or encoded polypeptide of this disclosure has “similar binding” or “similar activity” when the functional portion or fragment displays no more than a 50% reduction in performance in a selected assay as compared to the parent or reference polypeptide (preferably no more than 20% or 10%, or no more than a log difference as compared to the parent or reference with regard to affinity).

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “non-endogenous” or “exogenous” refers to any gene, protein, compound, nucleic acid molecule, or activity that is not native to a host cell or a subject, or any gene, protein, compound, nucleic acid molecule, or activity native to a host cell or a subject that has been altered. Heterologous, non-endogenous, or exogenous includes genes, proteins, compounds, or nucleic acid molecules that have been mutated or otherwise altered such that the structure, activity, or both is different as between the native and altered genes, proteins, compounds, or nucleic acid molecules. In certain embodiments, heterologous, non-endogenous, or exogenous genes, proteins, or nucleic acid molecules (e.g., receptors, ligands, etc.) may not be endogenous to a host cell or a subject, but instead nucleic acids encoding such genes, proteins, or nucleic acid molecules may have been added to a host cell by conjugation, transformation, transfection, electroporation, or the like, wherein the added nucleic acid molecule may integrate into a host cell genome or can exist as extra-chromosomal genetic material (e.g., as a plasmid or other self-replicating vector). The term “homologous” or “homolog” refers to a gene, protein, compound, nucleic acid molecule, or activity found in or derived from a host cell, species, or strain. For example, a heterologous or exogenous polynucleotide or gene encoding a polypeptide may be homologous to a native polynucleotide or gene and encode a homologous polypeptide or activity, but the polynucleotide or polypeptide may have an altered structure, sequence, expression level, or any combination thereof. A non-endogenous polynucleotide or gene, as well as the encoded polypeptide or activity, may be from the same species, a different species, or a combination thereof.

In certain embodiments, a nucleic acid molecule or portion thereof native to a host cell will be considered heterologous to the host cell if it has been altered or mutated, or a nucleic acid molecule native to a host cell may be considered heterologous if it has been altered with a heterologous expression control sequence or has been altered with an endogenous expression control sequence not normally associated with the nucleic acid molecule native to a host cell. In addition, the term “heterologous” can refer to a biological activity that is different, altered, or not endogenous to a host cell. As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof.

As used herein, the term “endogenous” or “native” refers to a polynucleotide, gene, protein, compound, molecule, or activity that is normally present in a host cell or a subject.

The term “expression”, as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As described herein, more than one heterologous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

The term “construct” refers to any polynucleotide that contains a recombinant nucleic acid molecule (or, when the context clearly indicates, a fusion protein of the present disclosure). A (polynucleotide) construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, a RNA vector or a linear or circular DNA or RNA molecule that may include chromosomal, non-chromosomal, semi-synthetic or synthetic nucleic acid molecules. Vectors of the present disclosure also include transposon systems (e.g., Sleeping Beauty, see, e.g., Geurts et al., Mol. Ther. 8:108, 2003: Mates et al., Nat. Genet. 41:753, 2009). Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself or deliver the polynucleotide contained in the vector into the genome without the vector sequence. In the present specification, “plasmid,” “expression plasmid,” “virus,” and “vector” are often used interchangeably.

The term “introduced” in the context of inserting a nucleic acid molecule into a cell, means “transfection,” “transformation,” or “transduction” and includes reference to the incorporation of a nucleic acid molecule into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

In certain embodiments, polynucleotides of the present disclosure may be operatively linked to certain elements of a vector. For example, polynucleotide sequences that are needed to effect the expression and processing of coding sequences to which they are ligated may be operatively linked. Expression control sequences may include appropriate transcription initiation, termination, promoter, and enhancer sequences; efficient RNA processing signals such as splicing and polyadenylation signals; sequences that stabilize cytoplasmic mRNA; sequences that enhance translation efficiency (e.g., Kozak consensus sequences); sequences that enhance protein stability; and possibly sequences that enhance protein secretion. Expression control sequences may be operatively linked if they are contiguous with the gene of interest and expression control sequences that act in trans or at a distance to control the gene of interest.

In certain embodiments, the vector comprises a plasmid vector or a viral vector (e.g., a lentiviral vector or a γ-retroviral vector). Viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox, and canarypox). Other viruses include, for example, Norwalk virus, togavirus, flavivirus, reoviruses, papovavirus, hepadnavirus, and hepatitis virus. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996).

“Retroviruses” are viruses having an RNA genome, which is reverse-transcribed into DNA using a reverse transcriptase enzyme, the reverse-transcribed DNA is then incorporated into the host cell genome. “Gammaretrovirus” refers to a genus of the retroviridae family. Examples of gammaretroviruses include mouse stem cell virus, murine leukemia virus, feline leukemia virus, feline sarcoma virus, and avian reticuloendotheliosis viruses.

“Lentiviral vectors” include HIV-based lentiviral vectors for gene delivery, which can be integrative or non-integrative, have relatively large packaging capacity, and can transduce a range of different cell types. Lentiviral vectors are usually generated following transient transfection of three (packaging, envelope, and transfer) or more plasmids into producer cells. Like HIV, lentiviral vectors enter the target cell through the interaction of viral surface glycoproteins with receptors on the cell surface. On entry, the viral RNA undergoes reverse transcription, which is mediated by the viral reverse transcriptase complex. The product of reverse transcription is a double-stranded linear viral DNA, which is the substrate for viral integration into the DNA of infected cells.

In certain embodiments, the viral vector can be a gammaretrovirus, e.g., Moloney murine leukemia virus (MLV)-derived vectors. In other embodiments, the viral vector can be a more complex retrovirus-derived vector, e.g., a lentivirus-derived vector. HIV-1-derived vectors belong to this category. Other examples include lentivirus vectors derived from HIV-2, FIV, equine infectious anemia virus, SIV, and Maedi-Visna virus (ovine lentivirus). Methods of using retroviral and lentiviral viral vectors and packaging cells for transducing mammalian host cells with viral particles containing transgenes are known in the art and have been previous described, for example, in: U.S. Pat. No. 8,119,772; Walchli et al., PLoS One 6:327930, 2011; Zhao et al., J. Immunol. 174:4415, 2005; Engels et al., Hum. Gene Ther. 14:1155, 2003; Frecha et al., Mol. Ther. 18:1748, 2010; and Verhoeyen et al., Methods Mol. Biol. 506:97, 2009. Retroviral and lentiviral vector constructs and expression systems are also commercially available. Other viral vectors also can be used for polynucleotide delivery including DNA viral vectors, including, for example adenovirus-based vectors and adeno-associated virus (AAV)-based vectors; vectors derived from herpes simplex viruses (HSVs), including amplicon vectors, replication-defective HSV and attenuated HSV (Krisky et al., Gene Ther. 5:1517, 1998).

Other vectors that can be used with the compositions and methods of this disclosure include those derived from baculoviruses and α-viruses. (Jolly, D J. 1999. Emerging Viral Vectors. pp 209-40 in Friedmann T. ed. The Development of Human Gene Therapy. New York: Cold Spring Harbor Lab), or plasmid vectors (such as sleeping beauty or other transposon vectors).

When a viral vector genome comprises a plurality of polynucleotides to be expressed in a host cell as separate transcripts, the viral vector may also comprise additional sequences between the two (or more) transcripts allowing for bicistronic or multicistronic expression. Examples of such sequences used in viral vectors include internal ribosome entry sites (IRES), furin cleavage sites, viral 2A peptide, or any combination thereof.

As used herein, the term “host” refers to a cell or microorganism targeted for genetic modification with a heterologous nucleic acid molecule to produce a polypeptide of interest (e.g., an antibody of the present disclosure).

A host cell may include any individual cell or cell culture which may receive a vector or the incorporation of nucleic acids or express proteins. The term also encompasses progeny of the host cell, whether genetically or phenotypically the same or different. Suitable host cells may depend on the vector and may include mammalian cells, animal cells, human cells, simian cells, insect cells, yeast cells, and bacterial cells. These cells may be induced to incorporate the vector or other material by use of a viral vector, transformation via calcium phosphate precipitation, DEAE-dextran, electroporation, microinjection, or other methods. See, for example, Sambrook et al., Molecular Cloning: A Laboratory Manual 2d ed. (Cold Spring Harbor Laboratory, 1989).

“Antigen” or “Ag”, as used herein, refers to an immunogenic molecule that provokes an immune response. This immune response may involve antibody production, activation of specific immunologically-competent cells, activation of complement, antibody dependent cytotoxicity, or any combination thereof. An antigen (immunogenic molecule) may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, stool samples, cells, biological fluids, or combinations thereof. Antigens can be produced by cells that have been modified or genetically engineered to express an antigen.

The term “epitope” or “antigenic epitope” includes any molecule, structure, amino acid sequence, or protein determinant that is recognized and specifically bound by a cognate binding molecule, such as an immunoglobulin, or other binding molecule, domain, or protein. Epitopic determinants generally contain chemically active surface groupings of molecules, such as amino acids or sugar side chains, and can have specific three dimensional structural characteristics, as well as specific charge characteristics. Where an antigen is or comprises a peptide or protein, the epitope can be comprised of consecutive amino acids (e.g., a linear epitope), or can be comprised of amino acids from different parts or regions of the protein that are brought into proximity by protein folding (e.g., a discontinuous or conformational epitope), or non-contiguous amino acids that are in close proximity irrespective of protein folding.

The terms “antigen-binding site” or “antigen binding moiety” are used interchangeably herein and refer to the part of the antibody and/or immunoglobulin molecule that participates in binding to an antigen and/or epitope. In human antibodies, the antigen binding site is formed by amino acid residues of the N-terminal variable (“V”) regions of the heavy (“H”) and light (“L”) chains. The “hypervariable regions” are three highly divergent stretches within the V regions of the heavy and light chains which are interposed between “framework regions,” (“FR”), which are relatively conserved flanking stretches. The term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen. The three hypervariable regions of each of the heavy (“H”) and light (“L”) chains are referred to as “complementarity-determining regions” or “CDRs.” Antigen-binding sites can exist in an intact antibody, in an antigen-binding fragment of an antibody that retains the antigen-binding surface, or in a recombinant polypeptide such as an scFv, using a peptide linker to connect the heavy chain variable domain to the light chain variable domain in a single polypeptide. An antigen binding site can comprise an antibody light chain variable region (VL) and an antibody heavy chain variable region (VH).

The term “antibody” as used herein encompasses various antibody structures, including but not limited to monoclonal antibodies, polyclonal antibodies, multispecific antibodies (e.g., bispecific antibodies, bifunctional antibodies), antibody fusion proteins, antibodies that for heterodimers in engineered proteins, and antibody fragments so long as they exhibit the desired antigen-binding activity.

An “antibody fragment” refers to a polypeptide or protein other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

Numbering of CDR and framework regions may be according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001)). Equivalent residue positions can be annotated and for different molecules to be compared using Antigen receptor Numbering and Receptor Classification (ANARCI) software tool (2016, Bioinformatics 15:298-300). The CDRs of an antigen-binding site can be determined according to known methods, such as the Kabat, Chothia, EU, IMGT, and AHo as described above. The CDRs determined under these definitions typically include overlapping or subsets of amino acid residues when compared against each other. The heavy chain CDRs and light chain CDRs of an antibody can be defined using different numbering conventions. For example, in certain embodiments, the heavy chain CDRs are defined according to Chothia, supra, and the light CDRs are defined according to Kabat, supra. CDRH1, CDRH2 and CDRH3 denote the heavy chain CDRs, and CDRL1, CDRL2 and CDRL3 denote the light chain CDRs.

As used herein, PD-L1 (also known as “programmed death-ligand 1” or CD274 in humans) refers to the protein of UniProt Accession No. Q0GN75 (human) and related isoforms and orthologs.

The term “substitution” or “residue substitution” as used herein refers to replacement of a native or wild-type residue with a different residue.

“Affinity” refers to the strength of the sum total of non-covalent interactions between a single binding site of a molecule (e.g., a ligand, antibody, scFv) and its binding partner (e.g., a receptor, antigen, epitope). Unless indicated otherwise, as used herein, “binding affinity” refers to intrinsic binding affinity, which reflects a 1:1 interaction between members of a binding pair (e.g., receptor and a ligand). The affinity of a molecule-X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by methods known by persons of skill in the art, including those described herein.

“Fc domain” or “Fc region” as used herein refers to a polypeptide derived from a C-terminal region of an immunoglobulin heavy chain that contains at least a portion of the constant region. The term includes polypeptides having a native sequence Fc region, or variants thereof. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Examples of Fc regions are disclosed in U.S. Pat. Nos. 7,317,091; 8,735,545; 7,371,826; 7,670,600; and 9,803,023; all of which are incorporated by reference in their entirety.

“Immunoglobulin” refers to a protein having the structure of an antibody. As an example, immunoglobulins of the IgG class are heterotetrameric glycoproteins with two light chains and two heavy chains that are joined by at least one disulfide-bond. From N- to C-terminus, the heavy chains each have a variable region (VH), also called a variable heavy domain or a heavy chain variable domain, followed by three constant domains (CH1, CH2, and CH3), also called a heavy chain constant region. Similarly, from N- to C-terminus, light chain each have a variable region (VL), also called a variable light domain or a light chain variable domain, followed by a constant light (CL) domain, also called a light chain constant region. The heavy chain of an immunoglobulin may be assigned to one of five classes, called α (IgA), δ (IgD), ε (IgE), γ (IgG), or μ (IgM), some of which may be further divided into subclasses, e.g., γ1 (IgG1), γ2 (IgG2), γ3 (IgG3), γ4 (IgG4), α1 (IgA1) and α2 (IgA2). The light chain of an immunoglobulin may be assigned to one of two types, called kappa (κ) and lambda (λ), based on the sequence of its constant domain. An immunoglobulin includes two Fab molecules and an Fc domain, linked via the immunoglobulin hinge region.

“Fab molecule” or “antigen binding fragment” is an antigen-binding fragment of an antibody that includes the variable domain and constant domain of a light chain, and a variable domain and a CH1 domain of a heavy chain.

“Single chain variable domain” or “scFv” refers to an antigen-binding moiety that includes variable regions of a heavy chain and light chain, which are linked by a linker peptide.

“Bispecific antibody,” “bifunctional protein,” and “bifunctional antibody,” are used interchangeably throughout the disclosure and refer to an artificial antibody with two different antigen-binding sites and/or an antibody-fusion protein comprising at least on antigen-binding site and at least one functional domain. Bispecific antibody can refer to a full immunoglobulin protein with two different antigen-binding sites, or can refer to other molecules having two antigen binding moieties, such as a fusion protein including two Fabs or two scFvs. In certain embodiments, a bifunctional protein and/or bifunctional antibody can refer to a protein, fusion protein, and/or heterodimeric protein pair that includes one or more functional domains. Examples of functional domains include an antigen-binding site, antibody fragments (e.g., Fab, scFv, etc.), an antibody heavy chain and light chain, cytokines (e.g., IL-2, IL-15). Bifunctional protein or bifunctional antibody can refer an antibody that comprises a fusion to a non-antibody polypeptide, such as a cytokine. For example, a bifunctional protein can include an antibody heavy chain and light chain wherein the heavy chain constant region is fused to an IL-2, engineered IL-2, IL-15, engineered IL-15, IL-15 receptor, or an engineered IL-15 receptor, or a function fragment thereof. In addition, a bifunctional protein can comprise an antibody heavy chain and light chain wherein the heavy chain constant region can form a heterodimer with polypeptide or protein that does not comprise an antigen binding site. For example, a bifunctional protein can comprise a heavy chain, a light chain, and an IL-2 or engineered IL-2 fusion protein that comprises an antibody Fc domain capable of forming a heterodimer with the Fc domain of the antibody heavy chain.

“Modulating an immune response” may include one or more of an increase in T effector cell response (e.g., cytotoxicity against tumor cells and virus infected cells), an increase in B cell activation, restoration of lymphocyte activation and proliferation, an increase in the expression of IL-2 receptors, an increase in T cell responsiveness, an increase in natural killer cell activity or lymphokine-activated killer (LAK) cell activity, a decrease in regulatory T cells response to other T cells, and the like.

“Treatment,” “treating” or “ameliorating” refers to medical management of a condition, disease, or disorder of a subject (e.g., patient), which may be therapeutic, result in a reduction in one or more symptoms, result in reduction in tumor size and/or severity, inhibit the measurable growth of a tumor or onset of symptoms, or any combination thereof.

An “effective amount” or a “therapeutically effective amount” may refer to an amount of therapeutic agent that provides a desired physiological change, such as immune modulation and/or an anti-cancer effect. The desired physiological change may, for example, be a decrease in symptoms of a disease, or a decrease in severity of a disease, or may be a reduction in the progression of a disease. With respect to cancer, the desired physiological changes may include, for example, tumor regression, a decreased rate of tumor progression, a reduced level of a cancer biomarker, reduced symptoms associated with cancer, a prevention or delay in metastasis, or clinical remission.

As used herein, the term “inhibit” refers the reduction of a specified activity (e.g., immune suppression or tumor growth). Unless specified otherwise, an activity can be considered inhibited if the activity is reduced by at least 10%, 20%, 30%, 40%, 50%, 60%, 70%, 80%, 90%, 95%, 98%, 99%, or 100%, as measured by the methods disclosed herein or known in the art.

“Cancer antigen” refers to a molecule that is preferentially expressed by cancer cells. Examples of cancer antigens include CD19, CD20, ROR1, fibroblast activation protein-α, and carcinoembryonic antigen (CEA).

“Tumor microenvironment inhibitor” refers to an agent that inhibits one or more conditions or cell types that promote tumor growth and are present in the local environment surrounding a tumor. For example, bevacizumab can inhibit the tumor microenvironment by reducing angiogenesis in a tumor microenvironment.

Recombinant DNA, molecular cloning, and gene expression techniques used in the present disclosure are known in the art and described in references, such as Sambrook et al., Molecular Cloning: A Laboratory Manual, 3^{rd} Ed., Cold Spring Harbor Laboratory, New York, 2001, and Ausubel et al., Current Protocols in Molecular Biology, John Wiley and Sons, Baltimore, MD, 1999.

Additional definitions are provided throughout the present disclosure.

Antigen-Binding Sites that Bind PD-L1

In one aspect, the present application provides an antigen-binding site that binds PD-L1 (e.g., human PD-L1). Example sequences that can be used in or as antigen binding sites to such as CDR, VH, VL, and scFv sequences are listed in Table 1. The amino acid positions and the CDR sequences are identified according to the IMGT numbering scheme.

TABLE 1
Sequences of Exemplary Antigen-Binding Sites that Bind PD-L1

| PD-L1 | | |
|---|---|---|
| binder | Heavy chain variable domain | Light chain variable domain |
| 2018EP164- | QVQLQQWGAGLLKPSETLSLTCAVY | QSALTQDPAVSVALGQTVRITCQG |
| F04 | GGSFSGYYWSWIRQPPGKGLEWIGEI | DSLRSHYASWYQQKPGQAPVVVV |
| | NHSGSTNYNPSLKSRVTISVDTSKNQF | YDNSDRPSGIPDRFSGSNSGNTATL |
| | SLKLSSVTAADTAVYYCARGRRLRYF | TISRVEVGDEADYYCQVWDSSSDH |
| | DWAAYNWFDPWGQGTLVTVSS (SEQ | YVFGTGTKV (SEQ ID NO: 2) |
| | ID NO: 1) | CDR1: SLRSHYAS (SEQ ID NO: 6) |
| | CDR1: GSFSGYYWS (SEQ ID NO: 3) | CDR2: VVVVYDNSDRPS (SEQ ID |
| | CDR2: WIGEINHSGSTNY (SEQ ID | NO: 7) |
| | NO: 4) | CDR3: QVWDSSSDHY (SEQ ID |
| | CDR3: RGRRLRYFDWAAYNWFDP | NO: 8) |
| | (SEQ ID NO: 5) | |
| 2018EP170- | EVQLVQSGAEVKKPGSSVKVSCKAS | QQSVLTQPASVSGSPGQSITISCTGT |
| E06 | GGTFSSYAISWVRQAPGQGLEWMGG | SSDIGSYNLVSWYQNHPGKAPKLIL |
| | IIPIFGTANYAQKFQGRVTITADESTST | YEITKRPSGVSSRFSGSKSGNAASLT |
| | AYMELSSLRSEDTAVYYCARDSRWG | ISGLQAEDEAEYFCCSYAGLSVFFV |
| | PAPHAFDIWGQGTMVTVSS (SEQ ID | FGGGTKL (SEQ ID NO: 10) |
| | NO: 9) | CDR1: SSDIGSYNLVS (SEQ ID |
| | CDR1: GTFSSYAIS (SEQ ID NO: 11) | NO: 14) |
| | CDR2: WMGGIIPIFGTANY (SEQ ID | CDR2: LILYEITKRPS (SEQ ID |
| | NO: 12) | NO: 15) |
| | CDR3: RDSRWGPAPHAFDI (SEQ ID | CDR3: CSYAGLSVFF (SEQ ID |
| | NO: 13) | NO: 16) |
| 2018EP161- | EVQLVESGGGLVQPGGSLRLSCAASG | VIWMTQSPSSLSASLGDRVTITCRA |
| G08 | FTFSSYAMSWVRQAPGKGLEWVSAIS | SQSVSTYINWYQQKSGKGPKLLIFN |
| | GSGGSTYYADSVKGRFTISRDNSKNT | ASTLQSGVPSRFSGSGSGTDFTLTIT |
| | LYLQMNSLRAEDTAVYYCAKASGW | SLQPDDSATYYCQQSYRVPLSFGGG |
| | YPWYFDYWGQGTLVTVSS (SEQ ID | TKVEIR (SEQ ID NO: 18) |
| | NO: 17) | CDR1: QSVSTYIN (SEQ ID NO: 22) |
| | CDR1: FTFSSYAMS (SEQ ID NO: 19) | CDR2: LLIFNASTLQS (SEQ ID |
| | CDR2: WVSAISGSGGSTYY (SEQ ID | NO: 23) |
| | NO: 20) | CDR3: QQSYRVPL (SEQ ID NO: 24) |
| | CDR3: KASGWYPWYFDY (SEQ ID | |
| | NO: 21) | |
| 2018EP161- | QVQLVESGGGLVQPGGSLRLSCAASG | DIVMTQSPSSLSASVGDRVTITCRAS |
| F08 | FTFSSYAMSWVRQAPGKGLEWVSAIS | QTVSTYINWYQQRSGKGPKLLIFDA |
| | GSGGSTYYADSVKGRFTISRDNSKNT | SRLQSGAPSRFSGSGSGTEFTLTITSL |
| | LYLQMNSLRAEDTAVYYCAKAGSW | QPDDSATYYCQQSYRIPLSFGGGTK |
| | YPYYFDYWGQGTLVTVSS (SEQ ID | VEIR (SEQ ID NO: 26) |
| | NO: 25) | CDR1: QTVSTYIN (SEQ ID NO: 28) |
| | CDR1: FTFSSYAMS (SEQ ID NO: 19) | CDR2: LLIFDASRLQS (SEQ ID |
| | CDR2: WVSAISGSGGSTYY (SEQ ID | NO: 29) |
| | NO: 20) | CDR3: QQSYRIPLS (SEQ ID NO: 30) |
| | CDR3: KAGSWYPYYFDY (SEQ ID | |
| | NO: 27) | |
| 2018EP161- | QMQLVQSGAEVKKPGATVKISCKVS | NFMLTQPSSLSASPGASASLTCTLPS |
| F04 | GYTFTDYYMHWVQQAPGKGLEWMG | GINVGPYGIFWYQQKPGSPPQYLLR |
| | LVDPEDGETIYAEKFQGRVTITADTST | YKSNSENHQGSGVPSRFSGSEDASA |
| | DTAYMELSSLRSEDTAVFYCATDGSG | NVGILLISGLQSEDEADYYCMIWHS |
| | SYYLTLGQGTLVTVSS (SEQ ID | SAWVFGGGTKVTVL (SEQ ID |
| | NO: 31) | NO: 32) |
| | CDR1: YTFTDYYMH (SEQ ID NO: 33) | CDR1: SGINVGPYGI (SEQ ID NO: 36) |
| | CDR2: WMGLVDPEDGETIYA (SEQ ID | CDR2: QYLLRYKSNSENHQGS (SEQ |
| | NO: 34) | ID NO: 37) |
| | CDR3: TDGSGSYYLTL (SEQ ID NO: 35) | CDR3: MIWHSSAWV (SEQ ID |
| | | NO: 38) |
| 2018EP280- | QVQLQQWGAGLLKPSETLSLTCAVY | QSALTQDPAVSVALGQTVRISCQGD |
| E04 | GGSFSGYYWSWIRQPPGKGLEWIGEI | SLRSHYASWYQQKPGQAPVVVVY |
| | NHIGSTNYNPSLKSRVTISVDTSNNQF | DNSDRPSGIPDRFSGSNSGNTATLTI |
| | SLKLSSVTAADTAVYYCARGRRLRYF | SRVEVGDEADYYCQVWDSSSDHY |
| | DWAAYNWFDPWGQGTLVTVSS (SEQ | VFGTGTKVTVL (SEQ ID NO: 40) |
| | ID NO: 39) | CDR1: SLRSHYAS (SEQ ID NO: 6) |
| | CDR1: GSFSGYYWS (SEQ ID NO: 3) | CDR2: VVVYDNSDRPS (SEQ ID |
| | CDR2: WIGEINHIGSTNY (SEQ ID | NO: 42) |
| | NO: 41) | CDR3: QVWDSSSDHYV (SEQ ID |
| | CDR3: RGRRLRYFDWAAYNWFDP | NO: 43) |
| | (SEQ ID NO: 5) | |
| 2018EP171- | QVQLVQSGAEVKKPGSSVKVSCKAS | QSVLTQPPSASGTPGQRVTISCSGSY |
| H02 | GGTFSSYAISWVRQAPGQGLEWMGG | SNIGSNFVNWYQQLPGTAPKLLIHF |
| | IIPIFGTANYAQKFQGRVTITADESTST | NNQRPSGVPDRFSGSRSGTSASLAIS |
| | AYMELSSLRSEDTAVYYCARDKGYG | GLQSEDEADYFCSTWDDSLKGPIFG |
| | SGWRGDYWGQGTLVTVSS (SEQ ID | GGTKLTVL (SEQ ID NO: 45) |
| | NO: 44) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFNNQRPS (SEQ ID |
| | NO: 12) | NO: 48) |
| | CDR3: RDKGYGSGWRGDY (SEQ ID | CDR3: STWDDSLKGP (SEQ ID |
| | NO: 46) | NO: 49) |
| 2018EP173- | QVQLVQSGGGLVKPGGSLRLSCAAS | QPVLTQPPSVSVAPGETATITCGGN |
| H11 | GFTFSSYSMNWVRQAPGKGLEWVSSI | NIGRKSVHWYLQKPGQAPVLVIGH |
| | SSSSSYIYYADSVKGRFTISRDNAKNS | DTDRPSGIPARFSGSNSGNTATLTIN |
| | LYLQMNSLKAEDTAVYYCARDSLQW | RVEAGDEADYYCQVWDTSGHHYV |
| | LALDYWGQGTLVTVSS (SEQ ID | FGTGTKVTVL (SEQ ID NO: 51) |
| | NO: 50) | CDR1: NIGRKSVH (SEQ ID NO: 55) |
| | CDR1: FTFSSYSMN (SEQ ID NO: 52) | CDR2: LVIGHDTDRPS (SEQ ID |
| | CDR2: WVSSISSSSSYIYY (SEQ ID | NO: 56) |
| | NO: 53) | CDR3: QVWDTSGHHY (SEQ ID |
| | CDR3: RDSLQWLALDY (SEQ ID | NO: 57) |
| | NO: 54) | |
| 2018EP172- | QMQLVQSGAEVKKPGSSVKVSCKAS | QSVLTQPPSASGTPGQRVTISCSGSY |
| F10 | GGTFSSYAISWVRQAPGQGLEWMGG | SNIGSNFVNWYQQLPGTAPKLLIHF |
| | IIPIFGTANYAQKFQGRVTITADESTST | DNQRPSGVPDRFSGSRSGTSASLAIS |
| | AYMELSSLRSEDTAVYYCARDKGYG | GLQSEDEADYFCSTWDDSLKGPIFG |
| | SGWRGDYWGQGTLVTVSS (SEQ ID | GGTKLTVL (SEQ ID NO: 59) |
| | NO: 58) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFDNQRPS (SEQ ID |
| | NO: 12) | NO: 60) |
| | CDR3: RDKGYGSGWRGDY (SEQ ID | CDR3: STWDDSLKGP (SEQ ID |
| | NO: 46) | NO: 49) |
| 2018EP280- | EVQLVESGGGLVKPGGSLRLSCAASG | QSVLTQPPSASATPGQRVTISCSGSD |
| E01 | LTFSNAWMSWVRQAPGKGLEWVGRI | SNIATNYVYWYQQFPGTAPKLLIYR |
| | KSKTDGGTTEYAAPVKGRFTISRDDS | NHQRPSGVPDRFSGSRSGTSASLAIS |
| | KNTLYLQMSSLKTEDTAVYYCTIVYY | GLRSEDEADYYCATWDDSVNSWV |
| | YDSSGPVFFQHWGQGTLVTVSS (SEQ | FGGGTKLTVL (SEQ ID NO: 62) |
| | ID NO: 61) | CDR1: DSNIATNYVY (SEQ ID |
| | CDR1: LTFSNAWMS (SEQ ID NO: 63) | NO: 66) |
| | CDR2: WVGRIKSKTDGGTTEY (SEQ | CDR2: LLIYRNHQRPS (SEQ ID |
| | ID NO: 64) | NO: 67) |
| | CDR3: IVYYYDSSGPVFFQH (SEQ ID | CDR3: ATWDDSVNSW (SEQ ID |
| | NO: 65) | NO: 68) |
| 2019EP69- | QVQLVQSGAEVKKPGSSVKVSCKAS | QSVLTQPPSASGTPGQRVTISCSGSY |
| E02 | GGTFSSYAISWVRQAPGQGLEWMGG | SNIGSNFVNWYQQLPGTAPKLLIHF |
| | IIPIFGTANYAQKFQGRVTITADESTST | NNQRPSGVPDRFSGSRSGTSASLAIS |
| | AYMELSSLRSEDTAVYYCARNKGYV | GLQSEDEADYFCSTWDDSLKGPIFG |
| | SGWRSDYWGQGTLVTVSS (SEQ ID | GGTKLTVL (SEQ ID NO: 70) |
| | NO: 69) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFNNQRPS (SEQ ID |
| | NO: 12) | NO: 48) |
| | CDR3: RNKGYVSGWRSDY (SEQ ID | CDR3: STWDDSLKGP (SEQ ID |
| | NO: 71) | NO: 49) |
| 2019EP69- | QVQLVQSGAEVKKPGSSVKVSCKAS | QSVLTQPPSASGTPGQRVTISCSGSY |
| E10 | GGTFSSYAISWVRQAPGQGLEWMGG | SNIGSNFVNWYQQLPGTAPKLLIHF |
| | IIPIFGTANYAQKFQGRVTITADESTST | NNQRPSGVPDRFSGSRSGTSASLAIS |
| | AYMELSSLRSEDTAVYYCARDKGFG | GLQSEDEADYFCSTWDDSLKGPIFG |
| | SGWRGDYWGQGTLVTVSS (SEQ ID | GGTKLTVL (SEQ ID NO: 73) |
| | NO: 72) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFNNQRPS (SEQ ID |
| | NO: 12) | NO: 48) |
| | CDR3: RDKGFGSGWRGDY (SEQ ID | CDR3: STWDDSLKGP (SEQ ID |
| | NO: 74) | NO: 49) |
| 2019EP69- | QVQLVQSGAEVKKPGSSVKVSCKAS | QSVLTQPPSASGTPGQRVTISCSGSY |
| C05 | GGTFSSYAISWVRQAPGQGLEWMGG | SNIGSNFVNWYQQLPGTAPKLLIHF |
| | IIPIFGTANYAQKFQGRVTITADESTST | NNQRPSGVPDRFSGSRSGTSASLAIS |
| | AYMELSSLRSEDTAVYYCARDKGYG | GLQSEDEADYFCSTWDDSLKGPIFG |
| | SGFRGDYWGQGTLVTVSS (SEQ ID | GGTKLTVL (SEQ ID NO: 76) |
| | NO: 75) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFNNQRPS (SEQ ID |
| | NO: 12) | NO: 48) |
| | CDR3: RDKGYGSGFRGDY (SEQ ID | CDR3: STWDDSLKGP (SEQ ID |
| | NO: 77) | NO: 49) |
| 2019EP69- | QVQLVQSGAEVKKPGSSVKVSCKAS | QSVLTQPPSASGTPGQRVTISCSGSY |
| F02 | GGTFSSYAISWVRQAPGQGLEWMGG | SNIGSNFVNWYQQLPGTAPKLLIHF |
| | IIPIFGTANYAQKFQGRVTITADESTST | NNQRPSGVPDRFSGSRSGTSASLAIS |
| | AYMELSSLRSEDTAVYYCARDKWYG | GLQSEDEADYFCSTWDDSLKGPIFG |
| | SGWRGDYWGQGTLVTVSS (SEQ ID | GGTKLTVL (SEQ ID NO: 79) |
| | NO: 78) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFNNQRPS (SEQ ID |
| | NO: 12) | NO: 48) |
| | CDR3: RDKWYGSGWRGDY (SEQ ID | CDR3: STWDDSLKGP (SEQ ID |
| | NO: 80) | NO: 49) |
| 2019EP69- | QVQLVQSGAEVKKPGSSVKVSCKAS | SVLTQPPSASGTPGQRVTISCSGSYS |
| B01 | GGTFSSYAISWVRQAPGQGLEWMGG | NIGSNFVNWYQQLPGTAPKLLIHFN |
| | IIPIFGTANYAQKFQGRVTITADESTST | NQRPSGVPDRFSGSRSGTSASLAISG |
| | AYMELSSLRSEDTAVYYCARRIRIGT | LQSEDEADYFCSTWDDSLKGPIFGG |
| | GWRGDYWGQGTLVTVSS (SEQ ID | GTKLTVL (SEQ ID NO: 82) |
| | NO: 81) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFNNQRPS (SEQ ID |
| | NO: 12) | NO: 48) |
| | CDR3: ARRIRIGTGWRGDY (SEQ ID | CDR3: STWDDSLKGP (SEQ ID |
| | NO: 83) | NO: 49) |
| 2019EP69- | QVQLVQSGAEVKKPGSSVKVSCKAS | QSVLTQPPSASGTPGQRVTISCSGSY |
| H10 | GGTFSSYAISWVRQAPGQGLEWMGG | SNIGSNFVNWYQQLPGTAPKLLIHF |
| | IIPIFGTANYAQKFQGRVTITADESTST | NNQRPSGVPDRFSGSRSGTSASLAIS |
| | AYMELSSLRSEDTAVYYCARDKWFG | GLQSEDEADYFCSTWDDSLKGPIFG |
| | SGWRGDYWGQGTLVTVSS (SEQ ID | GGTKLTVL (SEQ ID NO: 85) |
| | NO: 84) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFNNQRPS (SEQ ID |
| | NO: 12) | NO: 48) |
| | CDR3: RDKWFGSGWRGDY (SEQ ID | CDR3: STWDDSLKGP (SEQ ID |
| | NO: 86) | NO: 49) |
| 2019EP69- | QVQLVQSGAEVKKPGSSVKVSCKAS | QSVLTQPPSASGTPGQRVTISCSGSY |
| F03 | GGTFSSYAISWVRQAPGQGLEWMGG | SNIGSNFVNWYQQLPGTAPKLLIHF |
| | IIPIFGTANYAQKFQGRVTITADESTST | NNQRPSGVPDRFSGSRSGTSASLAIS |
| | AYMELSSLRSEDTAVYYCARDKWYG | GLQSEDEADYFCSTWDDSLKGPIFG |
| | SAWRGDYWGQGTLVTVSS (SEQ ID | GGTKLTVL (SEQ ID NO: 88) |
| | NO: 87) | CDR1: YSNIGSNFVN (SEQ ID |
| | CDR1: GTFSSYAIS (SEQ ID NO: 11) | NO: 47) |
| | CDR2: WMGGIIPIFGTANY (SEQ ID | CDR2: LLIHFNNQRPS (SEQ ID |
| | NO: 12) | NO: 48) |
| | CDR3: RDKWYGSAWRGDY (SEQ ID | CDR3: STWDDSLKGP (SEQ ID |
| | NO: 89) | NO: 49) |

In certain embodiments, the antigen-binding site that binds PD-L1, comprises an antibody a heavy chain variable domain (VH) comprising complementarity-determining region 1 (CDR1) sequence of SEQ ID NO: 11, 3, 19, 33, 52, or 63; complementarity-determining region 2 (CDR2) sequence of SEQ ID NO: 12, 4, 20, 34, 41, 53, or 64; and complementarity-determining region 3 (CDR3) sequence of SEQ ID NO: 89, 5, 13, 21, 27, 35, 46, 54, 65, 71, 74, 77, 80, 83, or 86; and a light chain variable domain (VL) comprising CDR1 sequence of SEQ ID NO: 47, 6, 14, 22, 28, 36, 55, or 66; CDR2 sequence of SEQ ID NO: 48, 7, 15, 23, 29, 37, 42, 56, 60, or 67; and CDR3 sequences of SEQ ID NO: 49, 8, 16, 24, 30, 38, 43, 57, or 68.

In certain embodiments, the antigen-binding site that binds PD-L1, comprises an antibody a heavy chain variable domain (VH) that comprises an amino acid sequence at least 90% (e.g., at least 91%, at least 92%, at least 93%, at least 94%, at least 95%, at least 96%, at least 97%, at least 98%, or at least 99%) identical to the VH of an antibody as disclosed in Table 1, and an antibody light chain variable domain (VL) that comprises an amino acid sequence at least 90% (e.g., at least 91%, at least 92%, at least 93%, at least 94%, at least 95%, at least 96%, at least 97%, at least 98%, or at least 99%) identical to the VL of the same antibody as disclosed in Table 1. In certain embodiments, the antigen-binding site comprises the heavy chain CDR1, CDR2, and CDR3 and the light chain CDR1, CDR2, and CDR3 of a VH and a VL sequence of an antibody as disclosed in Table 1 as determined under the IMGT system, EU system, Kabat system (see Kabat et al., (1991) Sequences of Proteins of Immunological Interest, NIH Publication No. 91-3242, Bethesda), Chothia system (see, e.g., Chothia C & Lesk A M, (1987), J. Mol. Biol. 196:901-917), MacCallum (see MacCallum R M et al., (1996) J. Mol. Biol. 262:732-745), or any other CDR determination method known in the art. In certain embodiments, the antigen-binding site comprises the heavy chain CDR1, CDR2, and CDR3 and the light chain CDR1, CDR2, and CDR3 of an antibody as disclosed in Table 1.

In certain embodiments, an antigen-binding site described in the present application is derived from 2018EP164-F04. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:1, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:2. In certain embodiments, the antigen-binding site comprises the heavy chain CDR1, CDR2, and CDR3 and the light chain CDR1, CDR2, and 1 and 2, respectively. In certain embodiments, the VH comprises CDR1, CDR2, and CDR3 comprising the amino acid sequences of SEQ ID NOS: 3, 4, and 5, respectively. In certain embodiments, the VL comprises CDR1, CDR2, and CDR3 comprising the amino acid sequences of SEQ ID NOS: 6, 7, and 8, respectively. In certain embodiments, the antigen-binding site comprises (a) a VH that comprises CDR1, CDR2, and CDR3 comprising the amino acid sequences of SEQ ID NOS: 3, 4, and 5, respectively; and (b) a VL that comprises CDR1, CDR2, and CDR3 comprising the amino acid sequences of SEQ ID NOS: 6, 7, and 8, respectively.

In certain embodiments, an antigen-binding site described in the present application is derived from 2018EP170-E06. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:9, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:10. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 9 and 10, respectively. In certain embodiments, the VH comprises CDR1, CDR2, and CDR3 comprising the amino acid sequences of SEQ ID NOS: 11, 12, and 13, respectively. In certain embodiments, the VL comprises CDR1, CDR2, and CDR3 comprising the amino acid sequences of SEQ ID NOS: 14, 15, and 16, respectively. In certain embodiments, the antigen-binding site comprises (a) a VH that comprises CDR1, CDR2, and CDR3 comprising the amino acid sequences of SEQ ID NOS: 11, 12, and 13, respectively; and (b) a VL that comprises CDR1, CDR2, and CDR3. comprising the amino acid sequences of SEQ ID NOS: 14, 15, and 16, respectively.

In certain embodiments, an antigen-binding site described in the present application is derived from 2018EP161-G08. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:17, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:18. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 17 and 18, respectively. In certain embodiments, the VH comprises CDR1, CDR2, and CDR3 comprising the amino acid sequences of SEQ ID NOS: 19, 20, and 21, respectively. In certain embodiments, the VL comprises CDR1, CDR2, and CDR3 comprising the amino acid sequences of SEQ ID NOS: 22, 23, and 24, respectively. In certain embodiments, the antigen-binding site comprises (a) a VH that comprises CDR1, CDR2, and CDR3 comprising the amino acid sequences of SEQ ID NOS: 19, 20, and 21, respectively; and (b) a VL that comprises CDR1, CDR2, and CDR3 comprising the amino acid sequences of SEQ ID NOS: 22, 23, and 24, respectively.

In certain embodiments, an antigen-binding site described in the present application is derived from 2018EP161-F08. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:25, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:26. In certain embodiments, the antigen-binding site comprises the heavy chain CDR1, CDR2, and CDR3 and the light chain CDR1, CDR2, and 25 and 26, respectively. In certain embodiments, the VH comprises CDR1, CDR2, and CDR3 comprising the amino acid sequences of SEQ ID NOS: 19, 20, and 27, respectively. In certain embodiments, the VL comprises CDR1, CDR2, and CDR3 comprising the amino acid sequences of SEQ ID NOS: 28, 29, and 30, respectively. In certain embodiments, the antigen-binding site comprises (a) a VH that comprises CDR1, CDR2, and CDR3 comprising the amino acid sequences of SEQ ID NOS: 19, 20, and 27, respectively; and (b) a VL that comprises CDR1, CDR2, and CDR3 comprising the amino acid sequences of SEQ ID NOS: 28, 29, and 30, respectively.

In certain embodiments, an antigen-binding site described in the present application is derived from 2018EP161-F04. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:31, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:32. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 31 and 32, respectively. In certain embodiments, the VH comprises CDR1, CDR2, and CDR3 comprising the amino acid sequences of SEQ ID NOS: 33, 34, and 35, respectively. In certain embodiments, the VL comprises CDR1, CDR2, and CDR3 comprising the amino acid sequences of SEQ ID NOS: 36, 37, and 38, respectively. In certain embodiments, the antigen-binding site comprises (a) a VH that comprises CDR1, CDR2, and CDR3 comprising the amino acid sequences of SEQ ID NOS: 33, 34, and 35, respectively; and (b) a VL that comprises CDR1, CDR2, and CDR3 comprising the amino acid sequences of SEQ ID NOS: 36, 37, and 38, respectively.

In certain embodiments, an antigen-binding site described in the present application is derived from 2018EP280-E04. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:39, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:40. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 39 and 40, respectively. In certain embodiments, the VH comprises CDR1, CDR2, and CDR3 comprising the amino acid sequences of SEQ ID NOS: 3, 41, and 5, respectively. In certain embodiments, the VL comprises CDR1, CDR2, and CDR3 comprising the amino acid sequences of SEQ ID NOS: 6, 42, and 43, respectively. In certain embodiments, the antigen-binding site comprises (a) a VH that comprises CDR1, CDR2, and CDR3 comprising the amino acid sequences of SEQ ID NOS: 3, 41, and 5, respectively; and (b) a VL that comprises CDR1, CDR2, and CDR3 comprising the amino acid sequences of SEQ ID NOS: 6, 42, and 43, respectively.

In certain embodiments, an antigen-binding site described in the present application is derived from 2018EP171-H02. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:44, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:45. In certain embodiments, the antigen-binding site comprises the heavy chain CDR1, CDR2, and CDR3 and the light chain CDR1, CDR2, and 44 and 45, respectively. In certain embodiments, the VH comprises CDR1, CDR2, and CDR3 comprising the amino acid sequences of SEQ ID NOS: 11, 12, and 46, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1, CDR2, and CDR3 comprising the amino acid sequences of SEQ ID NOS: 11, 12, and 46, respectively; and (b) a VL that comprises CDR1, CDR2, and CDR3 comprising the amino acid sequences of SEQ ID NOS: 47, 48, and 49, respectively.

In certain embodiments, an antigen-binding site described in the present application is derived from 2018EP173-H11. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:50, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:51. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 50 and 51, respectively. In certain embodiments, the VH comprises CDR1, CDR2, and CDR3 comprising the amino acid sequences of SEQ ID NOS: 52, 53, and 54, respectively. In certain embodiments, the VL comprises CDR1, CDR2, and CDR3 comprising the amino acid sequences of SEQ ID NOS: 55, 56, and 57, respectively. In certain embodiments, the antigen-binding site comprises (a) a VH that comprises CDR1, CDR2, and CDR3 comprising the amino acid sequences of SEQ ID NOS: 52, 53, and 54, respectively; and (b) a VL that comprises CDR1, CDR2, and CDR3 comprising the amino acid sequences of SEQ ID NOS: 55, 56, and 57, respectively.

In certain embodiments, an antigen-binding site described in the present application is derived from 2018EP172-F10. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:58, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:59. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 58 and 59, respectively. In certain embodiments, the VH comprises CDR1, CDR2, and CDR3 comprising the amino acid sequences of SEQ ID NOS: 11, 12, and 46, respectively. In certain embodiments, the VL comprises CDR1, CDR2, and CDR3 comprising the amino acid sequences of SEQ ID NOS: 47, 60, and 49, respectively. In certain embodiments, the antigen-binding site comprises (a) a VH that comprises CDR1, CDR2, and CDR3 comprising the amino acid sequences of SEQ ID NOS: 11, 12, and 46, respectively; and (b) a VL that comprises CDR1, CDR2, and CDR3 comprising the amino acid sequences of SEQ ID NOS: 47, 60, and 49, respectively.

In certain embodiments, an antigen-binding site described in the present application is derived from 2018EP280-E01. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:61, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:62. In certain embodiments, the antigen-binding site comprises the heavy chain CDR1, CDR2, and CDR3 and the light chain CDR1, CDR2, and 61 and 62, respectively. In certain embodiments, the VH comprises CDR1, CDR2, and CDR3 comprising the amino acid sequences of SEQ ID NOS: 63, 64, and 65, respectively. In certain embodiments, the VL comprises CDR1, CDR2, and CDR3 comprising the amino acid sequences of SEQ ID NOS: 66, 67, and 68, respectively. In certain embodiments, the antigen-binding site comprises (a) a VH that comprises CDR1, CDR2, and CDR3 comprising the amino acid sequences of SEQ ID NOS: 63, 64, and 65, respectively; and (b) a VL that comprises CDR1, CDR2, and CDR3 comprising the amino acid sequences of SEQ ID NOS: 66, 67, and 68, respectively.

In certain embodiments, an antigen-binding site described in the present application is derived from 2019EP69-E02. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:69, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:70. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 69 and 70, respectively. In certain embodiments, the VH comprises CDR1, CDR2, and CDR3 comprising the amino acid sequences of SEQ ID NOS: 11, 12, and 71, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1, CDR2, and CDR3 comprising the amino acid sequences of SEQ ID NOS: 11, 12, and 71, respectively; and (b) a VL that comprises CDR1, CDR2, and CDR3 comprising the amino acid sequences of SEQ ID NOS: 47, 48, and 49, respectively.

In certain embodiments, an antigen-binding site described in the present application is derived from 2019EP69-E10. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:72, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:73. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 72 and 73, respectively. In certain embodiments, the VH comprises CDR1, CDR2, and CDR3 comprising the amino acid sequences of SEQ ID NOS: 11, 12, and 74, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1, CDR2, and CDR3 comprising the amino acid sequences of SEQ ID NOS: 11, 12, and 74, respectively; and (b) a VL that comprises CDR1, CDR2, and CDR3 comprising the amino acid sequences of SEQ ID NOS: 47, 48, and 49, respectively.

In certain embodiments, an antigen-binding site described in the present application is derived from 2019EP69-C05. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:75, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:76. In certain embodiments, the antigen-binding site comprises the heavy chain CDR1, CDR2, and CDR3 and the light chain CDR1, CDR2, and 75 and 76, respectively. In certain embodiments, the VH comprises CDR1, CDR2, and CDR3 comprising the amino acid sequences of SEQ ID NOS: 11, 12, and 77, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1, CDR2, and CDR3 comprising the amino acid sequences of SEQ ID NOS: 11, 12, and 77, respectively; and (b) a VL that comprises CDR1, CDR2, and CDR3 comprising the amino acid sequences of SEQ ID NOS: 47, 48, and 49, respectively.

In certain embodiments, an antigen-binding site described in the present application is derived from 2019EP69-F02. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:78, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:79. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 78 and 79, respectively. In certain embodiments, the VH comprises CDR1, CDR2, and CDR3 comprising the amino acid sequences of SEQ ID NOS: 11, 12, and 80, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1, CDR2, and CDR3 comprising the amino acid sequences of SEQ ID NOS: 11, 12, and 80, respectively; and (b) a VL that comprises CDR1, CDR2, and CDR3 comprising the amino acid sequences of SEQ ID NOS: 47, 48, and 49, respectively.

In certain embodiments, an antigen-binding site described in the present application is derived from 2019EP69-B01. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:81, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:82. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 81 and 82, respectively. In certain embodiments, the VH comprises CDR1, CDR2, and CDR3 comprising the amino acid sequences of SEQ ID NOS: 11, 12, and 83, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1, CDR2, and CDR3 comprising the amino acid sequences of SEQ ID NOS: 11, 12, and 83, respectively; and (b) a VL that comprises CDR1, CDR2, and CDR3 comprising the amino acid sequences of SEQ ID NOS: 47, 48, and 49, respectively.

In certain embodiments, an antigen-binding site described in the present application is derived from 2019EP69-H10. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:84, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:85. In certain embodiments, the antigen-binding site comprises the heavy chain CDR1, CDR2, and CDR3 and the light chain CDR1, CDR2, and 84 and 85, respectively. In certain embodiments, the VH comprises CDR1, CDR2, and CDR3 comprising the amino acid sequences of SEQ ID NOS: 11, 12, and 86, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1, CDR2, and CDR3 comprising the amino acid sequences of SEQ ID NOS: 11, 12, and 86, respectively; and (b) a VL that comprises CDR1, CDR2, and CDR3 comprising the amino acid sequences of SEQ ID NOS: 47, 48, and 49, respectively.

In certain embodiments, an antigen-binding site described in the present application is derived from 2019EP69-F03. For example, in certain embodiments, an antigen-binding site described in the present application comprises a VH that comprises an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:87, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:88. In certain embodiments, the antigen-binding site comprises the heavy chain CDR1, CDR2, and CDR3 and the light chain CDR1, CDR2, and CDR3, determined under IMGT, EU, Kabat, Chothia, MacCallum, or any other CDR determination method known in the art, of the VH and VL sequences of SEQ ID NOS: 87 and 88, respectively. In certain embodiments, the VH comprises CDR1, CDR2, and CDR3 comprising the amino acid sequences of SEQ ID NOS: 11, 12, and 89, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1, CDR2, and CDR3 comprising the amino acid sequences of SEQ ID NOS: 11, 12, and 89, respectively; and (b) a VL that comprises CDR1, CDR2, and CDR3 comprising the amino acid sequences of SEQ ID NOS: 47, 48, and 49, respectively.

In certain embodiments, the antigen-binding site described herein comprises a VH that comprises an amino acid sequence at least 90% (e.g., at least 91%, at least 92%, at least 93%, at least 94%, at least 95%, at least 96%, at least 97%, at least 98%, or at least 99%) identical to the amino acid sequence of SEQ ID NO:87, and a VL that comprises an amino acid sequence at least 90% (e.g., at least 91%, at least 92%, at least 93%, at least 94%, at least 95%, at least 96%, at least 97%, at least 98%, at least 99%, or 100%) identical to SEQ ID NO:88, and the VH comprises a CDR3 comprising an amino acid sequence variant of SEQ ID NO:89 having at least 1, 2, 3, 4, or 5 amino acid substitutions compared to SEQ ID NO:89. In some embodiments, the VH CDR3 comprises or consists of SEQ ID NOS: 71, 74, 77, 80, 83, or 86. In certain embodiments, the VH further comprises a CDR1 and CDR2 comprising the amino acid sequences of SEQ ID NOS: 11 and 12, respectively. In certain embodiments, the VL comprises CDR1, CDR2, and CDR3 comprising the amino acid sequences of SEQ ID NOS: 47, 48, and 49, respectively. In certain embodiments, the antigen-binding site comprises (a) a VH that comprises CDR1 having the sequence of SEQ ID NO:11, CDR2 having the sequence of SEQ ID NO:12, and CDR3 comprising a variant of SEQ ID NO: 89 having at least 1, 2, 3, 4, or 5 amino acid substitutions compared to SEQ ID NO: 89; and (b) a VL that comprises CDR1, CDR2, and CDR3 comprising the amino acid sequences of SEQ ID NOS: 47, 48, and 49, respectively.

In each of the foregoing embodiments, it is contemplated herein that the VH and/or VL sequences that bind PD-L1 may contain amino acid alterations (e.g., at least 1, 2, 3, 4, 5, or 10 amino acid substitutions, deletions, or additions) in the framework regions of the VH and/or VL without affecting their ability to bind to PD-L1. For example, it is contemplated herein that VH and VL sequences (e.g., in an scFv) that bind PD-L1 may contain cysteine heterodimerization mutations, facilitating formation of a disulfide bridge between the VH and VL of the scFv.

In some embodiments, the antigen-binding site disclosed here binds human PD-L1 with a KD less than about 5 nM, less than about 4 nM, less than about 3 nM, less than about 2 nM, less than about 1 nM, less than about 0.8 nM, less than about 0.6 nM, less than about 0.4 nM, less than about 0.2 nM, or less than about 0.1 nM, as measured by surface plasmon resonance (SPR).

In some embodiments, the antigen-binding site disclosed herein binds human PD-L1 with an EC50 less than about 160 nM, less than about 10 nM, less than about 1.5 nM, less than about 1.2 nM, less than about 1.0 nM, less than about 0.8 nM, less than about 0.7 nM, less than about 0.6 nM, less than about 0.5 nM, or less than about 0.4 nM, as measured by enzyme-linked immunosorbent assay (ELISA).

In certain embodiments, the antigen-binding site disclosed herein binds human PD-L1 or the extracellular region thereof at a KD value less than or equal to (affinity greater than or equal to) 100 nM, 90 nM, 80 nM, 70 nM, 60 nM, 50 nM, 45 nM, 40 nM, 35 nM, 30 nM, 25 nM, 20 nM, 15 nM, 10 nM, 9 nM, 8 nM, 7 nM, 6 nM, 5 nM, 4 nM, 3 nM, 2 nM, 1 nM, or 0.5 nM. In certain embodiments, the antigen binding site of the present application binds human PD-L1 or the extracellular region thereof at a KD value less than or equal to (affinity greater than or equal to) 5 nM. In certain embodiments, an antigen-binding site of the present application binds human PD-L1 or the extracellular region thereof at a KD value less than or equal to (affinity greater than or equal to) about 2.0 nM, 2.1 nM, 2.2 nM, 2.3 nM, 2.4 nM, 2.5 nM, 2.6 nM, 2.7 nM, 2.8 nM, 2.9 nM, 3.0 nM, 3.1 nM, 3.2 nM, 3.3 nM, 3.4 nM, 3.5 nM, 3.6 nM, 3.7 nM, 3.8 nM, 3.9 nM, 4.0 nM, 4.1 nM, 4.2 nM, 4.3 nM, 4.4 nM, 4.5 nM, 4.6 nM, 4.7 nM, 4.8 nM, 4.9 nM or 5.0 nM. In certain embodiments, an antigen-binding site of the present application binds human PD-L1 or the extracellular region thereof at a KD value in the range of about 1.0-3.5 nM, 1.0-4.0 nM, 1.0-4.5 nM, 1.0-5.0 nM, 1.5-3.5 nM, 1.5-4.0 nM, 1.5-4.5 nM, 1.5-5.0 nM, 2.0-3.5 nM, 2.0-4.0 nM, 2.0-4.5 nM, 2.0-5.0 nM, 2.5-3.5 nM, 2.5-4.0 nM, 2.5-4.5 nM, 2.5-5.0 nM, 3.0-3.5 nM, 3.0-4.0 nM, 3.0-4.5 nM, or 3.0-5.0 nM. These KD values are as measured using standard binding assays, for example, SPR or ELISA.

In certain embodiments, the antigen-binding site disclosed herein binds cells expressing human PD-L1 with an EC50 less than about 40 nM, less than about 10 nM, less than about 8 nM, less than about 6 nM, less than about 4 nM, less than about 2 nM, less than about 1 nM, less than about 0.5 nM, less than about 0.3 nM, less than about 0.2 nM, less than about 0.1 nM, as measured by fluorescence-activated cell sorting (FACS).

In certain embodiments, the antigen-binding site disclosed herein binds human PD-L1 presented on the surface of a cell membrane (e.g., plasma membrane of a cell) at an EC50 value less than or equal to (affinity greater than or equal to) 50 nM, 45 nM, 40 nM, 35 nM, 30 nM, 25 nM, 20 nM, 15 nM, 10 nM, 9 nM, 8 nM, 7 nM, 6 nM, 5 nM, 4 nM, 3 nM, 2 nM, 1 nM, or 0.5 nM. In certain embodiments, the antigen-binding site disclosed herein binds human PD-L1 presented on the surface of a membrane (e.g., plasma membrane of a cell) at an EC50 value less than or equal to (affinity greater than or equal to) 1 nM. These EC50 values can be measured in a binding assay using cells recombinantly or endogenously expressing PD-L1, such as the assays disclosed in the example(s) below. In certain embodiments the antibody binds PD-L1 from a body fluid, tissue and/or cell of a subject.

In certain embodiments, the antigen-binding site disclosed herein competes with PD-1 for binding PD-L1 or inhibits PD-L1 binding to PD-1. In certain embodiments, the antigen-binding site disclosed herein is present as a single-chain fragment variable fragment (scFv). In certain embodiments, the antigen-binding site disclosed herein is present as an antigen-binding fragment (Fab).

Proteins with Antigen-Binding Sites that Bind PD-L1

In certain embodiments, the present disclosure provides a protein comprising an antigen-binding site disclosed herein that binds human PD-L1. In certain embodiments, the protein of present disclosure comprises one or more antibody heavy chain constant region. In certain embodiments, the antibody heavy chain constant region is a human IgG heavy chain constant region. In certain embodiments, the antibody heavy chain constant region is human IgG1 heavy chain constant region. In certain embodiments, the antibody heavy chain constant region comprises an amino acid sequence that is at least 90% identical to SEQ ID NO:90.

In certain embodiments, the antibody heavy chain constant region comprises, relative to a human IgG, one or more mutations selected from L234A, L235A, P329G, Y349C, S354C, T366S, T366W, L368A, F405K, K409A and Y407V, numbered according to the EU numbering system. In some embodiments, the human IgG is IgG1. In some embodiments, the human IgG comprises the sequence of SEQ ID NO: 90, or a sequence having at least 90%, at least 91%, at least 92%, at least 93%, at least 94%, at least 95%, at least 96%, at least 97%, at least 98%, or at least 99% sequence identity to SEQ ID NO:90. In some embodiments, the heavy chain constant region comprises LALAPG mutations. LALAPG mutations refer to L234A, L235A, and P329G changes in the CH2-CH3 region of human IgG heavy chain constant region, e.g., IgG1 (see, e.g., Schlothauer et al., Protein Engineering, Design & Selection. 29(10):457-466, 2016; Lo et al., J. Biol. Chem. 292(9):3900-3908, 2017, hereby incorporated by reference). In some embodiments, the antibody heavy chain constant region comprises knob mutations or hole mutations. Knob-into-hole mutations are modifications to the IgG constant domain that allow heterodimerization of Fc domains that comprise the knob and hole mutations, respectively. Knob or hole mutations allow for preferential heterodimer formation in vitro with low levels of homodimer contaminants. Knob-into-hole mutations are disclosed in Merchant et al. Nat. Biotechnol. 16:677-681, 1998; and Wei et al., Oncotarget. 8(31):51037-51049, 2017, which are hereby incorporated by reference. Examples of knob mutations include S354C, T366W and K409A mutations in an IgG heavy chain constant region. Examples of hole mutations include Y349C, T366S, L368A, F405K, and Y407V mutations in an IgG heavy chain constant region. In some embodiments, the antibody heavy chain constant region comprises LALAPG mutations and hole mutations or knob mutations. For example, in some embodiments, the antibody heavy chain constant region can comprise L234A, L235A, P329G, S354C, T366W and K409A mutations. In some embodiments, the antibody heavy chain constant region can comprise L234A, L235A, P329G, Y349C, T366S, L368A, F405K, and Y407V mutations. In certain embodiments, the antibody heavy chain constant region comprises the amino acid sequence of SEQ ID NO:91 or 92. In certain embodiments, the protein comprises a first antibody heavy chain constant region comprising, relative to SEQ ID NO:90, one or more mutations selected from S354C, T366W and K409A and a second antibody heavy chain constant region comprising, relative to SEQ ID NO:90, one or more mutations selected from Y349C, T366S, L368A, F405K and Y407V, numbered according to the EU numbering system. In certain embodiments, the protein comprises a first antibody heavy chain constant region comprising, relative to SEQ ID NO:90, one or more mutations selected from L234A, L235A, P329G, S354C, T366W and K409A and a second antibody heavy chain constant region comprising, relative to SEQ ID NO:90, one or more mutations selected from L234A, L235A, P329G, Y349C, T366S, L368A, F405K and Y407V, numbered according to the EU numbering system.

TABLE 2
Example IgG Sequences.

| | | |
|---|---|---|
| IgG1 WT CH2- | DKTHTCPPCPAPELLGGPSVFLFPPKPKDTLMISRTPEV | SEQ ID |
| CH3 | TCVVVDVSHEDPEVKFNWYVDGVEVHNAKTKPREEQ | NO: 90 |
| | YNSTYRVVSVLTVLHQDWLNGKEYKCKVSNKALPAPI | |
| | EKTISKAKGQPREPQVYTLPPSRDELTKNQVSLTCLVK | |
| | GFYPSDIAVEWESNGQPENNYKTTPPVLDSDGSFFLYS | |
| | KLTVDKSRWQQGNVFSCSVMHEALHNHYTQKSLSLSP | |
| | GK | |
| EP280 | DKTHTCPPCPAPEAAGGPSVFLFPPKPKDTLMISRTPEV | SEQ ID |
| IgG1, CH2-CH3, | TCVVVDVSHEDPEVKFNWYVDGVEVHNAKTKPREEQ | NO: 91 |
| hole with Y349C, | YNSTYRVVSVLTVLHQDWLNGKEYKCKVSNKALGAP | |
| T366S, L368A, | IEKTISKAKGQPREPQVCTLPPSRDELTKNQVSLSCAVK | |
| F405K, Y407V; | GFYPSDIAVEWESNGQPENNYKTTPPVLDSDGSFKLVS | |
| LALAPG | KLTVDKSRWQQGNVFSCSVMHEALHNHYTQKSLSLSP | |
| mutation | GK | |
| IgG1 Knob; with | DKTHTCPPCPAPEAAGGPSVFLFPPKPKDTLMISRTPEV | SEQ ID |
| S354C, T366W, | TCVVVDVSHEDPEVKFNWYVDGVEVHNAKTKPREEQ | NO: 92 |
| K409A; LALAPG | YNSTYRVVSVLTVLHQDWLNGKEYKCKVSNKALGAP | |
| mutation | IEKTISKAKGQPREPQVYTLPPCRDELTKNQVSLWCLV | |
| | KGFYPSDIAVEWESNGQPENNYKTTPPVLDSDGSFFLY | |
| | SALTVDKSRWQQGNVFSCSVMHEALHNHYTQKSLSLS | |
| | PGK | |

In some embodiments, the protein comprising an antigen-binding site that binds PD-L1 is an antibody. In some embodiments, the antibody comprises: (a) a heavy chain (HC) comprising or consisting of an amino acid sequence at least 90%, 91%, 92%, 93%, 94%, 95%, 96%, 97%, 98%, 99% identical to SEQ ID NO:148 and a light chain (LC) comprising or consisting of an amino acid sequence at least 90%, 91%, 92%, 93%, 94%, 95%, 96%, 97%, 98%, 99% identical to SEQ ID NO:149; (b) a HC comprising or consisting of an amino acid sequence at least 90%, 91%, 92%, 93%, 94%, 95%, 96%, 97%, 98%, 99% identical to SEQ ID NO:150 and a LC comprising or consisting of an amino acid sequence at least 90%, 91%, 92%, 93%, 94%, 95%, 96%, 97%, 98%, 99% identical to SEQ ID NO:151; (c) a HC comprising or consisting of an amino acid sequence at least 90%, 91%, 92%, 93%, 94%, 95%, 96%, 97%, 98%, 99% identical to SEQ ID NO:152 and a LC comprising or consisting of an amino acid sequence at least 90%, 91%, 92%, 93%, 94%, 95%, 96%, 97%, 98%, 99% identical to SEQ ID NO:153; (d) a HC comprising or consisting of an amino acid sequence at least 90%, 91%, 92%, 93%, 94%, 95%, 96%, 97%, 98%, 99% identical to SEQ ID NO:154 and a LC comprising or consisting of an amino acid sequence at least 90%, %, 91%, 92%, 93%, 94%, 95%, 96%, 97%, 98%, 99% identical to SEQ ID NO:155; (e) a HC comprising or consisting of an amino acid sequence at least 90%, 91%, 92%, 93%, 94%, 95%, 96%, 97%, 98%, 99% identical to SEQ ID NO:156 and a LC comprising or consisting of an amino acid sequence at least 90%, 91%, 92%, 93%, 94%, 95%, 96%, 97%, 98%, 99% identical to SEQ ID NO:157; or (f) a HC comprising or consisting of an amino acid sequence at least 90%, 91%, 92%, 93%, 94%, 95%, 96%, 97%, 98%, 99% identical to SEQ ID NO:159 and a LC comprising or consisting of an amino acid sequence at least 90%, 91%, 92%, 93%, 94%, 95%, 96%, 97%, 98%, 99% identical to SEQ ID NO:160.

In some embodiments, the antibody comprises: (a) a HC comprising or consisting of an amino acid sequence of SEQ ID NO: 148 and a LC comprising or consisting of an amino acid sequence of SEQ ID NO: 149; (b) a HC comprising or consisting of an amino acid sequence of SEQ ID NO:150 and a LC comprising or consisting of an amino acid sequence of SEQ ID NO:151; (c) a HC comprising or consisting of an amino acid sequence of SEQ ID NO: 152 and a LC comprising or consisting of an amino acid sequence of SEQ ID NO:153; (d) a HC comprising or consisting of an amino acid sequence of SEQ ID NO:154 and a LC comprising or consisting of an amino acid sequence of SEQ ID NO:155; (e) a HC comprising or consisting of an amino acid sequence of SEQ ID NO:156 and a LC comprising or consisting of an amino acid sequence of SEQ ID NO:157; or (f) a HC comprising or consisting of an amino acid sequence of SEQ ID NO: 159 and a LC comprising or consisting of an amino acid sequence of SEQ ID NO:160.

The HC and LC sequences of exemplary proteins with PD-L1 antigen-binding sites are listed in Table 3 below. The amino acid positions are identified according to the IMGT numbering scheme.

TABLE 3
Sequences of HC and LC that may form an anti-PD-L1 antibody

| | | |
|---|---|---|
| EP206 | QVQLVQSGAEVKKPGSSVKVSCKASGGTFSSYAISWVRQA | SEQ ID |
| 2019EP69-F03-HC | PGQGLEWMGGIIPIFGTANYAQKFQGRVTITADESTSTAYM | NO: 148 |
| | ELSSLRSEDTAVYYCARDKWYGSAWRGDYWGQGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSREEMTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| EP205 | QSVLTQPPSASGTPGQRVTISCSGSYSNIGSNFVNWYQQLPG | SEQ ID |
| 2019EP69-F03-LC | TAPKLLIHFNNQRPSGVPDRFSGSRSGTSASLAISGLQSEDE | NO: 149 |
| | ADYFCSTWDDSLKGPIFGGGTKLTVLGQPKANPTVTLFPPS | |
| | SEELQANKATLVCLISDFYPGAVTVAWKADGSPVKAGVETT | |
| | KPSKQSNNKYAASSYLSLTPEQWKSHRSYSCQVTHEGSTVE | |
| | KTVAPTECS | |
| EP480 | QVQLQQWGAGLLKPSETLSLTCAVYGGSFSGYYWSWIRQP | SEQ ID |
| 2018EP164-F04-HC | PGKGLEWIGEINHSGSTNYNPSLKSRVTISVDTSKNQFSLKL | NO: 150 |
| | SSVTAADTAVYYCARGRRLRYFDWAAYNWFDPWGQGTL | |
| | VTVSSASTKGPSVFPLAPSSKSTSGGTAALGCLVKDYFPEPV | |
| | TVSWNSGALTSGVHTFPAVLQSSGLYSLSSVVTVPSSSLGTQ | |
| | TYICNVNHKPSNTKVDKKVEPKSCDKTHTCPPCPAPELLGG | |
| | PSVFLFPPKPKDTLMISRTPEVTCVVVDVSHEDPEVKFNWY | |
| | VDGVEVHNAKTKPREEQYASTYRVVSVLTVLHQDWLNGK | |
| | EYKCKVSNKALPAPIEKTISKAKGQPREPQVYTLPPSREEMT | |
| | KNQVSLTCLVKGFYPSDIAVEWESNGQPENNYKTTPPVLDS | |
| | DGSFFLYSKLTVDKSRWQQGNVFSCSVMHEALHNHYTQKS | |
| | LSLSPGK | |
| EP481 | QSALTQDPAVSVALGQTVRITCQGDSLRSHYASWYQQKPG | SEQ ID |
| 2018EP164-F04-LC | QAPVVVVYDNSDRPSGIPDRFSGSNSGNTATLTISRVEVGDE | NO: 151 |
| | ADYYCQVWDSSSDHYVFGTGTKVEIRTVAAPSVFIFPPSDE | |
| | QLKSGTASVVCLLNNFYPREAKVQWKVDNALQSGNSQESV | |
| | TEQDSKDSTYSLSSTLTLSKADYEKHKVYACEVTHQGLSSP | |
| | VTKSFNRGEC | |
| EP482 | EVQLVESGGGLVQPGGSLRLSCAASGFTFSSYAMSWVRQA | SEQ ID |
| 2018EP161-G8-HC | PGKGLEWVSAISGSGGSTYYADSVKGRFTISRDNSKNTLYL | NO: 152 |
| | QMNSLRAEDTAVYYCAKASGWYPWYFDYWGQGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSREEMTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| EP483 | VIWMTQSPSSLSASLGDRVTITCRASQSVSTYINWYQQKSG | SEQ ID |
| 2018EP161-G8-LC | KGPKLLIFNASTLQSGVPSRFSGSGSGTDFTLTITSLQPDDSA | NO: 153 |
| | TYYCQQSYRVPLSFGGGTKVEIRTVAAPSVFIFPPSDEQLKS | |
| | GTASVVCLLNNFYPREAKVQWKVDNALQSGNSQESVTEQD | |
| | SKDSTYSLSSTLTLSKADYEKHKVYACEVTHQGLSSPVTKSF | |
| | NRGEC | |
| EP484 | QVQLVESGGGLVQPGGSLRLSCAASGFTFSSYAMSWVRQA | SEQ ID |
| 2018EP161-F08-HC | PGKGLEWVSAISGSGGSTYYADSVKGRFTISRDNSKNTLYL | NO: 154 |
| | QMNSLRAEDTAVYYCAKAGSWYPYYFDYWGQGTLVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICN | |
| | VNHKPSNTKVDKKVEPKSCDKTHTCPPCPAPELLGGPSVFLF | |
| | PPKPKDTLMISRTPEVTCVVVDVSHEDPEVKFNWYVDGVEV | |
| | HNAKTKPREEQYASTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |
| EP485 | DIVMTQSPSSLSASVGDRVTITCRASQTVSTYINWYQQRSG | SEQ ID |
| 2018EP161-F08-LC | KGPKLLIFDASRLQSGAPSRFSGSGSGTEFTLTITSLQPDDSA | NO: 155 |
| | TYYCQQSYRIPLSFGGGTKVEIRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| EP486 | QVQLQQWGAGLLKPSETLSLTCAVYGGSFSGYYWSWIRQP | SEQ ID |
| 2018EP280-E04-HC | PGKGLEWIGEINHIGSTNYNPSLKSRVTISVDTSNNQFSLKL | NO: 156 |
| | SSVTAADTAVYYCARGRRLRYFDWAAYNWFDPWGQGTL | |
| | VTVSSASTKGPSVFPLAPSSKSTSGGTAALGCLVKDYFPEPV | |
| | TVSWNSGALTSGVHTFPAVLQSSGLYSLSSVVTVPSSSLGTQ | |
| | TYICNVNHKPSNTKVDKKVEPKSCDKTHTCPPCPAPELLGG | |
| | PSVFLFPPKPKDTLMISRTPEVTCVVVDVSHEDPEVKFNWY | |
| | VDGVEVHNAKTKPREEQYASTYRVVSVLTVLHQDWLNGK | |
| | EYKCKVSNKALPAPIEKTISKAKGQPREPQVYTLPPSREEMT | |
| | KNQVSLTCLVKGFYPSDIAVEWESNGQPENNYKTTPPVLDS | |
| | DGSFFLYSKLTVDKSRWQQGNVFSCSVMHEALHNHYTQKS | |
| | LSLSPGK | |
| EP487 | QSALTQDPAVSVALGQTVRISCQGDSLRSHYASWYQQKPG | SEQ ID |
| 2018EP280-E04-LC | QAPVVVVYDNSDRPSGIPDRFSGSNSGNTATLTISRVEVGDE | NO: 157 |
| | ADYYCQVWDSSSDHYVFGTGTKVTVLGQPKANPTVTLFPP | |
| | SSEELQANKATLVCLISDFYPGAVTVAWKADGSPVKAGVET | |
| | TKPSKQSNNKYAASSYLSLTPEQWKSHRSYSCQVTHEGSTV | |
| | EKTVAPTECS | |
| EP488 | QVQLVQSGAEVKKPGSSVKVSCKASGGTFSSYAISWVRQA | SEQ ID |
| 2018EP171-H02-HC | PGQGLEWMGGIIPIFGTANYAQKFQGRVTITADESTSTAYM | NO: 158 |
| | ELSSLRSEDTAVYYCARDKGYGSGWRGDYWGQGTLVTVS | |
| | SASTKGPSVFPLAPSSKSTSGGTAALGCLVKDYFPEPVTVSW | |
| | NSGALTSGVHTFPAVLQSSGLYSLSSVVTVPSSSLGTQTYIC | |
| | NVNHKPSNTKVDKKVEPKSCDKTHTCPPCPAPELLGGPSVF | |
| | LFPPKPKDTLMISRTPEVTCVVVDVSHEDPEVKFNWYVDGV | |
| | EVHNAKTKPREEQYASTYRVVSVLTVLHQDWLNGKEYKC | |
| | KVSNKALPAPIEKTISKAKGQPREPQVYTLPPSREEMTKNQV | |
| | SLTCLVKGFYPSDIAVEWESNGQPENNYKTTPPVLDSDGSFF | |
| | LYSKLTVDKSRWQQGNVFSCSVMHEALHNHYTQKSLSLSP | |
| | GK | |
| EP489 | QSVLTQPPSASGTPGQRVTISCSGSYSNIGSNFVNWYQQLPG | SEQ ID |
| 2018EP171-H02-LC | TAPKLLIHFNNQRPSGVPDRFSGSRSGTSASLAISGLQSEDE | NO: 159 |
| | ADYFCSTWDDSLKGPIFGGGTKLTVLGQPKANPTVTLFPPS | |
| | SEELQANKATLVCLISDFYPGAVTVAWKADGSPVKAGVETT | |
| | KPSKQSNNKYAASSYLSLTPEQWKSHRSYSCQVTHEGSTVE | |
| | KTVAPTECS | |

In some embodiments, the antigen-binding site disclosed herein can be present in an antibody or antigen-binding fragment thereof. The antibody can be a monoclonal antibody, a chimeric antibody, a diabody, a Fab fragment, a Fab′ fragment, or F(ab′)2 fragment, an Fv, a bispecific antibody, bifunctional antibody, a bispecific Fab2, a bispecific (mab)2, a humanized antibody, an artificially-generated human antibody, bispecific T-cell engager, bispecific NK cell engager, a single chain antibody (e.g., single-chain Fv fragment or scFv), triomab, knobs-into-holes (KiH) IgG with common light chain, crossmab, ortho-Fab IgG, DVD-Ig, 2 in 1-IgG, IgG-scFv, sdFv2-Fc, bi-nanobody, dual affinity retargeting antibody (DART), DART-Fc, scFv-HSA-scFv (where HSA=human serum albumin), or dock-and-lock (DNL)-Fab3.

In certain embodiments, an antigen-binding site disclosed herein is linked to an amino acid sequence at least 90% (e.g., 90%, 91%, 92%, 93%, 94%, 95%, 96%, 97%, 98%, 99%, or 100%) identical to an antibody constant region, e.g., the heavy chain constant regions of IgG1, IgG2, IgG3, IgG4, IgM, IgA1, IgA2, IgD, or IgE; particularly, chosen from, e.g., the (e.g., human) heavy chain constant regions of IgG1, IgG2, IgG3, and IgG4. In another embodiment, an antigen-binding site disclosed herein can be linked to a light chain constant region chosen from, e.g., the (e.g., human) light chain constant regions of kappa or lambda. The constant region can be altered, e.g., mutated or engineered, to modify the properties of the antibody (e.g., to increase or decrease one or more of: Fc receptor binding, antibody glycosylation, the number of cysteine residues, effector cell function, and/or complement function). In some embodiments the antibody has effector function and can fix complement. In some embodiments the antibody does not recruit effector cells or fix complement. In some embodiments, the antibody has reduced or no ability to bind an Fc receptor. For example, it is an isotype or subtype, fragment or other mutant, which does not support binding to an Fc receptor, e.g., it has a mutagenized or deleted Fc receptor binding region.

In certain embodiments, the antigen-binding site is linked to an IgG constant region including hinge, CH2 and CH3 domains with or without a CH1 domain. In some embodiments, the amino acid sequence of the constant region is at least 90% (e.g., 90%, 91%, 92%, 93%, 94%, 95%, 96%, 97%, 98%, 99%, or 100%) identical to a human antibody constant region, such as an human IgG1 constant region, a human IgG2 constant region, a human IgG3 constant region, or a human IgG4 constant region. In some embodiments, the antibody Fc domain or a portion thereof sufficient to bind CD16 comprises an amino acid sequence at least 90% (e.g., at least 91%, at least 92%, at least 93%, at least 94%, at least 95%, at least 96%, at least 97%, at least 98%, at least 99%, or 100%) identical to a wild-type human IgG1 Fc comprising the sequence:

(SEQ ID NO: 90)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK.

In some embodiments, one or more mutations can be incorporated into the constant region as compared to human IgG1 constant region, for example at Q347, Y349, L351, S354, E356, E357, K360, Q362, S364, T366, L368, K370, N390, K392, T394, D399, S400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y407I, Y407V, K409F, K409W, K409D, T411D, T411E, K439D, and K439E.

In certain embodiments, the antigen-binding site is linked to a portion of an antibody Fc domain sufficient to bind CD16. Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793): 267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the CK of a human IgG1 constant region may be at amino acid E123, F116, S176, V163, S174, and/or T164.

In some embodiments, the antibody constant domain comprises a CH2 domain and a CH3 domain of an IgG antibody, for example, a human IgG1 antibody. In some embodiments, mutations are introduced in the antibody constant domain to enable heterodimerization with another antibody constant domain. For example, if the antibody constant domain is derived from the constant domain of a human IgG1, the antibody constant domain can comprise an amino acid sequence at least 90% (e.g., at least 91%, at least 92%, at least 93%, at least 94%, at least 95%, at least 96%, at least 97%, at least 98%, at least 99%, or 100%) identical to amino acids 234-332 of a human IgG1 antibody, and differs at one or more positions selected from the group consisting of Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

In some other embodiments, the amino acid sequence of the constant region is at least 90% (e.g., at least 91%, at least 92%, at least 93%, at least 94%, at least 95%, at least 96%, at least 97%, at least 98%, at least 99%, or 100%) identical to an antibody constant region from another mammal, such as rabbit, dog, cat, mouse, or horse.

The proteins described herein can be made using recombinant DNA technology known in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the first immunoglobulin light chain can be cloned into a third expression vector; a fourth nucleic acid sequence encoding the second immunoglobulin light chain can be cloned into a fourth expression vector; the first, second, third and fourth expression vectors can be stably transfected together into host cells to produce the multimeric proteins.

To achieve the highest yield of the proteins, different ratios of the first, second, third, and fourth expression vectors can be explored to determine the optimal ratio for transfection into the host cells. After transfection, single clones can be isolated for cell bank generation using methods known in the art, such as limited dilution, ELISA, FACS, microscopy.

Clones can be cultured under conditions suitable for bio-reactor scale-up and maintained expression of a protein comprising an antigen-binding site disclosed herein. The protein can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed mode chromatography.

Accordingly, in some embodiments, the present disclosure provides one or more isolated nucleic acids comprising sequences encoding an immunoglobulin heavy chain and/or immunoglobulin light chain variable region of any one of the antibodies disclosed herein. The disclosure provides one or more expression vectors that express the immunoglobulin heavy chain and/or immunoglobulin light chain variable region of any one of the antibodies disclosed herein. Similarly the application provides host cells comprising one or more of the foregoing expression vectors and/or isolated nucleic acids.

Competition assays for determining whether an antibody binds to the same epitope as, or competes for binding with a disclosed antibody are known in the art. Exemplary competition assays include immunoassays (e.g., ELISA assays, RIA assays), surface plasmon resonance (e.g., BIAcore analysis), bio-layer interferometry, and flow cytometry.

Typically, a competition assay involves the use of an antigen (e.g., a human PD-L1 protein or fragment thereof) bound to a solid surface or expressed on a cell surface, a test PD-L1 binding antibody and a reference antibody. The reference antibody is labeled and the test antibody is unlabeled. Competitive inhibition is measured by determining the amount of labeled reference antibody bound to the solid surface or cells in the presence of the test antibody. Usually the test antibody is present in excess (e.g., 1×, 5×, 10×, 20× or 100×). Antibodies identified by competition assay (e.g., competing antibodies) include antibodies binding to the same epitope, or similar (e.g., overlapping) epitopes, as the reference antibody, and antibodies binding to an adjacent epitope sufficiently proximal to the epitope bound by the reference antibody for steric hindrance to occur.

A competition assay can be conducted in both directions to ensure that the presence of the label does not interfere or otherwise inhibit binding. For example, in the first direction the reference antibody is labeled and the test antibody is unlabeled, and in the second direction, the test antibody is labeled and the reference antibody is unlabeled.

A test antibody competes with the reference antibody for specific binding to the antigen if an excess of one antibody (e.g., 1×, 5×, 10×, 20× or 100×) inhibits binding of the other antibody, e.g., by at least 50%, 75%, 90%, 95% or 99% as measured in a competitive binding assay.

Two antibodies may be determined to bind to the same epitope if essentially all amino acid mutations in the antigen that reduce or eliminate binding of one antibody reduce or eliminate binding of the other. Two antibodies may be determined to bind to overlapping epitopes if only a subset of the amino acid mutations that reduce or eliminate binding of one antibody reduce or eliminate binding of the other.

The antibodies disclosed herein may be further optimized (e.g., affinity-matured) to improve biochemical characteristics including affinity and/or specificity, improve biophysical properties including aggregation, stability, precipitation and/or non-specific interactions, and/or to reduce immunogenicity. For example, diversity can be introduced into an immunoglobulin heavy chain and/or an immunoglobulin light chain by DNA shuffling, chain shuffling, CDR shuffling, random mutagenesis and/or site-specific mutagenesis.

In certain embodiments, isolated human antibodies contain one or more somatic mutations. In these cases, antibodies can be modified to a human germline sequence to optimize the antibody (e.g., by a process referred to as germlining).

Generally, an optimized antibody has at least the same, or substantially the same, affinity for the antigen as the non-optimized (or parental) antibody from which it was derived. For example, in certain embodiments, an optimized antibody has a higher affinity for the antigen when compared to the parental antibody.

If the antibody is for use as a therapeutic, it can be conjugated to an effector agent such as a small molecule toxin or a radionuclide using standard in vitro conjugation chemistries. If the effector agent is a polypeptide, the antibody can be chemically conjugated to the effector or joined to the effector as a fusion protein. Construction of fusion proteins is within ordinary skill in the art.

The antibody can be conjugated to an effector moiety such as a small molecule toxin or a radionuclide using standard in vitro conjugation chemistries. If the effector moiety is a polypeptide, the antibody can be chemically conjugated to the effector or joined to the effector as a fusion protein. Construction of fusion proteins is within ordinary skill in the art.

In some embodiments, the proteins are antibodies that inhibit tumor growth in vivo.

In certain embodiments, the proteins are antibodies that induce IFNγ and TNFα secretion in vivo, at a comparable to or more enhanced level as compared to atezolizumab.

### Bifunctional Proteins

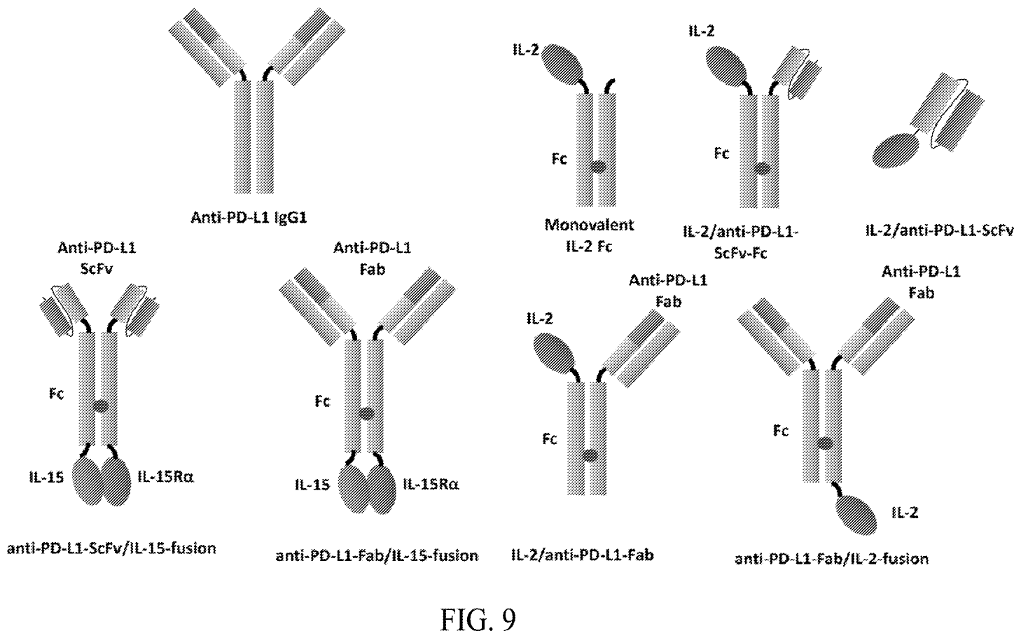

In certain embodiments, disclosed herein are bifunctional proteins, e.g., bifunctional antibodies. The bifunctional protein can comprise an antigen-binding site that binds PD-L1, an IgG Fc domain, and/or a functional domain. In some embodiments, the bifunctional protein is a single protein sequence. In other embodiments, the bifunctional protein is a heterodimer formed by at least two protein sequences. The antigen-binding site that binds PD-L1 can be any of the anti-PD-L1 antigen-binding sites, antibodies, and/or scFv sequences disclosed herein. In some embodiments, the antigen-binding site that binds PD-L1 can comprise an Fab formed by an antibody heavy chain and light chain. In some embodiments, the antigen-binding site that binds PD-L1 can comprise a scFv. The IgG Fc domain can comprise a wild type antibody constant region or a modified antibody constant region. Examples of modified antibody constant regions are provided herein and include, for example, knob or hole mutations, and LALAPG mutations. The functional domain can comprise a cytokine, cytokine receptor, or a functional fragment thereof. Examples of functional domains include an IL-2 polypeptide, such as the IL-2 and engineered IL-2 polypeptides disclosed in Table 4, IL-15, and IL-15Rα. Examples of bifunctional proteins are shown in FIG. 9 and Table 7, and include IL-2-Fc/anti-PD-L1-scFv-Fc heterodimers; IL-2-anti-PD-L1-scFv-fusion proteins that lack an Fc domain; IL-2-Fc/anti-PD-L1-Fab-Fc heterodimers; anti-PD-L1-Fab-Fc-IL-2-fusion/anti-PD-L1-Fab-Fc heterodimers; anti-PD-L1-Fab-Fc-IL-2-fusion/anti-PD-L1-Fab-Fc-IL-2-fusion dimers or heterodimers; anti-PD-L1-scFv-Fc-IL-2-fusion/anti-PD-L1-scFv-Fc-IL-2-fusion dimers or heterodimers; anti-PD-L1-scFv-Fc-IL-2-fusion/anti-PD-L1-scFv-Fc heterodimers; anti-PD-L1-scFv-Fc-IL-2-fusion/anti-PD-L1-Fab-Fc-IL-2-fusion heterodimers; anti-PD-L1-scFv-Fc/anti-PD-L1-Fab-Fc-IL-2-fusion heterodimers; anti-PD-L1-scFv-Fc-IL-2-fusion/anti-PD-L1-Fab-Fc heterodimers; anti-PD-L1-Fab-Fc-IL-15-fusion/anti-PD-L1-Fab-Fc-IL-15Rα-fusion heterodimers; anti-PD-L1-scFv-Fc-IL-15-fusion/anti-PD-L1-scFv-Fc-IL-15Rα-fusion heterodimers; anti-PD-L1-Fab-Fc-IL-15-fusion/anti-PD-L1-scFv-Fc-IL-15Rα-fusion heterodimers; and anti-PD-L1-scFv-Fc-IL-15-fusion/anti-PD-L1-Fab-Fc-IL-15Rα-fusion heterodimers.

In certain embodiments, the bifunctional protein, comprises an antigen-binding site that binds PD-L1 comprising: (i) a heavy chain variable domain (VH) comprising a CDR1 comprising the sequence of SEQ ID NO: 11, 3, 19, 33, 52, or 63; a CDR2 comprising the sequence of SEQ ID NO: 12, 4, 20, 34, 41, 53, or 64; and a CDR3 comprising the sequence of SEQ ID NO: 89, 5, 13, 21, 27, 35, 46, 54, 65, 71, 74, 77, 80, 83, or 86; and (ii) a light chain variable domain (VL) comprising a CDR1 comprising the sequence of SEQ ID NO: 47, 6, 14, 22, 28, 36, 55, or 66; a CDR2 comprising the sequence of SEQ ID NO: 48, 7, 15, 23, 29, 37, 42, 56, 60, or 67; and CDR3 comprising the sequence of SEQ ID NO: 49, 8, 16, 24, 30, 38, 43, 57, or 68. In certain embodiments, the antigen-binding site that binds PD-L1 comprises at least one of (a)-(q), wherein: (a) the VH comprises or consists of an amino acid sequence at least 90%, 91%, 92%, 93%, 94%, 95%, 96%, 97%, 98%, 99%, or 100% identical to SEQ ID NO: 1 and the VL comprises or consists of an amino acid sequence at least 90%, 91%, 92%, 93%, 94%, 95%, 96%, 97%, 98%, 99%, or 100% identical to SEQ ID NO:2; (b) the VH comprises or consists of an amino acid sequence at least 90%, 91%, 92%, 93%, 94%, 95%, 96%, 97%, 98%, 99%, or 100% identical to SEQ ID NO:9 and the VL comprises or consists of an amino acid sequence at least 90%, 91%, 92%, 93%, 94%, 95%, 96%, 97%, 98%, 99%, or 100% identical to SEQ ID NO:10; (c) the VH comprises or consists of an amino acid sequence at least 90%, 91%, 92%, 93%, 94%, 95%, 96%, 97%, 98%, 99%, or 100% identical to SEQ ID NO:17 and the VL comprises or consists of an amino acid sequence at least 90%, 91%, 92%, 93%, 94%, 95%, 96%, 97%, 98%, 99%, or 100% identical to SEQ ID NO:18; (d) the VH comprises or consists of an amino acid sequence at least 90%, 91%, 92%, 93%, 94%, 95%, 96%, 97%, 98%, 99%, or 100% identical to SEQ ID NO:25 and the VL comprises or consists of an amino acid sequence at least 90%, 91%, 92%, 93%, 94%, 95%, 96%, 97%, 98%, 99%, or 100% identical to SEQ ID NO:26; (e) the VH comprises or consists of an amino acid sequence at least 90%, 91%, 92%, 93%, 94%, 95%, 96%, 97%, 98%, 99%, or 100% identical to SEQ ID NO:31 and the VL comprises or consists of an amino acid sequence at least 90%, 91%, 92%, 93%, 94%, 95%, 96%, 97%, 98%, 99%, or 100% identical to SEQ ID NO:32; (f) the VH comprises or consists of an amino acid sequence at least 90%, 91%, 92%, 93%, 94%, 95%, 96%, 97%, 98%, 99%, or 100% identical to SEQ ID NO: 39 and the VL comprises or consists of an amino acid sequence at least 90%, 91%, 92%, 93%, 94%, 95%, 96%, 97%, 98%, 99%, or 100% identical to SEQ ID NO:40; (g) the VH comprises or consists of an amino acid sequence at least 90%, 91%, 92%, 93%, 94%, 95%, 96%, 97%, 98%, 99%, or 100% identical to SEQ ID NO:44 and the VL comprises or consists of an amino acid sequence at least 90%, 91%, 92%, 93%, 94%, 95%, 96%, 97%, 98%, 99%, or 100% identical to SEQ ID NO:45; (h) the VH comprises or consists of an amino acid sequence at least 90%, 91%, 92%, 93%, 94%, 95%, 96%, 97%, 98%, 99%, or 100% identical to SEQ ID NO:50 and the VL comprises or consists of an amino acid sequence at least 90%, 91%, 92%, 93%, 94%, 95%, 96%, 97%, 98%, 99%, or 100% identical to SEQ ID NO:51; (i) the VH comprises or consists of an amino acid sequence at least 90%, 91%, 92%, 93%, 94%, 95%, 96%, 97%, 98%, 99%, or 100% identical to SEQ ID NO:58 and the VL comprises or consists of an amino acid sequence at least 90%, 91%, 92%, 93%, 94%, 95%, 96%, 97%, 98%, 99%, or 100% identical to SEQ ID NO:59; (j) the VH comprises or consists of an amino acid sequence at least 90%, 91%, 92%, 93%, 94%, 95%, 96%, 97%, 98%, 99%, or 100% identical to SEQ ID NO:61 and the VL comprises or consists of an amino acid sequence at least 90%, 91%, 92%, 93%, 94%, 95%, 96%, 97%, 98%, 99%, or 100% identical to SEQ ID NO:62; (k) the VH comprises or consists of an amino acid sequence at least 90%, 91%, 92%, 93%, 94%, 95%, 96%, 97%, 98%, 99%, or 100% identical to SEQ ID NO: 69 and the VL comprises or consists of an amino acid sequence at least 90%, 91%, 92%, 93%, 94%, 95%, 96%, 97%, 98%, 99%, or 100% identical to SEQ ID NO:70; (l) the VH comprises or consists of an amino acid sequence at least 90%, 91%, 92%, 93%, 94%, 95%, 96%, 97%, 98%, 99%, or 100% identical to SEQ ID NO:72 and the VL comprises or consists of an amino acid sequence at least 90%, 91%, 92%, 93%, 94%, 95%, 96%, 97%, 98%, 99%, or 100% identical to SEQ ID NO:73; (m) the VH comprises or consists of an amino acid sequence at least 90%, 91%, 92%, 93%, 94%, 95%, 96%, 97%, 98%, 99%, or 100% identical to SEQ ID NO:75 and the VL comprises or consists of an amino acid sequence at least 90%, 91%, 92%, 93%, 94%, 95%, 96%, 97%, 98%, 99%, or 100% identical to SEQ ID NO:76; (n) the VH comprises or consists of an amino acid sequence at least 90%, 91%, 92%, 93%, 94%, 95%, 96%, 97%, 98%, 99%, or 100% identical to SEQ ID NO:78 and the VL comprises or consists of an amino acid sequence at least 90%, 91%, 92%, 93%, 94%, 95%, 96%, 97%, 98%, 99%, or 100% identical to SEQ ID NO:79; (o) the VH comprises or consists of an amino acid sequence at least 90%, 91%, 92%, 93%, 94%, 95%, 96%, 97%, 98%, 99%, or 100% identical to SEQ ID NO:81 and the VL comprises or consists of an amino acid sequence at least 90%, 91%, 92%, 93%, 94%, 95%, 96%, 97%, 98%, 99%, or 100% identical to SEQ ID NO:82; (p) the VH comprises or consists of an amino acid sequence at least 90%, 91%, 92%, 93%, 94%, 95%, 96%, 97%, 98%, 99%, or 100% identical to SEQ ID NO: 84 and the VL comprises or consists of an amino acid sequence at least 90%, 91%, 92%, 93%, 94%, 95%, 96%, 97%, 98%, 99%, or 100% identical to SEQ ID NO:85; and/or (q) the VH comprises or consists of an amino acid sequence at least 90%, 91%, 92%, 93%, 94%, 95%, 96%, 97%, 98%, 99%, or 100% identical to SEQ ID NO:87 and the VL comprises or consists of an amino acid sequence at least 90%, 91%, 92%, 93%, 94%, 95%, 96%, 97%, 98%, 99%, or 100% identical to SEQ ID NO:88.

In some embodiments, the bifunctional protein comprises an antibody heavy chain constant region is human IgG heavy chain constant region. In some embodiments, the antibody heavy chain constant region is human IgG1 heavy chain constant region. In some embodiments, the antibody heavy chain constant region comprises an amino acid sequence at least 90% identical to SEQ ID NO:90. In some embodiments, the antibody heavy chain constant region comprises, relative to SEQ ID NO: 90, one or more mutations selected from L234A, L235A, P329G, Y349C, S354C, T366S, T366W, L368A, F405K, K409A and Y407V, numbered according to the EU numbering system. In some embodiments, the antibody heavy chain constant region comprises, relative to SEQ ID NO:90, one or more mutations selected from S354C, T366W and K409A. In some embodiments, the antibody heavy chain constant region comprises, relative to SEQ ID NO:90, one or more mutations selected from Y349C, T366S, L368A, F405K and Y407V, numbered according to the EU numbering system. In some embodiments, the bifunctional protein is a heterodimer and comprises a first and a second antibody heavy chain constant region, wherein the first antibody heavy chain constant region comprises, relative to SEQ ID NO:90, mutations S354C, T366W and K409A; and second heavy chain constant region comprises, relative to SEQ ID NO: 90, mutations Y349C, T366S, L368A, F405K and Y407V, numbered according to the EU numbering system. In some embodiments, the antibody heavy chain constant region comprises L234A, L235A, and P329G mutations. In some embodiments, one of the first and the second antibody heavy chain constant regions comprises the amino acid sequence of SEQ ID NO:91 and the second antibody heavy chain constant region comprises the amino acid sequence of SEQ ID NO:92.

In certain embodiments, the bifunctional protein comprises a functional domain, wherein the functional domain is an IL-15 or an IL-15Rα, or functional fragment thereof. IL-15 is a member of the four a-helix bundle-containing cytokines. IL-15 is typically formed a complex with IL-15 receptor alpha expressed on APCs prior to binding to functional IL-15 receptor beta and gamma units on T cells and NK cells. IL-15 may be presented in trans to responsive cells expressing IL-2Rβ (IL15RB, or CD122) and IL-2Rγ (CD132) by cells expressing the cytokine itself bound to a membrane form of the receptor alpha chain. Without wishing to be limited by theory, the IL-15 receptor alpha sushi domain is a thought to be required to form a complex with IL-15 prior to proper engagement with receptor β and γ. IL-15 and IL-15Rα complex and IL-15/IL-15Rα sushi domain fusion protein were reported to be highly potent to stimulate CD8 T cells and NK cells.

In certain embodiments, the bifunctional protein disclosed herein comprises: (a) a first subunit comprising the antigen-binding site that binds PD-L1, a first antibody heavy chain constant region, and an IL-15 polypeptide or a functional fragment or variant thereof; and (b) a second subunit comprising the antigen-binding site that binds PD-L1, a second antibody heavy chain constant region, and an IL-15Rα polypeptide or functional fragment or variant thereof. In some embodiments, the IL-15 polypeptide comprises SEQ ID NO:93 or a functional fragment or variant thereof. In some embodiments, the IL-15 polypeptide comprises amino acids 50-162 of SEQ ID NO: 93 or a functional fragment or variant thereof. In some embodiments, the IL-15Rα polypeptide comprises SEQ ID NO:94 or a functional fragment or variant thereof. In some embodiments, the IL-15Rα polypeptide comprises amino acids 31-97 of SEQ ID NO: 94 or a functional fragment or variant thereof.

In some embodiments, the bifunctional protein disclosed herein is a heterodimer comprising an anti-PD-L1-Fab-Fc-IL-15-fusion protein and an anti-PD-L1-Fab-Fc-IL-15Rα-fusion protein. For example, in some embodiments, the bifunctional protein comprises a first subunit comprising an amino acid sequence at least 90% identical to SEQ ID NO:186 (EP203), a second subunit comprising an amino acid sequence at least 90% identical to SEQ ID NO:187 (EP204); and a third subunit comprising an amino acid sequence at least 90% identical to SEQ ID NO:181 (EP205). In some embodiments, the first subunit comprises or consists of the amino acid sequence of SEQ ID NO: 186, the second subunit comprises or consists of the amino acid sequence of SEQ ID NO:187; and the third subunit comprises or consists of the amino acid sequence of SEQ ID NO: 181.

In some embodiments, the bifunctional protein disclosed herein is a heterodimer comprising an anti-PD-L1-scFv-Fc-IL-15-fusion protein and an anti-PD-L1-scFv-Fc-IL-15Rα-fusion protein. For example, in some embodiments, the bifunctional protein comprises a first subunit comprising an amino acid sequence at least 90% identical to SEQ ID NO:188 (EP207) and a second subunit comprising an amino acid sequence at least 90% identical to SEQ ID NO:189 (EP208). In some embodiments, the bifunctional protein comprises a first subunit comprising or consisting of the amino acid sequence of SEQ ID NO: 188, and a second subunit comprising or consisting of the amino acid sequence of SEQ ID NO:189.

In certain embodiments, the bifunctional protein disclosed herein comprises a functional domain, wherein the functional domain is an IL-2, for example a wild-type IL-2 or an engineered IL-2, or functional fragment or variant thereof. The term “interleukin-2 or “IL-2” as used herein, refers to an IL-2 from any vertebrate source, including mammals such humans or mice, unless otherwise indicated. The term encompasses precursor or unprocessed IL-2, as well as any form of IL-2 that results from cellular processing. The term also encompasses naturally occurring variants of IL-2, such as splice variants or allelic variants. The amino acid sequence of an example mature human IL-2 is shown in SEQ ID NO: 191. “Wild-type” or “native” when used in reference to IL-2 is intended to mean the mature IL-2 molecule (e.g., SEQ ID NO: 191). The term “engineered IL-2” or “engineered IL-2 polypeptide” as used herein encompasses an IL-2 having at least one residue that differs from a native or wild-type IL-2, and includes full-length IL-2, truncated forms of IL-2, and forms where IL-2 is linked or fused with another molecule, such as another polypeptide. The various forms of engineered IL-2 are characterized in having at least one amino acid substitution affecting the interaction of IL-2 with IL-2Rβ and/or IL-2Rα. Identification of various engineered forms of IL-2 as described herein are made with respect to the sequence shown in, e.g., SEQ ID NO: 192. In some embodiments, IL-2 is modified to include a T3A substitution. The T3A substitution may be made to the wild-type sequence. In addition, the T3A substitution may be made to an engineered IL-2 sequence disclosed herein. Examples of wild-type and engineered IL-2 polypeptides are described in PCT/US2020/046244, which is hereby incorporated by reference herein in its entirety.

IL-2 modulates lymphocyte proliferation and activation. IL-2 mediates its action by binding to IL-2 receptors (IL-2R), which includes up to three individual subunits. Association of all three subunits, the interleukin-2 receptor alpha chain (IL-2Rα, or CD25), interleukin-2 receptor beta chain (IL-2Rβ, or CD122), and interleukin-2 receptor gamma chain (IL-2Rγ, or CD132), results in a trimeric IL-2Rαβγ, which is a high-affinity receptor for IL-2. Association of the IL-2Rβ and IL-2Rγ subunits results in the dimeric receptor IL-2Rβγ, and is termed an intermediate affinity IL-2R. The IL-2Rα subunit forms a monomeric low affinity IL-2 receptor. Expression of IL-2Rα is involved in the expansion of immunosuppressive regulatory T cells (Tregs); whereas dimeric IL-2Rβγ can result in cytolytic CD8⁺ T cell and NK cell proliferation and killing in the absence of IL-2Rα.

In some embodiments, the bifunctional protein comprises an antigen-binding site that binds PD-L1 and comprises a wild-type IL-2 (e.g., SEQ ID NO: 191). In some embodiments, the bifunctional protein comprises an antigen-binding site that binds PD-L1 and comprises an engineered IL-2 (e.g., SEQ ID NO: 192). In some embodiments, the bifunctional protein comprises an antigen-binding site that binds PD-L1 and comprises an engineered IL-2, wherein the engineered IL-2 polypeptide comprises:
(a) an IL-2 receptor α (IL-2Rα) binding region 1 comprising, relative to wild-type IL-2, one or more mutations at one or more positions selected from:
a mutation at position K35 selected from K35G, K35L, K35S, K35V, K35D, K35E, and K35C;
a mutation at position R38 selected from R38V, R38D, R38E, R38S, R38I, R38A, R38Y, R38G, R38C, and R38N;
a mutation at position F42 selected from F42A, F42R, F42G, F42I, F42L, F42P and F42H; and
a mutation at position Y45 selected from Y45S, Y45P, Y45A, Y45V, Y45C, Y45T, and Y45F, and/or
(b) an IL-2 receptor β (IL-2Rβ) binding region 2 motif comprising:

(SEQ ID NO: 95)
X1-X2-X3-D-X4-X-5-X6-N-X7-X8-X9-X10-X11-X12-X13,

wherein:
X1 is selected from C, T, G, W, I, S, E, and K;
X2 is selected from Y, P, V, W, L, A, and G;
X3 is selected from S, T, Q, G, M, E, R, and K;
X4 is selected from A, V, S, and T;
X5 is selected from I, L, T, and V;
X6 is selected from S, T, E, D, and R;
X7 is selected from I, A, M, and V;
X8 is selected from S, T, N, Q, I, G, E, K, and R;
X9 is selected from V, L, and I;
X10 is selected from N, T, I, and L;
X11 is selected from V, A, and I;
X12 is selected from and Q, L, G, K, and R; and
X13 is selected from A, D, and E.
   In some embodiments, the bifunctional protein comprises an antigen-binding site that binds PD-L1 and comprises an engineered IL-2, wherein the engineered IL-2 polypeptide comprises: (a) an IL-2Rα binding region 1 comprises an amino acid sequence at least 90% identical to an amino acid sequence selected from SEQ ID NOS: 124-147; and/or (b) an IL-2Rβ binding region 2 motif comprises an amino acid sequence at least 90% identical to an amino acid sequence selected from SEQ ID NOS: 96-123. In some embodiments, the bifunctional protein comprises an antigen-binding site that binds PD-L1 and comprises an engineered IL-2, wherein: (a) the IL-2Rα binding region 1 comprises an amino acid sequence selected from SEQ ID NOS: 124-147; and/or (b) the IL-2Rβ binding region 2 motif comprises an amino acid sequence selected from SEQ ID NOS: 96-123. In some embodiments, the antigen-binding site that binds PD-L1 is an scFv or an Fab.

In certain embodiments, the bifunctional protein is a fusion protein comprising: (a) a first subunit comprising the antigen-binding site that binds PD-L1 fused to (b) a second subunit comprising a wild-type or an engineered IL-2 polypeptide or a functional fragment or variant thereof. In some embodiments, the antigen-binding site that binds PD-L1 is an scFv or an Fab. In some embodiments, the bifunctional protein comprises an amino acid sequence at least 90% identical to an amino acid sequence selected from SEQ ID NOS: 160-164. In some embodiments, the bifunctional protein comprises an amino acid sequence selected from SEQ ID NOS: 160-164 (EP199, EP200, EP201, and EP461, respectively).

In certain embodiments, the bifunctional protein comprises a heterodimer comprising: (a) a first subunit comprising the antigen-binding site that binds PD-L1 and a first antibody heavy chain constant region; and (b) a second subunit comprising a wild-type or engineered IL-2 polypeptide or a functional fragment or variant thereof and a second antibody heavy chain constant region. In some embodiments, the antigen-binding site that binds PD-L1 is an scFv or an Fab. In some embodiments, the bifunctional protein comprises a heterodimer, wherein the first subunit is an anti-PD-L1-scFv-Fc fusion protein and the second subunit is an IL-2-Fc fusion protein. In some embodiments, the first subunit comprises an amino acid sequence at least 90% identical to SEQ ID NO:176 (EP326) and a second subunit comprising an amino acid sequence at least 90% identical to an amino acid sequence selected from SEQ ID NOS: 165-175 (EP290, EP291, EP297, EP412, EP413, EP414, EP415, EP416, EP417, EP418, EP419, respectively). In some embodiments, the first subunit comprises or consists of an amino acid sequence of SEQ ID NO: 176 and the second subunit comprises or consists of an amino acid sequence selected from SEQ ID NOS: 165-175. In some embodiments, the bifunctional protein comprises a heterodimer of proteins comprising: (a) SEQ ID NO:176 and SEQ ID NO:165; (b) SEQ ID NO:176 and SEQ ID NO: 166; (c) SEQ ID NO:176 and SEQ ID NO:167; (d) SEQ ID NO:176 and SEQ ID NO:168; (e) SEQ ID NO:176 and SEQ ID NO:169; (f) SEQ ID NO:176 and SEQ ID NO:170; (g) SEQ ID NO:176 and SEQ ID NO:171; (h) SEQ ID NO:176 and SEQ ID NO:172; (i) SEQ ID NO:176 and SEQ ID NO:173; (j) SEQ ID NO:176 and SEQ ID NO:174; or (k) SEQ ID NO:176 and SEQ ID NO:175.

In some embodiments, the bifunctional protein comprises a heterodimer, wherein the first subunit is an anti-PD-L1-Fab-Fc fusion protein and the second subunit is an IL-2-Fc fusion protein. In some embodiments, the first subunit comprises an amino acid sequence at least 90% identical to an amino acid sequence selected from SEQ ID NOS: 177-180 (EP325, EP462, EP463, EP464, respectively) and the first subunit further comprises LC domain comprising an amino acid sequence at least 90% identical to SEQ ID NO:181 (EP205), and the second subunit comprises an amino acid sequence at least 90% identical to an amino acid sequence selected from SEQ ID NOS: 165-175 (EP290, EP291, EP297, EP412, EP413, EP414, EP415, EP416, EP417, EP418, EP419, respectively). In some embodiments, the first subunit comprises or consists of an amino acid sequence selected from SEQ ID NOS: 177-180 and the LC domain comprises or consists of amino acid sequence of SEQ ID NO:181, and the second subunit comprises or consists of an amino acid sequence selected from SEQ ID NOS: 165-175. In some embodiments, the bifunctional protein comprises a heterodimer of proteins comprising: (a) a first subunit comprising SEQ ID NO:177 and SEQ ID NO: 181, SEQ ID NO:178 and SEQ ID NO:181; SEQ ID NO:179 and SEQ ID NO:181; or SEQ ID NO:180 and SEQ ID NO:181; and (b) a second subunit comprising an amino acid sequence selected from SEQ ID NO:165-175. In some embodiments, the bifunctional protein comprises a heterodimer of proteins comprising: polypeptides having the amino acid sequences selected from (a) SEQ ID NO:165, SEQ ID NO:177, and SEQ ID NO:181; (b) SEQ ID NO:166, SEQ ID NO:177, and SEQ ID NO:181; (c) SEQ ID NO:167, SEQ ID NO:177, and SEQ ID NO:181; (d) SEQ ID NO:168, SEQ ID NO: 177, and SEQ ID NO:181; (e) SEQ ID NO:169, SEQ ID NO:177, and SEQ ID NO: 181; (f) SEQ ID NO:170, SEQ ID NO: 177, and SEQ ID NO:181; (g) SEQ ID NO: 171, SEQ ID NO:177, and SEQ ID NO:181; (h) SEQ ID NO:172, SEQ ID NO:177, and SEQ ID NO:181; (i) SEQ ID NO:173, SEQ ID NO:177, and SEQ ID NO:181; (j) SEQ ID NO:174, SEQ ID NO:177, and SEQ ID NO:181; (k) SEQ ID NO: 175, SEQ ID NO: 177, and SEQ ID NO:181; (l) SEQ ID NO:171, SEQ ID NO:178, and SEQ ID NO: 181; (m) SEQ ID NO:171, SEQ ID NO:179, and SEQ ID NO:181; and (n) SEQ ID NO: 171, SEQ ID NO:180, and SEQ ID NO: 181.

In certain embodiments, the bifunctional proteins comprise: (a) a first subunit comprising the antigen-binding site that binds PD-L1 and a first antibody heavy chain constant region; and (b) a second subunit comprising the antigen-binding site that binds PD-L1, an engineered interleukin-2 (IL-2) polypeptide or a functional fragment or variant thereof and a second antibody heavy chain constant region. In some embodiments, the antigen-binding site that binds PD-L1 is an scFv or an Fab. In some embodiments, the bifunctional protein comprises a heterodimer, wherein the first subunit is an anti-PD-L1-Fab-Fc and the second subunit is an anti-PD-L1-Fab-Fc-IL-2-fusion protein. In some embodiments, the first subunit comprises an amino acid sequence at least 90% identical to SEQ ID NO:182 (EP362) and the second subunit comprises an amino acid sequence at least 90% identical to an amino acid sequence selected from SEQ ID NOS: 183-185 (EP363, EP364, EP365); optionally wherein the first and the second subunit, each independently, further comprises an amino acid sequence at least 90% identical to SEQ ID NO:181 (EP205). In some embodiments, the first subunit comprises or consists of the amino acid sequence of SEQ ID NO: 182 and the second subunit comprises or consists of an amino acid sequence selected from SEQ ID NOS: 183-185; optionally wherein the first and the second subunit, each independently, further comprises an amino acid sequence of SEQ ID NO:181. In some embodiments, the bifunctional protein comprises a heterodimer of proteins comprising: polypeptides having the amino acid sequences selected from (a) SEQ ID NO:182; SEQ ID NO: 183; and SEQ ID NO:181; (b) SEQ ID NO:182; SEQ ID NO:184; and SEQ ID NO: 181; and (c) SEQ ID NO:182; SEQ ID NO:185; and SEQ ID NO:181.

In some embodiments, the bifunctional protein binds human PD-L1 with a KD of less than about 1 nM or with a comparable or lower KD as compared to the comprised antigen-binding site that binds PD-L1, as measured by SPR. In some embodiments, the bifunctional protein binds IL-2Rβ with a K_{D} of less than about 65 nM or less than about 50 nM, as measured by SPR.

In some embodiments, the bifunctional protein binds IL-2Rα with a KD of less than about 40 nM, as measured by SPR. In some embodiments, the bifunctional protein binds IL-2Rβ with a K_{D} of less than about 100 nM, less than about 80 nM, less than about 50 nM, less than about 10 nM, or less than about 5 nM, as measured by SPR. In some embodiments, the bifunctional protein binds PD-L1 with a K_{D} of less than about 0.5 nM or less than about 0.1 nM, as measured by SPR. In some embodiments, the bifunctional protein binds IL-2Rα with a EC50 of less than about 1 nM, as measured by ELISA. In some embodiments, the bifunctional protein binds IL-2Rβ with a EC50 of less than about 5 nM, less than about 2.5 nM, less than about 1.5 nM, less than about 1 nM, or less than about 0.6 nM, as measured by ELISA. In some embodiments, the bifunctional protein binds PD-L1 with a EC50 of less than about 0.4 nM, as measured by ELISA.

In some embodiments, the bifunctional protein competes with PD-1 for binding PD-L1 or inhibits PD-L1 binding to PD-1.

In some embodiments, the bifunctional protein induces p-STAT5 expression in immune cells. In some embodiments, the bifunctional protein induces p-STAT5 expression in immune cells with EC50 of less than about 1 nM, less than about 0.6 nM, 0.5 nM, or less than about 0.1 nM, as measured in isolated human peripheral blood mononuclear cells (PBMCs), and wherein the immune cells are T cells, NK cells, or Tregs. In certain embodiments, the bifunctional protein induces p-STAT5 expression in immune cells as measured in mouse splenocytes, and wherein the immune cells are T cells, NK cells, or Tregs.

In some embodiments, the bifunctional protein inhibits tumor growth in vivo.

In some embodiments, the bifunctional protein induces immune cell proliferation in vivo. In some embodiments, the immune cell is T cell or NK cell. In some embodiments, the T cell is CD8+ T cell.

TABLE 4
Sequences of IL-2 subunits.

| | | |
|---|---|---|
| Wild-Type Human IL-2 | APTSSSTKKTQLQLEHLLLDLQMILNGINNY | SEQ ID NO: 191 |
| | KNPKLTRMLTFKFYMPKKATELKHLQCLEE | |
| | ELKPLEEVLNLAQSKNFHLRPRDLISNINVIV | |
| | LELKGSETTFMCEYADETATIVEFLNRWITF | |
| | CQSIISTLT | |
| IL-2 with modified IL-2R | APTSSSTKKTQLQLEHLLLDLQMILNGINNY | SEQ ID NO: 192 |
| binding region 2 | KNPKLTRMLTFKFYMPKKATELKHLQCLEE | |
| | ELKPLEEVLNLAQSKNFHLX₁X₂X₃DX₄X₅X₆N | |
| | X₇X₈X₉X₁₀X₁₁X₁₂X₁₃LKGSETTFMCEYADETA | |
| | TIVEFLNRWITFCQSIISTLT | |
| IL-2Rβ binding region 2 | X₁-X₂-X₃-D-X₄-X-₅-X₆-N-X₇-X₈-X₉-X₁₀-X₁₁- | SEQ ID NO: 95 |
| | X₁₂-X₁₃ | |
| IL-2Rβ binding region 2 | GVTDSISNAIVLARE | SEQ ID NO: 96 |
| IL-2Rβ binding region 2 | KWGDAVSNARVLAGE | SEQ ID NO: 97 |
| IL-2Rβ binding region 2 | KWGDAVSNARVLAGA | SEQ ID NO: 98 |
| IL-2Rβ binding region 2 | TLMDTTDNIGVLVRE | SEQ ID NO: 99 |
| IL-2Rβ binding region 2 | EPSDVISNINVLVQE | SEQ ID NO: 100 |
| IL-2Rβ binding region 2 | SPQDSIENISVLVRE | SEQ ID NO: 101 |
| IL-2Rβ binding region 2 | WASDSIENITLLIQE | SEQ ID NO: 102 |
| IL-2Rβ binding region 2 | CPTDTIENITVLIQE | SEQ ID NO: 103 |
| IL-2Rβ binding region 2 | RYKDSLENMQIIIQE | SEQ ID NO: 104 |
| IL-2Rβ binding region 2 | TARDAVDNMRVIIQE | SEQ ID NO: 105 |
| IL-2Rβ binding region 2 | TPRDVVENMNVLVLE | SEQ ID NO: 106 |
| IL-2Rβ binding region 2 | TPSDVIENMEVLILD | SEQ ID NO: 107 |
| IL-2Rβ binding region 2 | TPSDAIENINVLIRE | SEQ ID NO: 108 |
| IL-2Rβ binding region 2 | TPSDVIENITVLVQE | SEQ ID NO: 109 |
| IL-2Rβ binding region 2 | GVGDTIDNINVLVKE | SEQ ID NO: 110 |
| IL-2Rβ binding region 2 | IGRDSIDNIKVIVQE | SEQ ID NO: 111 |
| IL-2Rβ binding region 2 | WATDTIRNVEVLVQE | SEQ ID NO: 112 |
| IL-2Rβ binding region 2 | TAEDVVTNITVLVQE | SEQ ID NO: 113 |
| IL-2Rβ binding region 2 | TAEDVISNIRVNVQE | SEQ ID NO: 114 |
| IL-2Rβ binding region 2 | TPSDVIDNVSITVQE | SEQ ID NO: 115 |
| IL-2Rβ binding region 2 | TARDAISNIRVIVQE | SEQ ID NO: 116 |
| IL-2Rβ binding region 2 | RARDAIDNIRVIVQE | SEQ ID NO: 117 |
| IL-2Rβ binding region 2 | TPRDAIDNINVIIQE | SEQ ID NO: 118 |
| IL-2Rβ binding region 2 | TPRDAIDNIRVIVQE | SEQ ID NO: 119 |
| IL-2Rβ binding region 2 | TPRDAIDNIRVIILE | SEQ ID NO: 120 |
| IL-2Rβ binding region 2 | TARDAISNINVIIQE | SEQ ID NO: 121 |
| IL-2Rβ binding region 2 | TARDAIDNINVIVOE | SEQ ID NO: 122 |
| IL-2Rβ binding region 2 | TARDAIDNIRVIVLE | SEQ ID NO: 123 |
| IL-2Rα binding region 1 | PVLTRMLTIKFY | SEQ ID NO: 124 |
| IL-2Rα binding region 1 | PKLTRMLTLKFP | SEQ ID NO: 125 |
| IL-2Rα binding region 1 | PDLTSMLAFKFY | SEQ ID NO: 126 |
| IL-2Rα binding region 1 | PGLTEMLTFKFY | SEQ ID NO: 127 |
| IL-2Rα binding region 1 | PSLTRMLTGKFY | SEQ ID NO: 128 |
| IL-2Rα binding region 1 | PELTIMLTPKFY | SEQ ID NO: 129 |
| IL-2Rα binding region 1 | PCLTAMLTLKFA | SEQ ID NO: 130 |
| IL-2Rα binding region 1 | PKLTRMLTHKFV | SEQ ID NO: 131 |
| IL-2Rα binding region 1 | PCLTDMLTFKFY | SEQ ID NO: 132 |
| IL-2Rα binding region 1 | PLLTDMLTRKFY | SEQ ID NO: 133 |
| IL-2Rα binding region 1 | PLLTDMLTFKFY | SEQ ID NO: 134 |
| IL-2Rα binding region 1 | PKLTDMLTFKFS | SEQ ID NO: 135 |
| IL-2Rα binding region 1 | PKLTYMLTRKFY | SEQ ID NO: 136 |
| IL-2Rα binding region 1 | PKLTRMLTFKFC | SEQ ID NO: 137 |
| IL-2Rα binding region 1 | PKLTSMLTFKFS | SEQ ID NO: 138 |
| IL-2Rα binding region 1 | PKLTYMLTFKFS | SEQ ID NO: 139 |
| IL-2Rα binding region 1 | PKLTGMLTFKFS | SEQ ID NO: 140 |
| IL-2Rα binding region 1 | PKLTVMLTFKFT | SEQ ID NO: 141 |
| IL-2Rα binding region 1 | PKLTVMLTFKFS | SEQ ID NO: 142 |
| IL-2Rα binding region 1 | PKLTVMLTFKFP | SEQ ID NO: 143 |
| IL-2Rα binding region 1 | PKLTVMLTFKFF | SEQ ID NO: 144 |
| IL-2Rα binding region 1 | PKLTCMLTFKFA | SEQ ID NO: 145 |
| IL-2Rα binding region 1 | PKLTNMLTFKFA | SEQ ID NO: 146 |
| IL-2Rα binding region 1 | PKLTNMLTFKFS | SEQ ID NO: 147 |

TABLE 5
Sequences of Bifunctional Proteins Comprising an anti-PD-L1 scFv and
IL-2 subunit.

| | | |
|---|---|---|
| EP199 | QVQLVQSGAEVKKPGSSVKVSCKASGGTFSSYAISWVRQAPGQGLEW | SEQ ID |
| 2019EP69-F03; IL2 | MGGIIPIFGTANYAQKFQGRVTITADESTSTAYMELSSLRSEDTAVYYC | NO: 160 |
| with C125S | ARDKWYGSAWRGDYWGQGTLVTVSSGGGGSGGGGSGGGGSQSVLT | |
| | QPPSASGTPGQRVTISCSGSYSNIGSNFVNWYQQLPGTAPKLLIHFNNQ | |
| | RPSGVPDRFSGSRSGTSASLAISGLQSEDEADYFCSTWDDSLKGPIFGG | |
| | GTKLTVLGGGGSGGGGSGGGGSGGGGSAPTSSSTKKTQLQLEHLLLDL | |
| | QMILNGINNYKNPKLTRMLTFKFYMPKKATELKHLQCLEEELKPLEEV | |
| | LNLAQSKNFHLRPRDLISNINVIVLELKGSETTFMCEYADETATIVEFLN | |
| | RWITFAQSIISTLT | |
| EP200 | APTSSSTKKTQLQLEHLLLDLQMILNGINNYKNPKLTRMLTFKFYMPK | SEQ ID |
| 2019EP69-F03; IL2 | KATELKHLQCLEEELKPLEEVLNLAQSKNFHLRPRDLISNINVIVLELKG | NO: 161 |
| with C125S | SETTFMCEYADETATIVEFLNRWITFAQSIISTLTGGGGSGGGGSGGGG | |
| | SGGGGSQVQLVQSGAEVKKPGSSVKVSCKASGGTFSSYAISWVRQAP | |
| | GQGLEWMGGIIPIFGTANYAQKFQGRVTITADESTSTAYMELSSLRSED | |
| | TAVYYCARDKWYGSAWRGDYWGQGTLVTVSSGGGGSGGGGSGGGG | |
| | SQSVLTQPPSASGTPGQRVTISCSGSYSNIGSNFVNWYQQLPGTAPKLLI | |
| | HFNNQRPSGVPDRFSGSRSGTSASLAISGLQSEDEADYFCSTWDDSLKG | |
| | PIFGGGTKLTVL | |
| EP201 | QVQLVQSGAEVKKPGSSVKVSCKASGGTFSSYAISWVRQAPGQGLEW | SEQ ID |
| 2019EP69-F03; IL2 | MGGIIPIFGTANYAQKFQGRVTITADESTSTAYMELSSLRSEDTAVYYC | NO: 162 |
| with SEQ ID NO: 105 | ARDKWYGSAWRGDYWGQGTLVTVSSGGGGSGGGGSGGGGSQSVLT | |
| and C125S | QPPSASGTPGQRVTISCSGSYSNIGSNFVNWYQQLPGTAPKLLIHFNNQ | |
| | RPSGVPDRFSGSRSGTSASLAISGLQSEDEADYFCSTWDDSLKGPIFGG | |
| | GTKLTVLGGGGSGGGGSGGGGSGGGGSAPTSSSTKKTQLQLEHLLLDL | |
| | QMILNGINNYKNPKLTEMLTFKFYMPKKATELKHLQCLEEELKPLEEV | |
| | LNLAQSKNFHLTARDAVDNMRVIIQELKGSETTFMCEYADETATIVEF | |
| | LNRWITFAQSIISTLT | |
| EP202 | APTSSSTKKTQLQLEHLLLDLQMILNGINNYKNPKLTEMLTFKFYMPK | SEQ ID |
| 2019EP69-F03; IL2 | KATELKHLQCLEEELKPLEEVLNLAQSKNFHLTARDAVDNMRVIIQEL | NO: 163 |
| with SEQ ID NO: 105 | KGSETTFMCEYADETATIVEFLNRWITFAQSIISTLTGGGGSGGGGSGG | |
| and C125S | GGSGGGGSQVQLVQSGAEVKKPGSSVKVSCKASGGTFSSYAISWVRQ | |
| | APGQGLEWMGGIIPIFGTANYAQKFQGRVTITADESTSTAYMELSSLRS | |
| | EDTAVYYCARDKWYGSAWRGDYWGQGTLVTVSSGGGGSGGGGSGG | |
| | GGSQSVLTQPPSASGTPGQRVTISCSGSYSNIGSNFVNWYQQLPGTAPK | |
| | LLIHFNNQRPSGVPDRFSGSRSGTSASLAISGLQSEDEADYFCSTWDDS | |
| | LKGPIFGGGTKLTVL | |
| EP461 | QVQLVQSGAEVKKPGSSVKVSCKASGGTFSSYAISWVRQAPGQGLEW | SEQ ID |
| 2019EP69-F03; IL2 | MGGIIPIFGTANYAQKFQGRVTITADESTSTAYMELSSLRSEDTAVYYC | NO: 164 |
| with | ARDKWYGSAWRGDYWGQGTLVTVSSGGGGGGGGSGGGGSQSVLT | |
| K35L/R38D/F42R | QPPSASGTPGQRVTISCSGSYSNIGSNFVNWYQQLPGTAPKLLIHFNNQ | |
| mutations | RPSGVPDRFSGSRSGTSASLAISGLQSEDEADYFCSTWDDSLKGPIFGG | |
| | GTKLTVLGGGGSGGGGSGGGGSGGGGSAPTSSSTKKTQLQLEHLLLDL | |
| | QMILNGINNYKNPLLTDMLTRFKFYMPKKATELKHLQCLEEELKPLEE | |
| | VLNLAQSKNFHLRPRDLISNINVIVLELKGSETTFMCEYADETATIVEFL | |
| | NRWITFAQSIISTLT | |

TABLE 6
Sequences of Component Parts of Bifunctional Proteins Comprising an IgG1
Fc Knob or Hole Mutations, anti-PD-L1 antigen-Binding Site, and/or IL-2, IL-15, or
IL15Rα.

| | | |
|---|---|---|
| EP290 | APASSSTKKTQLQLEHLLLDLQMILNGINNYKNPKLTR | SEQ ID |
| IL2 fused to IgG1 Fc with | MLTFKFYMPKKATELKHLQCLEEELKPLEEVLNLAQSK | NO: 165 |
| Knob and LALAPG | NFHLRPRDLISNINVIVLELKGSETTFMCEYADETATIVE | |
| mutation | FLNRWITFSQSIISTLTDKTHTCPPCPAPEAAGGPSVFLFP | |
| | PKPKDTLMISRTPEVTCVVVDVSHEDPEVKFNWYVDG | |
| | VEVHNAKTKPREEQYNSTYRVVSVLTVLHQDWLNGKE | |
| | YKCKVSNKALGAPIEKTISKAKGQPREPQVYTLPPCRDE | |
| | LTKNQVSLWCLVKGFYPSDIAVEWESNGQPENNYKTTP | |
| | PVLDSDGSFFLYSALTVDKSRWQQGNVFSCSVMHEAL | |
| | HNHYTQKSLSLSPGK | |
| EP291 | APASSSTKKTQLQLEHLLLDLQMILNGINNYKNPKLTR | SEQ ID |
| IL2 EP003 fused to IgG1 | MLTFKFYMPKKATELKHLQCLEEELKPLEEVLNLAQSK | NO: 166 |
| Fc with Knob and | NFHLGVTDSISNAIVLARELKGSETTFMCEYADETATIV | |
| LALAPG mutation | EFLNRWITFSQSIISTLTDKTHTCPPCPAPEAAGGPSVFLF | |
| | PPKPKDTLMISRTPEVTCVVVDVSHEDPEVKFNWYVDG | |
| | VEVHNAKTKPREEQYNSTYRVVSVLTVLHQDWLNGKE | |
| | YKCKVSNKALGAPIEKTISKAKGQPREPQVYTLPPCRDE | |
| | LTKNQVSLWCLVKGFYPSDIAVEWESNGQPENNYKTTP | |
| | PVLDSDGSFFLYSALTVDKSRWQQGNVFSCSVMHEAL | |
| | HNHYTQKSLSLSPGK | |
| EP297 | APASSSTKKTQLQLEHLLLDLQMILNGINNYKNPKLTE | SEQ ID |
| IL2 EP001 fused to IgG1 | MLTFKFYMPKKATELKHLQCLEEELKPLEEVLNLAQSK | NO: 167 |
| Fc with Knob and | NFHLTARDAVDNMRVIIQELKGSETTFMCEYADETATI | |
| LALAPG mutation | VEFLNRWITFSQSIISTLTDKTHTCPPCPAPEAAGGPSVF | |
| | LFPPKPKDTLMISRTPEVTCVVVDVSHEDPEVKFNWYV | |
| | DGVEVHNAKTKPREEQYNSTYRVVSVLTVLHQDWLN | |
| | GKEYKCKVSNKALGAPIEKTISKAKGQPREPQVYTLPPC | |
| | RDELTKNQVSLWCLVKGFYPSDIAVEWESNGQPENNY | |
| | KTTPPVLDSDGSFFLYSALTVDKSRWQQGNVFSCSVMH | |
| | EALHNHYTQKSLSLSPGK | |
| EP412 | APASSSTKKTQLQLEHLLLDLQMILNGINNYKNPLLTD | SEQ ID |
| IL2 with | MLTRKFYMPKKATELKHLQCLEEELKPLEEVLNLAQSK | NO: 168 |
| T3A/K35L/R38D/F42R | NFHLRPRDLISNINVIVLELKGSETTFMCEYADETATIVE | |
| fused to IgG1 Fc with | FLNRWITFSQSIISTLTDKTHTCPPCPAPEAAGGPSVFLFP | |
| Knob and LALAPG | PKPKDTLMISRTPEVTCVVVDVSHEDPEVKFNWYVDG | |
| mutation | VEVHNAKTKPREEQYNSTYRVVSVLTVLHQDWLNGKE | |
| | YKCKVSNKALGAPIEKTISKAKGQPREPQVYTLPPCRDE | |
| | LTKNQVSLWCLVKGFYPSDIAVEWESNGQPENNYKTTP | |
| | PVLDSDGSFFLYSALTVDKSRWQQGNVFSCSVMHEAL | |
| | HNHYTQKSLSLSPGK | |
| EP413 | APASSSTKKTQLQLEHLLLDLQMILNGINNYKNPLLTD | SEQ ID |
| IL2 EP001 carrying | MLTRKFYMPKKATELKHLQCLEEELKPLEEVLNLAQSK | NO: 169 |
| K35L/R38D/F42R fused | NFHLTARDAVDNMRVIIQELKGSETTFMCEYADETATI | |
| to IgG1 Fc with Knob and | VEFLNRWITFSQSIISTLTDKTHTCPPCPAPEAAGGPSVF | |
| LALAPG mutation | LFPPKPKDTLMISRTPEVTCVVVDVSHEDPEVKFNWYV | |
| | DGVEVHNAKTKPREEQYNSTYRVVSVLTVLHQDWLN | |
| | GKEYKCKVSNKALGAPIEKTISKAKGQPREPQVYTLPPC | |
| | RDELTKNQVSLWCLVKGFYPSDIAVEWESNGQPENNY | |
| | KTTPPVLDSDGSFFLYSALTVDKSRWQQGNVFSCSVMH | |
| | EALHNHYTQKSLSLSPGK | |
| EP414 | APASSSTKKTQLQLEHLLLDLQMILNGINNYKNPLLTD | SEQ ID |
| IL2 EP003 carrying | MLTRKFYMPKKATELKHLQCLEEELKPLEEVLNLAQSK | NO: 170 |
| K35L/R38D/F42R fused | NFHLGVTDSISNAIVLARELKGSETTFMCEYADETATIV | |
| to IgG1 Fc with Knob and | EFLNRWITFSQSIISTLTDKTHTCPPCPAPEAAGGPSVFLF | |
| LALAPG mutation | PPKPKDTLMISRTPEVTCVVVDVSHEDPEVKFNWYVDG | |
| | VEVHNAKTKPREEQYNSTYRVVSVLTVLHQDWLNGKE | |
| | YKCKVSNKALGAPIEKTISKAKGQPREPQVYTLPPCRDE | |
| | LTKNQVSLWCLVKGFYPSDIAVEWESNGQPENNYKTTP | |
| | PVLDSDGSFFLYSALTVDKSRWQQGNVFSCSVMHEAL | |
| | HNHYTQKSLSLSPGK | |
| EP415 | APASSSTKKTQLQLEHLLLDLQMILNGINNYKNPLLTD | SEQ ID |
| IL2 EP337 fused to IgG1 | MLTRKFYMPKKATELKHLQCLEEELKPLEEVLNLAQSK | NO: 171 |
| Fc with Knob and | NFHLTARDAIDNINVIVQELKGSETTFMCEYADETATIV | |
| LALAPG mutation | EFLNRWITFSQSIISTLTEPKSSDKTHTCPPCPAPEAAGGP | |
| | SVFLFPPKPKDTLMISRTPEVTCVVVDVSHEDPEVKFN | |
| | WYVDGVEVHNAKTKPREEQYNSTYRVVSVLTVLHQD | |
| | WLNGKEYKCKVSNKALGAPIEKTISKAKGQPREPQVYT | |
| | LPPCRDELTKNQVSLWCLVKGFYPSDIAVEWESNGQPE | |
| | NNYKTTPPVLDSDGSFFLYSALTVDKSRWQQGNVFSCS | |
| | VMHEALHNHYTQKSLSLSPGK | |
| EP416 | APASSSTKKTQLQLEHLLLDLQMILNGINNYKNPLLTD | SEQ ID |
| IL2 EP338 fused to IgG1 | MLTRKFYMPKKATELKHLQCLEEELKPLEEVLNLAQSK | NO: 172 |
| Fc with Knob and | NFHLTARDAISNINVIIQELKGSETTFMCEYADETATIVE | |
| LALAPG mutation | FLNRWITFSQSIISTLTEPKSSDKTHTCPPCPAPEAAGGPS | |
| | VFLFPPKPKDTLMISRTPEVTCVVVDVSHEDPEVKFNW | |
| | YVDGVEVHNAKTKPREEQYNSTYRVVSVLTVLHQDW | |
| | LNGKEYKCKVSNKALGAPIEKTISKAKGQPREPQVYTL | |
| | PPCRDELTKNQVSLWCLVKGFYPSDIAVEWESNGQPEN | |
| | NYKTTPPVLDSDGSFFLYSALTVDKSRWQQGNVFSCSV | |
| | MHEALHNHYTQKSLSLSPGK | |
| EP417 | APASSSTKKTQLQLEHLLLDLQMILNGINNYKNPLLTD | SEQ ID |
| IL2 EP339 fused to IgG1 | MLTRKFYMPKKATELKHLQCLEEELKPLEEVLNLAQSK | NO: 173 |
| Fc with Knob and | NFHLTPRDAIDNIRVIVQELKGSETTFMCEYADETATIV | |
| LALAPG mutation | EFLNRWITFSQSIISTLTEPKSSDKTHTCPPCPAPEAAGGP | |
| | SVFLFPPKPKDTLMISRTPEVTCVVVDVSHEDPEVKFN | |
| | WYVDGVEVHNAKTKPREEQYNSTYRVVSVLTVLHQD | |
| | WLNGKEYKCKVSNKALGAPIEKTISKAKGQPREPQVYT | |
| | LPPCRDELTKNQVSLWCLVKGFYPSDIAVEWESNGQPE | |
| | NNYKTTPPVLDSDGSFFLYSALTVDKSRWQQGNVFSCS | |
| | VMHEALHNHYTQKSLSLSPGK | |
| EP418 | APASSSTKKTQLQLEHLLLDLQMILNGINNYKNPLLTD | SEQ ID |
| IL2 EP340 fused to IgG1 | MLTRKFYMPKKATELKHLQCLEEELKPLEEVLNLAQSK | NO: 174 |
| Fc with Knob and | NFHLTPRDAIDNIRVIILELKGSETTFMCEYADETATIVE | |
| LALAPG mutation | FLNRWITFSQSIISTLTEPKSSDKTHTCPPCPAPEAAGGPS | |
| | VFLFPPKPKDTLMISRTPEVTCVVVDVSHEDPEVKFNW | |
| | YVDGVEVHNAKTKPREEQYNSTYRVVSVLTVLHQDW | |
| | LNGKEYKCKVSNKALGAPIEKTISKAKGQPREPQVYTL | |
| | PPCRDELTKNQVSLWCLVKGFYPSDIAVEWESNGQPEN | |
| | NYKTTPPVLDSDGSFFLYSALTVDKSRWQQGNVFSCSV | |
| | MHEALHNHYTQKSLSLSPGK | |
| EP419 | APASSSTKKTQLQLEHLLLDLQMILNGINNYKNPKLTD | SEQ ID |
| IL2 EP343 fused to IgG1 | MLTFKFSMPKKATELKHLQCLEEELKPLEEVLNLAQSK | NO: 175 |
| Fc with Knob and | NFHLTPRDAIDNIRVIVQELKGSETTFMCEYADETATIV | |
| LALAPG mutation | EFLNRWITFSQSIISTLTEPKSSDKTHTCPPCPAPEAAGGP | |
| | SVFLFPPKPKDTLMISRTPEVTCVVVDVSHEDPEVKFN | |
| | WYVDGVEVHNAKTKPREEQYNSTYRVVSVLTVLHQD | |
| | WLNGKEYKCKVSNKALGAPIEKTISKAKGQPREPQVYT | |
| | LPPCRDELTKNQVSLWCLVKGFYPSDIAVEWESNGQPE | |
| | NNYKTTPPVLDSDGSFFLYSALTVDKSRWQQGNVFSCS | |
| | VMHEALHNHYTQKSLSLSPGK | |
| EP326 | QVQLVQSGAEVKKPGSSVKVSCKASGGTFSSYAISWVR | SEQ ID |
| 2019EP69-F03, ScFv- | QAPGQGLEWMGGIIPIFGTANYAQKFQGRVTITADESTS | NO: 176 |
| IgG1, with hole and | TAYMELSSLRSEDTAVYYCARDKWYGSAWRGDYWGQ | |
| LALAPG mutation | GTLVTVSSGGGGSGGGGSGGGGSQSVLTQPPSASGTPG | |
| | QRVTISCSGSYSNIGSNFVNWYQQLPGTAPKLLIHFNNQ | |
| | RPSGVPDRFSGSRSGTSASLAISGLQSEDEADYFCSTWD | |
| | DSLKGPIFGGGTKLTVLDKTHTCPPCPAPEAAGGPSVFL | |
| | FPPKPKDTLMISRTPEVTCVVVDVSHEDPEVKFNWYVD | |
| | GVEVHNAKTKPREEQYNSTYRVVSVLTVLHQDWLNG | |
| | KEYKCKVSNKALGAPIEKTISKAKGQPREPQVCTLPPSR | |
| | DELTKNQVSLSCAVKGFYPSDIAVEWESNGQPENNYKT | |
| | TPPVLDSDGSFKLVSKLTVDKSRWQQGNVFSCSVMHE | |
| | ALHNHYTQKSLSLSPGK | |
| EP325 | QVQLVQSGAEVKKPGSSVKVSCKASGGTFSSYAISWVR | SEQ ID |
| 2019EP69-F03, Anti- | QAPGQGLEWMGGIIPIFGTANYAQKFQGRVTITADESTS | NO: 177 |
| PDL1, HC-IgG1, hole | TAYMELSSLRSEDTAVYYCARDKWYGSAWRGDYWGQ | |
| with Y349C, T366S, | GTLVTVSSASTKGPSVFPLAPSSKSTSGGTAALGCLVKD | |
| L368A, F405K, Y407V; | YFPEPVTVSWNSGALTSGVHTFPAVLQSSGLYSLSSVVT | |
| LALAPG mutation | VPSSSLGTQTYICNVNHKPSNTKVDKKVEPKSCDKTHT | |
| | CPPCPAPEAAGGPSVFLFPPKPKDTLMISRTPEVTCVVV | |
| | DVSHEDPEVKFNWYVDGVEVHNAKTKPREEQYNSTYR | |
| | VVSVLTVLHQDWLNGKEYKCKVSNKALGAPIEKTISKA | |
| | KGQPREPQVCTLPPSRDELTKNQVSLSCAVKGFYPSDIA | |
| | VEWESNGQPENNYKTTPPVLDSDGSFKLVSKLTVDKSR | |
| | WQQGNVFSCSVMHEALHNHYTQKSLSLSPGK | |
| EP462 | QVQLVQSGAEVKKPGSSVKVSCKASGGTFSSYAISWVR | SEQ ID |
| 2018EP171-H02, HC- | QAPGQGLEWMGGIIPIFGTANYAQKFQGRVTITADESTS | NO: 178 |
| IgG1, with hole and | TAYMELSSLRSEDTAVYYCARDKGYGSGWRGDYWGQ | |
| LALAPG mutation | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPEAAGGPSVFLFPPKPKDTLMISRTPEVTCVVV | |
| | DVSHEDPEVKFNWYVDGVEVHNAKTKPREEQYNSTYR | |
| | VVSVLTVLHQDWLNGKEYKCKVSNKALGAPIEKTISKA | |
| | KGQPREPQVCTLPPSRDELTKNQVSLSCAVKGFYPSDIA | |
| | VEWESNGQPENNYKTTPPVLDSDGSFKLVSKLTVDKSR | |
| | WQQGNVFSCSVMHEALHNHYTQKSLSLSPGK | |
| EP463 | QVQLVQSGAEVKKPGSSVKVSCKASGGTFSSYAISWVR | SEQ ID |
| 2019EP69-E10, HC- | QAPGQGLEWMGGIIPIFGTANYAQKFQGRVTITADESTS | NO: 179 |
| IgG1, with hole and | TAYMELSSLRSEDTAVYYCARDKGFGSGWRGDYWGQ | |
| LALAPG mutation | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPEAAGGPSVFLFPPKPKDTLMISRTPEVTCVVV | |
| | DVSHEDPEVKFNWYVDGVEVHNAKTKPREEQYNSTYR | |
| | VVSVLTVLHQDWLNGKEYKCKVSNKALGAPIEKTISKA | |
| | KGQPREPQVCTLPPSRDELTKNQVSLSCAVKGFYPSDIA | |
| | VEWESNGQPENNYKTTPPVLDSDGSFKLVSKLTVDKSR | |
| | WQQGNVFSCSVMHEALHNHYTQKSLSLSPGK | |
| EP464 | QVQLVQSGAEVKKPGSSVKVSCKASGGTFSSYAISWVR | SEQ ID |
| 2019EP69-F02, HC-IgG1, | QAPGQGLEWMGGIIPIFGTANYAQKFQGRVTITADESTS | NO: 180 |
| with hole and LALAPG | TAYMELSSLRSEDTAVYYCARDKWYGSGWRGDYWGQ | |
| mutation | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPEAAGGPSVFLFPPKPKDTLMISRTPEVTCVVV | |
| | DVSHEDPEVKFNWYVDGVEVHNAKTKPREEQYNSTYR | |
| | VVSVLTVLHQDWLNGKEYKCKVSNKALGAPIEKTISKA | |
| | KGQPREPQVCTLPPSRDELTKNQVSLSCAVKGFYPSDIA | |
| | VEWESNGQPENNYKTTPPVLDSDGSFKLVSKLTVDKSR | |
| | WQQGNVFSCSVMHEALHNHYTQKSLSLSPGK | |
| EP205 | QSVLTQPPSASGTPGQRVTISCSGSYSNIGSNFVNWYQQ | SEQ ID |
| 2019EP69-F03, Anti- | LPGTAPKLLIHFNNQRPSGVPDRFSGSRSGTSASLAISGL | NO: 181 |
| PDL1, LC | QSEDEADYFCSTWDDSLKGPIFGGGTKLTVLGQPKANP | |
| | TVTLFPPSSEELQANKATLVCLISDFYPGAVTVAWKAD | |
| | GSPVKAGVETTKPSKQSNNKYAASSYLSLTPEQWKSHR | |
| | SYSCQVTHEGSTVEKTVAPTECS | |
| EP362 | QVQLVQSGAEVKKPGSSVKVSCKASGGTFSSYAISWVR | SEQ ID |
| 2019EP69-F03 HC-IgG1 | QAPGQGLEWMGGIIPIFGTANYAQKFQGRVTITADESTS | NO: 182 |
| with Knob and LALAPG | TAYMELSSLRSEDTAVYYCARDKWYGSAWRGDYWGQ | |
| mutation | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPEAAGGPSVFLFPPKPKDTLMISRTPEVTCVVV | |
| | DVSHEDPEVKFNWYVDGVEVHNAKTKPREEQYNSTYR | |
| | VVSVLTVLHQDWLNGKEYKCKVSNKALGAPIEKTISKA | |
| | KGQPREPQVYTLPPCRDELTKNQVSLWCL VKGFYPSDI | |
| | AVEWESNGQPENNYKTTPPVLDSDGSFFLYSALTVDKS | |
| | RWQQGNVFSCSVMHEALHNHYTQKSLSLSPGK | |
| EP363 | QVQLVQSGAEVKKPGSSVKVSCKASGGTFSSYAISWVR | SEQ ID |
| 2019EP69-F03 HC-IgG1 | QAPGQGLEWMGGIIPIFGTANYAQKFQGRVTITADESTS | NO: 183 |
| with hole and LALAPG | TAYMELSSLRSEDTAVYYCARDKWYGSAWRGDYWGQ | |
| mutation; fused with IL2; | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPEAAGGPSVFLFPPKPKDTLMISRTPEVTCVVV | |
| | DVSHEDPEVKFNWYVDGVEVHNAKTKPREEQYNSTYR | |
| | VVSVLTVLHQDWLNGKEYKCKVSNKALGAPIEKTISKA | |
| | KGQPREPQVCTLPPSRDELTKNQVSLSCAVKGFYPSDIA | |
| | VEWESNGQPENNYKTTPPVLDSDGSFKLVSKLTVDKSR | |
| | WQQGNVFSCSVMHEALHNHYTQKSLSLSPGKGGGGSG | |
| | GGGSGGGGSGGGGSAPASSSTKKTQLQLEHLLLDLQMI | |
| | LNGINNYKNPKLTRMLTFKFYMPKKATELKHLQCLEEE | |
| | LKPLEEVLNLAQSKNFHLRPRDLISNINVIVLELKGSETT | |
| | FMCEYADETATIVEFLNRWITFSQSIISTLT | |
| EP364 | QVQLVQSGAEVKKPGSSVKVSCKASGGTFSSYAISWVR | SEQ ID |
| 2019EP69-F03 HC-IgG1 | QAPGQGLEWMGGIIPIFGTANYAQKFQGRVTITADESTS | NO: 184 |
| with hole and LALAPG | TAYMELSSLRSEDTAVYYCARDKWYGSAWRGDYWGQ | |
| mutation; fused with | GTLVTVSSASTKGPSVFPLAPSSKSTSGGTAALGCLVKD | |
| EP001 | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPEAAGGPSVFLFPPKPKDTLMISRTPEVTCVVV | |
| | DVSHEDPEVKFNWYVDGVEVHNAKTKPREEQYNSTYR | |
| | VVSVLTVLHQDWLNGKEYKCKVSNKALGAPIEKTISKA | |
| | KGQPREPQVCTLPPSRDELTKNQVSLSCAVKGFYPSDIA | |
| | VEWESNGQPENNYKTTPPVLDSDGSFKLVSKLTVDKSR | |
| | WQQGNVFSCSVMHEALHNHYTQKSLSLSPGKGGGGSG | |
| | GGGSGGGGSGGGGSAPASSSTKKTQLQLEHLLLDLQMI | |
| | LNGINNYKNPKLTRMLTFKFYMPKKATELKHLQCLEEE | |
| | LKPLEEVLNLAQSKNFHLTARDAVDNMRVIIQELKGSE | |
| | TTFMCEYADETATIVEFLNRWITFSQSIISTLT | |
| EP365 | QVQLVQSGAEVKKPGSSVKVSCKASGGTFSSYAISWVR | SEQ ID |
| 2019EP69-F03 HC-IgG1 | QAPGQGLEWMGGIIPIFGTANYAQKFQGRVTITADESTS | NO: 185 |
| with hole and LALAPG | TAYMELSSLRSEDTAVYYCARDKWYGSAWRGDYWGQ | |
| mutation; fused with IL2 | GTLVTVSSASTKGPSVFPLAPSSKSTSGGTAALGCLVKD | |
| EP250 carrying | YFPEPVTVSWNSGALTSGVHTFPAVLQSSGLYSLSSVVT | |
| T3A/R38V/F45S | VPSSSLGTQTYICNVNHKPSNTKVDKKVEPKSCDKTHT | |
| | CPPCPAPEAAGGPSVFLFPPKPKDTLMISRTPEVTCVVV | |
| | DVSHEDPEVKFNWYVDGVEVHNAKTKPREEQYNSTYR | |
| | VVSVLTVLHQDWLNGKEYKCKVSNKALGAPIEKTISKA | |
| | KGQPREPQVCTLPPSRDELTKNQVSLSCAVKGFYPSDIA | |
| | VEWESNGQPENNYKTTPPVLDSDGSFKLVSKLTVDKSR | |
| | WQQGNVFSCSVMHEALHNHYTQKSLSLSPGKGGGGSG | |
| | GGGSGGGGSGGGGSAPASSSTKKTQLQLEHLLLDLQMI | |
| | LNGINNYKNPKLTVMLTFKFSMPKKATELKHLQCLEEE | |
| | LKPLEEVLNLAQSKNFHLTPRDAISNIRVIIQELKGSETT | |
| | FMCEYADETATIVEFLNRWITFSQSIISTLT | |
| EP203 | QVQLVQSGAEVKKPGSSVKVSCKASGGTFSSYAISWVR | SEQ ID |
| 2019EP69-F03 HC-IgG1 | QAPGQGLEWMGGIIPIFGTANYAQKFQGRVTITADESTS | NO: 186 |
| with Knob mutations; | TAYMELSSLRSEDTAVYYCARDKWYGSAWRGDYWGQ | |
| fused with IL15 (50-162) | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKAK | |
| | GQPREPQVYTLPPCRDELTKNQVSLWCLVKGFYPSDIA | |
| | VEWESNGQPENNYKTTPPVLDSDGSFFLYSALTVDKSR | |
| | WQQGNVFSCSVMHEALHNHYTQKSLSLSPGKGGGGSG | |
| | GGGSGGGGSGGGGSWVNVISDLKKIEDLIQSMHIDATL | |
| | YTESDVHPSCKVTAMKCFLLELQVISLESGDASIHDTVE | |
| | NLIILANNSLSSNGNVTESGCKECEELEEKNIKEFLQSFV | |
| | HIVQMFINTS | |
| EP204 | QVQLVQSGAEVKKPGSSVKVSCKASGGTFSSYAISWVR | SEQ ID |
| 2019EP69-F03 HC-IgG1 | QAPGQGLEWMGGIIPIFGTANYAQKFQGRVTITADESTS | NO: 187 |
| with hole mutations; | TAYMELSSLRSEDTAVYYCARDKWYGSAWRGDYWGQ | |
| fused with IL15Rα (31-97) | GTLVTVSSASTKGPSVFPLAPSSKSTSGGTAALGCLVKD | |
| | YFPEPVTVSWNSGALTSGVHTFPAVLQSSGLYSLSSVVT | |
| | VPSSSLGTQTYICNVNHKPSNTKVDKKVEPKSCDKTHT | |
| | CPPCPAPELLGGPSVFLFPPKPKDTLMISRTPEVTCVVVD | |
| | VSHEDPEVKFNWYVDGVEVHNAKTKPREEQYNSTYRV | |
| | VSVLTVLHQDWLNGKEYKCKVSNKALPAPIEKTISKAK | |
| | GQPREPQVCTLPPSRDELTKNQVSLSCAVKGFYPSDIAV | |
| | EWESNGQPENNYKTTPPVLDSDGSFKLVSKLTVDKSR | |
| | WQQGNVFSCSVMHEALHNHYTQKSLSLSPGKGGGGSG | |
| | GGGSGGGGSGGGGSITCPPPMSVEHADIWVKSYSLYSR | |
| | ERYICNSGFKRKAGTSSLTECVLNKATNVAHWTTPSLK | |
| | CIRDP | |
| EP207 | QVQLVQSGAEVKKPGSSVKVSCKASGGTFSSYAISWVR | SEQ ID |
| 2019EP69-F03 ScFv- | QAPGQGLEWMGGIIPIFGTANYAQKFQGRVTITADESTS | NO: 188 |
| IgG1-Fc with Knob | TAYMELSSLRSEDTAVYYCARDKWYGSAWRGDYWGQ | |
| mutations; fused with | GTLVTVSSGGGGSGGGGSGGGGSQSVLTQPPSASGTPG | |
| IL15 (50-162) | QRVTISCSGSYSNIGSNFVNWYQQLPGTAPKLLIHENNQ | |
| | RPSGVPDRFSGSRSGTSASLAISGLQSEDEADYFCSTWD | |
| | DSLKGPIFGGGTKLTVLDKTHTCPPCPAPELLGGPSVFL | |
| | FPPKPKDTLMISRTPEVTCVVVDVSHEDPEVKFNWYVD | |
| | GVEVHNAKTKPREEQYNSTYRVVSVLTVLHQDWLNG | |
| | KEYKCKVSNKALPAPIEKTISKAKGQPREPQVYTLPPCR | |
| | DELTKNQVSLWCLVKGFYPSDIAVEWESNGQPENNYK | |
| | TTPPVLDSDGSFFLYSALTVDKSRWQQGNVFSCSVMHE | |
| | ALHNHYTQKSLSLSPGKGGGGSGGGGSGGGGGGGGS | |
| | WVNVISDLKKIEDLIQSMHIDATLYTESDVHPSCKVTA | |
| | MKCFLLELQVISLESGDASIHDTVENLIILANNSLSSNGN | |
| | VTESGCKECEELEEKNIKEFLQSFVHIVQMFINTS | |
| EP208 | QVQLVQSGAEVKKPGSSVKVSCKASGGTFSSYAISWVR | SEQ ID |
| 2019EP69-F03 ScFv- | QAPGQGLEWMGGIIPIFGTANYAQKFQGRVTITADESTS | NO: 189 |
| IgG1-Fc with hole | TAYMELSSLRSEDTAVYYCARDKWYGSAWRGDYWGQ | |
| mutations; fused with | GTLVTVSSGGGGSGGGGSGGGGSQSVLTQPPSASGTPG | |
| IL15Rα (31-97) | QRVTISCSGSYSNIGSNFVNWYQQLPGTAPKLLIHFNNQ | |
| | RPSGVPDRFSGSRSGTSASLAISGLQSEDEADYFCSTWD | |
| | DSLKGPIFGGGTKLTVLDKTHTCPPCPAPELLGGPSVFL | |
| | FPPKPKDTLMISRTPEVTCVVVDVSHEDPEVKFNWYVD | |
| | GVEVHNAKTKPREEQYNSTYRVVSVLTVLHQDWLNG | |
| | KEYKCKVSNKALPAPIEKTISKAKGQPREPQVCTLPPSR | |
| | DELTKNQVSLSCAVKGFYPSDIAVEWESNGQPENNYKT | |
| | TPPVLDSDGSFKLVSKLTVDKSRWQQGNVFSCSVMHE | |
| | ALHNHYTQKSLSLSPGKGGGGSGGGGSGGGGSGGGGSI | |
| | TCPPPMSVEHADIWVKSYSLYSRERYICNSGFKRKAGTS | |
| | SLTECVLNKATNVAHWTTPSLKCIRDP | |

Examples of the various antibody, bifunctional proteins, and alternative formats of the present disclosure are shown in FIG. 9 and provided in Table 7.

TABLE 7
Example Antibody. Bifunctional Antibody, and Alternative Formats.

| Format | Parts | IL-2 or IL-15/IL-15Rα | PD-L1 |
|---|---|---|---|
| Antigen-binding | | NA | See below |
| site (e.g., scFv) | | | |
| IL-2/anti-PD-L1 | EP290/EP326 | IL-2 Fc | 2019EP69-F03 |
| scFv Fc | EP291/EP326 | EP003 | clone |
| | EP297/EP326 | EP001 | |
| | EP412/EP326 | WT, | |
| | | K35L/R38D/F42R | |
| | EP413/EP326 | EP001, | |
| | | K35L/R38D/F42R | |
| | EP414/EP326 | EP003, | |
| | | K35L/R38D/F42R | |
| | EP415/EP326 | EP337, | |
| | | K35L/R38D/F42R | |
| | EP416/EP326 | EP338, | |
| | | K35L/R38D/F42R | |
| | EP417/EP326 | EP339, | |
| | | K35L/R38D/F42R | |
| | EP418/EP326 | EP340, | |
| | | K35L/R38D/F42R | |
| | EP419/EP326 | EP343, | |
| | | K35L/R38D/F42R | |
| anti-PD-L1 Fab/IL- | EP203/EP204/EP205 | WT IL15 & IL15Rα | 2019EP69-F03 |
| 15 fusion | | | clone |
| anti-PD-L1 | EP207/EP208 | WT IL15 & IL15Rα | 2019EP69-F03 |
| scFv/IL-15 fusion | | | clone |
| IL-2/anti-PD-L1 | EP199 | WT with C125S | 2019EP69-F03 |
| scFv fusion | EP200 | WT with C125S | clone |
| | EP201 | EP001 with C125S | |
| | EP202 | EP001 with C125S | |
| | EP461 | WT with | |
| | | K35L/R38D/F42R/C125S | |
| IL-2/anti-PD-L1 | EP290/EP325/EP205 | WT | 2019EP69-F03 |
| Fab | EP291/EP325/EP205 | EP003 | clone |
| | EP297/EP325/EP205 | EP001 | |
| | EP412/EP325/EP205 | WT, | |
| | | K35L/R38D/F42R | |
| | EP413/EP325/EP205 | EP001, | |
| | | K35L/R38D/F42R | |
| | EP414/EP325/EP205 | EP003, | |
| | | K35L/R38D/F42R | |
| | EP415/EP325/EP205 | EP337, | |
| | | K35L/R38D/F42R | |
| | EP416/EP325/EP205 | EP338, | |
| | | K35L/R38D/F42R | |
| | EP417/EP325/EP205 | EP339, | |
| | | K35L/R38D/F42R | |
| | EP418/EP325/EP205 | EP340, | |
| | | K35L/R38D/F42R | |
| | EP419/EP325/EP205 | EP343, | |
| | | K35L/R38D/F42R | |
| | EP415/EP462/EP205 | EP337, | 2018EP171-H02 |
| | | K35L/R38D/F42R | |
| | EP415/EP463/EP205 | EP337, | 2019EP69-E10 |
| | | K35L/R38D/F42R | |
| | EP415/EP464/EP205 | EP337, | 2019EP69-F02 |
| | | K35L/R38D/F42R | |
| anti-PD-L1 Fab/IL- | EP362/EP363/EP205 | WT | 2019EP69-F03 |
| 2 fusion | EP362/EP364/EP205 | EP001 | clone |
| | EP362/EP365/EP205 | EP250, R38V/F45S | |
| anti-PD-L1 IgG1 | EP206/EP205 | NA | 2019EP69-F03 |
| antibody | | | clone |

### Vectors and Methods of Production

In certain embodiments, the present disclosure further includes an isolated polynucleotide encoding an antigen-binding site, a protein and/or antibody, and/or a bifunctional protein and/or antibody as disclosed herein, or any fragment, variant, or combination thereof. In certain embodiments, the present disclosure further includes an expression vector comprising the polynucleotide of the present disclosure. In certain embodiments, the present disclosure further includes modified cell comprising the isolated polynucleotide of the present disclosure or the expression vector of the present disclosure.

In some embodiments, the present disclosure provides isolated polynucleotides that encode any of the presently disclosed PD-L1-antigen-binding site, specific antibody, antigen-binding fragment, or variants thereof. In certain embodiments, the polynucleotide is codon-optimized for expression in a host cell. Once a coding sequence is known or identified, codon optimization can be performed using known techniques and tools, e.g., using the GenScript OptimiumGene tool; see also Scholten et al., Clin. Immunol. 119:135, 2006). Codon-optimized sequences include sequences that are partially codon-optimized (i.e., one or more codon is optimized for expression in the host cell) and those that are fully codon-optimized.

It will also be appreciated that polynucleotides encoding PD-L1 antigen-binding site, specific antibody, antigen-binding fragment, or variants thereof of the present disclosure may possess different nucleotide sequences while still encoding a same PD-L1 antigen-binding site, specific antibody, antigen-binding fragment, or variants thereof due to, for example, the degeneracy of the genetic code, splicing, and the like.

Vectors are also provided, wherein the vectors comprise or contain a polynucleotide as disclosed herein (i.e., a polynucleotide that encodes a PD-L1 antigen-binding site-specific antibody, antigen-binding fragment, or variants thereof). A vector can comprise any one or more of the vectors disclosed herein.

In a further aspect, the present disclosure also provides a host cell expressing PD-L1 antigen-binding site, specific antibody, antigen-binding fragment, or variants thereof according to the present disclosure; or comprising or containing a vector or polynucleotide according the present disclosure.

Examples of such cells include but are not limited to, eukaryotic cells, e.g., yeast cells, animal cells, insect cells, plant cells; and prokaryotic cells, including E. coli. In some embodiments, the cells are mammalian cells. In certain such embodiments, the cells are a mammalian cell line such as CHO cells (e.g., DHFR-CHO cells (Urlaub et al., PNAS 77:4216 (1980)), human embryonic kidney cells (e.g., HEK293T cells), PER.C6 cells, Y0 cells, Sp2/0 cells. NS0 cells, human liver cells, e.g. Hepa RG cells, myeloma cells or hybridoma cells. Other examples of mammalian host cell lines include mouse sertoli cells (e.g., TM4 cells); monkey kidney CV1 line transformed by SV40 (COS-7); baby hamster kidney cells (BHK); African green monkey kidney cells (VERO-76); monkey kidney cells (CV1); human cervical carcinoma cells (HELA); human lung cells (W138); human liver cells (Hep G2); canine kidney cells (MDCK; buffalo rat liver cells (BRL 3A); mouse mammary tumor (MMT 060562); TRI cells; MRC 5 cells; and FS4 cells. Mammalian host cell lines suitable for antibody production also include those described in, for example, Yazaki and Wu, Methods in Molecular Biology, Vol. 248 (B. K. C. Lo, ed., Humana Press, Totowa, N.J.), pp. 255-268 (2003).

In certain embodiments, a host cell is a prokaryotic cell, such as an E. coli. The expression of peptides in prokaryotic cells such as E. coli is well established (see, e.g., Pluckthun, A. Bio/Technology 9:545-551 (1991). For example, antibodies may be produced in bacteria, in particular when glycosylation and Fc effector function are not needed. For expression of antibody fragments and polypeptides in bacteria, see, e.g., U.S. Pat. Nos. 5,648,237; 5,789,199; and 5,840,523.

In particular embodiments, the cell may be transfected with a vector according to the present description with an expression vector. The term “transfection” refers to the introduction of nucleic acid molecules, such as DNA or RNA (e.g. mRNA) molecules, into cells, such as into eukaryotic cells. In the context of the present description, the term “transfection” encompasses any method known to the skilled person for introducing nucleic acid molecules into cells, such as into eukaryotic cells, including into mammalian cells. Such methods encompass, for example, electroporation, lipofection, e.g., based on cationic lipids and/or liposomes, calcium phosphate precipitation, nanoparticle based transfection, virus based transfection, or transfection based on cationic polymers, such as DEAE-dextran or polyethylenimine, etc. In certain embodiments, the introduction is non-viral.

Moreover, host cells of the present disclosure may be transfected stably or transiently with a vector according to the present disclosure, e.g. for expressing PD-L1 antigen-binding site-specific antibody, antigen-binding fragment, or variants thereof, according to the present disclosure. In such embodiments, the cells may be stably transfected with the vector as described herein. Alternatively, cells may be transiently transfected with a vector according to the present disclosure encoding an antibody or antigen-binding fragment as disclosed herein. In any of the presently disclosed embodiments, a polynucleotide may be heterologous to the host cell.

Accordingly, the present disclosure also provides recombinant host cells that heterologously express PD-L1 antigen-binding site, specific antibody, antigen-binding fragment, or variants thereof of the present disclosure. For example, the cell may be of a species that is different to the species from which the antibody was fully or partially obtained (e.g., CHO cells expressing a human antibody or an engineered human antibody). In some embodiments, the cell type of the host cell does not express the PD-L1 antigen-binding site, specific antibody, antigen-binding fragment, or variants thereof in nature. Moreover, the host cell may impart a post-translational modification (PTM; e.g., glycosylation or fucosylation) on the PD-L1 antigen-binding site-specific antibody, antigen-binding fragment, or variants thereof that is not present in a native state of the antibody or antigen-binding fragment (or in a native state of a parent antibody from which the antibody or antigen binding fragment was engineered or derived). Such a PTM may result in a functional difference (e.g., reduced immunogenicity). Accordingly, PD-L1 antigen-binding site, specific antibody, antigen-binding fragment, or variants thereof of the present disclosure that is produced by a host cell as disclosed herein may include one or more post-translational modification that is distinct from the antibody (or parent antibody) in its native state (e.g., a human antibody produced by a CHO cell can comprise a more post-translational modification that is distinct from the antibody when isolated from the human and/or produced by the native human B cell or plasma cell).

In a related aspect, the present disclosure provides methods for producing PD-L1 antigen-binding site, specific antibody, antigen-binding fragment, or variants thereof, wherein the methods comprise culturing a host cell of the present disclosure under conditions and for a time sufficient to produce PD-L1 antigen-binding site, specific antibody, antigen-binding fragment, or variants thereof. Methods useful for isolating and purifying recombinantly produced PD-L1 antigen-binding site, specific antibody, antigen-binding fragment, or variants thereof, by way of example, may include obtaining supernatants from suitable host cell/vector systems that secrete the recombinant antibody into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of the isolated/recombinant antibody described herein include batch cell culture, which is monitored and controlled to maintain appropriate culture conditions. Purification of soluble PD-L1 antigen-binding site-specific antibody, antigen-binding fragment, or variants thereof may be performed according to methods described herein and known in the art and that comport with laws and guidelines of domestic and foreign regulatory agencies.

### Pharmaceutical Compositions

In certain embodiments, the present disclosure further provides pharmaceutical composition comprising an antigen-binding site of the present disclosure, a protein and/or antibody of the present disclosure, or a bifunctional protein and/or antibody of the present disclosure, and a pharmaceutically acceptable diluent(s), excipient(s), or carrier(s). In some embodiments, the pharmaceutical compositions comprise an additional therapeutic agent (e.g., combination therapy). The pharmaceutical compositions may be formulated in a conventional manner using one or more pharmaceutically acceptable carriers comprising excipients and auxiliaries which facilitate processing of the antigen-binding site of the present disclosure, protein and/or antibody of the present disclosure, or bifunctional protein and/or antibody of the present disclosure into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999). Examples of IL-2 compositions are described in U.S. Pat. Nos. 4,604,377 and 4,766,106, which are incorporated by reference herein.

As used herein, “pharmaceutically acceptable carrier” and “physiologically acceptable carriers” are used interchangeably and include any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art and are molecular entities and compositions that are generally non-toxic to recipients at the dosages and concentrations employed, i.e., do not produce an adverse, allergic or other untoward reaction when administered to an animal, such as, for example, a human, as appropriate (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The pharmaceutical composition may comprise different types of carriers depending on whether it is to be administered in solid, liquid or aerosol form, and whether it need to be sterile for such routes of administration as injection. An antigen-binding site of the present disclosure, a protein and/or antibody of the present disclosure, or a bifunctional protein and/or antibody of the present disclosure (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the antigen-binding sites of the present disclosure, the proteins and/or antibodies of the present disclosure, or the bifunctional proteins and/or antibodies of the present disclosure.

### Methods of Treatment or Use

In some embodiments, provided herein is a method treatment or use, comprising administering an therapeutically effective amount of an antigen-binding site disclosed herein, a protein or antibody disclosed herein, or a bifunctional protein or antibody disclosed herein to a subject in need thereof, thereby modulating an immune response. In some embodiments, modulating the immune response comprises at least one of enhancing T cell activity or enhancing NK cell activity. In some embodiments, the method of treatment or use, is treating a disease in a subject in need thereof. In certain embodiments, the disease is a cancer. In some embodiments, the cancer comprises breast cancer, pancreatic cancer, lung cancer, glioblastoma, renal cell carcinoma, or melanoma. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, glioblastoma, prostate cancer, blood cancer, skin cancer, squamous cell carcinoma, skin cancer, melanoma, bone cancer, renal cell carcinoma, and kidney cancer. Also included are pre-cancerous conditions or lesions and cancer metastases. Other cell proliferation disorders include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Similarly, other cell proliferation disorders can also be treated, such as hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other cell proliferation disease, besides neoplasia, located in an organ system listed above.

In some embodiments, the use or method of treatment or modulating the immune response further comprises administering to the subject a therapeutically effective amount of at least one additional therapeutic agent (e.g., a combination therapy). In certain embodiments, the additional therapeutic agent is an anti-cancer agent. Examples of anti-cancer agents include checkpoint inhibitors (e.g., anti-PD1 antibodies), chemotherapeutic agents, agents that inhibit a tumor microenvironment, cancer vaccines (e.g., Sipuleucel-T), oncolytic viruses (e.g., talimogene laherparepvec), immune cells expressing a chimeric antigen receptor, and tumor infiltrating lymphocytes. In certain embodiments, the additional therapeutic agent is a molecule including an antigen binding moiety. In certain specific embodiments, the antigen binding moiety is selected from a single domain antibody, a Fab molecule, an scFv, a diabody, a nanobody, a bi-specific T cell engager, or an immunoglobulin. In certain embodiments, the antigen binding moiety is specific to a tumor antigen (e.g., carcinoembryonic antigen, fibroblast activation protein-a, CD20) or a check point protein (e.g., CTLA-4, PD-1). In some embodiments, the additional therapeutic agent comprises an immune cell expressing a chimeric antigen receptor, an immune cell expressing an engineered T cell receptor, or a tumor infiltrating lymphocyte.

Other embodiments will be clear from the disclosure infra.

### EXAMPLES

The following examples are offered by way of illustration and not by way of limitation.

### Example 1. Development of Anti-PD-L1 Antibody

This example describes the development and characterization of antibodies that specifically bind to human PD-L1.

Scfv mRNA Display Screening and Selection

mRNA display technology was used for the identification of PD-L1 binders from 10¹²⁻¹³ natural human scFv libraries. Briefly, the DNA libraries were first transcribed into mRNA libraries and then translated into mRNA-scFv fusion libraries by covalent coupling through a puromycin-linker, similar to the reported procedure (U.S. Pat. No. 6,258,558). The fusion libraries were first counter selected with human IgGs (negative proteins) to remove non-specific binders, followed by selection against recombinant PD-L1-Fc fusion protein, then captured on Protein G magnetic beads. To enrich for scFvs that block PD-1 and PD-L1 interaction, PD-1 was utilized to compete off the PD-L1 binders from Protein G beads and binders were enriched by PCR amplification with library specific oligos. Total of 4 rounds of selections executed to generate highly enriched PD-L1 binding pool for screening.

Identifications and Characterization of Anti-PD-L1 scFvs

After 4 rounds of selections, the PD-L1 enriched scFv library was cloned into bacterial periplasmic expression vector pET22b and transformed into TOP10 competent cells. Each of the scFv molecule was engineered to have a C-terminal flag and 6×His tag for purification and assay detection. Clones from TOP10 cells were pooled and the miniprep DNA were prepared and subsequently transformed into bacterial Rosetta II strain for expression. Single clones were picked, grown, and induced with 0.1 mM IPTG in 96 well plate for expression. The supernatant was collected after 16-24 hours induction at 30° C. for assays to identify anti-PD-L1 scFvs.

PD-L1 binding screening ELISA was developed for the identification of individual anti-PD-L1 scFvs. Briefly, 384 well plate was immobilized with human Fc and human PD-L1-Fc, at final concentration of 2 μg/mL in 1×PBS in total volume of 25 μL per well. The plate was incubated overnight at 4° C. followed by blocking with 80 μL of superblock per well for 1 hour. 25 μL of supernatant was added to the human Fc and human PD-L1-Fc immobilized wells and incubated for 1 hour with shaking. The PD-L1 binding was detected by adding 25 μL of anti-FLAG HRP diluted at 1:5000 in 1×PBST. In between each step, the plate was washed 3 times with 1×PBST in a plate washer. The plate was then developed with 20 μL of TMB substrate for 5 min and stopped by adding 20 μL of 2N sulfuric acid. The plate was read at OD450 nm Biotek plate reader and the binding and selectivity was analyzed with Excel bar graph. Clones with PD-L1 target binding of greater than 2-fold compared to human Fc control were subjected for DNA sequencing. The unique clones were produced and purified for further characterization.

ScFv Production in E. coli

The specified anti-PD-L1 clone was picked from a glycerol stock plate and grown overnight into a 5 mL culture in a Thomson 24-well plate with a breathable membrane. This culture, and all subsequent cultures described below were grown at 37° C. and shaking at 225 rpm in Terrific Broth Complete plus 100 μg/mL carbenicillin and 34 μg/mL chloramphenicol, with 1:5,000 dilution of antifoam-204 also added, unless specified otherwise. This overnight starter culture was then used to inoculate the larger culture, 1:100 dilution of starter culture into the designated production culture and grown until OD600 was between 0.5-0.8. At this point, the culture was induced with a final concentration of IPTG at 0.1 mM and incubated over night at 30° C. The following day, the cultures were centrifuged for 30 min at 5,000×g, to pellet the cells and then the supernatant was filter sterilized through a 0.2 μm sterilizing PES membrane.

For the purification, 3 μL Ni Sepharose Excel resin (GE Healthcare) per 1 mL of filtered supernatant was used. Disposable 10 mL or 20 mL BioRad Econo-Pac columns were used. The resin was equilibrated with at least 20 column volume (CV) buffer A (1×PBS, pH 7.4 with extra NaCl added to 500 mM). The filter sterilized supernatant was purified by gravity flow by either controlling the flow to 1 mL/min or pouring over two times, over the same packed resin bed. The column was then washed with buffers 10 CV buffer A and 20 CV buffer B (1×PBS, pH 7.4 with extra NaCl to 500 mM, and 30 mM imidazole). The two Detox buffers were used to remove endotoxin as an optional step if needed. For 250 mL expression culture purifications, antibody bound column was washed sequentially with 20 CV buffer C (1×PBS, pH 7.4 with extra NaCl to 500 mM, 1% TX114), 20 CV buffer D (1×PBS, pH 7.4 with extra NaCl to 500 mM, 1% TX100+0.2% TNBP), and 40 CV buffer E (1×PBS, pH 7.4 with extra NaCl to 500 mM). The protein was eluted with Eluting buffer F (1×PBS, pH 7.4 with extra NaCl to 500 mM, and 500 mM imidazole) in a total of six fractions (0.5 CV pre elute, 5×1 CV elute). Fractions were run on a Bradford assay (100 μL diluted Bradford solution+10 μL sample). Fractions with bright blue color were pooled. Protein concentration was measured by A280 extension coefficient. SDS-PAGE gel was used to analyze the purity of the purified antibodies. In most cases, thermal shift assay was performed to measure the thermal stability of the purified antibodies.

### PD-L1 Binding ELISA

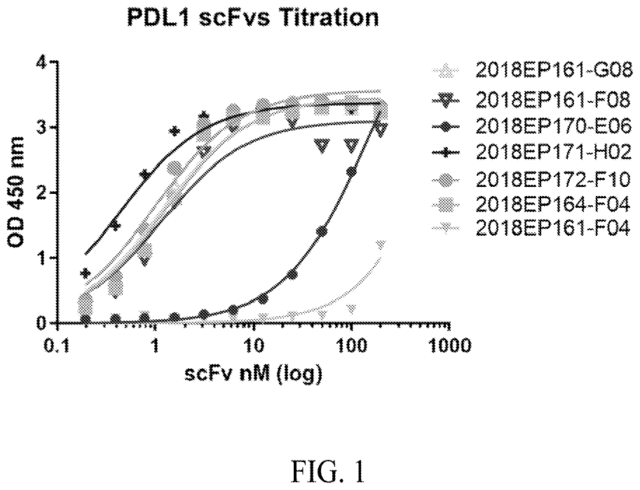

An ELISA assay was developed to determine the EC50 of anti-PD-L1 scFvs. Briefly, 384 well plate was immobilized with human PD-L1-Fc at final concentration of 2 μg/mL in 1×PBS in total volume of 25 μL per well. The plate was incubated overnight at 4° C. followed by blocking with 80 μL of superblock per well for 1 hour. Purified anti-PD-L1 scFvs were 2-fold serial titrated from 200 nM. 25 μL was added to human PD-L1 immobilized wells and incubated for 1 hour with shaking. The PD-L1 binding was detected by adding 25 μL of anti-FLAG HRP diluted at 1:5000 in 1×PBST. In between each step, the plate was washed 3 times with 1×PBST in a plate washer. The plate was then developed with 20 μL of TMB substrate for 5 min and stopped by adding 20 μL of 2N sulfuric acid. The plate was read at OD450 nm Biotek plate reader and then plotted in Prism 8.1 software. EC50 was calculated and shown in Table 8. FIG. 1 shows the PD-L1 binding ELISA curve for selected scFvs.

TABLE 8
EC50 values for anti-PD-L1 scFvs binding ELISA.

| scFv | EC₅₀ (nM) |
|---|---|
| 2018EP161-G08 | 1.161 |
| 2018EP161-F08 | 1.152 |
| 2018EP170-E06 | 157.7 |
| 2018EP171-H02 | 0.4292 |
| 2018EP172-F10 | 0.6152 |
| 2018EP164-F04 | 1.271 |
| 2018EP161-F04 | ND |
| 2018EP280-E04 | NA |
| 2018EP173-H11 | NA |
| 2018EP280-E01 | 8.095 |

ND: not determined,
NA: not available.

### ScFv Binding to PD-L1 in SPR

Kinetic analysis of anti-PD-L1 scFvs have been assessed by surface plasmon resonance (SPR) technology with Biacore T200. The assay was run with Biacore T200 control software version 2.0. Anti-human Fc antibody was immobilized on flow cell 1 and 2 of CM5 sensor chip. For each cycle, 1 μg/mL of human PD-L1-Fc protein was captured for 60 seconds at flow rate of 10 μl/min on flow cell 2 in 1×HBSP buffer on anti-hFc sensor chip. 2-fold serial diluted HIS tag purified anti-PD-L1 scFv was injected onto both reference flow cell 1 and PD-L1-Fc captured flow cell 2 for 150 seconds at flow rate of 30 μl/min followed by wash for 300 seconds. The flow cells were then regenerated with antibody regeneration buffer (GE Healthcare) for 30 seconds at flow rate of 30 μl/min. 8 concentration points from 300 nM-0 nM was assayed per anti-PD-L1 scFv in a 96 well plate. The kinetics of scFvs binding to PD-L1 protein was analyzed with Biacore T200 evaluation software version 3.0. The specific binding response unit was derived from subtraction of binding to reference flow cell 1 from PD-L1 captured flow cell 2. The binding kinetics (Kₐ, K_{d}, and K_{D}) of selected scFvs were determined from the sensorgram analyses and are shown in Table 9.

TABLE 9
Kinetic parameters of anti-PD-L1 scFv binding to PD-L1 in SPR.

| scFv | Kₐ (1/ms) | K_{d} (1/s) | K_{D} (M) |
|---|---|---|---|
| 2018EP161-G08 | NA | NA | NA |
| 2018EP161-F08 | 2.51E+05 | 1.25E−03 | 4.97E−09 |
| 2018EP170-E06 | NA | NA | NA |
| 2018EP171-H02 | 1.06E+06 | 1.90E−03 | 1.81E−09 |
| 2018EP172-F10 | 9.29E+05 | 1.99E−03 | 2.14E−09 |
| 2018EP164-F04 | 2.45E+05 | 7.22E−04 | 2.94E−09 |
| 2018EP161-F04 | NA | NA | NA |
| 2018EP280-E04 | NA | NA | NA |
| 2018EP173-H11 | NA | NA | NA |
| 2018EP280-E01 | NA | NA | NA |

### ScFv Binding to Cell Surface PD-L1 in FACS

K562 cells (ATCC) were transfected with a construct encoding the full-length human PD-L1 with C-terminal FLAG and Myc tags in pCMV6-Entry vector. G418 drug selection process yielded a polyclonal, drug resistant pool of PD-L1 target-expressing cells. In parallel, the empty vector transfected parental line was generated as a negative control. The PD-L1 target-expressing cells were sorted by FACS to yield a PD-L1 target expressing polyclonal pool. The pool was expanded under G418 drug selection. Single cell sorting then was performed followed by further drug selection to form clonal cell lines. The clonal lines were screened for PD-L1 expression by FACS. The high expression PD-L1 cell line was then used for screening and assays.

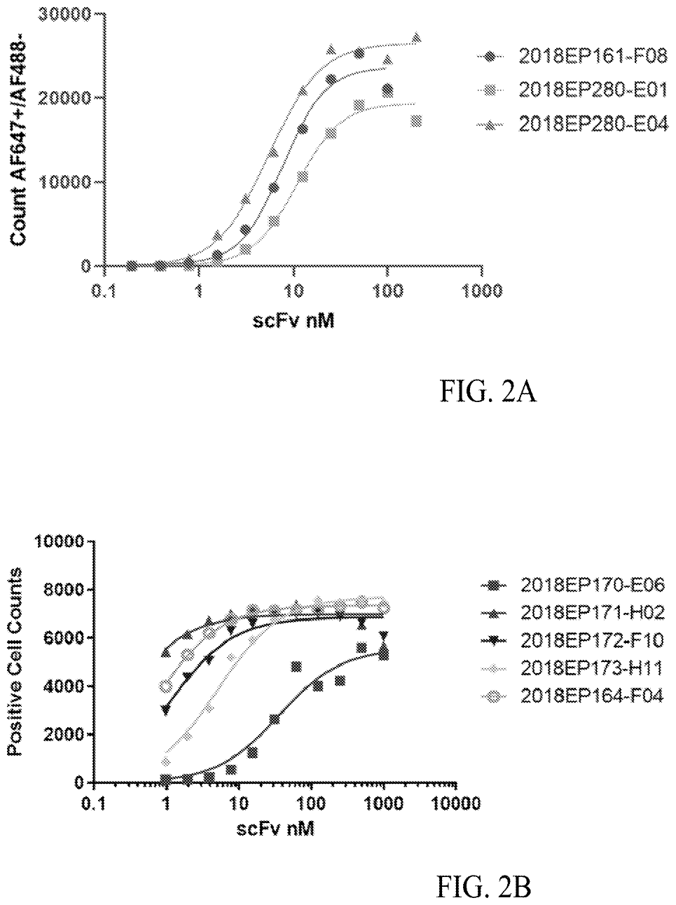

To determine whether anti-PD-L1 scFvs bind to PD-L1 expressing cells, 200 nM of purified anti-PD-L1 scFvs were diluted in full medium and incubated with PD-L1/K562 and K562 cells in 96 wells plate on ice for 1 hour. Cells were spun down at 1200 rpm for 5 minutes at 4° C. to remove primary antibodies. Cells were then washed once with 200 μL of full medium per well. Samples were detected with premixed anti-His Biotin Streptavidin Alexa Fluor 647 by adding 100 μL of diluted secondary antibody and incubated at 4° C. for 30 minutes in the dark. Samples were spun down at 1200 rpm for 5 minutes at 4° C. and washed twice with 200 μL of 1×PBS per well. Samples were reconstituted in 200 μL of 1×PBS and were read on Attune NxT cytometer. Analysis was done by Attune NXT software plotting the overlay histogram of anti-PD-L1 scFvs binding onto both negative and target cell lines. The cell surface PD-L1 binding EC50 for selected scFvs was calculated and shown in the Table 10. FIG. 2A-B shows the PD-L1/K562 cell surface binding FACS curve for selected scFvs.

TABLE 10
EC50 values of anti-PD-L1 scFvs binding to K562 cells engineered
to express high levels of cell surface human PD-L1.

| | PD-L1/K562 Binding |
|---|---|
| | FACS EC50 (nM) |
| 2018EP161-G08 | NA |
| 2018EP161-F08 | 7.72 |
| 2018EP170-E06 | 35.9 |
| 2018EP171-H02 | 0.2498 |
| 2018EP172-F10 | <1 |
| 2018EP164-F04 | <1 |
| 2018EP161-F04 | 5.8 |
| 2018EP280-E04 | 5.509 |
| 2018EP173-H11 | 5.102 |
| 2018EP280-E01 | 10.83 |

ScFv Competition with PD-L1 and PD-1 Interaction in HTRF

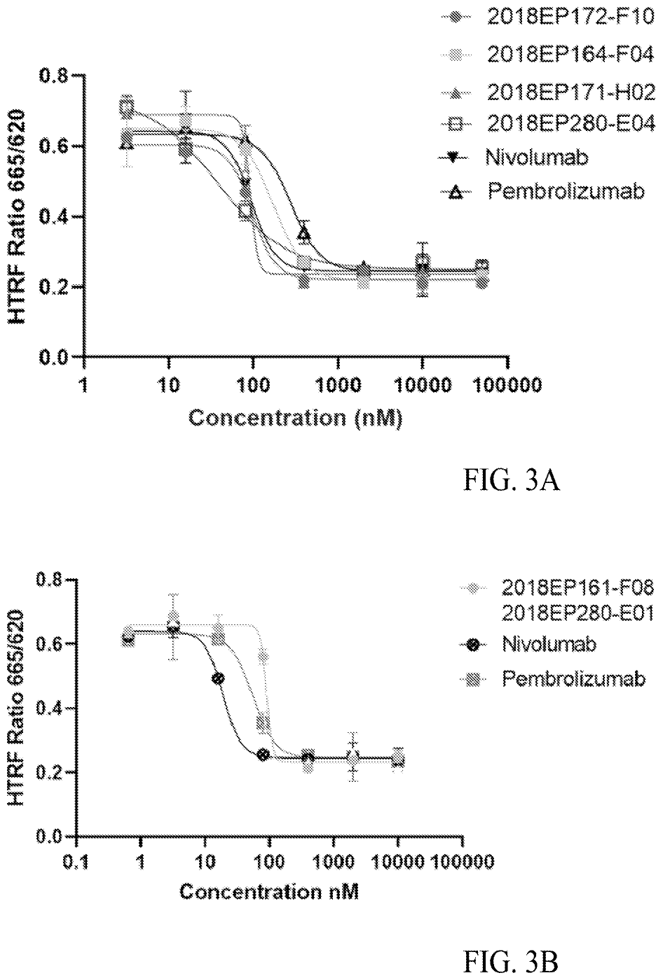

PD-1/PD-L1 TR-FRET (HTRF) assay kit (BPS, 72038) was used to evaluate the PD-1 neutralizing activity by anti-PD-L1 scFvs. Briefly, 1× Immuno buffer 1 was made. Dye-labeled acceptor, PD-1-Eu, PD-L1 biotinylated and scFvs were diluted with buffer 1. For each well in 384-well plate, serial diluted scFvs and regents were added according to manufacturer's recommended volume. Samples were incubated at room temperature for 1.5 hours then the fluorescent intensity was read with Biotek Neo2 plate reader using manufacture's instrument setting for the assay. Data analysis was performed using the TR-FRET ratio of 665 nm emission/620 nm emission and plotted with Prism 8.0. The PD-1 and PD-L1 competition IC50 for selected scFvs was calculated and shown in Table 11. FIG. 3A-B shows the PD-1 and PD-L1 competition results for selected scFvs.

TABLE 11
HTRF IC50 of PD-1 and PD-L1 competition for scFv.

| | PD-1 and PD-L1 |
|---|---|
| | HTRF IC50 (nM) |
| 2018EP161-G08 | NA |
| 2018EP161-F08 | 90.79 |
| 2018EP170-E06 | NA |
| 2018EP171-H02 | 91.85 |
| 2018EP172-F10 | 96.97 |
| 2018EP164-F04 | 172.9 |
| 2018EP161-F04 | NA |
| 2018EP280-E04 | 38.51 |
| 2018EP173-H11 | NA |
| 2018EP280-E01 | 76.80 |

### ScFv in Jurkat Cell NFAT Reporter Assay

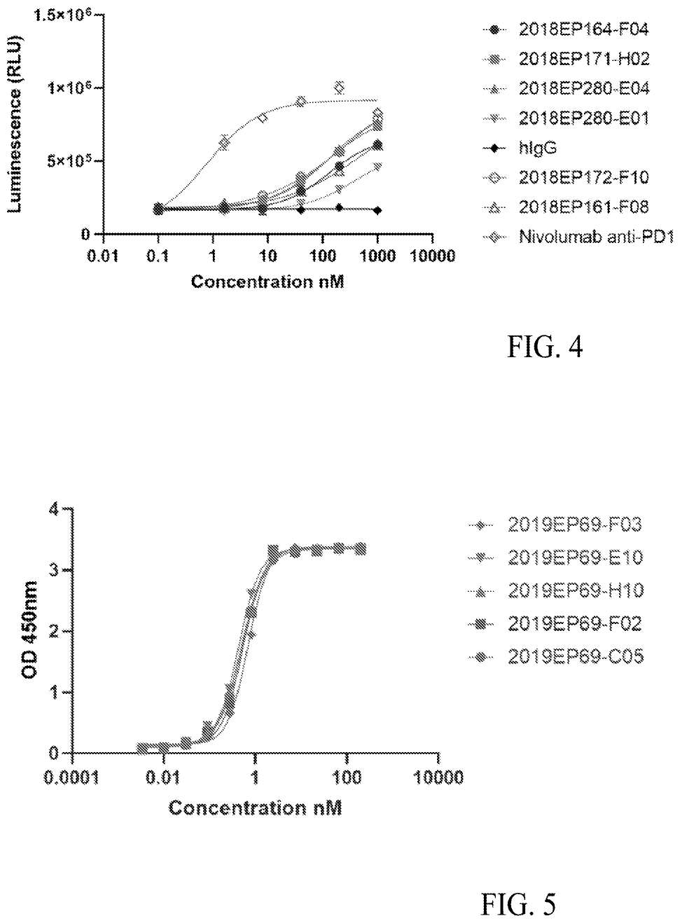

PD-1 neutralizing activities of anti-PD-L1 scFvs were also evaluated in cellular assay using PD-1/PD-L1 blockade bioassay kit (Promega, J1250). Following manufacture's protocol, PD-L1 aAPC/CHOK 1 cells were thawed out and plated on white flat bottom assay plates. Cells were incubated at 37° C. with CO₂ 5% overnight. The next day, media was removed from each well. PD-L1 scFv clones were 5 fold serial diluted starting from 500 nM and 40 μL of 2× concentration of diluted scFvs and controls were added to the cells. The plate was incubated at 37° C. for 30 minutes to allow scFv binding onto PD-L1 cell surface. PD-1 effector cells were thawed following manufacture's protocol and 40 μL added to each well containing PD-L1 aAPC/CHOK1 cells with anti-PD-L1 scFvs. The plate was incubated at 37° C. with 5% CO₂ for 16 hours. The assay plate was then was equilibrated to room temperature for 10 minutes. 80 μL of room temperature Bio-Glo reagent was added to each well and control wells. The plate was incubated at room temperature with shaking for 30 minutes and protected it from light. The luminescence was quantified with Biotek Neo2 plate reader and plotted with Prism 8.0 software. The NFAT reporter assay EC50 for selected scFvs was calculated and provided in Table 12. FIG. 4 shows the reporter assay results.

TABLE 12
EC50 values of Jurkat NFAT reporter assay for anti-PD-L1 scFv.

| Seq. ID | EC50 (nM) |
|---|---|
| 2018EP164-F04 | 129.3 |
| 2018EP280-E01 | 399.9 |
| 2018EP172-F10 | 365.1 |
| 2018EP161-F08 | 426.7 |
| 2018EP170-E06 | NA |
| 2018EP161-G08 | NA |
| 2018EP161-F04 | NA |
| 2018EP173-H11 | NA |
| 2018EP280-E04 | 133.0 |
| 2018EP171-H02 | 115.3 |

Affinity Maturation of Anti-PD-L1 scFv 2018EP171-H02

To further improve the affinity and biological activity of anti-PD-L1 scFv, rational designed HCDR3 mutagenesis scFv library of 2018EP171-H02 was constructed and subjected to stringent selection conditions. HCDR3 (SEQ ID NO:46) residues were mutagenized at ratio of WT vs mutant of 70% vs 30%, gtg tat tac tgt gcg aga gat aaa ggg tat ggc agt ggc tgg agg ggt gac tac tgg ggc cag gga (SEQ ID NO:190). Mutagenic library was constructed by overlap PCR with framework and VL. mRNA display was used to enrich for higher affinity scFv binders through two rounds of selections. The first round was selected on PD-L1/CHOK1 cell line to ensure the molecules binding on native epitope of PD-L1. The second round was bound with 10 nM PD-L1-Fc protein followed by off-rate selection for 16 hours in the presence of 500 nM of immobilized PD-L1 as competitor. The immobilized PD-L1 together with weaker binders were removed. The PD-L1 Fc with higher affinity scFv binders were then captured with Protein G beads and eluted off and PCR amplified.

After 2 rounds of mutagenic library selections, the pool was cloned into pET22b vectors and screened as described above.

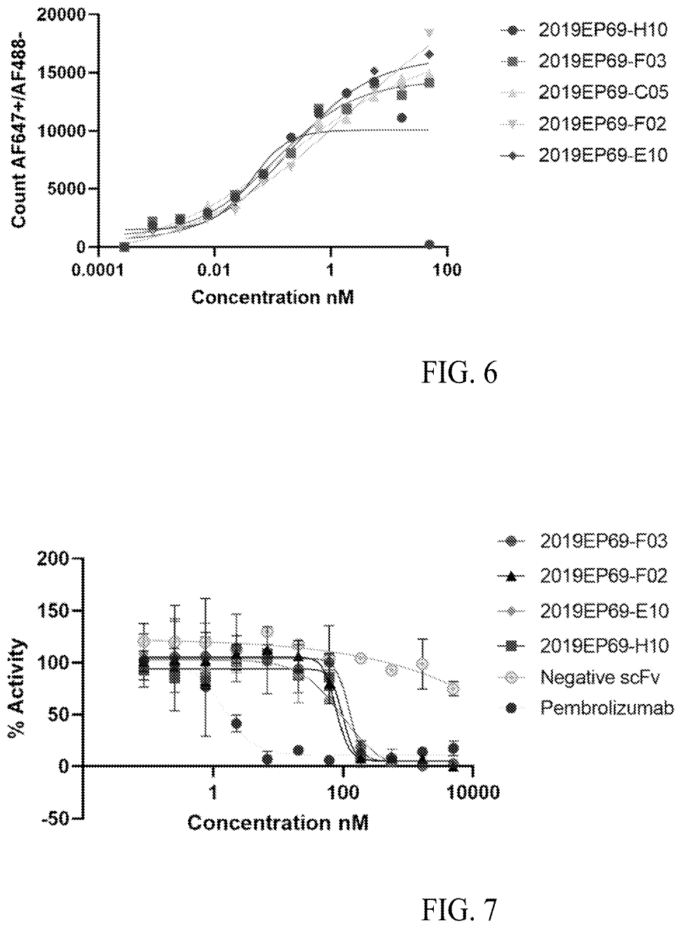

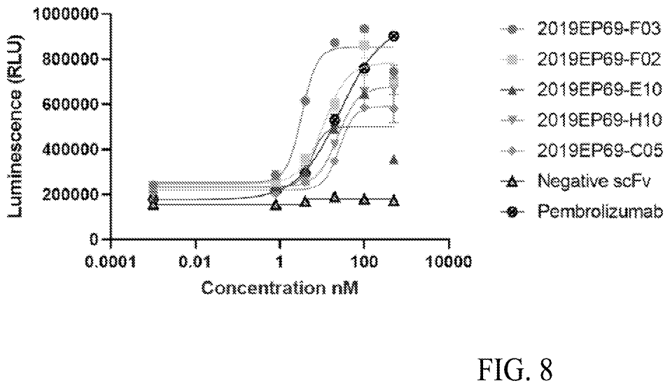

The production, the binding, and all of the characterization assays for selected affinity matured scFv clones were performed as described above. The data were analyzed and provided in Table 13 for the ELISA binding, Table 14 for the SPR kinetics analysis, Table 15 for the PD-L1/K6562 cell surface FACS binding, and Table 16 for the Jurkat NFAT reporter assay surface PD-L1 binding result. FIG. 5 shows ELISA binding results. FIG. 6 shows PD-L1/K6562 cell surface FACS binding. FIG. 7 shows PD-1 and PD-L1 competition HTRF results. FIG. 8 shows the Jurkat NFAT reporter assay results for selected optimized scFvs.

TABLE 13
EC50 values of anti-PD-L1 scFvs binding ELISA.

| | PD-L1 Binding |
|---|---|
| Seq. ID | ELISA (nM) |
| 2018EP171-H02 | 0.4292 |
| 2019EP69-E02 | NA |
| 2019EP69-E10 | 0.4352 |
| 2019EP69-C05 | 0.5073 |
| 2019EP69-F02 | 0.5447 |
| 2019EP69-B01 | NA |
| 2019EP69-H10 | 0.5004 |
| 2019EP69-F03 | 0.6963 |

TABLE 14
Kinetic parameters of anti-PD-L1 scFv binding to PD-L1 in SPR.

| | Kₐ (1/ms) | K_{d} (1/s) | K_{D} (M) |
|---|---|---|---|
| 2018EP171-H02 | 7.09E+05 | 1.753E−3 | 2.47E−09 |
| 2019EP69-E02 | NA | NA | NA |
| 2019EP69-E10 | 1.16E+06 | 1.137E−3 | 9.83E−10 |
| 2019EP69-C05 | 1.76E+06 | 4.541E03 | 2.58E−09 |
| 2019EP69-F02 | 3.93E+05 | 2.44E−04 | 6.22E−10 |
| 2019EP69-B01 | NA | NA | NA |
| 2019EP69-H10 | 9.41E+05 | 1.226E−3 | 1.30E−09 |
| 2019EP69-F03 | 1.24E+06 | 9.87E−05 | 7.97E−11 |

TABLE 15
EC50 values of anti-PD-L1 scFvs binding to K562 cells engineered
to express high levels of cell surface human PD-L1.

| Seq. ID | PD-L1 Binding FACS (nM) |
|---|---|
| 2018EP171-H02 | 0.25 |
| 2019EP69-E02 | NA |
| 2019EP69-E10 | 0.207 |
| 2019EP69-C05 | 0.221 |
| 2019EP69-F02 | 2.004 |
| 2019EP69-B01 | NA |
| 2019EP69-H10 | 0.0799 |
| 2019EP69-F03 | 0.115 |

TABLE 16
EC50 values of Jurkat NFAT reporter assay for anti-PD-L1 scFv.

| Seq. ID | Jurkat NFAT Reporter assay EC50 (nM) |
|---|---|
| 2019EP69-E02 | NA |
| 2019EP69-E10 | 96.07 |
| 2019EP69-C05 | ~61.09 |
| 2019EP69-F02 | 78.52 |
| 2019EP69-H10 | 97.71 |
| 2019EP69-F03 | 122.7 |

### Example 2. Antibody and Bifunctional Antibody Structure

The variable VH and VL sequences of 2019EP69-F03 were fused to the constant frame sequence of human heavy chain IgG1 backbone and light chain lambda backbone (EP206 and EP205, respectively) to generate an anti-PD-L1 monoclonal antibody.

To generate anti-PD-L1/IL-15 fusion bifunctional antibody, the C-terminus of one heavy chain of the anti-PD-L1 was fused to human IL-15 (50-162) [P40933] with a (G₄S)₄ linker. The S354C, T366W, and K409A mutations (Wei et al., Oncotarget, 2017; Xu et al., mAbs, 2015) were introduced to make the chain as a knob molecule (EP203). The C-terminus of another heavy chain of the anti-PD-L1 was fused to human IL-15Rα (31-97) [Q13261] with a (G₄S)₄ linker. The Y349C, T366S, L368A, F405K, and Y407V mutations (Wei et al., Oncotarget, 2017; Xu et al., mAbs, 2015) were introduced to the make the chain as a hole molecule (EP204). The light chain EP205 was used to pair with the knob and hole anti-PD-L1 chains, respectively.

In another case, the entire scFv chain of 2019EP69-F03 was fused to the constant Fc frame sequence of the human heavy chain IgG1 backbone. The C-terminus of one of the scFv-Fc molecules was fused to human IL-15 (50-162) [P40933] with a (G4S) 4 linker. This molecule carried the S354C, T366W, and K409A mutations as a knob molecule. The C-terminus of another scFv-Fc molecule was fused to human IL-15Rα (31-97) [Q13261] with a (G4S) 4 linker (EP207). This molecule carried Y349C, T366S, L368A, F405K, and Y407V mutations as a hole molecule (seq. EP208). L234A, L235A, and P329G mutations in both the knob and hole molecules were introduced to eliminate complement binding and Fc-γ dependent antibody-dependent cell-mediated cytotoxicity (ADCC) effects (Lo et al., JBC 2017).

In another example, The S354C, T366W, and K409A mutations were introduced to the heavy chain of anti-PD-L1 sequence (e.g., EP205) to generate a knob molecule (e.g., EP362). The protein sequences encoding engineered IL-2 polypeptides were fused to the C-terminal site of the constant frame sequence of the heavy chain of anti-PD-L1 sequence (e.g., EP205) with a (G4S) 4 linker. The Y349C, T366S, L368A, F405K, and Y407V mutations were introduced to make the chain as a hole molecule (e.g., EP363, EP364, EP365). In certain embodiments, L234A, L235A, and P329G mutations in the Fc were introduced to eliminate complement binding and Fc-γ dependent antibody-dependent cell-mediated cytotoxicity (ADCC) effects (Lo et al., JBC 2017).

In various formats, the C-terminus of one heavy chain of the anti-PD-L1 was fused to human IL-15 (50-162) with a (G4S) 4 linker. The S354C, T366W, and K409A mutations were introduced to make the chain as a knob molecule (e.g., EP203). The C-terminus of another heavy chain of the anti-PD-L1 was fused to human IL-15Rα (31-97) with a (G₄S)₄ linker. The Y349C, T366S, L368A, F405K, and Y407V mutations were introduced to the make the chain as a hole molecule (e.g., EP204). L234A, L235A, and P329G mutations in both the knob and hole molecules were introduced to eliminate complement binding and Fc-γ dependent antibody-dependent cell-mediated cytotoxicity (ADCC) effects (Lo et al., JBC 2017). The light chain EP205 was used to pair with the knob and hole anti-PD-L1 chains, respectively.

The DNA encoding the entire above designed sequences were then synthesized with codon optimized for mammalian cell expression, and subcloned to pCDNA3.4 (Invitrogen). FIG. 9 shows the schematic diagram of the formats for selected anti-PD-L1 antibodies.

### Example 3. IL-2-Fc and Antibodies Production

The anti-PD-L1 monoclonal antibody was expressed transiently in ExpiHEK293-F cells in free style system (Invitrogen) according to standard protocol with a ratio of the plasmid DNA of heavy chain and light chain of 1:2. The cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH 2.5+150 mM NaCl and quickly neutralized with 20 mM citrate pH 5.0+300 mM NaCl. The antibody was then further purified using a Superdex 200 16/600 column. The monomeric peak fractions were pooled and concentrated. The final purified protein had a final endotoxin levels lower than 10 EU/mg and was kept in 20 mM histidine pH 6.0+150 mM NaCl.

For anti-PD-L1/IL-15 bifunctional fusion antibody production, the “knob” and “hole” constructs in respective IgG1 backbone formats were transfected to ExpiHEK293-F cells in free style system (Invitrogen) according to standard protocol. Cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH2.5+150 mM NaCl and quickly neutralized with 20 mM citrate pH 5.0+300 mM NaCl. The antibody was then further purified by a Superdex 200 Increase 16/600 column. The monomeric peak fractions were pooled and concentrated. The final purified protein had a final endotoxin levels lower than 10 EU/mg and kept in 1×PBS buffer.

For monovalent IL-2-Fc fusion protein production, the “knob” and “hole” constructs in respective IgG1 backbone format were transfected to ExpiHEK293-F cells with the ratio of 1:1. The cells were grown for five days and the supernatant was collected by centrifugation and filtered through a 0.2 μm PES membrane. The Fc fusion agonist first was purified by MabSelect PrismA protein A resin (GE Health). The protein was eluted with 100 mM Gly pH2.5+150 mM NaCl and quickly neutralized with 20 mM citrate pH 5.0+300 mM NaCl. The agonist protein was then concentrated to 1 mL and further purified by a Superdex 200 16/600 gel filtration column. The monomeric peak fractions were pooled and concentrated. The final purified protein had a final endotoxin levels lower than 10 EU/mg and was kept in 1×PBS. The purified monovalent IL-2-Fc fusion agonists were run on an SDS gel (4-12% Bis-Tris Bolt gel, with MES running buffer).

For the monovalent (in Fab format) anti-PD-L1/IL-2 bifunctional antibody production, the “knob” and “hole” constructs in respective IgG1 backbone formats together with the corresponding light chain constructs were transfected to ExpiHEK293-F cells in free style system (Invitrogen) according to standard protocol with ratio of knob:hole:light chain of 1:4:4. Cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH 2.5+150 mM NaCl and quickly neutralized with 20 mM histidine pH 5.0+150 mM NaCl. The antibody was then further purified by a Superdex 200 16/600 column. The monomeric peak fractions were pooled and concentrated. The final purified protein had a final endotoxin levels lower than 10 EU/mg and was kept in 20 mN histidine, 150 mM NaCl buffer.

For the monovalent (in scFv format) anti-PD-L1/IL-2 bifunctional antibody production, the “knob” and “hole” constructs in respective IgG1 backbone formats were transfected to ExpiHEK293-F cells in free style system (Invitrogen) according to standard protocol with ratio of knob:hole of 1:2. Cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH 2.5+150 mM NaCl and quickly neutralized with 20 mM histidine pH 5.0+150 mM NaCl. The antibody was then further purified by a Superdex 200 16/600 column. The monomeric peak fractions were pooled and concentrated. The final purified protein had a final endotoxin levels lower than 10 EU/mg and was kept in 20 mN histidine, 150 mM NaCl buffer.

For the bivalent (in Fab format) anti-PD-L1/IL-2 bifunctional antibody production, the “knob” and “hole” constructs in respective IgG1 backbone formats together with the corresponding light chain constructs were transfected to ExpiHEK293-F cells in free style system (Invitrogen) according to standard protocol with ratio of knob:hole:light chain of 1:2:2. Cells were grown for five days before harvesting. The supernatant was collected by centrifugation and filtered through a 0.2 μm PES membrane. The antibody was purified by MabSelect PrismA protein A resin (GE Health). The protein was eluted with 100 mM Gly pH 2.5+150 mM NaCl and quickly neutralized with 20 mM histidine pH 5.0+150 mM NaCl. The antibody was then further purified by a Superdex 200 16/600 column. The monomeric peak fractions were pooled and concentrated. The final purified protein had a final endotoxin level lower than 10 EU/mg and was kept in 20 mN histidine, 150 mM NaCl buffer.

For the anti-PD-L1/IL-2 fusion antibody production, the constructs were transfected to ExpiHEK293-F cells in free style system (Invitrogen) according to standard protocol. Cells were grown for five days before harvesting. The supernatant was collected by centrifugation and filtered through a 0.2 μm PES membrane. The antibodies were purified by Ni-Sepharose (GE Healthcare) affinity column according to the manufacturer's protocol. The antibody was further purified by a Superdex 200 16/600 column. The high homogeneous monomeric peak fractions of the agonists were each pooled and concentrated. The final endotoxin level was less than 10 EU/mg. The proteins were each stored in 1×PBS buffer for binding and functional analysis.

### Example 4. Anti-PD-L1 Antibody Igg Characterization

### Anti-PD-L1 IgG Antibody Binding to PD-L1 in ELISA

An ELISA assay was developed to determine the EC50 of anti-PD-L1 IgG antibodies. Briefly, 384 well plate was immobilized with human PD-L1-HIS tagged recombinant protein at final concentration of 2 μg/mL in 1×PBS in total volume of 25 μL per well. The plate was incubated overnight at 4° C. followed by blocking with 80 μL of superblock per well for 1 hour. Titration of purified PD-L1 IgG starting at 200 nM 2 fold serial dilution, 25 μL was added to human PD-L1 immobilized wells and incubated for 1 hour with shaking. The PD-L1 binding was detected by adding 25 μL of anti-hFc HRP diluted at 1:5000 in 1×PBST. In between each step, the plate was washed 3 times with 1×PBST in a plate washer. The plate was then developed with 20 μL of TMB substrate for 5 min and stopped by adding 20 μL of 2N sulfuric acid. The plate was read at OD450 nm Biotek plate reader and then plotted in Prism 8.1 software. Table 17 shows the ELISA binding EC50 for anti-PD-L1 IgG antibody.

TABLE 17
ELISA binding EC50 for anti-PD-L1 IgG antibody.

| Antibody | Construct pairs | PD-L1 Binding ELISA (nM) |
|---|---|---|
| PD-L1 | EP205/EP206 | 0.1635 |
| Atezolizumab | — | 0.1571 |

### Binding Kinetics of Anti-PD-L1 IgG Antibody to PD-L1 in SPR

Kinetic analysis of anti-PD-L1 IgG has been assessed by SPR technology with Biacore T200. The assay was run with Biacore T200 control software version 2.0. For each cycle, 1 μg/mL of anti-hPD-L1-IgG was captured for 60 seconds at flow rate of 10 μl/min on flow cell 2 in 1×HBSP buffer on Protein A sensor chip. 2-fold serial hPD-L1-HIS tagged protein was injected onto both reference flow cell 1 and anti-PD-L1 IgG captured flow cell 2 for 150 seconds at flow rate of 30 μl/min followed by wash for 300 seconds. The flow cells were then regenerated with Glycine pH 2 for 60 seconds at flow rate of 30 μl/min. 8 concentration points from 100 nM-0 nM were assayed per anti-PD-L1 IgG in a 96 well plate. The kinetics of Anti-PD-L1 IgG binding to PD-L1 protein was analyzed with Biacore T200 evaluation software version 3.0. The specific binding response unit was derived from subtraction of binding to reference flow cell 1 from antibody captured flow cell 2. Table 18 below shows the binding kinetics of the anti-PD-L1 IgG antibody by SPR.

TABLE 18
The binding kinetics of the anti-PD-L1 IgG antibody by SPR.

| Antibody | Construct pairs | Kₒₙ | K_{off} | K_{D} (nM) |
|---|---|---|---|---|
| 2019EP69-F03 | EP205/EP206 | 9.561E+5 | 3.472E−4 | 3.631E−10 |
| 2018EP164-F04 | EP480/EP481 | 1.245E+5 | 7.142E−4 | 5.734E−9 |
| 2018EP280-E04 | EP486/EP489 | 2.741E+5 | 7.565E−4 | 2.760E−9 |
| 2018EP171-H02 | EP488/EP489 | 4.093E+5 | 0.003889 | 9.501E−9 |
| Atezolizumab | — | NA | NA | NA |

NA: data not available

### Anti-PD-L1 IgG Antibody Binding to Cell Surface PD-L1 in FACS

200 nM of purified anti-PD-L1 IgG antibodies were diluted in full medium and incubated with PD-L1/K562 and K562 cells in 96 wells plate on ice for 1 hour. Cells were spun down at 1200 rpm for 5 minutes at 4° C. to remove primary antibodies. Cells were then washed once with 200 μL of full medium per well. Samples were detected with anti-hFc Alexa Fluor 647 by adding 100 μL of diluted secondary antibody and incubated at 4° C. for 30 minutes in the dark. Samples were spun down at 1200 rpm for 5 minutes at 4° C. and washed twice with 200 μL of 1×PBS per well. Reconstituted samples in 200 μL of 1×PBS and read on Attune NxT cytometer. Analysis was done by Attune NXT software plotting the overlay histogram of anti-PD-L1 antibody binding onto both negative and target cell lines.

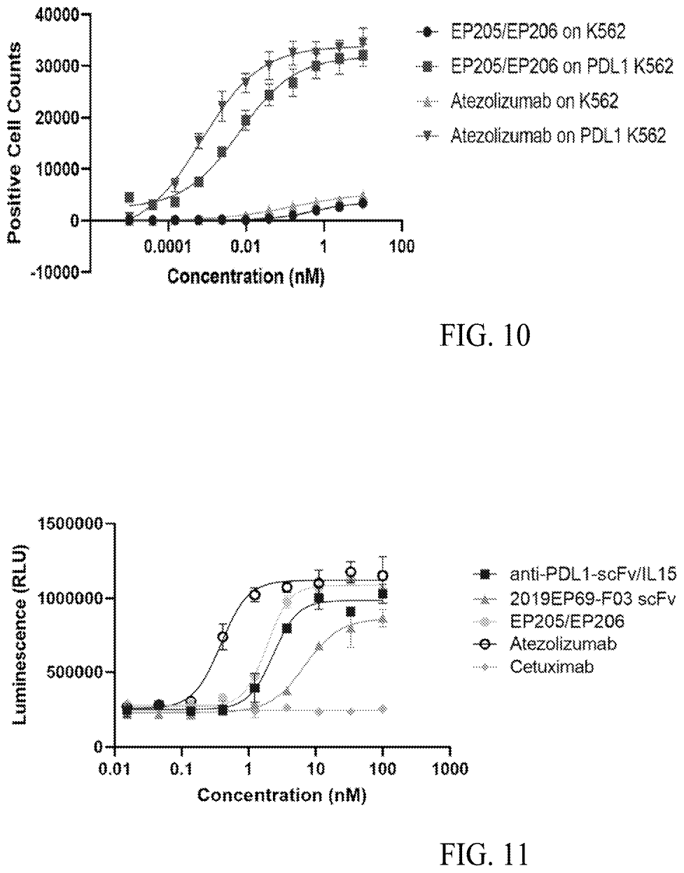

The binding competition of the anti-PD-L1 IgG antibody to PD-L1 and PD-1 interaction in HTRF assay and the activation of immune cells in Jurkat cell NFAT reporter assay were the same as described in scFv characterization. Table 19 below shows the Jurkat cell reporter assay EC50, the PD-L1/K562 cell surface binding EC50 and HTRF PD-1, and PD-L1 competition results for the IgG antibody. FIG. 10 shows the PD-L1/K562 cell surface FACS binding results.

TABLE 19
The Jurkat cell reporter assay EC50, the PD-L1/K562
cell surface binding EC50, and HTRF PD-1 and PD-L1
competition results for the IgG antibody.

| | | Jurkat | | |
|---|---|---|---|---|
| | | Reporter | HTRF | PD-L1/K562 |
| | Construct | assay EC50 | IC50 | FACS EC50 |
| Antibody | pairs | (nM) | (nM) | (pM) |
| PD-L1 | EP205/EP206 | 0.55 | 128 | 6.68 |
| Atezolizumab | — | 0.355 | 8 | 0.73 |

### Example 5. Anti-PD-L1/IL-15 Fusion Antibody Characterization

### Anti-PD-L1/IL-15 Binding to PD-L1 and IL-15 Receptors in ELISA

An ELISA assay was developed to determine the EC50 of anti-PD-L1 antibodies. Briefly, 384 well plate was immobilized with human IL-15Rα-His tagged or PD-L1 HIS tagged protein at final concentration of 2 μg/mL in 1×PBS in total volume of 25 μL per well. The plate was incubated overnight at 4° C. followed by blocking with 80 μL of superblock per well for 1 hour. Purified anti-PD-L1/IL-15 was 3-fold serial titrated from 200 nM. 25 μL was added to human PD-L1/IL-15 immobilized wells and incubated for 1 hour with shaking. The IL-15 or PD-L1 binding was detected by adding 25 μL of anti-Human HRP diluted at 1:10000 in 1×PBST. In between each step, the plate was washed 3 times with 1×PBST in a plate washer. The plate was then developed with 20 μL of TMB substrate for 5 minutes and stopped by adding 20 μL of 2N sulfuric acid. The plate was read at OD450 nm Biotek plate reader and then plotted in Prism 8.1 software to calculate EC50. Table 20 shows the PD-L1 ELISA binding EC50 for the antibody.

TABLE 20
Anti-PD-L1/IL-15 binding to PD-L1
and IL-15 receptors in ELISA.

| | | PD-L1 Binding |
|---|---|---|
| Bifunctional | Construct pairs | ELISA (nM) |
| PD-L1-Fab/IL-15 | EP203/EP204/EP205 | 0.061 |
| PD-L1-scFv/IL-15 | EP207/EP208 | 0.11 |

### Binding Kinetics of Anti-PD-L1/IL-15 to IL-2Rb and PD-L1 in SPR

Kinetic analysis of anti-PD-L1-IgG/IL-15 and anti-PD-L1-scFv-Fc/IL-15 bifunctional to IL-15Rβ and PD-L1 has been assessed by SPR technology with Biacore T200. Note, the IL-15 receptor shares the same Beta subunit with IL-2 receptor. Therefore, IL-15Rβ is also referred to as IL-2Rβ and CD122. The assay was run with Biacore T200 control software version 2.0. For each cycle, 1 μg/mL of anti-hPD-L1-IgG/IL-15 or anti-PD-L1 scFv-Fc/IL-15 was captured for 60 seconds at flow rate of 10 μl/min on flow cell 2 in 1×HBSP buffer on Protein A sensor chip. 2-fold serial diluted IL-15Rβ-HIS or hPD-L1-HIS tagged protein was injected onto both reference flow cell 1 and anti-PD-L1/IL-15 bifunctional captured flow cell 2 for 150 seconds at flow rate of 30 μl/min followed by wash for 300 seconds. The flow cells were then regenerated with Glycine pH 2 for 60 seconds at flow rate of 30 μl/min. 8 concentration points from 100 nM-0 nM (IL-15Rβ-HIS) or 300 nM-0 nM (PD-L1-HIS) was assayed per anti-PD-L1 IgG in a 96 well plate. The kinetics of anti-PD-L1/IL-15 bifunctional binding to IL-15RB and PD-L1 proteins were analyzed with Biacore T200 evaluation software version 3.0. The specific binding response unit was derived from subtraction of binding to reference flow cell 1 from antibody captured flow cell 2. Table 21 shows results for the binding kinetics of anti-PD-L1/IL-15 to IL-2Rβ and PD-L1.

TABLE 21
Binding kinetics of anti-PD-L1/IL-15 to IL-2Rβ and PD-L1 in SPR.

| | Construct | IL-2Rβ Binding SPR | | | PD-L1 Binding SPR | | |
|---|---|---|---|---|---|---|---|
| Antibody | pairs | kₐ (1/Ms) | k_{d} (1/s) | K_{D} (M) | kₐ (1/Ms) | k_{d} (1/s) | K_{D} (M) |
| PD-L1- | EP203/ | 6.017E+5 | 3.204E−2 | 5.324E−8 | 9.561E+5 | 3.472E−4 | 3.631E−10 |
| Fab/IL-15 | EP204/ | | | | | | |
| | EP205 | | | | | | |
| PD-L1- | EP207/ | 4.732E+5 | 2.983E−2 | 6.304E−8 | 9.234E+5 | 4.699E−4 | 5.088E−10 |
| scFv/IL-15 | EP208 | | | | | | |
| PD-L1 | EP205/ | NA | NA | NA | 9.561E+5 | 3.472E−4 | 3.631E−10 |
| | EP206 | | | | | | |

### Anti-PD-L1/IL-15 Antibodies in NFAT Reporter Assay

The activation of immune cells in Jurkat cell NFAT reporter assay (Promega, J1250) was the same as described in scFv characterization. The EC50 for the bifunctional antibodies is shown in Table 22. FIG. 11 shows the NFAT reporter assay results.

TABLE 22
EC50 of bifunctional anti-PD-L1/IL-15
antibodies in NFAT reporter assay.

| Bifunctional | Construct pairs | NFAT reporter EC50 (nM) |
|---|---|---|
| PD-L1-Fab/IL-15 | EP203/EP204/EP205 | NA |
| PD-L1-scFv/IL-15 | EP207/EP208 | 2.297 |

### P-STAT5 Activity of Anti-PD-L1/IL-15 Antibodies

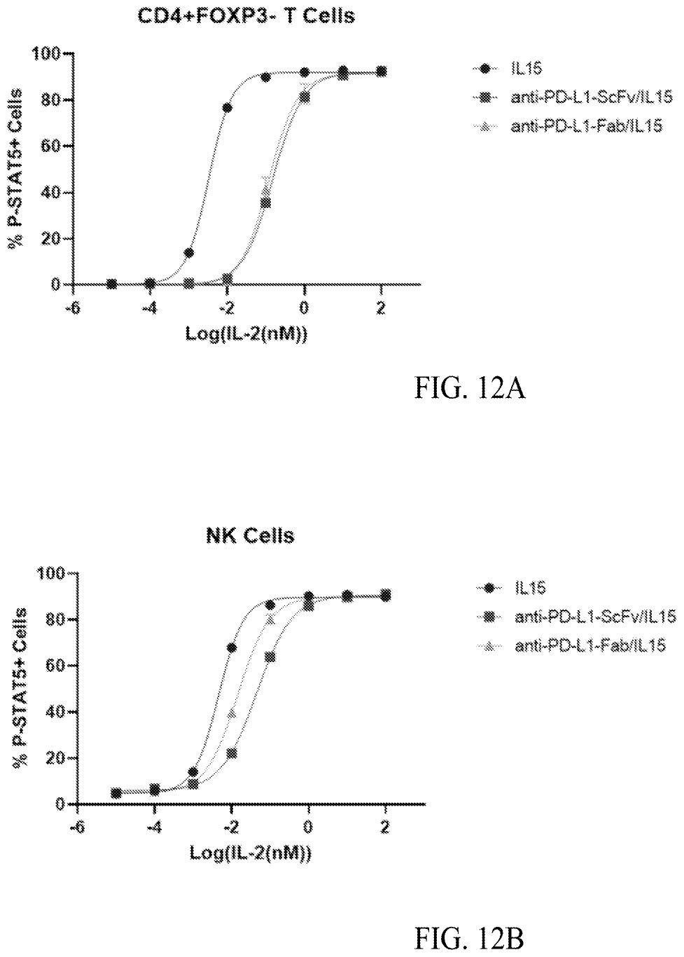

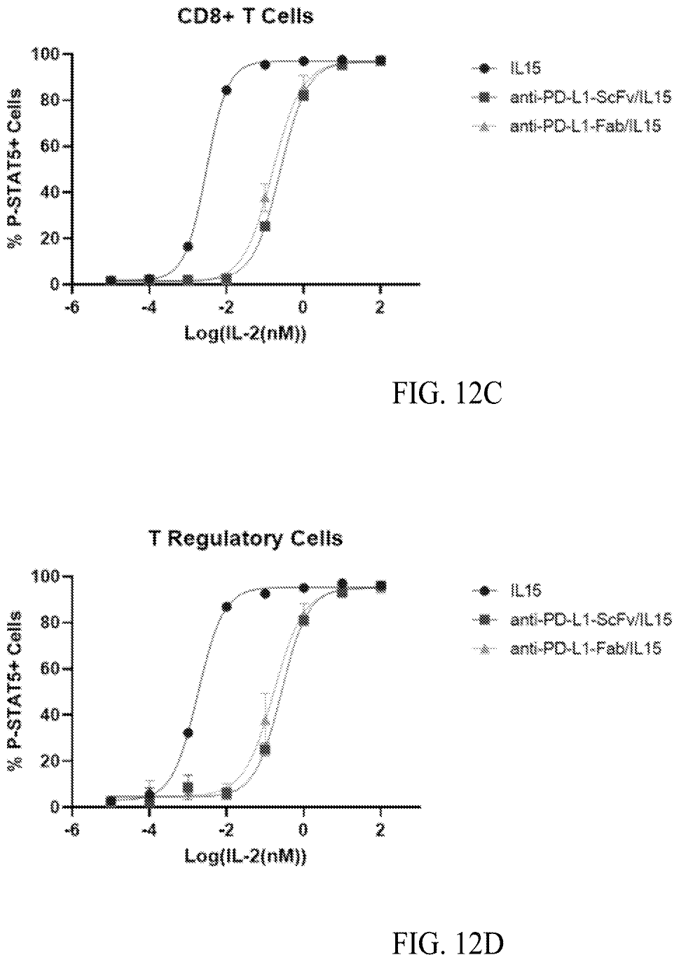

Human PBMCs were isolated from Leukocyte Reduction System (LRS) cones of two separate donors and plated at 250,000 cells/well in a 96-well plate in 90 μL of media. Cells were rested 1 hr at 37° C. Cells were stimulated with anti-PD-L1/IL-15 antibodies at 10× concentration in 10 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each antibody or IL-15 (Peprotech) titration. EC50 values for p-STAT5 activation were determined using Prism software. EC50 of p-STAT5 is shown in Table 23. FIG. 12 shows the p-STAT5 activation results in CD4+ T cells, CD8+ T cells, NK cells, and T regulatory cells.

TABLE 23
P-STAT5 activity of anti-PD-L1/IL-15 antibodies.

| | | EC50 (nM) | | |
|---|---|---|---|---|
| | Construct | CD8+ | NK | Treg |
| | pairs | T cell | Cell | Cell |
| PD-L1-Fab/IL-15 | EP203/EP204/ | 0.1571 | 0.01433 | 0.167 |
| | EP205 | | | |
| PD-L1-scFv/IL-15 | EP207/EP208 | 0.2482 | 0.04478 | 0.2627 |
| IL-15 | — | 0.002991 | 0.004617 | 0.00179 |

### Example 6. Monovalent IL-2-Fc Characterization

### Binding to IL-2Rα and IL-2Rβ Receptor ELISA

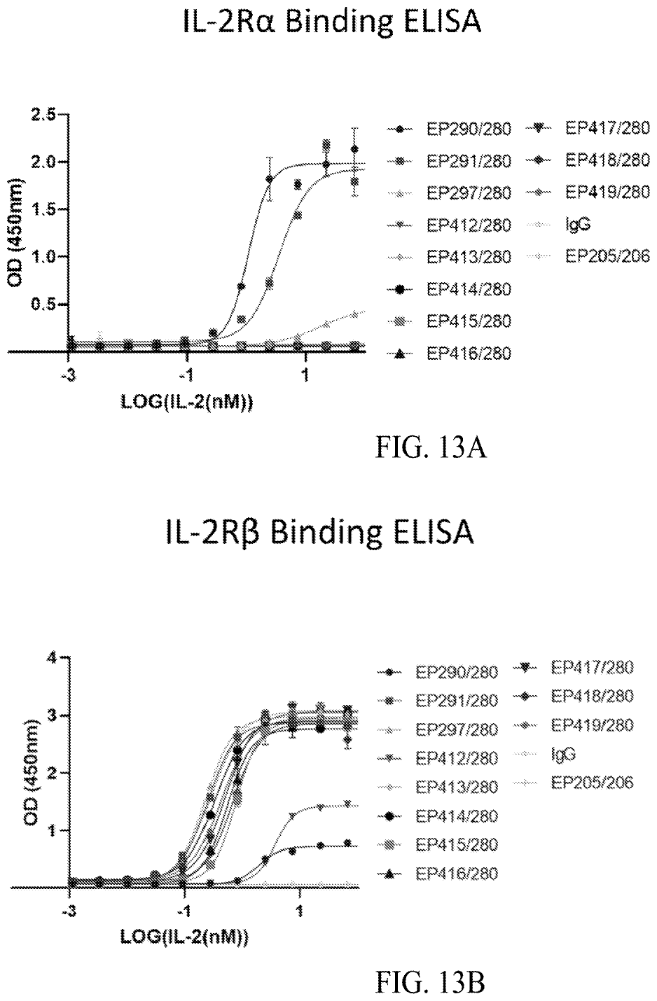

For monovalent IL-2 Fc-fusion proteins, recombinant His-tagged human IL-2Rα and IL-2Rβ were added in 25 μL of 1×PBS to wells of 384-well plate and incubated overnight at 4° C. to coat the plates. Plates were washed three times with 0.05% Tween20/1×PBS. Plates were blocked with 100 μL of SuperBlock for 1 hr at RT and then washed 3 times with 0.05% Tween20/1×PBS. IL-2 mutants were diluted in 0.05% Tween 20/1×PBS from 1000 nM to 0 nM and added to plates for 2 hrs at room temperature. Plates were then washed 6 times with 0.05% Tween20/1×PBS. Anti-HisTag-HRP was diluted 1:5000 in 0.05% Tween20/1×PBS and added to plates for 1 hr at RT. Plates were then washed 6 times with 0.05% Tween 20/1×PBS, and TMB was added to develop blue color. Reactions were stopped with 2N hydrogen sulfide and light absorbance at 450 nm was read on a BioTek plate reader. Absorbance versus IL-2 concentration is graphed for human IL-2Rα and IL-2Rβ. A summary of the EC50 values of ELISA binding is shown in Table 24. FIG. 13A and FIG. 13B show the binding of monovalent IL-2 Fc proteins to IL-2Rα and IL-2Rβ receptors, respectively.

TABLE 24
EC50 values of ELISA for monovalent IL-2 Fc binding
to IL-2Rα and IL-2Rβ receptors.

| | | IL-2Rα binding | IL-2Rβ binding |
|---|---|---|---|
| Format | Construct pair | ELISA (nM) | ELISA (nM) |
| IL-2-Fc | EP290/EP280 | 1.082 | 1.901 |
| | EP291/EP280 | 3.552 | 0.2479 |
| | EP297/EP280 | ND | 0.2283 |
| | EP412/EP280 | ND | 3.656 |
| | EP413/EP280 | ND | 0.2818 |
| | EP414/EP280 | ND | 0.3323 |
| | EP415/EP280 | ND | 0.7775 |
| | EP416/EP280 | ND | 0.5447 |
| | EP417/EP280 | ND | 0.5076 |
| | EP418/EP280 | ND | 0.6797 |
| | EP419/EP280 | ND | 0.4357 |

### Binding to IL-2Rα and IL-2Rβ Receptor SPR

Binding kinetics of monovalent IL-2Rβ Fc fusion proteins have been analyzed by SPR technology with Biacore T200. Briefly, anti-hFc antibody was immobilized on flow cell 1 and 2. For each cycle, 1 μg/mL of IL-2 Fc fusion protein was captured for 60 seconds at flow rate of 10 μL/min on flow cell 2 in 1×HBSP buffer on anti-hFc immobilized chip. 100 nM IL-2Rα-HIS tagged or IL-2Rβ-HIS tagged was 2-fold serial diluted and injected onto both reference flow cell 1 and IL-2 Fc fusion protein were captured at flow cell 2 for 150 seconds at flow rate of 30 μL/min. 300 seconds wash was applied after the last injection. The assay was set up with 8 serial diluted concentration points in 96 well format. The kinetics data was analyzed with Biacore T200 evaluation software 3.0. The specific binding response unit was derived from subtraction of binding to reference flow cell 1 from target flow cell 2. A summary of the binding kinetics of monovalent IL-2-Fc fusion proteins to IL-2 receptors is shown in Table 25.

TABLE 25
Binding to IL-2Rα and IL-2Rβ receptor SPR.

| | Construct | IL-2Rα binding | | | IL-2Rβ binding | | |
|---|---|---|---|---|---|---|---|
| Format | pair | ka (1/Ms) | kd (1/s) | KD (M) | ka (1/Ms) | kd (1/s) | KD (M) |
| IL-2 Fc | EP290/EP280 | NA | NA | 1.115E−8* | 1.960E+5 | 1.960E+5 | 2.004E−6 |
| | EP291/EP280 | NA | NA | NA | NA | NA | NA |
| | EP297/EP280 | ND | ND | ND | 3.280E+6 | 0.008194 | 2.498E−9 |
| | EP412/EP280 | ND | ND | ND | NA | NA | NA |
| | EP413/EP280 | ND | ND | ND | 7.815E+6 | 0.01109 | 1.419E−9 |
| | EP414/EP280 | ND | ND | ND | 4.555E+6 | 0.001084 | 2.379E−10 |
| | EP415/EP280 | ND | ND | ND | 1.324E+7 | 0.02207 | 1.666E−9 |
| | EP416/EP280 | ND | ND | ND | 4.215E+6 | 0.03518 | 8.346E−9 |
| | EP417/EP280 | ND | ND | ND | 7.609E+6 | 0.04376 | 5.752E−9 |
| | EP418/EP280 | ND | ND | ND | NA | NA | 2.250E−8* |
| | EP419/EP280 | ND | ND | ND | NA | NA | 6.280E−9* |

*Steady state Affinity.
ND: non-detectable binding

### P-STAT5 Profile

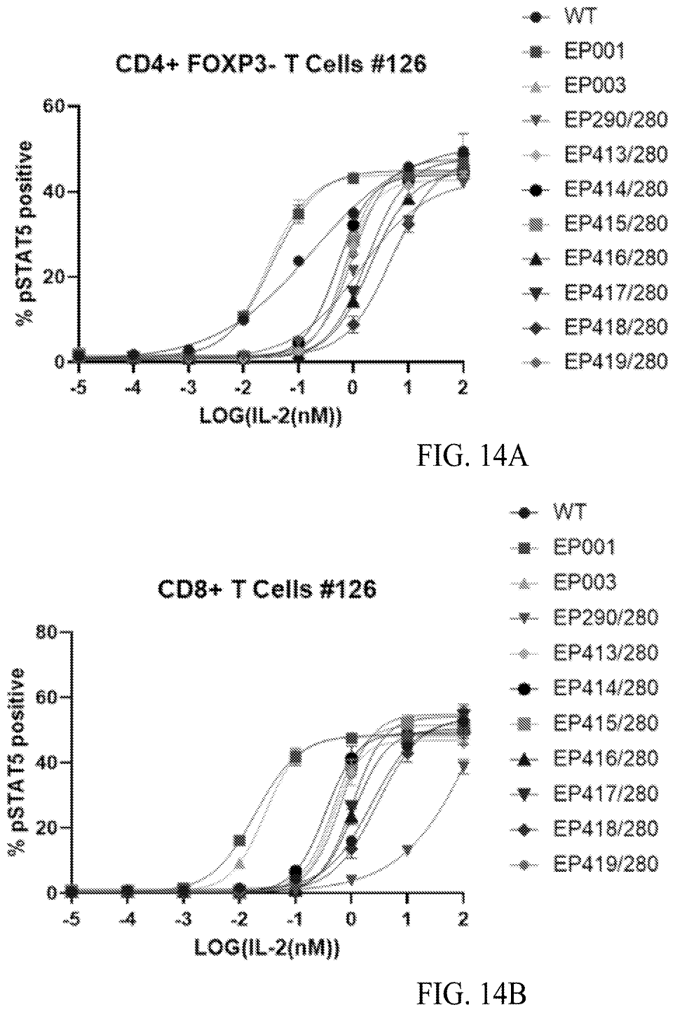

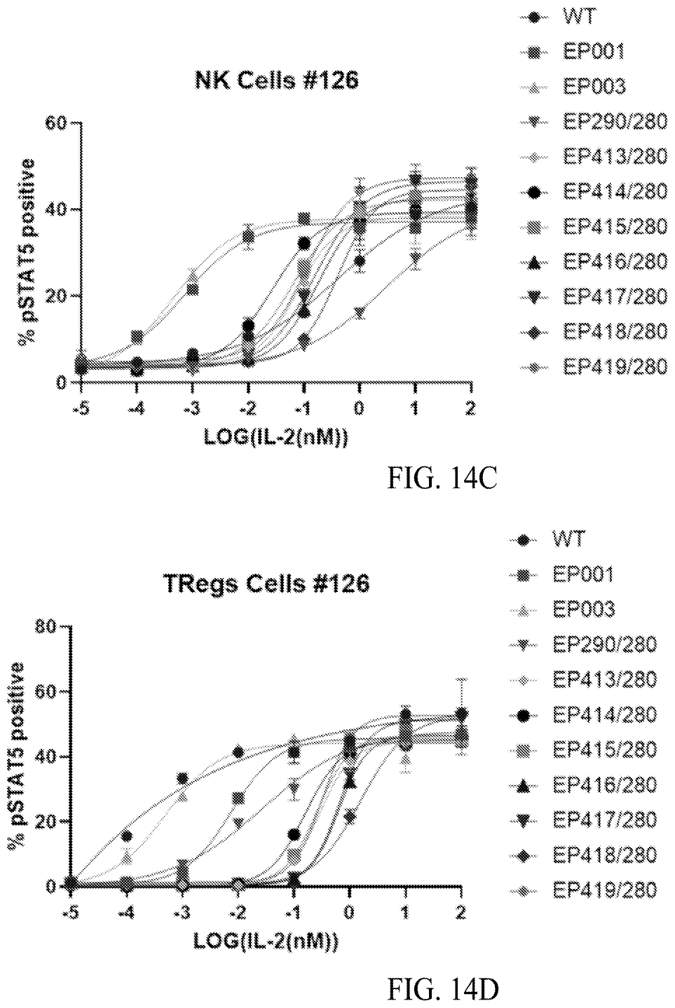

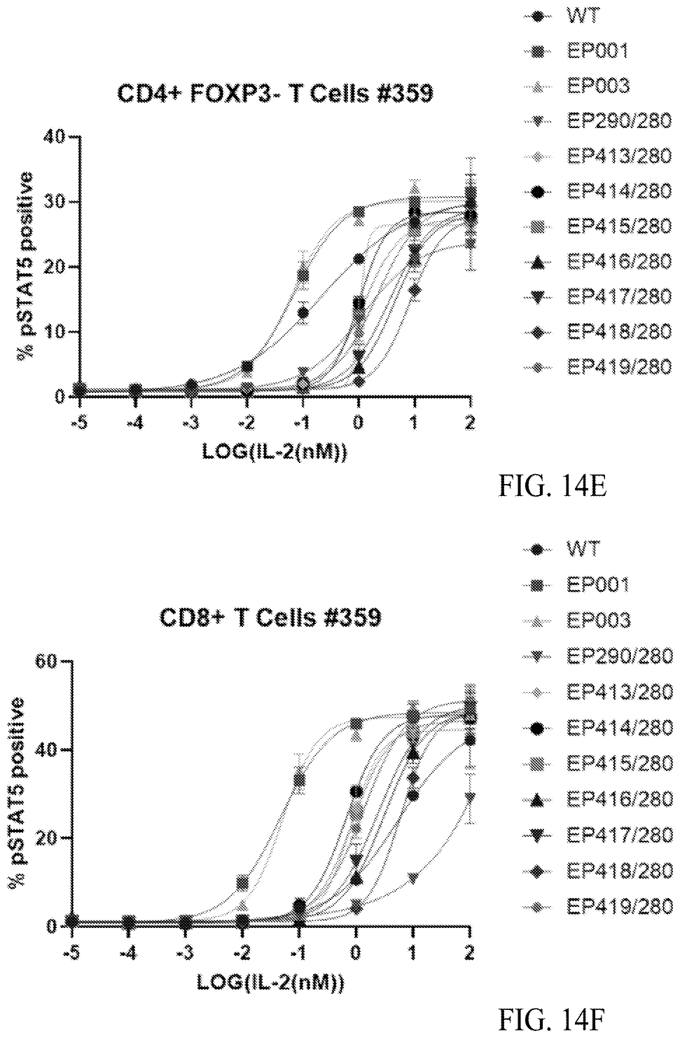

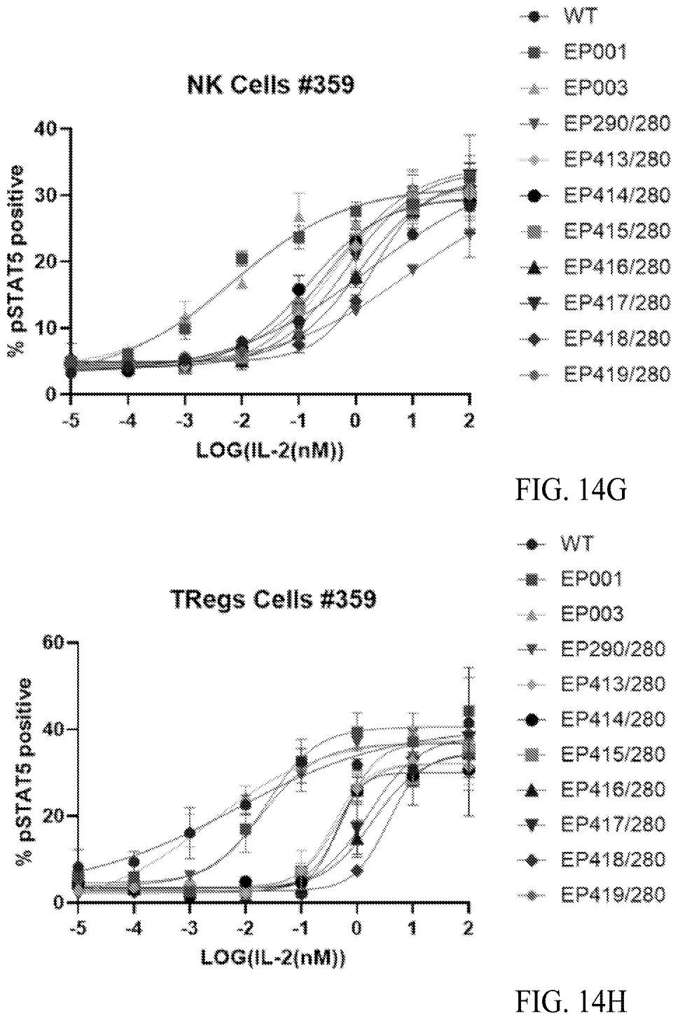

Human PBMCs were isolated from LRS cones of two separate donors and plated at 250,000 cells/well in a 96-well plate in 90 μL of media. Cells were rested 1 hr at 37° C. Cells were stimulated with human IL-2-Fc WT and engineered IL-2-Fc mutants at 10× concentration in 10 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The % of cells that were p-STAT5+ was determined and graphed versus each IL-2 titration. EC50 values for p-STAT5 activation were determined using Prism software and shown in Table 26. FIGS. 14A-D show the p-STAT5 profiling curves for CD4+ T cells, CD8+ T cells, NK cells, and T regulatory cells from donor-126. FIGS. 14E-H show the p-STAT5 profiling curves for CD4+ T cells, CD8+ T cells, NK cells, and T regulatory cells from donor-359.

TABLE 26
EC50 values for p-STAT5 activation.

| | | Donor-126 EC50 (nM) | | | Donor-359 EC50 (nM) | | |
|---|---|---|---|---|---|---|---|
| | Construct | CD8+ | NK | Treg | CD8+ | NK | Treg |
| Format | pair | T cell | Cell | Cell | T cell | Cell | Cell |
| IL-2-Fc | EP290/EP280 | 3.095 | 306.5 | 0.02665 | weak | 7.618 | 0.01938 |
| | EP291/EP280 | NA | NA | NA | NA | NA | NA |
| | EP297/EP280 | NA | NA | NA | NA | NA | NA |
| | EP412/EP280 | NA | NA | NA | NA | NA | NA |
| | EP413/EP280 | 0.07649 | 0.4673 | 0.2848 | 0.8045 | 0.337 | 0.3602 |
| | EP414/EP280 | 0.02651 | 0.3441 | 0.1588 | 0.6643 | 0.1479 | 0.4323 |
| | EP415/EP280 | 0.06504 | 0.4719 | 0.2630 | 0.8691 | 0.2325 | 0.4172 |
| | EP416/EP280 | 0.1842 | 1.116 | 0.6288 | 3.064 | 1.145 | 1.923 |
| | EP417/EP280 | 0.1595 | 1.036 | 0.6506 | 2.397 | 0.6835 | 1.577 |
| | EP418/EP280 | 0.3956 | 2.848 | 1.564 | 5.951 | 1.936 | 3.609 |
| | EP419/EP280 | 0.1088 | 0.6566 | 0.3199 | 1.34 | 0.4319 | 0.5882 |

### Example 7. IL-2/Anti-PD-L1-scFv Bifunctional Characterization

IL-2 Receptors and PD-L1 Binding Activity of IL-2/Anti-PD-L1 scFv Antibody in ELISA

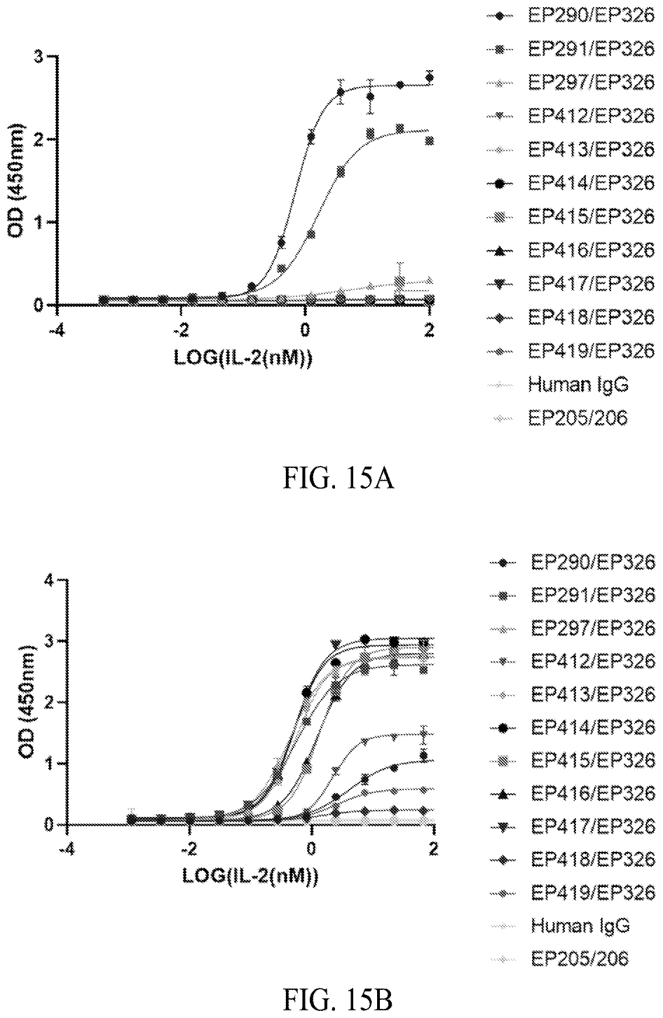

A 384 well plate was immobilized with anti-human Fc at final concentration of 2 μg/mL in 1×PBS in total volume of 25 μL per well. The plate was incubated overnight at 4C followed by blocking with 80 μL of superblock per well for 1 hour. A titration of IL-2/anti-PD-L1 scFv starting at 50 nM in volumes of 25 μL was added to wells and incubated for 1 hour with shaking. After washing, 25 μL of 30 nM biotinylated PD-L1 was added to each well. The PD-L1 binding by IL-2/anti-PD-L1 scFv the was detected by adding 25 μL of Streptavidin HRP diluted at 1:10000 in 1×PBST. In between each step, the plate was washed 3 times with 1×PBST in a plate washer. The plate was then developed with 25 μL of TMB substrate for 5 min and stopped by adding 25 μL of 2N sulfuric acid. The plate was read at OD450 nm Biotek plate reader and the binding correlation was plotted with Prism 8.1 software. The ELISA binding assays of the antibodies to IL-2Rα and IL-2Rβ receptors were performed as described in Example 6. The ELISA binding EC50 values were shown in Table 27. FIG. 15A, FIG. 15B, and FIG. 15C show the ELISA binding curves of the antibodies to IL-2Rα, IL-2Rβ and PD-L1, respectively.

TABLE 27
IL-2 receptors and PD-L1 binding activity
of IL-2/anti-PD-L1 scFv antibody in ELISA.

| | | IL-2Rα | IL-2Rβ | PD-L1 |
|---|---|---|---|---|
| | | binding | binding | binding |
| | Construct | ELISA | ELISA | ELISA |
| Format | pair | (nM) | (nM) | (nM) |
| IL-2/ | EP290/EP326 | 0.275 | 4.013 | 0.1405 |
| Anti-PD-L1 | EP291/EP326 | 1.013 | 0.552 | 0.246 |
| FcFv | EP297/EP326 | ND | 0.4302 | 0.1076 |
| | EP412/EP326 | ND | 2.182 | 0.1557 |
| | EP413/EP326 | ND | 0.5449 | 0.3455 |
| | EP414/EP326 | ND | 0.5103 | 0.2122 |
| | EP415/EP326 | ND | 1.333 | 0.2268 |
| | EP416/EP326 | ND | 1.235 | 0.2981 |
| | EP417/EP326 | ND | 0.5153 | 0.1878 |
| | EP418/EP326 | ND | NA | 0.2868 |
| | EP419/EP326 | ND | 0.6128 | 0.2058 |
| Anti-PD-L1 | EP205/EP206 | — | — | 0.1635 |
| Atezolizumab | — | — | — | 0.1571 |

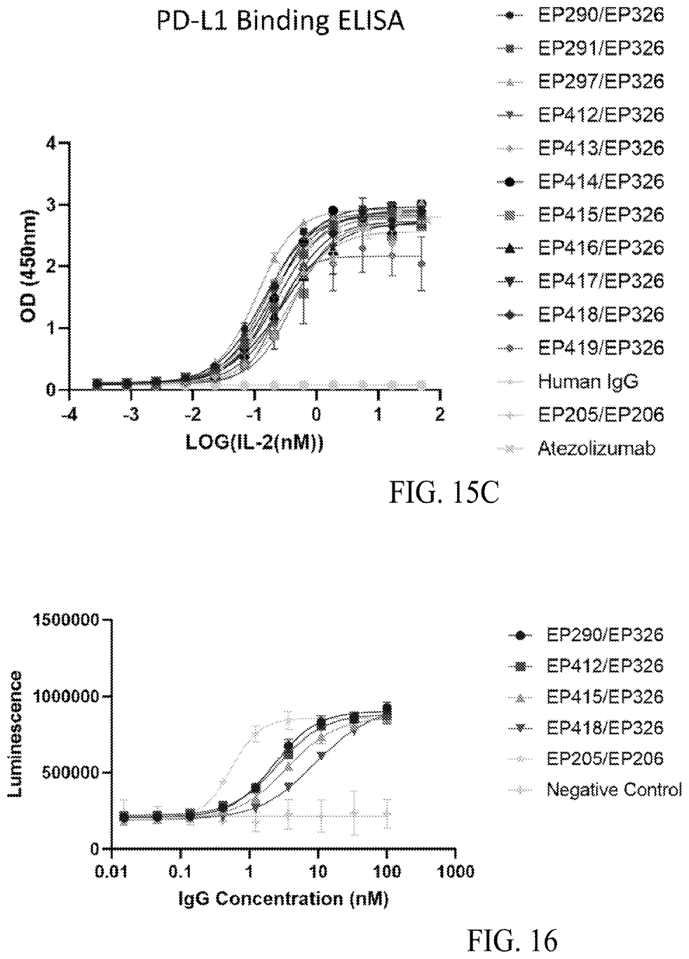

The binding competition of the anti-PD-L1 IgG antibody to PD-L1 and PD-1 interaction in HTRF assay and the activation of immune cells in Jurkat cell NFAT reporter assay were performed as described in scFv characterization. The HTRF IC50 and NFAT reporter assay EC50 values were shown in Table 28. FIG. 16 shows the NFAT reporter assay results.

TABLE 28
Binding competition of IL-2/anti-PD-L1 ScFv and anti-PD-L1 IgG
antibody to PD-L1 and PD-1 interaction in HTRF assay and the
activation of immune cells in Jurkat cell NFAT reporter assay.

| Bifunctional | HTRF IC50 (nM) | NFAT reporter EC50 (nM) |
|---|---|---|
| EP290/EP326 | 176.3 | 2.301 |
| EP412/EP326 | 134.1 | 2.606 |
| EP415/EP326 | 2281 | 3.46 |
| EP418/EP326 | 346.4 | 9.066 |
| EP205/EP206 | 128 | 0.5487 |

### Binding to IL-2Rα, IL-2Rβ Receptor and PD-L1 in SPR

Kinetic analysis of anti-PD-L1/IL-2 bifunctional to IL-2Rα, IL-2Rβ and PD-L1 has been assessed by SPR technology with Biacore T200. The assay was run with Biacore T200 control software version 2.0. For each cycle, 1 μg/mL of anti-hPD-L1/IL-2 was captured for 60 seconds at flow rate of 10 μL/min on flow cell 2 in 1×HBSP buffer on Protein A sensor chip. 2-fold serial diluted IL-2Rα-HIS or IL-2Rβ-HIS or hPD-L1-HIS tagged protein was injected onto both reference flow cell 1 and anti-PD-L1/IL-2 bifunctional captured flow cell 2 for 150 seconds at flow rate of 30 μl/min followed by wash for 300 seconds. The flow cells were then regenerated with Glycine pH2 for 60 seconds at flow rate of 30 μL/min. 8 concentration points from 100 nM-0 nM (IL-2Rα-HIS and IL-2Rβ-HIS) or 300 nM-0 nM (PD-L1-HIS) was assayed per anti-PD-L1 IgG in a 96 well plate. The kinetics of anti-PD-L1/IL-2 bifunctional binding to IL-2Rα, IL-2Rβ and PD-L1 proteins was analyzed with Biacore T200 evaluation software version 3.0. The specific binding response unit was derived from subtraction of binding to reference flow cell 1 from antibody captured flow cell 2. The binding kinetics of antibodies to respective IL-2 and PD-L1 were shown in Table 29.

TABLE 29
Kinetic analysis of IL-2/anti-PD-L1 ScFv bifunctional to IL-2Rα, IL-2Rβ and PD-L1.

| | | IL-2Rα binding | | | IL-2Rβ binding | | | PD-L1 Binding | | |
|---|---|---|---|---|---|---|---|---|---|---|
| | Construct | ka | kd | KD | ka | kd | KD | ka | kd | KD |
| Format | pair | (1/Ms) | (1/s) | (M | (1/Ms) | (1/s) | (M) | (1/Ms) | (1/s) | (M) |
| IL-2/ | EP290/ | NA | NA | NA | 8.855E+5 | 0.05082 | 5.740E−8 | 5.003E+5 | 2.320E−4 | 4.638E−10 |
| PD-L1 | EP326 | | | | | | | | | |
| scFv | EP291/ | NA | NA | Weak | 4.103E+5 | 7.663E−4 | 1.867E−9 | 5.736E+5 | 1.949E−4 | 3.397E−10 |
| | EP326 | | | | | | | | | |
| | EP297/ | NA | NA | Weak | 2.570E+6 | 0.008542 | 3.323E−9 | 7.822E+5 | 1.488E−4 | 1.902E−10 |
| | EP326 | | | | | | | | | |
| | EP412/ | ND | ND | ND | 2.988E+4 | 0.3573 | 1.196E−5 | 4.030E+5 | 2.389E−4 | 5.929E−10 |
| | EP326 | | | | | | | | | |
| | EP413/ | ND | ND | ND | 1.648E+6 | 0.01072 | 6.502E−9 | 4.868E+5 | 2.468E−4 | 5.069E−10 |
| | EP326 | | | | | | | | | |
| | EP414/ | NA | NA | NA | NA | NA | NA | NA | NA | NA |
| | EP326 | | | | | | | | | |
| | EP415/ | ND | ND | ND | 3.416E+6 | 0.02107 | 6.168E−9 | 4.865E+5 | 2.261E−4 | 4.648E−10 |
| | EP326 | | | | | | | | | |
| | EP416/ | ND | ND | ND | 1.281E+6 | 0.03393 | 2.648E−8 | 5.194E+5 | 2.024E−4 | 3.897E−10 |
| | EP326 | | | | | | | | | |
| | EP417/ | ND | ND | ND | 2.263E+6 | 0.04966 | 2.194E−8 | 4.974E+5 | 2.136E−4 | 4.295E−10 |
| | EP326 | | | | | | | | | |
| | EP418/ | ND | ND | ND | NA | NA | 7.579E−8* | 5.093E+5 | 2.786E−4 | 5.470E−10 |
| | EP326 | | | | | | | | | |
| | EP419/ | NA | NA | NA | NA | NA | NA | NA | NA | NA |
| | EP326 | | | | | | | | | |

*Steady state Affinity
ND: non-detectable binding

IL-2/Anti-PD-L1 scFv p-STAT5 Activation in Human PBMCs

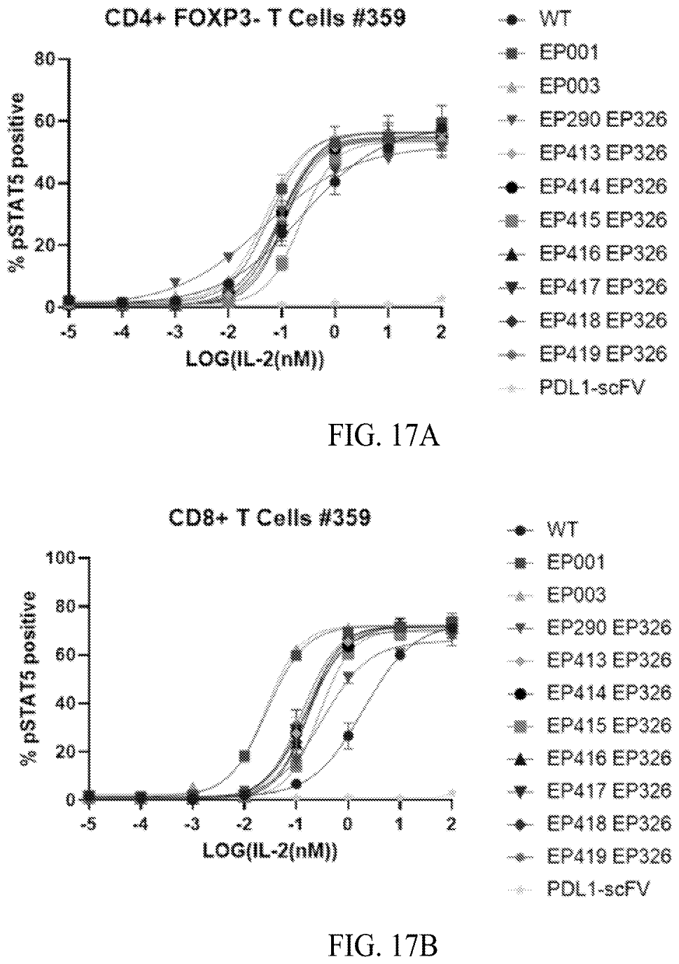

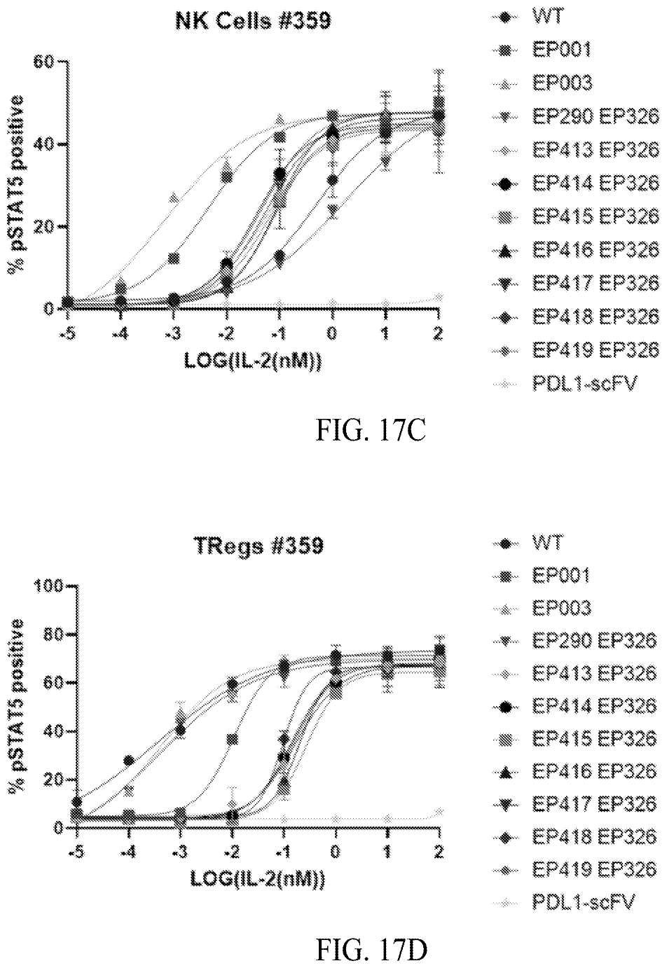

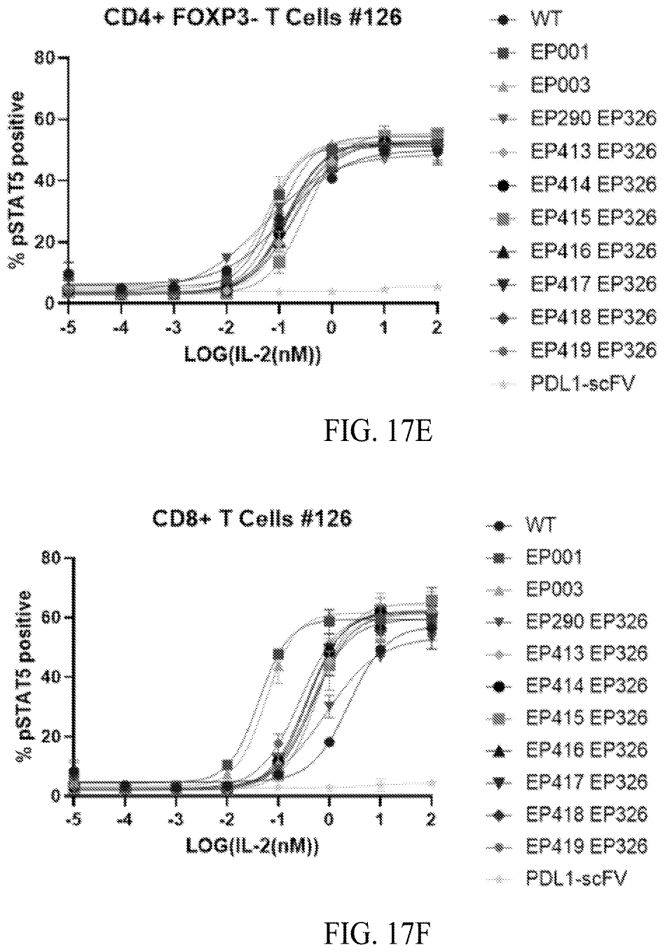

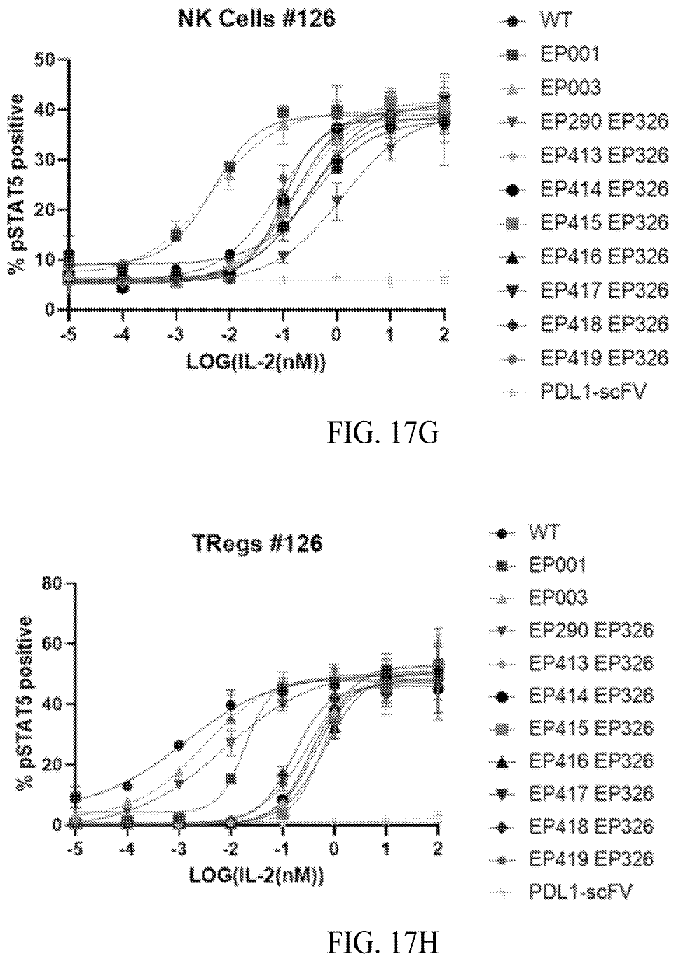

Human PBMCs were isolated from LRS cones of two separate donors and plated at 250,000 cells/well in a 96-well plate in 90 μL of media. Cells were rested 1 hr at 37° C. Cells were stimulated with human IL-2/anti-PD-L1 scFv WT and engineered IL-2/anti-PD-L1 scFv mutants at 10× concentration in 10 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The percentage of cells that were p-STAT5+ was determined and graphed versus each IL-2 titration. EC50 values for p-STAT5 activation were determined using Prism software and shown in Table 30. FIGS. 17A-D show p-STAT5 results of PBMC cells from donor-359. FIGS. 17E-H show the p-STAT5 results of PBMC cells from donor-126.

TABLE 30
IL-2/anti-PD-L1 scFv p-STAT5 activation in human
PBMCs from donors #359 and #126.

| | | Donor-359 EC50 (nM) | | | Donor-126 EC50 (nM) | | |
|---|---|---|---|---|---|---|---|
| | Construct | CD8+ | NK | Treg | CD8+ | NK | Treg |
| Format | pair | T cell | Cell | Cell | T cell | Cell | Cell |
| IL-2/ | EP290/EP326 | 0.3218 | 1.635 | 0.000397 | 0.8547 | 1.215 | 0.007386 |
| Anti-PD- | EP413/EP326 | 0.1467 | 0.06049 | 0.172 | 0.4499 | 0.1682 | 0.4269 |
| L1 | EP414/EP326 | 0.1574 | 0.03597 | 0.1531 | 0.3805 | 0.0979 | 0.3445 |
| ScFv | EP415/EP326 | 0.2758 | 0.0645 | 0.268 | 0.5887 | 0.1675 | 0.6262 |
| | EP416/EP326 | 0.1709 | 0.08715 | 0.205 | 0.4946 | 0.3076 | 0.6471 |
| | EP417/EP326 | 0.1476 | 0.05406 | 0.1674 | 0.3524 | 0.1553 | 0.4123 |
| | EP418/EP326 | 0.17 | 0.07943 | 0.09565 | 0.4444 | 0.2756 | 0.1619 |
| | EP419/EP326 | 0.1291 | 0.04648 | 0.1381 | 0.2709 | 0.07921 | 0.3268 |

### Example 8. IL-2/Anti-PD-L1-Fab Bifunctional and Anti-PD-L1/IL-2 Fusion Characterization

### IL-2/Anti-PD-L1 Fab PD-L1 Binding ELISA, HTRF Competition and Jurkat Reporter Assay

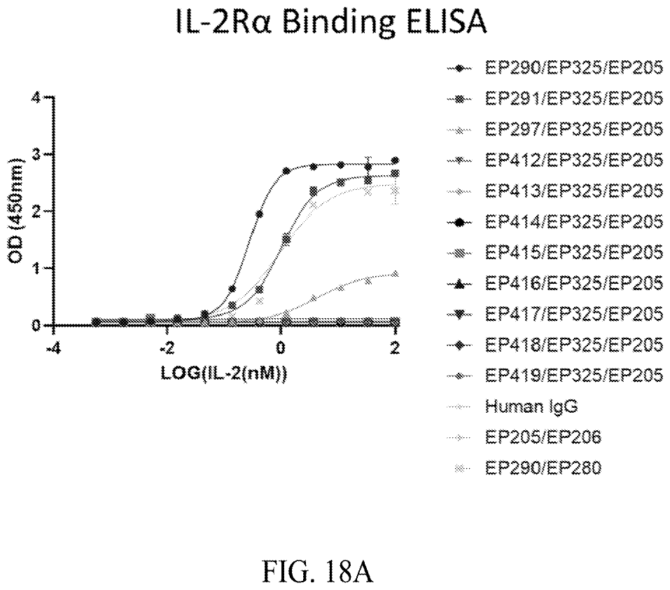

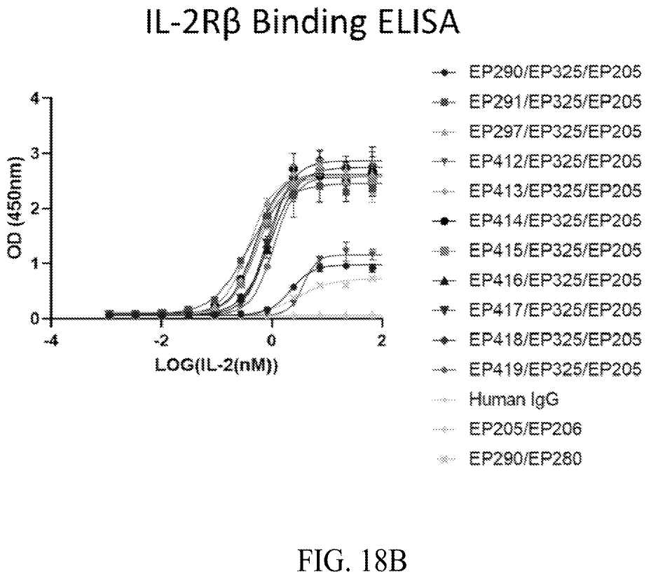

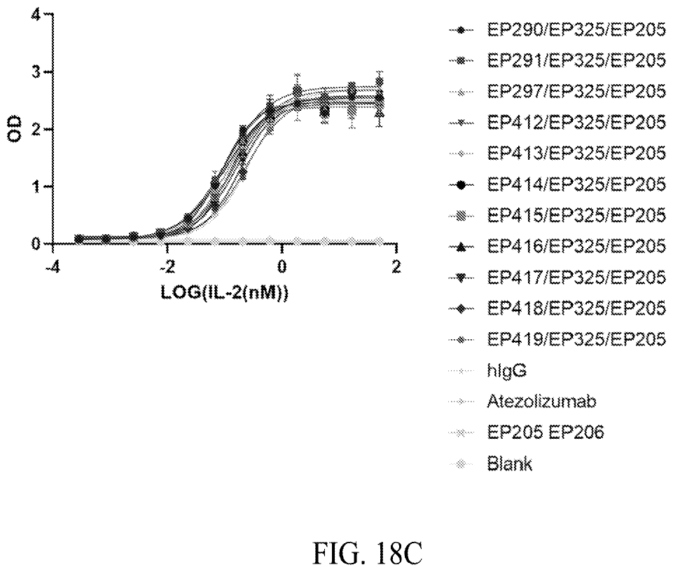

A 384 well plate was immobilized with anti-human Fc at final concentration of 2 μg/mL in 1×PBS in total volume of 25 μL per well. The plate was incubated overnight at 4° C. followed by blocking with 80 μL of superblock per well for 1 hour. A titration of IL-2/anti-PD-L1 FAB starting at 50 nM in volumes of 25 μL was added to wells and incubated for 1 hour with shaking. After washing, 25 μL of 30 nM biotinylated PD-L1 was added to each well. The PD-L1 binding by IL-2/anti-PD-L1 FAB the was detected by adding 25 μL of Streptavidin HRP diluted at 1:10000 in 1×PBST. In between each step, the plate was washed 3 times with 1×PBST in a plate washer. The plate was then developed with 25 μL of TMB substrate for 5 mins and stopped by adding 25 μL of 2N sulfuric acid. The ELISA binding assays of the antibodies to IL-2Rα and IL-2Rβ receptors were the same as described in Example 6. The plate was read at OD450 nm Biotek plate reader and the binding correlation was plotted with Prism 8.1 software. The ELISA binding EC50 values were shown in in Table 31. FIG. 18A, FIG. 18B, and FIG. 18C show ELISA binding curves of the IL-2 Fc/anti-PD-L1 Fab variants to IL-2Rα, IL-2Rβ and PD-L1, respectively.

TABLE 31
EC50 values from ELISA binding assays
of IL-2 Fc/anti-PD-L1 Fab.

| | | IL-2Rα | IL-2Rβ | PD-L1 |
|---|---|---|---|---|
| | | binding | binding | binding |
| | | ELISA | ELISA | ELISA |
| Format | Construct pair | (nM) | (nM) | (nM) |
| IL-2/ | EP290/EP325/EP205 | 0.275 | 2.213 | 0.08776 |
| Anti-PD-L1 | EP291/EP325/EP205 | 1.013 | 0.3669 | 0.1041 |
| Fab | EP297/EP325/EP205 | ND | 0.3833 | 0.09717 |
| | EP412/EP325/EP205 | ND | 3.737 | 0.1529 |
| | EP413/EP325/EP205 | ND | 0.5279 | 0.1294 |
| | EP414/EP325/EP205 | ND | 0.5013 | 0.1024 |
| | EP415/EP325/EP205 | ND | 0.5625 | 0.106 |
| | EP416/EP325/EP205 | ND | 0.9404 | 0.1206 |
| | EP417/EP325/EP205 | ND | 0.782 | 0.1618 |
| | EP418/EP325/EP205 | ND | 0.9196 | 0.1796 |
| | EP419/EP325/EP205 | ND | 1.086 | 0.2311 |
| Anti-PD-L1 | EP205/EP206 | — | — | 0.2599 |
| atezolizumab | — | — | — | 0.1999 |
| Human IgG | — | — | — | ND |

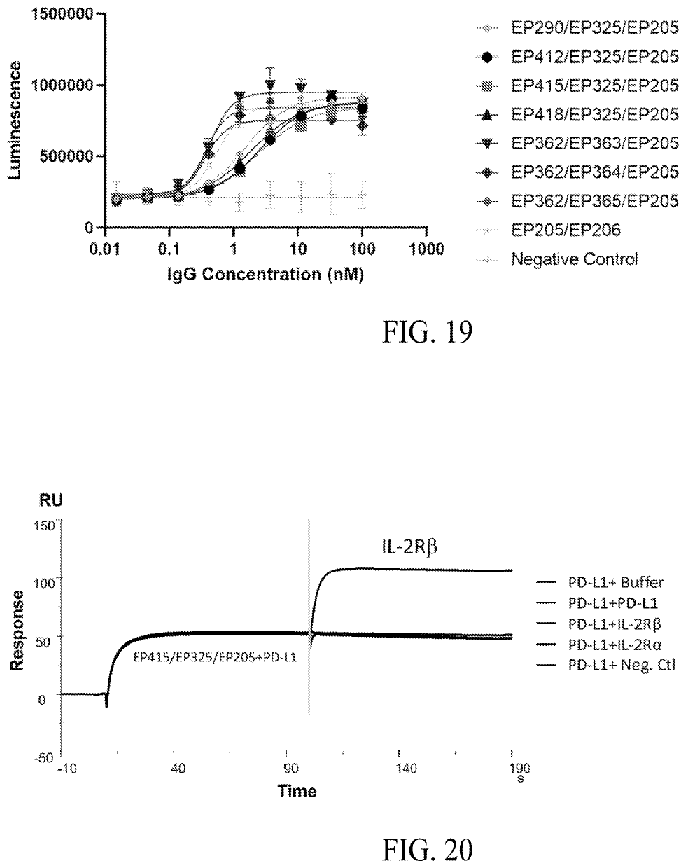

The binding competition of the bifunctional proteins to PD-L1 and PD-1 interaction in HTRF assay and the activation of immune cells in Jurkat cell NFAT reporter assay were the same as described in scFv characterization. The HTRF IC50 and NFAT reporter assay EC50 values were shown in Table 32. FIG. 19 shows the NFAT reporter assay curves.

TABLE 32
HTRF IC50 and NFAT reporter assay EC50 values.

| Bifunctional | HTRF IC50 (nM) | NFAT reporter EC50 (nM) |
|---|---|---|
| EP290/EP325/EP205 | 136.6 | 1.504 |
| EP412/EP325/EP205 | 188.5 | 2.522 |
| EP415/EP325/EP205 | 98.35 | 2.478 |
| EP418/EP325/EP205 | 89.41 | 1.871 |
| EP362/EP363/EP205 | 149.8 | 0.427 |
| EP362/EP364/EP205 | 128 | 0.356 |
| EP362/EP365/EP205 | 99.21 | 0.362 |
| EP205/EP206 | 128 | 0.5487 |

### Binding to IL-2Rα, IL-2Rβ Receptor and PD-L1 SPR

Kinetic analysis of anti-PD-L1/IL-2 bifunctional to IL-2Rα, I2Rβ and PD-L1 has been assessed by SPR technology with Biacore T200. The assay was run with Biacore T200 control software version 2.0. For each cycle, 1 μg/mL of anti-hPD-L1/IL-2 was captured for 60 seconds at flow rate of 10 μl/min on flow cell 2 in 1×HBSP buffer on Protein A sensor chip. 2-fold serial diluted IL-2Rα-HIS or IL-2Rβ-HIS or hPD-L1-HIS tagged protein was injected onto both reference flow cell 1 and anti-PD-L1/IL-2 bifunctional captured flow cell 2 for 150 seconds at flow rate of 30 μl/min followed by wash for 300 seconds. The flow cells were then regenerated with Glycine pH2 for 60 seconds at flow rate of 30 μl/min. 8 concentration points from 100 nM-0 nM (IL-2Rα-HIS and IL-2Rβ-HIS) or 300 nM-0 nM (PD-L1-HIS) was assayed per anti-PD-L1 IgG in a 96 well plate. The kinetics of anti-PD-L1/IL-2 bifunctional binding to IL-2Rα, IL-2Rβ and PD-L1 proteins was analyzed with Biacore T200 evaluation software version 3.0. The specific binding response unit was derived from subtraction of binding to reference flow cell 1 from antibody captured flow cell 2.

To evaluate whether the bifunctional antibody can bind IL-2 receptor and PD-L1 simultaneously, the anti-PDL1/IL2 bifunctional was captured for 60 seconds at flow rate of 10 μL/min on flow cell 2 in 1×HBSP buffer on Protein A sensor chip. The hPD-L1-HIS tagged protein was injected onto both reference flow cell 1 and anti-PD-L1/IL-2 bifunctional captured flow cell 2 for 60 seconds followed injecting IL-2Rα-HIS or IL-2Rβ-HIS protein onto both reference flow cell 1 and hPD-L1 bound anti-PD-L1/IL-2 bifunctional captured flow cell 2 for 90 seconds at flow rate of 30 μl/min followed by wash for 120 seconds. FIG. 20 shows simultaneous binding of PD-L1 and IL-2Rα and IL-2Rβ to the antibodies at single concentration. The binding kinetics of the anti-PDL1/IL2 bifunctionals to IL-2 receptors and PD-L1 were measured as described above and data shown in Table 33.

TABLE 33
Binding kinetics of IL-2 Fc/anti-PD-L1 Fab to IL-2Rα, IL-2Rβ, and PD-L1 in SPR.

| | Construct | IL-2Rα binding | | | IL-2Rβ binding | | | PD-L1 Binding | | |
|---|---|---|---|---|---|---|---|---|---|---|
| Format | pair | Kon | Koff | KD | Kon | Koff | KD | Kon | Koff | KD |
| IL-2/ | EP290/ | | | *3.865E−8 | 1.122E+6 | 0.1288 | 1.149E−7 | 3.951E+5 | 3.655E−4 | 9.252E−10 |
| anti-PD- | EP325/ | | | | | | | | | |
| L1 Fab | EP205 | | | | | | | | | |
| | EP291/ | | | Weak | 6.198E+5 | 7.568E−4 | 1.221E−9 | 7.417E+5 | 2.692E−4 | 3.629E−10 |
| | EP325/ | | | | | | | | | |
| | EP205 | | | | | | | | | |
| | EP297/ | ND | ND | ND | 2.882E+6 | 0.008365 | 2.902E−9 | 7.702E+5 | 2.433E−4 | 3.160E−10 |
| | EP325/ | | | | | | | | | |
| | EP205 | | | | | | | | | |
| | EP412/ | ND | ND | ND | 1.063E+6 | 0.1017 | 9.566E−8 | 5.033E+5 | 4.092E−4 | 8.130E−10 |
| | EP325/ | | | | | | | | | |
| | EP205 | | | | | | | | | |
| | EP413/ | | | | 1.419E+6 | 0.007727 | 5.444E−9 | 4.278E+5 | 4.134E−4 | 9.662E−10 |
| | EP325/ | | | | | | | | | |
| | EP205 | | | | | | | | | |
| | EP414/ | NA | NA | NA | NA | NA | NA | NA | NA | NA |
| | EP325/ | | | | | | | | | |
| | EP205 | | | | | | | | | |
| | EP415/ | ND | ND | ND | 4.667E+6 | 0.02365 | 5.068E−9 | 5.485E+5 | 5.421E−4 | 9.885E−10 |
| | EP325/ | | | | | | | | | |
| | EP205 | | | | | | | | | |
| | EP416/ | ND | ND | ND | 1.316E+6 | 0.03229 | 2.453E−8 | 4.607E+5 | 3.085E−4 | 6.696E−10 |
| | EP325/ | | | | | | | | | |
| | EP205 | | | | | | | | | |
| | EP417/ | ND | ND | ND | NA | NA | *2.544E−8 | 4.822E+5 | 2.866E−4 | 5.944E−10 |
| | EP325/ | | | | | | | | | |
| | EP205 | | | | | | | | | |
| | EP418/ | ND | ND | ND | NA | NA | *7.225E−8 | 4.788E+5 | 3.663E−4 | 7.649E−10 |
| | EP325/ | | | | | | | | | |
| | EP205 | | | | | | | | | |
| | EP419/ | NA | NA | NA | NA | NA | NA | NA | NA | NA |
| | EP325/ | | | | | | | | | |
| | EP205 | | | | | | | | | |

*Steady state Affinity.
ND: non-detectable binding

IL-2/Anti-PD-L1 Fab p-STAT5 Activation in Human PBMCs

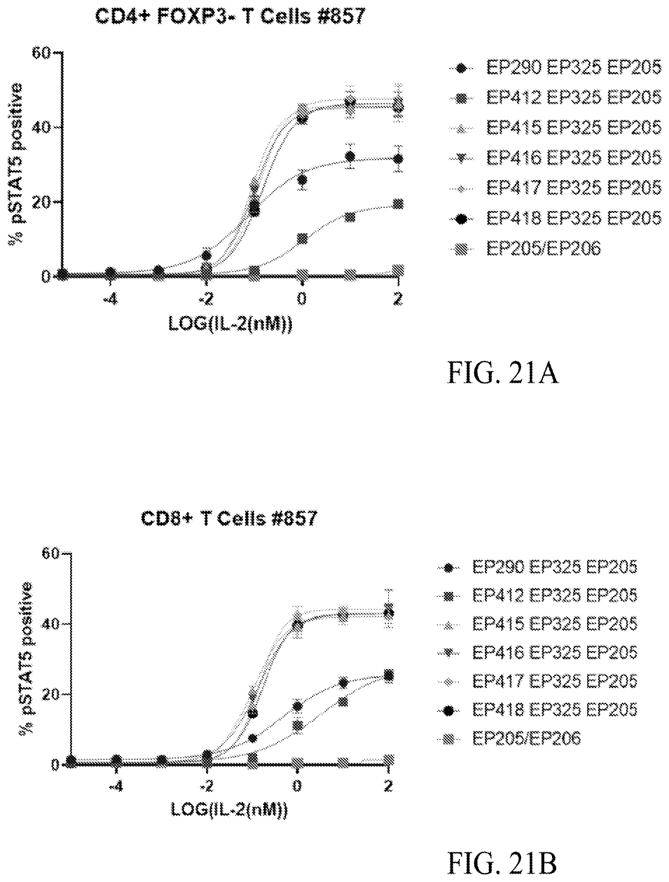

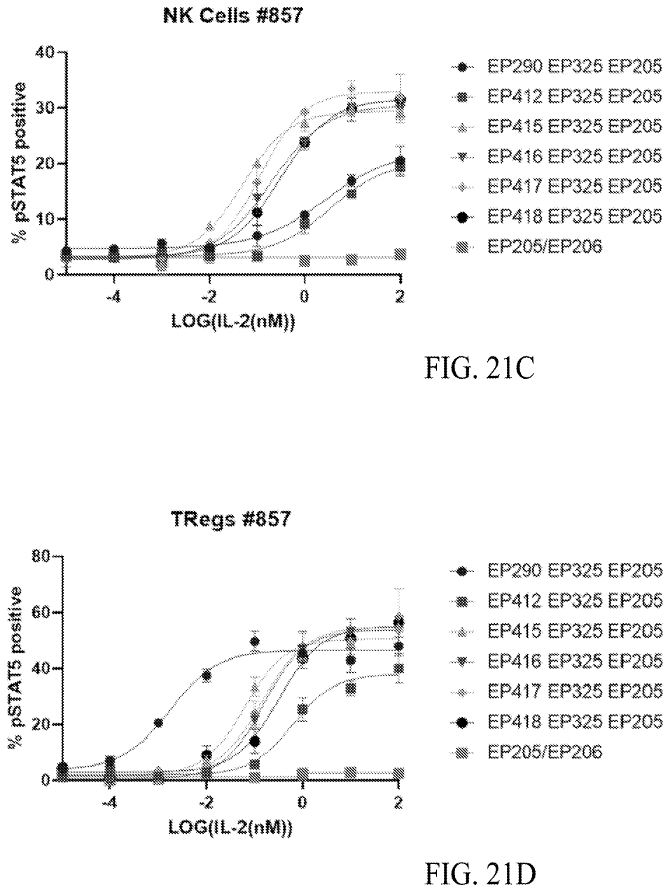

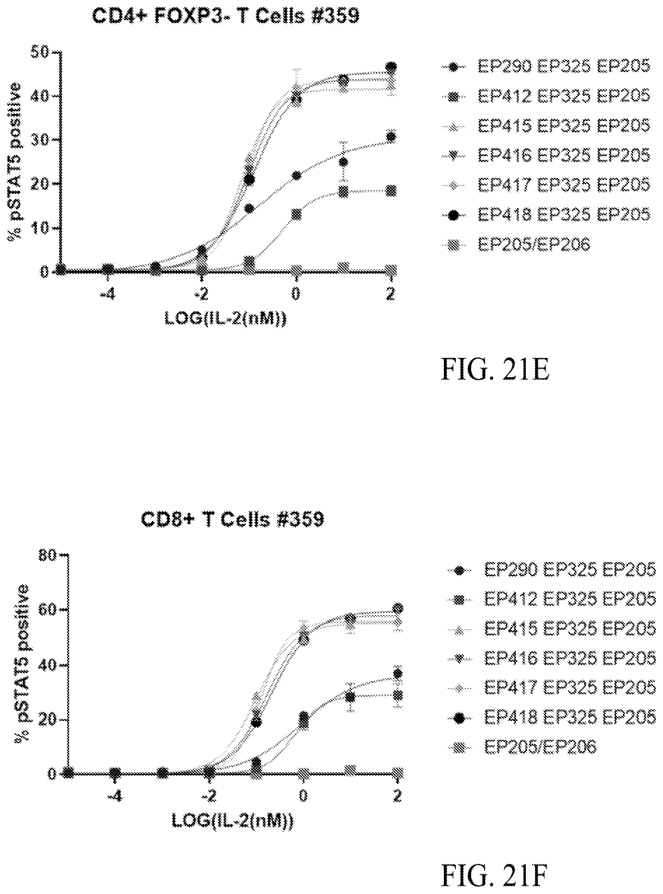

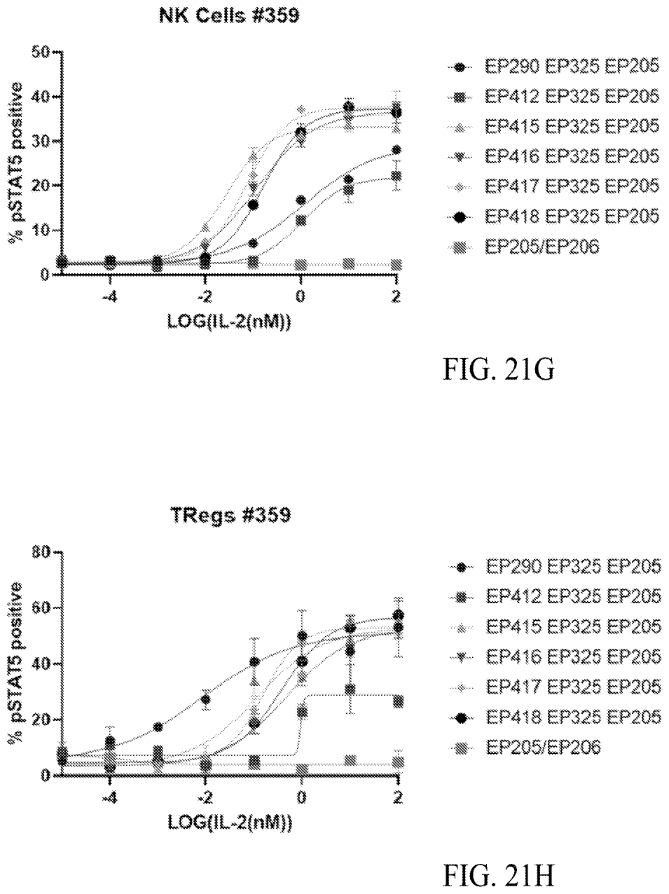

Human PBMCs were isolated from LRS cones of two separate donors and plated at 250,000 cells/well in a 96-well plate in 90 μL of media. Cells were rested 1 hr at 37° C. Cells were stimulated with human IL-2/anti-PD-L1 Fab WT and engineered IL-2/anti-PD-L1 Fab mutants at 10× concentration in 10 μL for 20 min at 37° C. Stimulated PBMCs were immediately fixed, permeabilized, stained for cell lineage markers (CD3, CD56, CD4, CD8, FOXP3) and p-STAT5 and visualized on the Attune flow cytometer. CD8+ T cells were defined as CD3+CD56−CD4−CD8+. NK cells were defined as CD3−CD56+. T regulatory cells were defined as CD3+CD56−CD4+CD8−FOXP3+. The percentage of cells that were p-STAT5+ was determined and graphed versus each IL-2 titration. EC50 values for p-STAT5 activation were determined using Prism software and were shown in Table 34. FIGS. 21A-D shows the p-STAT5 activation curves in donor-857. FIG. 21E-H shows the p-STAT5 activation curves in donor-359.

TABLE 34
IL-2/anti-PD-L1 FAB p-STAT5 activation in human PBMCs.

| | Construct | Donor-857 EC50 (nM) | | | Donor-359 EC50 (nM) | | |
|---|---|---|---|---|---|---|---|
| Format | pair | CD8+ T cell | NK Cell | Treg Cell | CD8+ T cell | NK Cell | Treg Cell |
| IL-2/ | EP290/ | 0.4814 | 2.787 | 0.001738 | 0.886 | 1.196 | 0.008621 |
| Anti- | EP325/ | | | | | | |
| PD-L1 | EP205 | | | | | | |
| Fab | EP412/ | 3.176 | 3.486 | 0.627 | 0.6505 | 1.09 | ~0.9344 |
| | EP325/ | | | | | | |
| | EP205 | | | | | | |
| | EP415/ | 0.1031 | 0.05031 | 0.06279 | 0.09958 | 0.02746 | 0.1182 |
| | EP325/ | | | | | | |
| | EP205 | | | | | | |
| | EP416/ | 0.1314 | 0.198 | 0.1544 | 0.1691 | 0.1197 | 0.4638 |
| | EP325/ | | | | | | |
| | EP205 | | | | | | |
| | EP417/ | 0.1424 | 0.1177 | 0.1436 | 0.1271 | 0.07436 | 0.1518 |
| | EP325/ | | | | | | |
| | EP205 | | | | | | |
| | EP418/ | 0.1667 | 0.3104 | 0.3188 | 0.2146 | 0.1652 | 0.372 |
| | EP325/ | | | | | | |
| | EP205 | | | | | | |

IL-2/Anti-PD-L1 Fab p-STAT5 Activation in Mouse Splenocytes

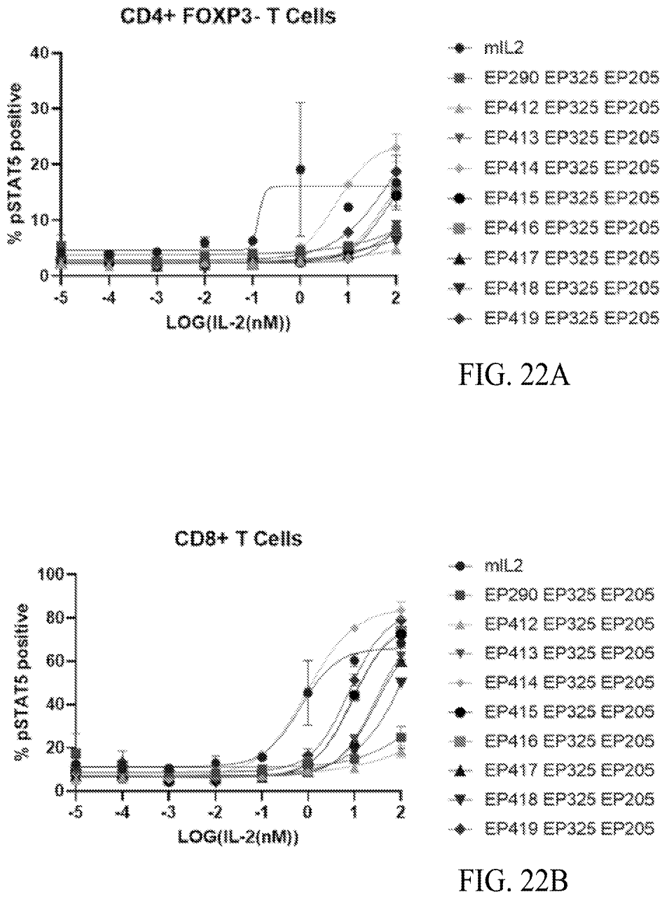

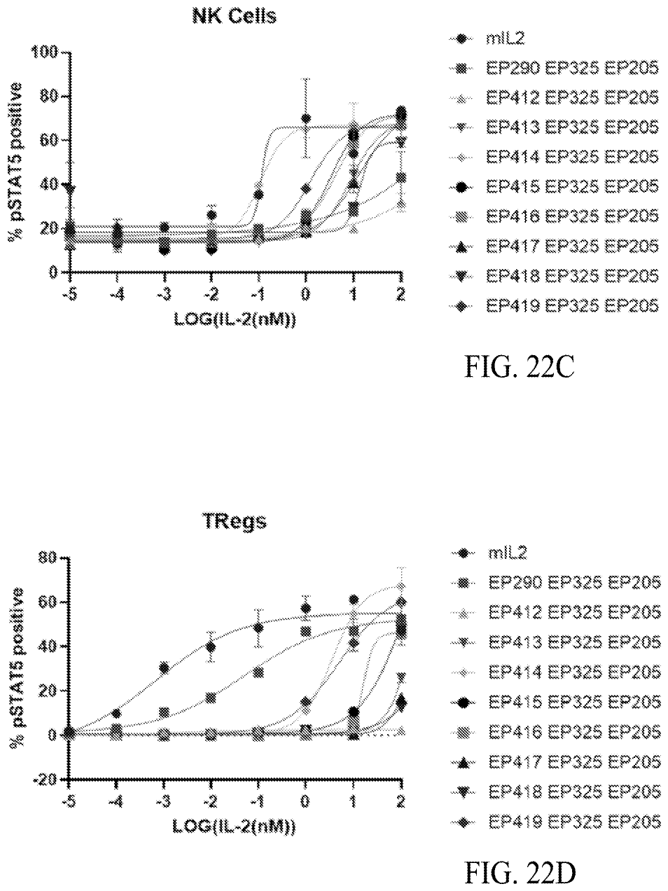

Purchased mouse splenocytes were plated at 250,000 cells/well in a 96-well plate in 90 μL of media. Cells were rested 1 hr at 37° C. Cells were stimulated with murine IL-2, human IL-2/anti-PD-L1 Fab WT and engineered IL-2/anti-PD-L1 Fab mutants at 10× concentration in 10 μL for 20 min at 37° C. Stimulated PBMCs were immediately fixed, permeabilized, stained for cell lineage markers (CD3, NKp46, CD4, CD8, FOXP3) and p-STAT5 and visualized on the Attune flow cytometer. CD8+ T cells were defined as CD3+NKp46−CD4−CD8+. NK cells were defined as CD3−NKp46+. T regulatory cells were defined as CD3+NKp46−CD4+CD8−FOXP3+. The % of cells that were p-STAT5+ was determined and graphed versus each IL-2 titration. EC50 values for p-STAT5 activation were determined using Prism software were shown in Table 35. Mouse IL2 (PeproTech, mIL-2) was used as a reference. FIGS. 22A-D show the p-STAT5 activation curves in mouse splenocytes.

TABLE 35
IL-2/anti-PD-L1 FAB p-STAT5 activation in mouse splenocytes.

| | | EC50 (nM) | | |
|---|---|---|---|---|
| | | mCD8+ | mNK | mTreg |
| Format | Construct pair | T cell | Cell | Cell |
| IL-2/ | EP290/EP325/EP205 | 128.9 | weak | 0.05156 |
| Anti- | EP412/EP325/EP205 | weak | weak | 0.001403 |
| PD-L1 | EP413/EP325/EP205 | 27.04 | 8.486 | weak |
| Fab | EP414/EP325/EP205 | 0.949 | 0.1087 | 3.532 |
| | EP415/EP325/EP205 | 8.703 | 3.173 | 134.7 |
| | EP416/EP325/EP205 | 9.712 | 3.89 | ~16.14 |
| | EP417/EP325/EP205 | 28.65 | 12.92 | weak |
| | EP418/EP325/EP205 | weak | ~13.49 | weak |
| | EP419/EP325/EP205 | 7.696 | 1.226 | 4.893 |
| mIL-2 | — | 0.691 | ~0.1144 | 0.000685 |

### Example 9. Anti-PD-L1 Mab Tumor Growth Inhibition (MB-231 Tumor)

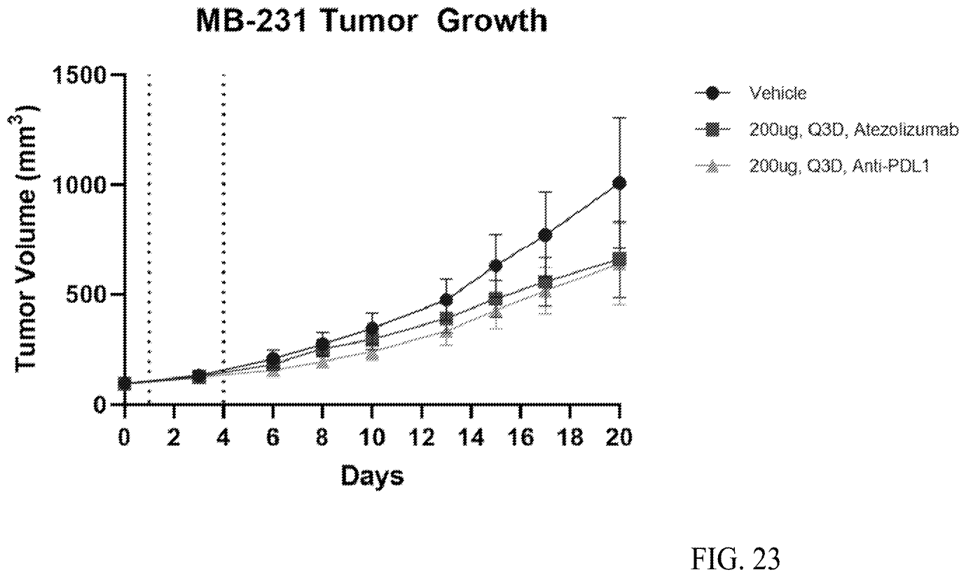

7-week old, female NCG mice, humanized with CD34+ cord blood from a single donor, were injected with 500,000 MDA-MB-231 cells in 50% matrigel subcutaneously on their back flank. Tumors were measured with calipers. Upon reaching an average volume of 100 mm³, mice were treated with 200 μg Atezolizumab or engineered anti-PD-L1 mAb EP204/EP206 Q3D for 20 days. Tumor volume and mouse weight was measured 3 times per week. Tumor volume and body weight was plotted over time compared to vehicle treated group. FIG. 23 shows the tumor growth inhibition curves.

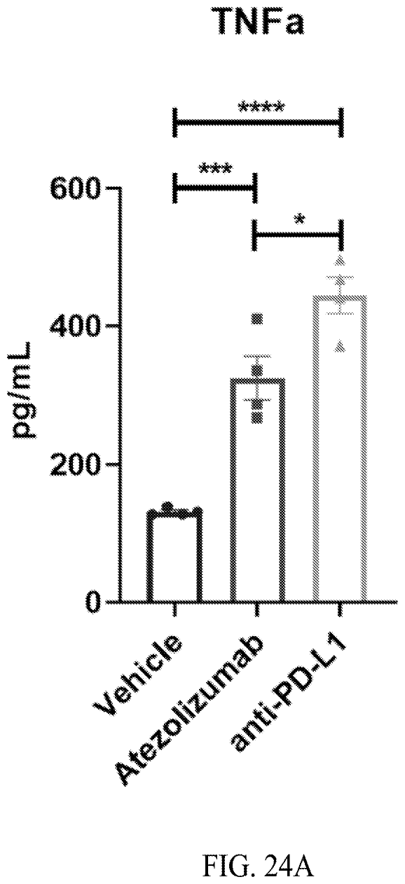

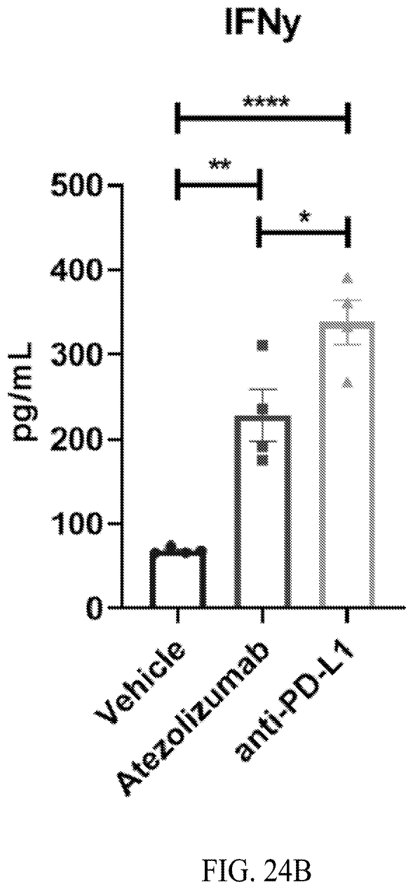

For cytokine profiling, mice were sacrificed by terminal bleed, and the blood was immediately centrifuged to separate out plasma. The concentration of human IFNγ and TNFα in the plasma was determined using the Duoset Human TNFα and IFNγ ELISA Kits. FIG. 24A and FIG. 24B show the human TNFα and IFNγ level in the plasma, respectively.

### Example 10. IL-2/Anti-PD-L1-Fab Candidates Blood and Splenocyte Immune Profiling in MC38 Tumor Mice

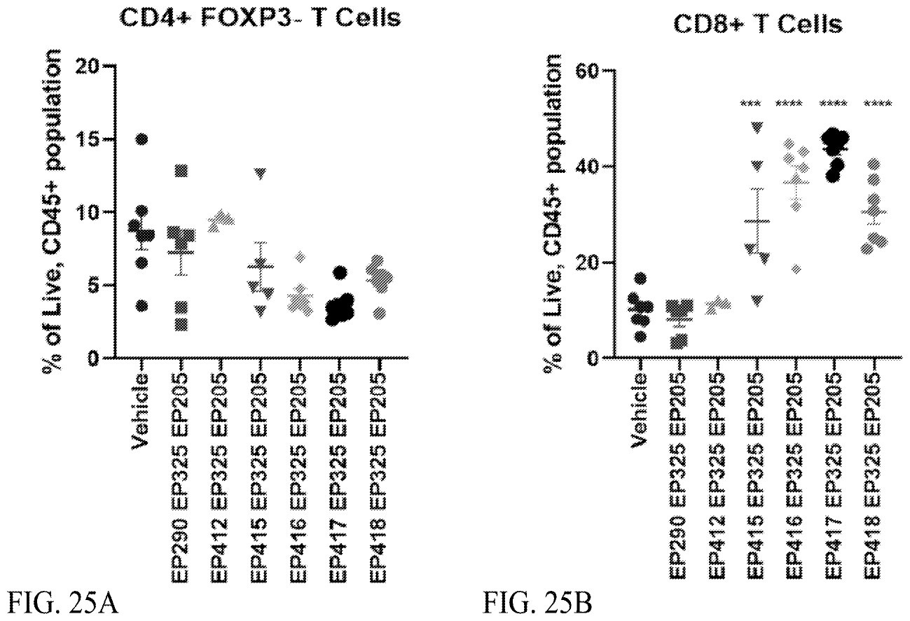

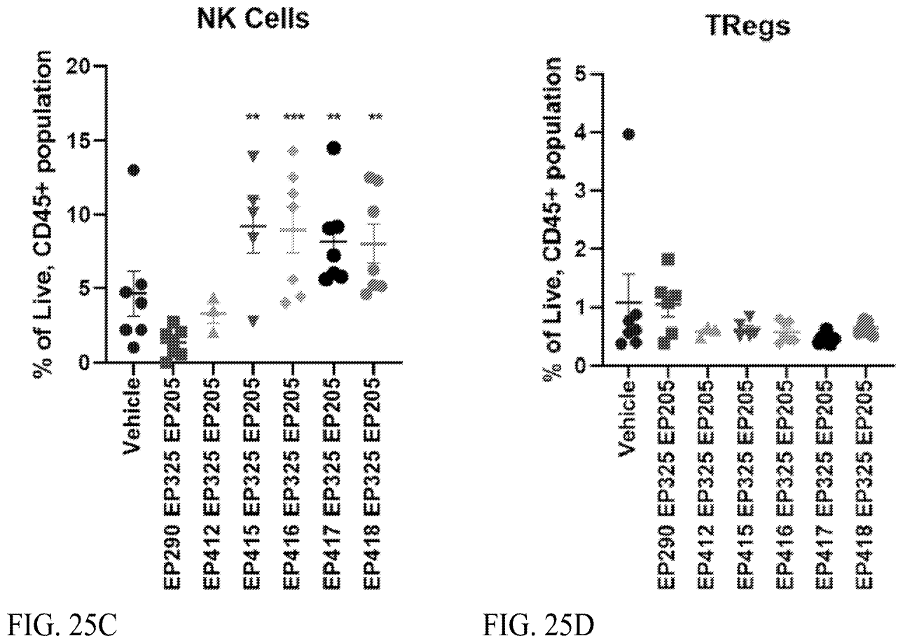

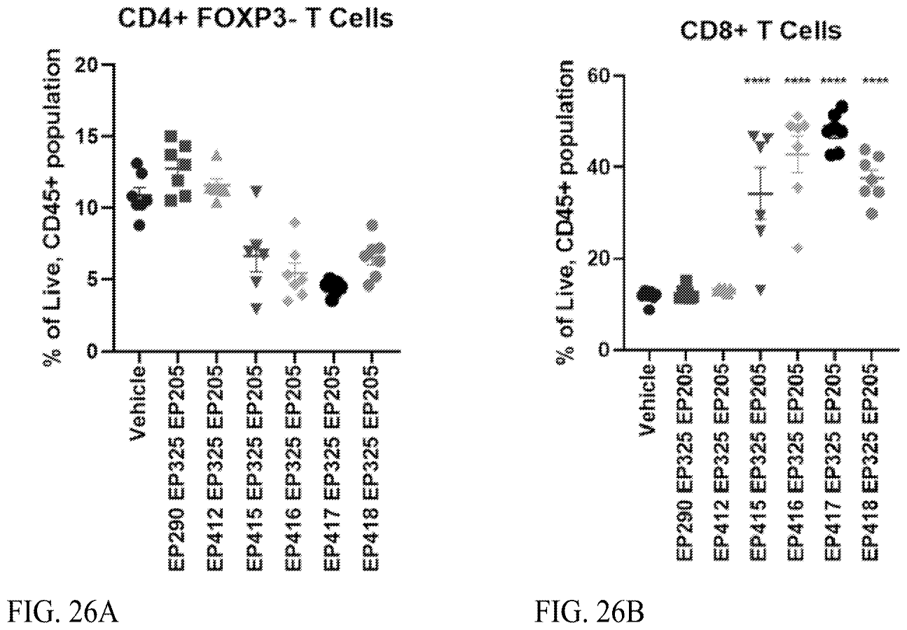

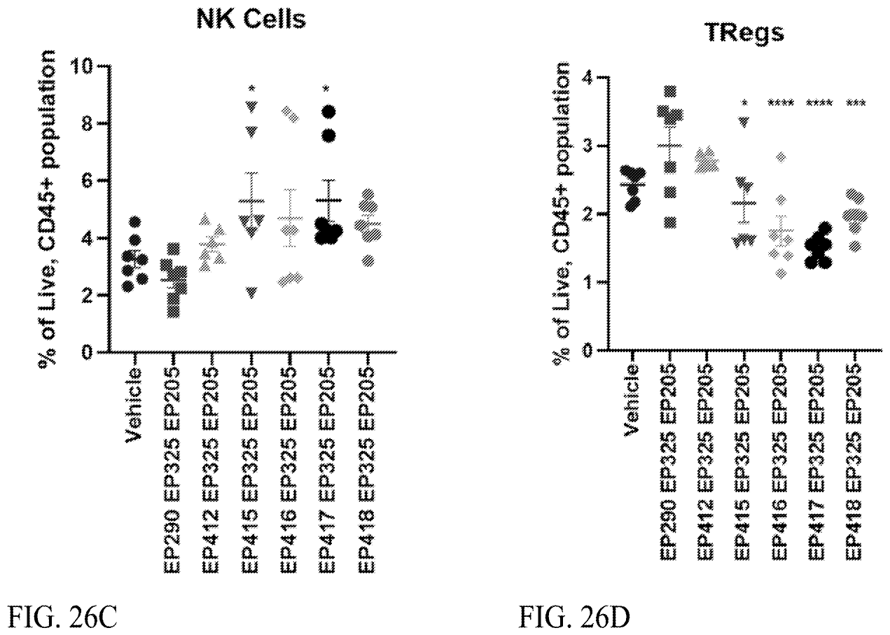

7-week old, female C57BL/6 mice were injected with 100,000 MC38 cells in 50% matrigel subcutaneously on their back flank. Tumors were measured with calipers. Upon reaching an average volume of 100 mm³, mice were treated with 2 μg human IL-2/anti-PD-L1 Fab WT (EP290/EP325/EP205) or engineered IL-2/anti-PD-L1 Fab mutants (EP412/EP325/EP205; EP415/EP325/EP205; EP416/EP325/EP205; EP417/EP325/EP205; and EP418/EP325/EP205) Q4D for 11 days. On day 11 mice were sacrificed and peripheral blood was isolated via a tail vein bleed. Red blood cells were lysed, and immune cells were analyzed using an Attune flow cytometer. CD4+ T cells were defined as CD45+CD3+NKp46−CD4+CD8−. CD8+ T cells were defined as CD45+CD3+NKp46−CD4−CD8+. NK cells were defined as CD45+CD3−NKp46+. T regulatory cells NK cells were defined as CD45+CD3+NKp46−CD4+CD8−FOXP3+. The percentage of each subtype within the CD45+ population was plotted for each treatment group. FIGS. 25A-D show the immune cell profiling results in blood. FIGS. 26A-D show the immune cell profiling results in splenocytes.

### Example 11. IL-2/Anti-PD-L1-Fab Candidates Tumor Growth Inhibition (B16F10-HPD-L1 Tumor in HPD-1 Transgenic Mice)

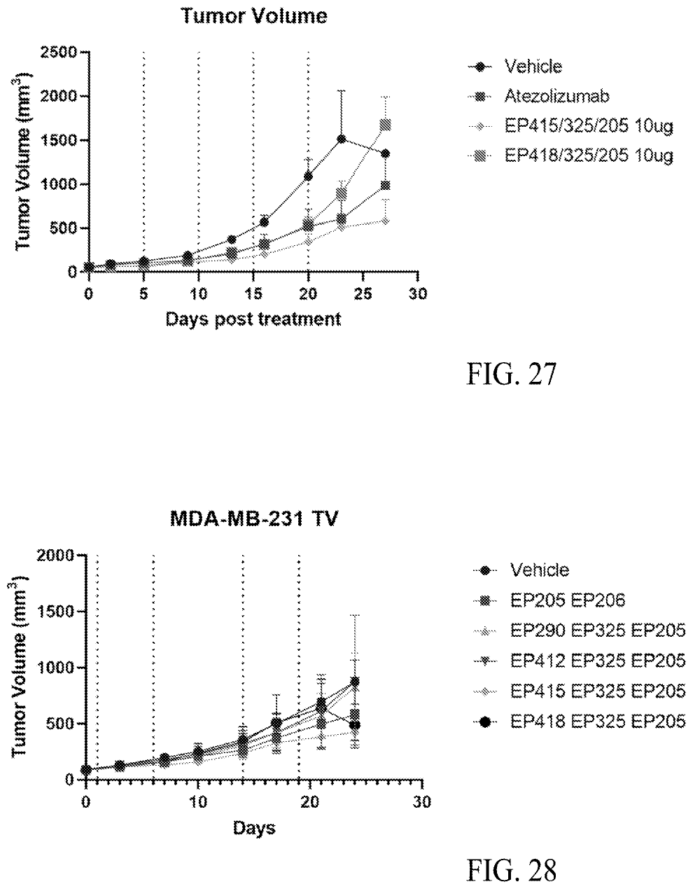

The following example describes experiments to assess tumor growth inhibition by IL-2/anti-PD-L1-Fab in an in vivo murine model. 7-week old, female hPD-1 transgenic mice were injected with 500,000 B16F10-hPD-L1 cells in 50% matrigel subcutaneously on their back flank. Tumors were measured with calipers. Upon reaching an average volume of 70-90 mm³, mice were treated with 200 μg atezolizumab Q3D or 10 μg engineered IL-2/anti-PD-L1-Fab bifunctional proteins (EP415/EP325/EP205; EP418/EP325/EP205) Q5D for 20 days. Tumor volume and mouse weight was measured 3 times per week. Tumor volume and body weight was plotted over time compared to vehicle treated group. FIG. 27 shows the tumor growth inhibition curves.

### Example 12. IL-2/Anti-PD-L1-Fab Candidates Tumor Growth Inhibition (MB-231 Tumor)

The following example describes experiments to assess tumor growth inhibition by IL-2/anti-PD-L1-Fab in an in vivo murine model. 7-week old, female NCG mice, humanized with CD34+ cord blood from a single donor, were injected with 500,000 MDA-MB-231 cells in 50% matrigel subcutaneously on their back flank. Tumors were measured with calipers. Upon reaching an average volume of 80-100 mm³, mice were treated with 200 μg engineered anti-PD-L1 mAb EP205/EP206 Q3D or 5 μg IL-2/anti-PD-L1-Fab bifunctional proteins (EP290/EP325/EP205; EP412/EP325/EP205; EP415/EP325/EP205; EP418/EP325/EP205) Q5D for 20 days. Tumor volume and mouse weight was measured 3 times per week. Tumor volume and body weight was plotted over time compared to vehicle treated group. FIG. 28 shows the tumor growth inhibition curves.

### Example 13. Treatment of Human PBMC by IL2/Anti-PD-L1-Fab Antibody Leads to Increased PD-1 Expression in CD8+ T Cells In Vitro

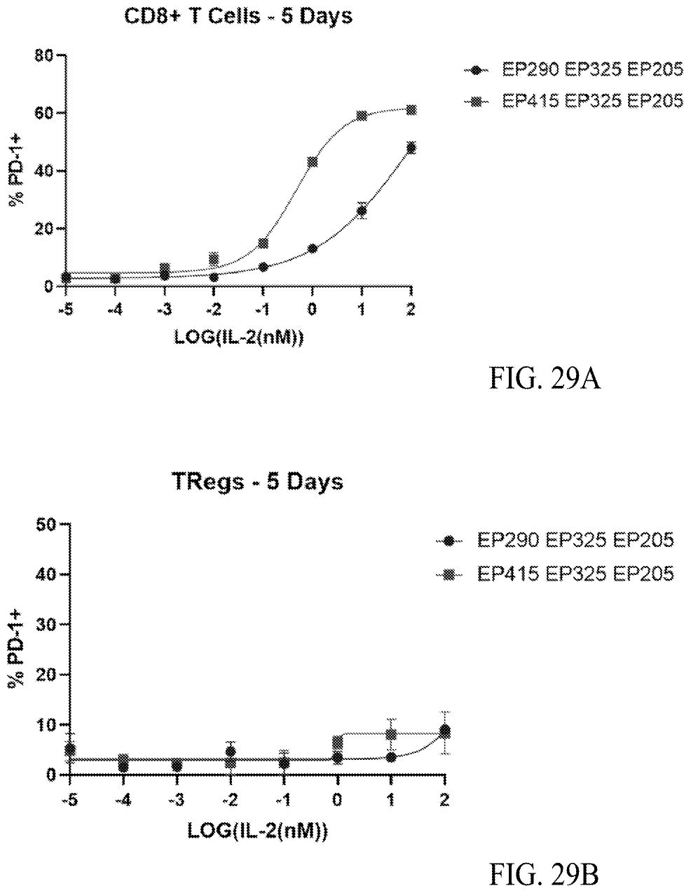

Human PBMCs were isolated from an LRS cone from a single donor and plated at 100,000 cells/well in a 96-well plate in 180 μL of complete media. Cells were rested at 37° C. for 1 hour in a 5% CO2 incubator. Cells were stimulated by adding 20 μL of IL-2 Fc/anti-PD-L1 Fabs comprising the combined components of EP415/EP325/EP205 or EP290/EP325/EP205 diluted in media giving a final concentration in the well of between 100 nM and 0.0001 nM of IL2. Cells were left in the incubator 5 days undisturbed. After 5 days, the PBMCs were washed in PBS, and incubated in a viability dye. After subsequent washing, the cells were fixed and permeabilized, and then stained for cell lineage markers (CD3, CD56, CD4, CD8, FOXP3) and PD-1, and visualized on the Attune flow cytometer. CD8+ T cells were defined as CD3+CD56−CD4−CD8+, TRegs were defined as CD3+CD56−CD4+CD8−FOXP3+. The percentage of CD8+ T cells and TRegs that expressed PD-1 was calculated and plotted against IL2 concentration. FIG. 29A shows the dose dependent PD-1 positive CD8+ T cells after treatment of EP415/EP325/EP205 and EP290/EP325/EP205, respectively. FIG. 29B shows the PD-1 positive Treg cells after treatment of EP415/EP325/EP205 and EP290/EP325/EP205, respectively.

### Example 14. EP415/EP325/EP205 is a Strong IL2RB Receptor Agonist

HEK-Blue IL-2 cells were purchased from InvivoGen and cultured according to manufacturer's instructions. To quantify the surface expression of CD25, CD122, and PD-L1 by these cells, HEK Blue IL2 cells were seeded in a 96-well plate at a density of 100,000 cells per well in 200 μL of media. The cells were allowed to recover in the incubator for at least 1 hour. The cells were then washed and stained with fluorescent antibodies for CD25, CD122, or PD-L1. At the same time, Quantum Simply Cellular microsphere standards (Bangs Laboratories, Inc.) were also stained with the same antibodies. After washing again, the cells and the microspheres were visualized on an Attune flow cytometer. The Median Fluorescent Intensity (MFI) for each microsphere in the appropriate fluorescent channel was calculated, allowing the generation of standard curves of MFI vs Antibody Binding Content (ABC). The MFI from the cells for each antibody was compared to the appropriate standard, and median receptor number was calculated and plotted.

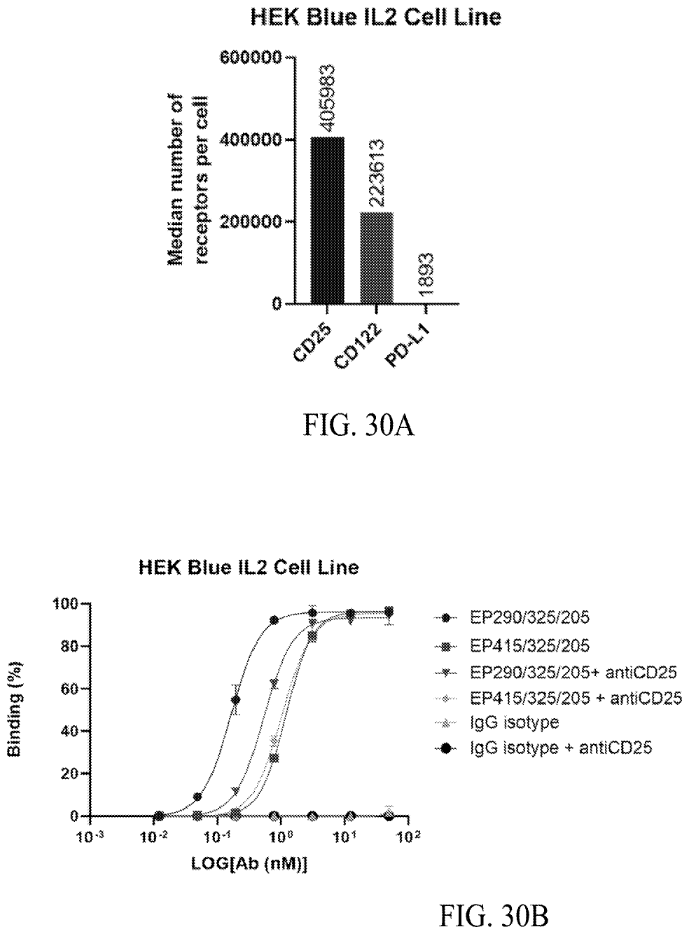

HEK-Blue IL-2 cells were seeded in a 96-well plate at a density of 100,000 cells per well in 100 μL of media. The cells were allowed to recover in the incubator for at least 1 hour. The plate was spun down, media was removed, and the cells were resuspended in fresh media either containing an anti-CD25 antibody at a 1:100 dilution, or media alone. The cells were incubated at 4° C. shaking for 30 minutes. After incubation, cells were spun down, the media was discarded, and they were resuspended in fresh media containing a titration of either EP415/EP325/EP205 or EP290/EP325/EP205. The cells were then incubated at 4° C. shaking for 1 hour. The media was then removed and replaced with media containing anti-Human-Fc antibodies conjugated with Alex Fluor 647. The cells were incubated at 4° C. shaking for 30 minutes. The cells were washed, and the presence of bound EP415/EP325/EP205 or EP290/EP325/EP205 was determined using the Attune flow cytometer. The percentage of HEK Blue IL2 cells with bound EP415/EP325/EP205 or EP290/EP325/EP205 was plotted against IL2 concentration. FIG. 30A shows the expression level of CD25, CD122, and PD-L1 on HEK-Blue IL-2 cells. FIG. 30B shows the FACS binding activities of EP415/EP325/EP205 and EP290/EP325/EP205 to HEK-Blue IL-2 cells with and without interference of anti-CD25 antibody. The CD-25 antibody reduced the binding of EP290/EP325/EP205 to HEK-Blue IL-2 cells, whereas it has no impacts to EP415/EP325/EP205 binding. This data suggests that EP415/325/205 prefers IL2Rβγ receptor binding because the anti-CD25 antibody does not appear to interfere the interaction of EP415/325/205 with the cell line. In contrasting, EP290/EP325/EP205 demonstrates higher levels of binding to IL2Rαβγ due to the presence of a IL2-WT sequence and reduction in binding due to interference of the anti-CD25 antibody.

### Example 15. EP415/EP325/EP205 is Tumor Localized In Vivo

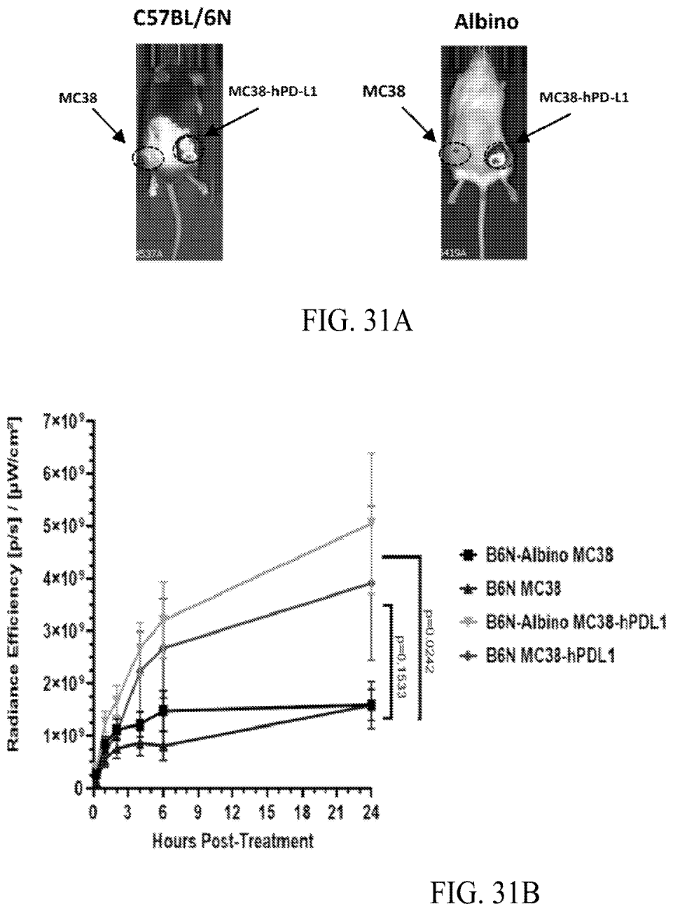

C57BL/6N and B6N Albino mice were inoculated subcutaneously bilaterally with both 250,000 MC38 cells (lower left flank), and 1,000,000 MC38-hPD-L1 cells (lower right flank). Once the volume of both tumors reached a minimum of 300 mm³, the mice were dosed via intraperitoneal injection (IP) with 1 mg/kg of EP415/EP325/EP205 that had been fluorescently labelled with Alexa Fluor 750. The presence of fluorescently labelled EP415/EP325/EP205 at the tumor site was determined using an IVIS Lumina III LT (Perkin Elmer) system. Mice were maintained on 3% isoflurane via nose cones attached to the internal anesthesia manifold during imaging and placed on the heated (37° C.) shelf of the imaging chamber for epi-illumination image acquisition. Following the scan, mice were removed and placed back into their respective cages for recovery. Mice were imaged at the following time points after dosing: 15 minutes, 1 hour, 2 hours, 4 hours, 6 hours, and 24 hours. The Total Flux for each tumor was calculated and plotted against time. FIG. 31A shows the representative imaging results of tumor bearing mice at 24 hours after EP415/EP325/EP205 injection. FIG. 31B shows the time dependent enrichment of EP415/EP325/EP205 to MC-38-hPD-L1 and MC-38 tumor sites, respectively. These results indicate that EP415/EP325/EP205 is preferentially localized in MC-38-hPD-L1 tumor site, but not the MC-38 tumor site.

### Example 16. EP415/EP325/EP205 Efficacious in MDA-MB-231 Cell Hot Tumor Model

28-29 week old, female NCG mice, humanized with CD34+ cord blood from a 2-3 donors, were injected with 500,000 MDA-MB-231 cells in 50% Matrigel subcutaneously on their back flank. Tumors were measured with calipers. Upon reaching an average tumor volume of 100 mm³, mice were treated IP with anti-PD-L1 EP205/EP206 (200 μg, Q3D), EP290/EP325/EP205 IL2/anti-PD-L1 (5 ug, Q5D), or EP415/EP325/EP205 IL2/anti-PD-L1 (5 ug, Q5D). Tumor volume was measured 2-3 times per week and was plotted over time compared to vehicle control group.

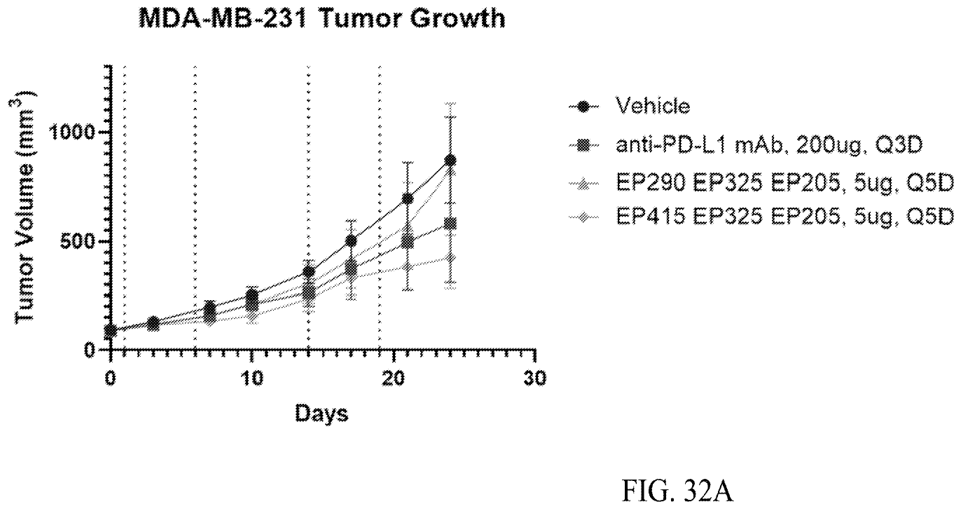

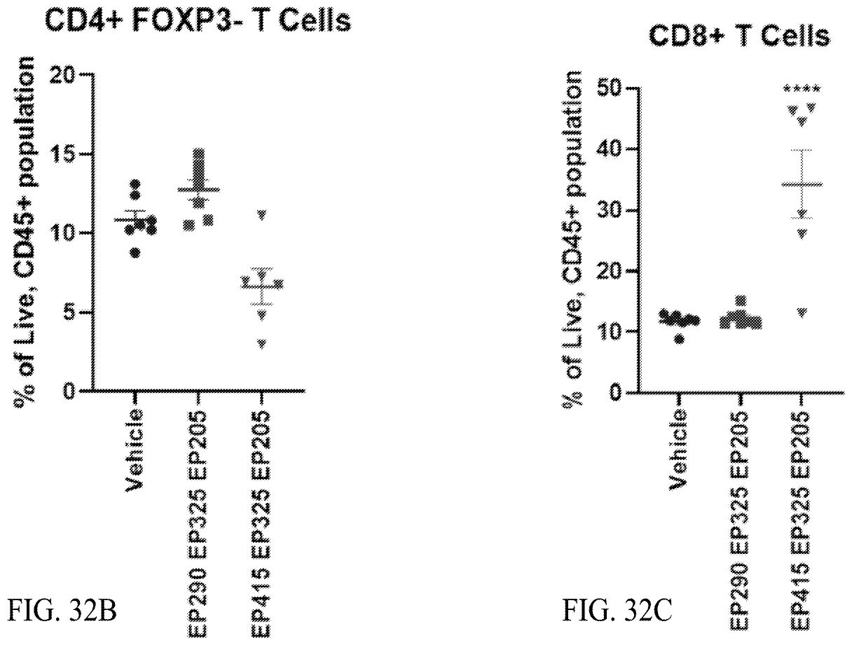

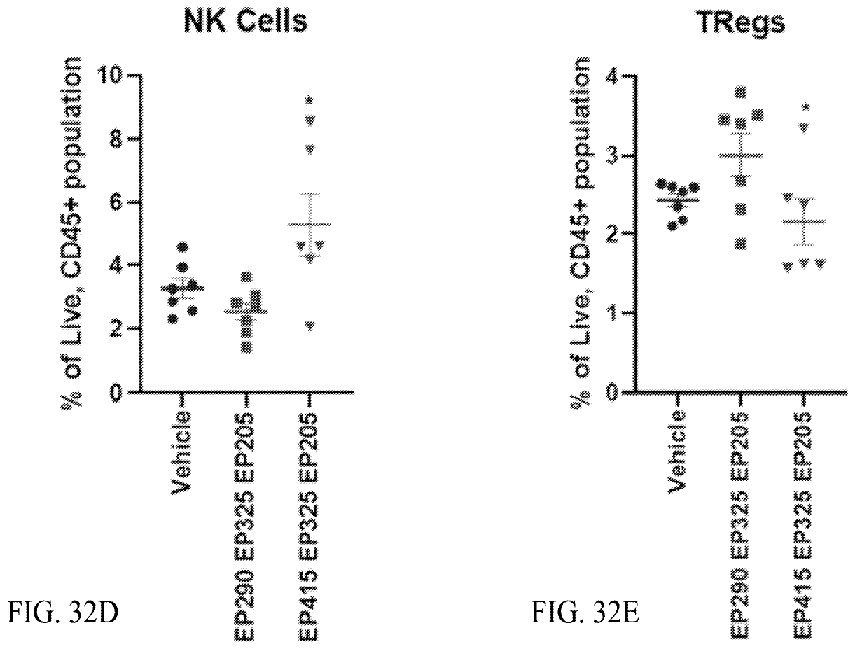

On day 24 after dosing initiation, the mice were sacrificed, and tumors were taken. The tumors were dissociated into a single cell suspension and incubated with a viability dye. After washing, tumor cells were fixed, permeabilized, and stained for cell lineage markers (CD3, CD56, CD4, CD8, FOXP3). The percentage of CD4+ T Cells (CD3+CD56−CD4+CD8−FOXP3−), CD8+ T Cells (CD3+CD56−CD4−CD8+), NK cells (CD3−CD56+), and TRegs (CD3+CD56−CD4+CD8−FOXP3+) as a percentage of the total live cells was calculated for the EP415/EP325/EP205 and EP290/EP325/EP205 treated groups and compared to the vehicle treated mice. FIG. 32A shows the tumor growth inhibition curve of EP415/EP325/EP205 against anti-PD1/PD-L1 responsive cancer cell MDA-MB-231 in the humanized NCG mice. EP415/EP325/EP205 shows superior tumor growth inhibition activities than both anti-PD-L1 antibody and the EP290/EP325/EP205. Accordingly, FIG. 32B-E show the level of immune cell in the tumor site. The activation of CD8 T and NK cells in the tumor site is correlated with the observed efficacy.

### Example 17. EP415/EP325/EP205 is Efficacious in a COLO205 Cell Cold Tumor Model

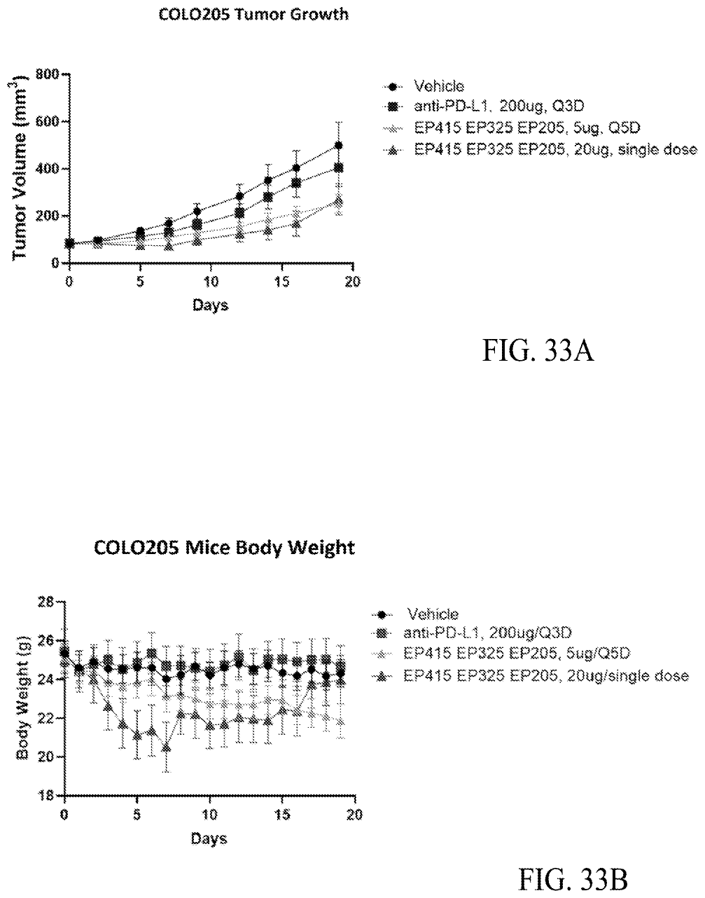

28-29 week old, female NCG mice, humanized with CD34+ cord blood from 2-3 donors, were injected with 5,000,000 COLO205 cells in 50% Matrigel subcutaneously on their back flank. Tumors were measured with calipers. Upon reaching an average tumor volume of 100 mm³, mice were treated IP with either anti-PD-L1 (200 ug, EP205/EP206, Q3D), EP415/EP325/EP205 (5 ug, Q5D), or EP415/EP325/EP205 (20 ug single dose). Tumor volume was measured 2-3 times per week and was plotted over time compared to vehicle control group. Mouse body weight was measure daily and was plotted against time. FIG. 33A shows the tumor growth inhibition curve of EP415/EP325/EP205 against cancer cell COLO205 in the humanized mice. FIG. 33B shows the corresponding body weight change of the mice. The data in FIGS. 33A-D support that EP415/EP325/EP205 is more efficacious in suppressing tumor growth than that of anti-PD-L1 antibodies.

### Example 18. EP415/EP325/EP205 Efficacious in Anti-PD1 Resistant NCI-H1975 Cell Tumor

20-24 week old, female NOG mice, humanized with CD34+ cord blood from a 2-3 donors, were injected with 8×10⁶ NCI-H1975 cells in 100 μL serum-free medium mixed with Matrigel (v/v 1:1) subcutaneously on their back right flank. Tumors were measured with calipers. Upon reaching an average tumor volume of 100 mm³, mice were treated IP with either EP415/EP325/EP205 (10 ug, Q10D), EP415/EP325/EP205 (5 ug, Q6D), or Pembrolizumab (200 ug, Q4D). Tumor volume was measured twice per week and was plotted over time compared to vehicle control group. Mouse body weight was measure 3-5 times per week and was plotted against time.

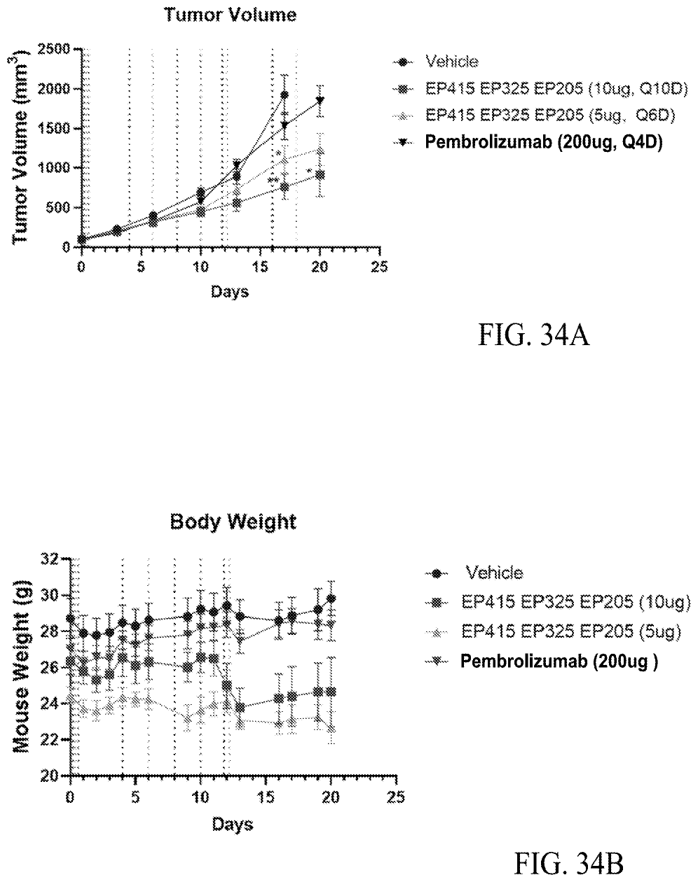

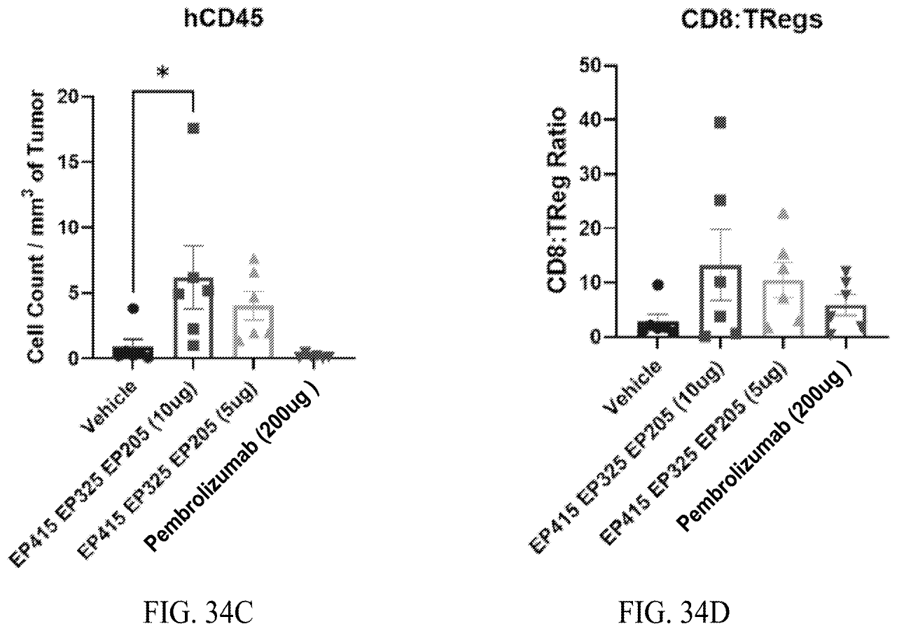

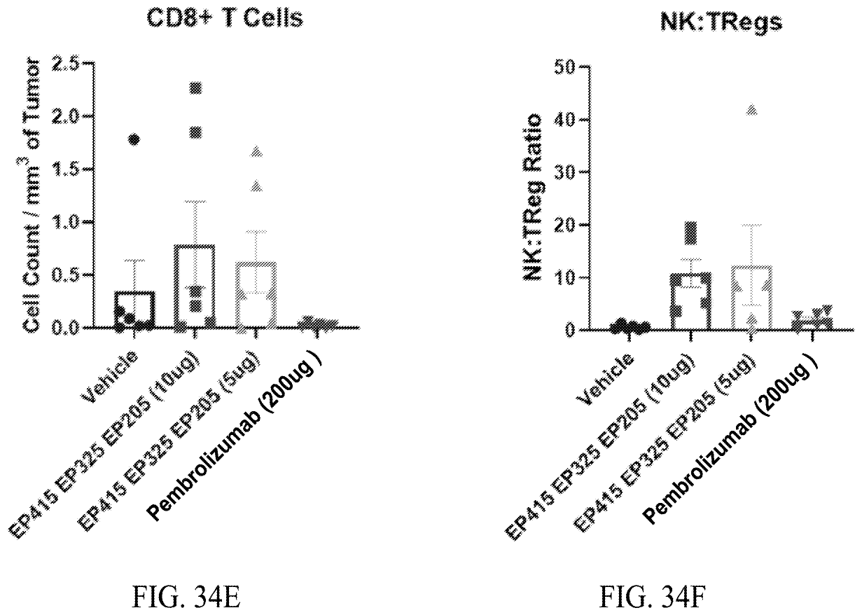

On day 20 after dosing initiation, the mice were sacrificed, and tumors were taken. The tumors were dissociated into a single cell suspension and incubated with a viability dye. After washing, cells were fixed, permeabilized, and stained for cell lineage markers (mCD45, hCD45, hCD3, hCD56, hCD4, hCD8, hFOXP3). The number of live hCD45 cells (mCD45−hCD45+) and live hCD8+ T Cells (mCD45−hCD45+hCD3+hCD56−hCD4−hCD8+) per mm3 of tumor was calculated for each of the treatment groups and compared to the vehicle control group. The ratio of hCD8+ T Cells and NK cells (mCD45−hCD45+hCD3−hCD56+) with hTRegs (mCD45−hCD45+hCD3+hCD56−hCD8−hCD4+hFOXP3+) within the tumors was also calculate for each of the treatment groups and plotted compared with the vehicle control group. FIG. 34A shows the tumor growth inhibition curve of EP415/EP325/EP205 against cancer cell H1975 in the humanized mice. FIG. 34B shows the corresponding body weight change of the mice. FIG. 34C-F shows the profiling results of the tumor infiltrated immune cells. EP415/EP325/EP205 retains its tumor growth inhibition activity against H1975 cells, which are resistant to the anti-PD1 antibody pembrolizumab.

### Example 19. EP415/EP325/EP205 Cynomolgus Monkey PK/PD Studies

2-5 year old non-naïve cynomolgus monkeys with a weight range of 3.0-5.0 kg were dosed IV bolus with a single dose of either 0.1 mg/kg or 0.5 mg/kg EP415/EP325/EP205. After dosing, blood was collected at each selected time point. The concentrations of constructs in plasma were quantified by ELISA assay and expressed in ng/ml. The concentration of EP415/EP325/EP205 detected in the monkey plasma was plotted against time. Plasma concentrations at each time point from each animal were used for pharmacokinetic (PK) calculation. Area under the curve (AUC) was estimated using the trapezoid method. Other parameters were estimated using established equations.

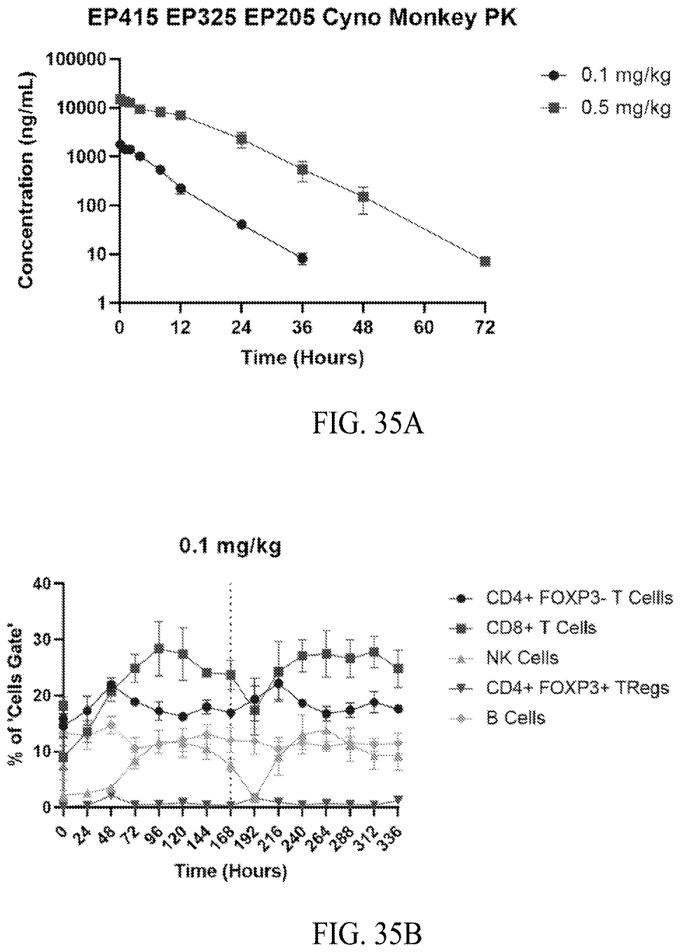

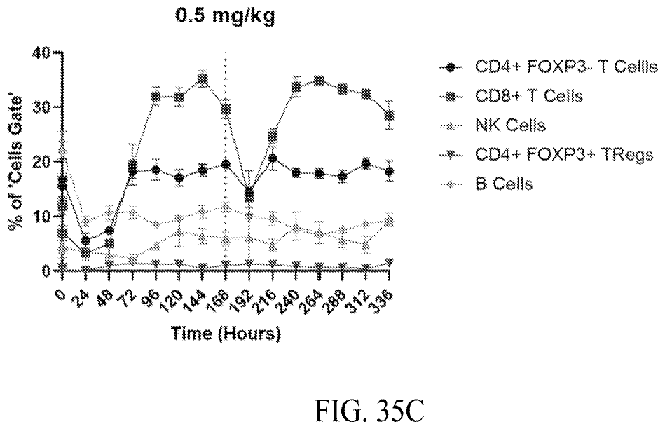

For pharmacodynamic (PD) analysis, blood was isolated each day (prior to dosing for days 0 and 7), and stained for immune cell-lineage markers (CD3, CD4, CD8, CD16, CD45, FOXP3, CD20) and quantified by flow cytometry. The following immune populations were quantified: CD4+ FOXP3− T cells (CD45+CD3+CD16−CD4+CD8−FOXP3−), CD4+FOXP3+T regulatory cells (CD45+CD3+CD16−CD4+CD8−Foxp3+), CD8+ T cells (CD45+CD3+CD16−CD4−CD8+), B cells (CD45+CD3−CD16−CD20+), and Natural killer (NK) cells (CD45+CD3−CD16+CD20−). Raw numbers for each of these populations were normalized to the total number of cellular events observed and plotted against time. FIG. 35A shows the concentration of EP415/EP325/EP205 detected in monkey plasma by ELISA and plotted against time. FIGS. 35B-C show the percentage of immune cell populations in monkey blood after dosing with EP415/EP325/EP205. While increases in the CD8+ T cell population were observed after each dose, the levels of CD4+FOXP3+ Tregs remained at baseline.

### INCORPORATION BY REFERENCE

Unless stated to the contrary, the entire disclosure of each of the patent documents and scientific articles referred to herein is incorporated by reference for all purposes.

### EQUIVALENTS

An antigen-binding site described in application may be embodied in other specific forms without departing from the spirit or essential characteristics thereof. The foregoing embodiments are therefore to be considered in all respects illustrative rather than limiting on an antigen-binding site described herein. Scope of the present application is thus indicated by the appended claims rather than by the foregoing description, and all changes that come within the meaning and the range of equivalency of the claims are intended to be embraced therein.

This application claims the benefit of priority to U.S. Provisional Application No. 63/140,749, filed Jan. 22, 2021, which application is hereby incorporated by reference in its entirety.

1. An antigen-binding site that binds PD-L1, comprising:
(a) a heavy chain variable region (VH) comprising a complementarity determining region 1 (CDR1) sequence, a complementarity determining region 2 (CDR2) sequence, and a complementarity determining region (CDR3) sequence of SEQ ID NOS: 11, 12, and 89, respectively; and a light chain variable region (VL) comprising a CDR1 sequence, a CDR2 sequence, and a CDR3 sequence of SEQ ID NOS: 47, 48, and 49, respectively
(b) VH comprising CDR1, CDR2, and CDR3 sequences of SEQ ID NOS: 3, 4, and 5, respectively; and a VL comprising CDR1, CDR2, and CDR3 sequences of SEQ ID NOS: 6, 7, and 8, respectively;
(c) VH comprising CDR1, CDR2, and CDR3 sequences of SEQ ID NOS: 11, 12, and 13, respectively; and a VL comprising CDR1, CDR2, and CDR3 sequences of SEQ ID NOS: 14, 15, and 16, respectively;
(d) VH comprising CDR1, CDR2, and CDR3 sequences of SEQ ID NOS: 19, 20, and 21, respectively; and a VL comprising CDR1, CDR2, and CDR3 sequences of SEQ ID NOS: 22, 23, and 24, respectively;
(e) VH comprising CDR1, CDR2, and CDR3 sequences of SEQ ID NOS: 19, 20, and 27, respectively; and a VL comprising CDR1, CDR2, and CDR3 sequences of SEQ ID NOS: 28, 29, and 30, respectively;
(f) VH comprising CDR1, CDR2, and CDR3 sequences of SEQ ID NOS: 33, 34, and 35, respectively; and a VL comprising CDR1, CDR2, and CDR3 sequences of SEQ ID NOS: 36, 37, and 38, respectively;
(g) VH comprising CDR1, CDR2, and CDR3 sequences of SEQ ID NOS: 3, 41, and 5, respectively; and a VL comprising CDR1, CDR2, and CDR3 sequences of SEQ ID NOS: 6, 42, and 43, respectively;
(h) VH comprising CDR1, CDR2, and CDR3 sequences of SEQ ID NOS: 11, 12, and 46, respectively; and a VL comprising CDR1, CDR2, and CDR3 sequences of SEQ ID NOS: 47, 48, and 49, respectively;
(i) VH comprising CDR1, CDR2, and CDR3 sequences of SEQ ID NOS: 52, 53, and 54, respectively; and a VL comprising CDR1, CDR2, and CDR3 sequences of SEQ ID NOS: 55, 56, and 57, respectively;
(j) VH comprising CDR1, CDR2, and CDR3 sequences of SEQ ID NOS: 11, 12, and 46, respectively; and a VL comprising CDR1, CDR2, and CDR3 sequences of SEQ ID NOS: 47, 60, and 49, respectively;
(k) VH comprising CDR1, CDR2, and CDR3 sequences of SEQ ID NOS: 63, 64, and 65, respectively; and a VL comprising CDR1, CDR2, and CDR3 sequences of SEQ ID NOS: 66, 67, and 68, respectively;
(l) VH comprising CDR1, CDR2, and CDR3 sequences of SEQ ID NOS: 11, 12, and 71, respectively; and a VL comprising CDR1, CDR2, and CDR3 sequences of SEQ ID NOS: 47, 48, and 49, respectively;
(m) VH comprising CDR1, CDR2, and CDR3 sequences of SEQ ID NOS: 11, 12, and 74, respectively; and a VL comprising CDR1, CDR2, and CDR3 sequences of SEQ ID NOS: 47, 48, and 49, respectively;
(n) VH comprising CDR1, CDR2, and CDR3 sequences of SEQ ID NOS: 11, 12, and 77, respectively; and a VL comprising CDR1, CDR2, and CDR3 sequences of SEQ ID NOS: 47, 48, and 49, respectively;
(o) VH comprising CDR1, CDR2, and CDR3 sequences of SEQ ID NOS: 11, 12, and 80, respectively; and a VL comprising CDR1, CDR2, and CDR3 sequences of SEQ ID NOS: 47, 48, and 49, respectively;
(p) VH comprising CDR1, CDR2, and CDR3 sequences of SEQ ID NOS: 11, 12, and 83, respectively; and a VL comprising CDR1, CDR2, and CDR3 sequences of SEQ ID NOS: 47, 48, and 49, respectively; or
(q) VH comprising CDR1, CDR2, and CDR3 sequences of SEQ ID NOS: 11, 12, and 86, respectively; and a VL comprising CDR1, CDR2, and CDR3 sequences of SEQ ID NOS: 47, 48, and 49, respectively.

2. (canceled)

3. The antigen-binding site of claim 1, wherein:
(a) the VH comprises an amino acid sequence at least 90% identical to SEQ ID NO: 87 and the VL comprises an amino acid sequence at least 90% identical to SEQ ID NO: 88;
(b) the VH comprises an amino acid sequence at least 90% identical to SEQ ID NO: 1 and the VL comprises an amino acid sequence at least 90% identical to SEQ ID NO: 2;
(c) the VH comprises an amino acid sequence at least 90% identical to SEQ ID NO: 9 and the VL comprises an amino acid sequence at least 90% identical to SEQ ID NO: 10;
(d) the VH comprises an amino acid sequence at least 90% identical to SEQ ID NO: 17 and the VL comprises an amino acid sequence at least 90% identical to SEQ ID NO: 18;
(e) the VH comprises an amino acid sequence at least 90% identical to SEQ ID NO: 25 and the VL comprises an amino acid sequence at least 90% identical to SEQ ID NO: 26;
(f) the VH comprises an amino acid sequence at least 90% identical to SEQ ID NO: 31 and the VL comprises an amino acid sequence at least 90% identical to SEQ ID NO: 32;
(g) the VH comprises an amino acid sequence at least 90% identical to SEQ ID NO: 39 and the VL comprises an amino acid sequence at least 90% identical to SEQ ID NO: 40;
(h) the VH comprises an amino acid sequence at least 90% identical to SEQ ID NO: 44 and the VL comprises an amino acid sequence at least 90% identical to SEQ ID NO: 45;
(i) the VH comprises an amino acid sequence at least 90% identical to SEQ ID NO: 50 and the VL comprises an amino acid sequence at least 90% identical to SEQ ID NO: 51;
(j) the VH comprises an amino acid sequence at least 90% identical to SEQ ID NO: 58 and the VL comprises an amino acid sequence at least 90% identical to SEQ ID NO: 59;
(k) the VH comprises an amino acid sequence at least 90% identical to SEQ ID NO: 61 and the VL comprises an amino acid sequence at least 90% identical to SEQ ID NO: 62;
(l) the VH comprises an amino acid sequence at least 90% identical to SEQ ID NO: 69 and the VL comprises an amino acid sequence at least 90% identical to SEQ ID NO: 70;
(m) the VH comprises an amino acid sequence at least 90% identical to SEQ ID NO: 72 and the VL comprises an amino acid sequence at least 90% identical to SEQ ID NO: 73;
(n) the VH comprises an amino acid sequence at least 90% identical to SEQ ID NO: 75 and the VL comprises an amino acid sequence at least 90% identical to SEQ ID NO: 76;
(o) the VH comprises an amino acid sequence at least 90% identical to SEQ ID NO: 78 and the VL comprises an amino acid sequence at least 90% identical to SEQ ID NO: 79;
(p) the VH comprises an amino acid sequence at least 90% identical to SEQ ID NO: 81 and the VL comprises an amino acid sequence at least 90% identical to SEQ ID NO: 82;
(q) the VH comprises an amino acid sequence at least 90% identical to SEQ ID NO: 84 and the VL comprises an amino acid sequence at least 90% identical to SEQ ID NO: 85;
(r) the VH comprises or consists of the amino acid sequence of SEQ ID NO: 87 and the VL comprises or consists of the amino acid sequence of SEQ ID NO:88
(s) the VH comprises or consists of the amino acid sequence of SEQ ID NO:1 and the VL comprises or consists of the amino acid sequence of SEQ ID NO:2;
(t) the VH comprises or consists of the amino acid sequence of SEQ ID NO:9 and the VL comprises or consists of the amino acid sequence of SEQ ID NO:10;
(u) the VH comprises or consists of the amino acid sequence of SEQ ID NO: 17 and the VL comprises or consists of the amino acid sequence of SEQ ID NO:18;
(v) the VH comprises or consists of the amino acid sequence of SEQ ID NO: 25 and the VL comprises the amino acid sequence of SEQ ID NO:26;
(w) the VH comprises or consists of the amino acid sequence of SEQ ID NO: 31 and the VL comprises or consists of the amino acid sequence of SEQ ID NO:32;
(x) the VH comprises or consists of the amino acid sequence of SEQ ID NO: 39 and the VL comprises or consists of the amino acid sequence of SEQ ID NO:40;
(y) the VH comprises or consists of the amino acid sequence of SEQ ID NO: 44 and the VL comprises or consists of the amino acid sequence of SEQ ID NO:45;
(z) the VH comprises or consists of the amino acid sequence of SEQ ID NO: 50 and the VL comprises or consists of the amino acid sequence of SEQ ID NO:51;
(aa) the VH comprises or consists of the amino acid sequence of SEQ ID NO: 58 and the VL comprises or consists of the amino acid sequence of SEQ ID NO:59;
(bb) the VH comprises or consists of the amino acid sequence of SEQ ID NO: 61 and the VL comprises or consists of the amino acid sequence of SEQ ID NO:62;
(cc) the VH comprises or consists of the amino acid sequence of SEQ ID NO: 69 and the VL comprises or consists of the amino acid sequence of SEQ ID NO:70;
(dd) the VH comprises or consists of the amino acid sequence of SEQ ID NO: 72 and the VL comprises or consists of the amino acid sequence of SEQ ID NO:73;
(ee) the VH comprises or consists of the amino acid sequence of SEQ ID NO: 75 and the VL comprises or consists of the amino acid sequence of SEQ ID NO:76;
(ff) the VH comprises or consists of the amino acid sequence of SEQ ID NO: 78 and the VL comprises or consists of the amino acid sequence of SEQ ID NO:79;
(gg) the VH comprises or consists of the amino acid sequence of SEQ ID NO: 81 and the VL comprises or consists of the amino acid sequence of SEQ ID NO:82; or
(hh) the VH comprises or consists of the amino acid sequence of SEQ ID NO: 84 and the VL comprises or consists of the amino acid sequence of SEQ ID NO:85.

4. (canceled)

5. The antigen-binding site of claim 1, wherein the antigen-binding site is a single-chain fragment variable fragment (scFv) or antigen-binding fragment (Fab).

6. (canceled)

7. The antigen-binding site of claim 1, wherein:
(a) the antigen-binding site binds human PD-L1 with an KD less than about 5 nM, less than about 4 nM, less than about 3 nM, less than about 2 nM, less than about 1 nM, less than about 0.8 nM, less than about 0.6 nM, less than about 0.4 nM, less than about 0.2 nM, or less than about 0.1 nM, as measured by surface plasmon resonance (SPR);
(b) the antigen-binding site binds human PD-L1 with an EC50 less than about 160 nM, less than about 10 nM, less than about 1.5 nM, less than about 1.2 nM, less than about 1.0 nM, less than about 0.8 nM, less than about 0.7 nM, less than about 0.6 nM, less than about 0.5 nM, or less than about 0.4 nM, as measured by enzyme-linked immunosorbent assay (ELISA);
(c) the antigen-binding site binds cells expressing human PD-L1 with an EC50 less than about 40 nM, less than about 10 nM, less than about 8 nM, less than about 6 nM, less than about 4 nM, less than about 2 nM, less than about 1 nM, less than about 0.5 nM, less than about 0.3 nM, less than about 0.2 nM, less than about 0.1 nM, as measured by fluorescence-activated cell sorting (FACS); and/or
(d) wherein the antigen-binding site competes with PD-1 for binding PD-L1 or inhibits PD-L1 binding to PD-1.

8-10. (canceled)

11. A protein comprising the antigen-binding site of claim 1, optionally wherein the protein comprises one or more antibody heavy chain constant regions, further optionally wherein:
(a) the antibody heavy chain constant region is human IgG heavy chain constant region;
(b) the antibody heavy chain constant region is human IgG1 heavy chain constant region;
(c) the antibody heavy chain constant region comprises an amino acid sequence at least 90% identical to SEQ ID NO:90;
(d) the antibody heavy chain constant region comprises, relative to SEQ ID NO: 90, one or more mutations selected from L234A, L235A, P329G, Y349C, S354C, T366S, T366W, L368A, F405K, K409A and Y407V, numbered according to the EU numbering system;
(e) the antibody heavy chain constant region comprises an amino acid sequence of SEQ ID NO:91 or SEQ ID NO:92; and/or
(f) the protein comprises a first antibody heavy chain constant region comprising, relative to SEQ ID NO:90, one or more mutations selected from S354C, T366W and K409A and a second antibody heavy chain constant region comprising, relative to SEQ ID NO:90, one or more mutations selected from Y349C, T366S, L368A, F405K and Y407V, numbered according to the EU numbering system.

12-18. (canceled)

19. The protein of claim 11, wherein the protein is an antibody, optionally wherein the antibody comprises:
(a) a heavy chain (HC) comprising an amino acid sequence at least 90% identical to SEQ ID NO:148 and a light chain (LC) comprising an amino acid sequence at least 90% identical to SEQ ID NO:149;
(b) a HC comprising an amino acid sequence at least 90% identical to SEQ ID NO: 150 and a LC comprising an amino acid sequence at least 90% identical to SEQ ID NO: 151;
(c) a HC comprising an amino acid sequence at least 90% identical to SEQ ID NO: 152 and a LC comprising an amino acid sequence at least 90% identical to SEQ ID NO: 153;
(d) a HC comprising an amino acid sequence at least 90% identical to SEQ ID NO: 154 and a LC comprising an amino acid sequence at least 90% identical to SEQ ID NO: 155;
(e) a HC comprising an amino acid sequence at least 90% identical to SEQ ID NO: 156 and a LC comprising an amino acid sequence at least 90% identical to SEQ ID NO: 157;
(f) a HC comprising an amino acid sequence at least 90% identical to SEQ ID NO: 159 and a LC comprising an amino acid sequence at least 90% identical to SEQ ID NO: 160;
(g) a HC comprising or consists of the amino acid sequence of SEQ ID NO: 148 and a LC comprising or consists of the amino acid sequence of SEQ ID NO: 149;
(h) a HC comprising or consists of the amino acid sequence of SEQ ID NO: 150 and a LC comprising or consists of the amino acid sequence of SEQ ID NO:151;
(i) a HC comprising or consists of the amino acid sequence of SEQ ID NO: 152 and a LC comprising or consists of the amino acid sequence of SEQ ID NO:153;
(j) a HC comprising or consists of the amino acid sequence of SEQ ID NO: 154 and a LC comprising or consists of the amino acid sequence of SEQ ID NO:155;
(k) a HC comprising or consists of the amino acid sequence of SEQ ID NO: 156 and a LC comprising or consists of the amino acid sequence of SEQ ID NO: 157; or
(l) a HC comprising or consists of the amino acid sequence of SEQ ID NO: 159 and a LC comprising or consists of the amino acid sequence of SEQ ID NO:160.

20-21. (canceled)

22. The protein of claim 19, wherein;
(a) the antibody binds human PD-L1 with an KD of less than about 10 nM, less than about 6 nM, less than about 3 nM, less than about 1 nM, or less than about 0.4 nM, as measured by SPR;
(b) the antibody binds human PD-L1 with an EC50 of less than about 0.2 nM, as measured by ELISA;
(c) the antibody binds cells expressing human PD-L1 with an EC50 of less than about 7 nM, as measured by FACS;
(d) the antibody inhibits tumor growth in vivo; and/or
(e) the antibody induces IFNγ and TNFα secretion in vivo, at a comparable to or more enhanced level as compared to atezolizumab.

23-26. (canceled)

27. A bifunctional protein, comprising:
(a) an antigen-binding site that binds PD-L1, comprising:
(i) a heavy chain variable domain (VH) comprising complementarity-determining region 1 (CDR1) of SEQ ID NO: 11, 3, 19, 33, 52, or 63; complementarity-determining region 2 (CDR2) of SEQ ID NO: 12, 4, 20, 34, 41, 53, or 64; and complementarity-determining region 3 (CDR3) of SEQ ID NO: 89, 5, 13, 21, 27, 35, 46, 54, 65, 71, 74, 77, 80, 83, or 86; and
(ii) a light chain variable domain (VL) comprising CDR1 sequence of SEQ ID NO: 47, 6, 14, 22, 28, 36, 55, or 66; CDR2 sequence of SEQ ID NO: 48, 7, 15, 23, 29, 37, 42, 56, 60, or 67; and CDR3 sequences of SEQ ID NO: 49, 8, 16, 24, 30, 38, 43, 57, or 68; and
(b) an interleukin-15 (IL-15) polypeptide, an interleukin-15 receptor alpha (IL-15Rα) polypeptide, a wild-type interleukin-2 (IL-2) polypeptide, or an engineered IL-2 polypeptide, or a functional fragment or variant thereof, optionally further comprising one or more antibody heavy chain constant regions.

28. (canceled)

29. The bifunctional protein of claim 27, wherein the antigen-binding site that binds PD-L1 comprises:
(a) the VH comprises an amino acid sequence at least 90% identical to SEQ ID NO: 87 and the VL comprises an amino acid sequence at least 90% identical to SEQ ID NO: 88;
(b) the VH comprises an amino acid sequence at least 90% identical to SEQ ID NO: 1 and the VL comprises an amino acid sequence at least 90% identical to SEQ ID NO: 2;
(c) the VH comprises an amino acid sequence at least 90% identical to SEQ ID NO: 9 and the VL comprises an amino acid sequence at least 90% identical to SEQ ID NO: 10;
(d) the VH comprises an amino acid sequence at least 90% identical to SEQ ID NO: 17 and the VL comprises an amino acid sequence at least 90% identical to SEQ ID NO: 18;
(e) the VH comprises an amino acid sequence at least 90% identical to SEQ ID NO: 25 and the VL comprises an amino acid sequence at least 90% identical to SEQ ID NO: 26;
(f) the VH comprises an amino acid sequence at least 90% identical to SEQ ID NO: 31 and the VL comprises an amino acid sequence at least 90% identical to SEQ ID NO: 32;
(g) the VH comprises an amino acid sequence at least 90% identical to SEQ ID NO: 39 and the VL comprises an amino acid sequence at least 90% identical to SEQ ID NO: 40;
(h) the VH comprises an amino acid sequence at least 90% identical to SEQ ID NO: 44 and the VL comprises an amino acid sequence at least 90% identical to SEQ ID NO: 45;
(i) the VH comprises an amino acid sequence at least 90% identical to SEQ ID NO: 50 and the VL comprises an amino acid sequence at least 90% identical to SEQ ID NO: 51;
(j) the VH comprises an amino acid sequence at least 90% identical to SEQ ID NO: 58 and the VL comprises an amino acid sequence at least 90% identical to SEQ ID NO: 59;
(k) the VH comprises an amino acid sequence at least 90% identical to SEQ ID NO: 61 and the VL comprises an amino acid sequence at least 90% identical to SEQ ID NO: 62;
(l) the VH comprises an amino acid sequence at least 90% identical to SEQ ID NO: 69 and the VL comprises an amino acid sequence at least 90% identical to SEQ ID NO: 70;
(m) the VH comprises an amino acid sequence at least 90% identical to SEQ ID NO: 72 and the VL comprises an amino acid sequence at least 90% identical to SEQ ID NO: 73;
(n) the VH comprises an amino acid sequence at least 90% identical to SEQ ID NO: 75 and the VL comprises an amino acid sequence at least 90% identical to SEQ ID NO: 76;
(o) the VH comprises an amino acid sequence at least 90% identical to SEQ ID NO: 78 and the VL comprises an amino acid sequence at least 90% identical to SEQ ID NO: 79;
(p) the VH comprises an amino acid sequence at least 90% identical to SEQ ID NO: 81 and the VL comprises an amino acid sequence at least 90% identical to SEQ ID NO: 82;
(q) the VH comprises an amino acid sequence at least 90% identical to SEQ ID NO: 84 and the VL comprises an amino acid sequence at least 90% identical to SEQ ID NO: 85
(r) the VH comprises or consists of the amino acid sequence of SEQ ID NO: 87 and the VL comprises or consists of the amino acid sequence of SEQ ID NO:88
(s) the VH comprises or consists of the amino acid sequence of SEQ ID NO:1 and the VL comprises or consists of the amino acid sequence of SEQ ID NO:2;
(t) the VH comprises or consists of the amino acid sequence of SEQ ID NO:9 and the VL comprises or consists of the amino acid sequence of SEQ ID NO: 10;
(u) the VH comprises or consists of the amino acid sequence of SEQ ID NO: 17 and the VL comprises or consists of the amino acid sequence of SEQ ID NO:18;
(v) the VH comprises or consists of the amino acid sequence of SEQ ID NO: 25 and the VL comprises the amino acid sequence of SEQ ID NO:26;
(w) the VH comprises or consists of the amino acid sequence of SEQ ID NO: 31 and the VL comprises or consists of the amino acid sequence of SEQ ID NO:32;
(x) the VH comprises or consists of the amino acid sequence of SEQ ID NO: 39 and the VL comprises or consists of the amino acid sequence of SEQ ID NO:40;
(y) the VH comprises or consists of the amino acid sequence of SEQ ID NO: 44 and the VL comprises or consists of the amino acid sequence of SEQ ID NO:45;
(z) the VH comprises or consists of the amino acid sequence of SEQ ID NO: 50 and the VL comprises or consists of the amino acid sequence of SEQ ID NO:51;
(aa) the VH comprises or consists of the amino acid sequence of SEQ ID NO: 58 and the VL comprises or consists of the amino acid sequence of SEQ ID NO:59;
(bb) the VH comprises or consists of the amino acid sequence of SEQ ID NO: 61 and the VL comprises or consists of the amino acid sequence of SEQ ID NO:62;
(cc) the VH comprises or consists of the amino acid sequence of SEQ ID NO: 69 and the VL comprises or consists of the amino acid sequence of SEQ ID NO:70;
(dd) the VH comprises or consists of the amino acid sequence of SEQ ID NO: 72 and the VL comprises or consists of the amino acid sequence of SEQ ID NO:73;
(ee) the VH comprises or consists of the amino acid sequence of SEQ ID NO: 75 and the VL comprises or consists of the amino acid sequence of SEQ ID NO:76;
ff) the VH comprises or consists of the amino acid sequence of SEQ ID NO: 78 and the VL comprises or consists of the amino acid sequence of SEQ ID NO:79;
(gg) the VH comprises or consists of the amino acid sequence of SEQ ID NO: 81 and the VL comprises or consists of the amino acid sequence of SEQ ID NO:82; or
(hh) the VH comprises or consists of the amino acid sequence of SEQ ID NO: 84 and the VL comprises or consists of the amino acid sequence of SEQ ID NO:85.

30. (canceled)

31. The bifunctional protein of claim 27, comprising:
(a) a first subunit comprising the antigen-binding site that binds PD-L1, an interleukin-15 (IL-15) polypeptide or a functional fragment or variant thereof, and a first antibody heavy chain constant region; and
(b) a second subunit comprising the antigen-binding site that binds PD-L1, an interleukin-15 receptor alpha (IL-15Rα) polypeptide or functional fragment or variant thereof, and a second antibody heavy chain constant region.

32. The bifunctional protein of claim 27, wherein:
(a) the IL-15 polypeptide comprises an amino acids 50-162 of SEQ ID NO:93 or a functional fragment or variant thereof; and/or
(b) the IL-15Rα polypeptide comprises an amino acids 31-97 of SEQ ID NO: 94 or a functional fragment or variant thereof.

33. (canceled)

34. The bifunctional protein of claim 27, wherein:
(a) the first subunit comprises an amino acid sequence at least 90% identical to SEQ ID NO:186 and the second subunit comprises an amino acid sequence at least 90% identical to SEQ ID NO:187; optionally wherein the first subunit further comprises an amino acid sequence at least 90% identical to SEQ ID NO:181, optionally wherein the first subunit comprises an amino acid sequence of SEQ ID NO:186 and the second subunit comprises an amino acid sequence of SEQ ID NO:187; optionally wherein the first subunit further comprises an amino acid sequence of SEQ ID NO: 181; or
(b) the first subunit comprising an amino acid sequence at least 90% identical to SEQ ID NO:188 and the second subunit comprising an amino acid sequence at least 90% identical to SEQ ID NO:189, optionally wherein the first subunit comprises or consists of the amino acid sequence of SEQ ID NO:188, and the second subunit comprises or consists of the amino acid sequence of SEQ ID NO: 189.

35-37. (canceled)

38. The bifunctional protein of claim 27, wherein the engineered IL-2 polypeptide comprises:
(a) an IL-2 receptor a (IL-2Rα) binding region 1 comprising, relative to wild-type IL-2, one or more mutations at one or more positions selected from:
a mutation at position K35 selected from K35G, K35L, K35S, K35V, K35D, K35E, and K35C;
a mutation at position R38 selected from R38V, R38D, R38E, R38S, R38I, R38A, R38Y, R38G, R38C, and R38N;
a mutation at position F42 selected from F42A, F42R, F42G, F42I, F42L, F42P and F42H; and
a mutation at position Y45 selected from Y45S, Y45P, Y45A, Y45V, Y45C, Y45T, and Y45F, and/or
(b) an IL-2 receptor β (IL-2Rβ) binding region 2 motif comprising:
(SEQ ID NO: 95)
X1-X2-X3-D-X4-X-5-X6-N-X7-X8-X9-X10-X11-X12-X13,
wherein:
X1 is selected from C, T, G, W, I, S, E, and K;
X2 is selected from Y, P, V, W, L, A, and G;
X3 is selected from S, T, Q, G, M, E, R, and K;
X4 is selected from A, V, S, and T;
X5 is selected from I, L, T, and V;
X6 is selected from S, T, E, D, and R;
X7 is selected from I, A, M, and V;
X8 is selected from S, T, N, Q, I, G, E, K, and R;
X9 is selected from V, L, and I;
X10 is selected from N, T, I, and L;
X11 is selected from V, A, and I;
X12 is selected from and Q, L, G, K, and R; and
X13 is selected from A, D, and E.

39. The bifunctional protein of claim 38, wherein the engineered IL-2 polypeptide comprises:
(a) an IL-2Rα binding region 1 comprises an amino acid sequence at least 90% identical to an amino acid sequence selected from SEQ ID NOS: 124-147, optionally wherein the IL-2Rα binding region 1 comprises an amino acid sequence selected from SEQ ID NOS: 124-147; and/or
(b) an IL-2Rβ binding region 2 motif comprises an amino acid sequence at least 90% identical to an amino acid sequence selected from SEQ ID NOS: 96-123, optionally wherein the IL-2Rβ binding region 2 motif comprises an amino acid sequence selected from SEQ ID NOS: 96-123.

40. (canceled)

41. The bifunctional protein of claim 27, comprising:
(a) an amino acid sequence at least 90% identical to an amino acid sequence selected from SEQ ID NOS: 160-164, or
(b) an amino acid sequence selected from SEQ ID NOS: 160-164.

42. (canceled)

43. The bifunctional protein of claim 27, comprising:
(a) a first subunit comprising the antigen-binding site that binds PD-L1 and a first antibody heavy chain constant region; and
(b) a second subunit comprising a wild-type interleukin-2 (IL-2) or an engineered IL-2 polypeptide or a functional fragment or variant thereof and a second antibody heavy chain constant region, optionally wherein:
(i) the first subunit comprises an amino acid sequence at least 90% identical to SEQ ID NO:176 and the second subunit comprises an amino acid sequence at least 90% identical to an amino acid sequence selected from SEQ ID NOS: 165-175, further optionally wherein the first subunit comprises an amino acid sequence of SEQ ID NO: 176 and the second subunit comprises an amino acid sequence selected from SEQ ID NOS: 165-175; or
(ii) the first subunit comprises an amino acid sequence at least 90% identical to an amino acid sequence selected from SEQ ID NOS: 177-180 and the second subunit comprises an amino acid sequence at least 90% identical to an amino acid sequence selected from SEQ ID NOS: 165-175, optionally wherein the first subunit further comprises an amino acid sequence at least 90% identical to SEQ ID NO: 181,
further optionally wherein:
(A) the first subunit comprises an amino acid sequence selected from SEQ ID NOS: 177-180 and the second subunit comprises an amino acid sequence selected from SEQ ID NOS: 165-175, optionally wherein the first subunit further comprises an amino acid sequence of SEQ ID NO: 181; or
(B) the first subunit comprises the amino acid sequence of SEQ ID NO: 177 and SEQ ID NO:181, and the second subunit comprises the amino acid sequence of SEQ ID NO:171.

44-48. (canceled)

49. The bifunctional protein of claim 27, comprising:
(a) a first subunit comprising the antigen-binding site that binds PD-L1 and a first antibody heavy chain constant region; and
(b) a second subunit comprising the antigen-binding site that binds PD-L1, a wild-type interleukin-2 (IL-2) or an engineered IL-2 polypeptide or a functional fragment or variant thereof, and a second antibody heavy chain constant region
wherein optionally, the first subunit comprises an amino acid sequence at least 90% identical to SEQ ID NO:182 and the second subunit comprises an amino acid sequence at least 90% identical to an amino acid sequence selected from SEQ ID NOS: 183-185; optionally wherein the first and the second subunit, each independently, further comprises an amino acid sequence at least 90% identical to SEQ ID NO:181, wherein further optionally the first subunit comprises an amino acid sequence of SEQ ID NO:182 and the second subunit comprises an amino acid sequence selected from SEQ ID NOS: 183-185; optionally wherein the first and the second subunit, each independently, further comprises an amino acid sequence of SEQ ID NO: 181.

50-51. (canceled)

52. The bifunctional protein of claim 27 comprising one or more antibody heavy chain constant regions, wherein the antibody heavy chain constant region is human IgG heavy chain constant region, optionally wherein:
(a) the antibody heavy chain constant region is human IgG1 heavy chain constant region;
(b) the antibody heavy chain constant region comprises an amino acid sequence of at least 90% identity to SEQ ID NO:90;
(c) the antibody heavy chain constant region comprises, relative to SEQ ID NO: 90, one or more mutations selected from L234A, L235A, P329G, Y349C, S354C, T366S, T366W, L368A, F405K, K409A and Y407V, numbered according to the EU numbering system;
(d) one of the first and the second antibody heavy chain constant regions comprises, relative to SEQ ID NO:90, one or more mutations selected from S354C, T366W and K409A; and the other antibody heavy chain constant region comprises, relative to SEQ ID NO:90, one or more mutations selected from Y349C, T366S, L368A, F405K and Y407V, numbered according to the EU numbering system;
(e) one of the first and the second antibody heavy chain constant regions comprises, relative to SEQ ID NO:90, mutations S354C, T366W and K409A; and the other antibody heavy chain constant region comprises, relative to SEQ ID NO:90, mutations Y349C, T366S, L368A, F405K and Y407V, numbered according to the EU numbering system; and/or
(f) one of the first and the second antibody heavy chain constant regions comprises an amino acid sequence of SEQ ID NO:91 and the other antibody heavy chain constant region comprises an amino acid sequence of SEQ ID NO:92.

53-58. (canceled)

59. The bifunctional protein of claim 27, wherein:
(a) the bifunctional protein binds human PD-L1 with a KD of less than about 1 nM or with a comparable or lower KD as compared to the comprised antigen-binding site that binds PD-L1, as measured by SPR;
(b) the bifunctional protein binds PD-L1 with a KD of less than about 0.5 nM or less than about 0.1 nM, as measured by SPR;
(c) the bifunctional protein binds PD-L1 with a EC50 of less than about 0.4 nM, as measured by ELISA;
(d) the bifunctional protein competes with PD-1 for binding PD-L1 or inhibits PD-L1 binding to PD-1; and/or
(e) the bifunctional protein inhibits PD-L1 binding to PD-1.

60-63. (canceled)

64. The bifunctional protein of claim 27, wherein:
(a) the bifunctional protein binds IL-2Rβ with a KD of less than about 100 nM, less than about 80 nM, less than about 50 nM, less than about 10 nM, or less than about 5 nM, as measured by SPR,
(b) the bifunctional protein binds IL-2Rβ with a KD of less than about 65 nM or less than about 50 nM, as measured by SPR;
(c) the bifunctional protein binds IL-2Rα with a KD of less than about 40 nM, as measured by SPR;
(d) the bifunctional protein binds IL-2Rα with a EC50 of less than about 1 nM, as measured by ELISA; and/or
(e) the bifunctional protein binds IL-2Rβ with a EC50 of less than about 5 nM, less than about 2.5 nM, less than about 1.5 nM, less than about 1 nM, or less than about 0.6 nM, as measured by ELISA.

65-68. (canceled)

69. The bifunctional protein of claim 27, wherein:
(a) the bifunctional protein induces p-STAT5 expression in immune cells;
(b) the bifunctional protein induces p-STAT5 expression in immune cells with EC50 of less than about 1 nM, less than about 0.6 nM, or less than about 0.1 nM, as measured in isolated human peripheral blood mononuclear cells (PBMCs), and wherein the immune cells are T cells, NK cells, or Tregs;
(c) the bifunctional protein induces p-STAT5 expression in immune cells with EC50 of less than about 0.5 nM or less than about 0.1 nM, as measured in isolated PBMCs, and wherein the immune cells are T cells, NK cells, or Tregs;
(d) the bifunctional protein induces p-STAT5 expression in immune cells as measured in mouse splenocytes, and wherein the immune cells are T cells, NK cells, or Tregs;
(e) the bifunctional protein inhibits tumor growth in vivo; and/or
(f) the bifunctional protein induces immune cell proliferation in vivo, optionally wherein the immune cell is T cell or NK cell, further optionally wherein the T cell is CD8+ T cell.

70-76. (canceled)

77. An antibody, comprising a VH and VL, wherein the VH comprises the polypeptide sequence of SEQ ID NO:87 and the VH comprises the polypeptide sequence of SEQ ID NO:88, optionally wherein the antibody comprises the polypeptide sequences of SEQ ID NO: 177 and SEQ ID NO: 181.

78. (canceled)

79. An isolated polynucleotide encoding an antigen-binding site of claim 1.

80. An expression vector comprising the polynucleotide of claim 79.

81. A modified cell comprising the isolated polynucleotide of claim 79.

82. A pharmaceutical composition comprising the an antigen-binding site of claim 1, and a pharmaceutically acceptable carrier.

83-88. (canceled)

89. A method of modulating an immune response in a subject in need thereof, the method comprising administering to the subject a therapeutically effective amount of an antigen-binding site of claim 1 or a pharmaceutical composition thereof, optionally wherein the modulating the immune response comprises at least one of enhancing T cell activity or enhancing NK cell activity, further optionally wherein the subject is treated with an additional therapeutic agent.

90. (canceled)

91. A method of treating cancer in a subject in need thereof, the method comprising administering to the subject a therapeutically effective amount of an antigen-binding site of claim 1 or a pharmaceutical composition thereof, optionally wherein the cancer comprises breast cancer, pancreatic cancer, lung cancer, glioblastoma, renal cell carcinoma, or melanoma.

92-93. (canceled)


SEQUENCE LISTING (192 sequences)

SEQ ID NO: 1 - protein, 125 aa, Artificial sequence
  Synthetic sequence
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRRLRYFDWAAYNWFDPWGQGTLVTVSS

SEQ ID NO: 2 - protein, 105 aa, Artificial sequence
  Synthetic sequence
QSALTQDPAVSVALGQTVRITCQGDSLRSHYASWYQQKPGQAPVVVVYDNSDRPSGIPDRFSGSNSGNTATLTISRVEVGDEADYYCQVWDSSSDHYVFGTGTKV

SEQ ID NO: 3 - protein, 9 aa, Artificial sequence
  Synthetic sequence
GSFSGYYWS

SEQ ID NO: 4 - protein, 13 aa, Artificial sequence
  Synthetic sequence
WIGEINHSGSTNY

SEQ ID NO: 5 - protein, 18 aa, Artificial sequence
  Synthetic sequence
RGRRLRYFDWAAYNWFDP

SEQ ID NO: 6 - protein, 8 aa, Artificial sequence
  Synthetic sequence
SLRSHYAS

SEQ ID NO: 7 - protein, 12 aa, Artificial sequence
  Synthetic sequence
VVVVYDNSDRPS

SEQ ID NO: 8 - protein, 10 aa, Artificial sequence
  Synthetic sequence
QVWDSSSDHY

SEQ ID NO: 9 - protein, 122 aa, Artificial sequence
  Synthetic sequence
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDSRWGPAPHAFDIWGQGTMVTVSS

SEQ ID NO: 10 - protein, 109 aa, Artificial sequence
  Synthetic sequence
QQSVLTQPASVSGSPGQSITISCTGTSSDIGSYNLVSWYQNHPGKAPKLILYEITKRPSGVSSRFSGSKSGNAASLTISGLQAEDEAEYFCCSYAGLSVFFVFGGGTKL

SEQ ID NO: 11 - protein, 9 aa, Artificial sequence
  Synthetic sequence
GTFSSYAIS

SEQ ID NO: 12 - protein, 14 aa, Artificial sequence
  Synthetic sequence
WMGGIIPIFGTANY

SEQ ID NO: 13 - protein, 14 aa, Artificial sequence
  Synthetic sequence
RDSRWGPAPHAFDI

SEQ ID NO: 14 - protein, 11 aa, Artificial sequence
  Synthetic sequence
SSDIGSYNLVS

SEQ ID NO: 15 - protein, 11 aa, Artificial sequence
  Synthetic sequence
LILYEITKRPS

SEQ ID NO: 16 - protein, 10 aa, Artificial sequence
  Synthetic sequence
CSYAGLSVFF

SEQ ID NO: 17 - protein, 120 aa, Artificial sequence
  Synthetic sequence
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKASGWYPWYFDYWGQGTLVTVSS

SEQ ID NO: 18 - protein, 107 aa, Artificial sequence
  Synthetic sequence
VIWMTQSPSSLSASLGDRVTITCRASQSVSTYINWYQQKSGKGPKLLIFNASTLQSGVPSRFSGSGSGTDFTLTITSLQPDDSATYYCQQSYRVPLSFGGGTKVEIR

SEQ ID NO: 19 - protein, 9 aa, Artificial sequence
  Synthetic sequence
FTFSSYAMS

SEQ ID NO: 20 - protein, 14 aa, Artificial sequence
  Synthetic sequence
WVSAISGSGGSTYY

SEQ ID NO: 21 - protein, 12 aa, Artificial sequence
  Synthetic sequence
KASGWYPWYFDY

SEQ ID NO: 22 - protein, 8 aa, Artificial sequence
  Synthetic sequence
QSVSTYIN

SEQ ID NO: 23 - protein, 11 aa, Artificial sequence
  Synthetic sequence
LLIFNASTLQS

SEQ ID NO: 24 - protein, 8 aa, Artificial sequence
  Synthetic sequence
QQSYRVPL

SEQ ID NO: 25 - protein, 120 aa, Artificial sequence
  Synthetic sequence
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKAGSWYPYYFDYWGQGTLVTVSS

SEQ ID NO: 26 - protein, 107 aa, Artificial sequence
  Synthetic sequence
DIVMTQSPSSLSASVGDRVTITCRASQTVSTYINWYQQRSGKGPKLLIFDASRLQSGAPSRFSGSGSGTEFTLTITSLQPDDSATYYCQQSYRIPLSFGGGTKVEIR

SEQ ID NO: 27 - protein, 12 aa, Artificial sequence
  Synthetic sequence
KAGSWYPYYFDY

SEQ ID NO: 28 - protein, 8 aa, Artificial sequence
  Synthetic sequence
QTVSTYIN

SEQ ID NO: 29 - protein, 11 aa, Artificial sequence
  Synthetic sequence
LLIFDASRLQS

SEQ ID NO: 30 - protein, 9 aa, Artificial sequence
  Synthetic sequence
QQSYRIPLS

SEQ ID NO: 31 - protein, 118 aa, Artificial sequence
  Synthetic sequence
QMQLVQSGAEVKKPGATVKISCKVSGYTFTDYYMHWVQQAPGKGLEWMGLVDPEDGETIYAEKFQGRVTITADTSTDTAYMELSSLRSEDTAVFYCATDGSGSYYLTLGQGTLVTVSS

SEQ ID NO: 32 - protein, 115 aa, Artificial sequence
  Synthetic sequence
NFMLTQPSSLSASPGASASLTCTLPSGINVGPYGIFWYQQKPGSPPQYLLRYKSNSENHQGSGVPSRFSGSEDASANVGILLISGLQSEDEADYYCMIWHSSAWVFGGGTKVTVL

SEQ ID NO: 33 - protein, 9 aa, Artificial sequence
  Synthetic sequence
YTFTDYYMH

SEQ ID NO: 34 - protein, 15 aa, Artificial sequence
  Synthetic sequence
WMGLVDPEDGETIYA

SEQ ID NO: 35 - protein, 11 aa, Artificial sequence
  Synthetic sequence
TDGSGSYYLTL

SEQ ID NO: 36 - protein, 10 aa, Artificial sequence
  Synthetic sequence
SGINVGPYGI

SEQ ID NO: 37 - protein, 16 aa, Artificial sequence
  Synthetic sequence
QYLLRYKSNSENHQGS

SEQ ID NO: 38 - protein, 9 aa, Artificial sequence
  Synthetic sequence
MIWHSSAWV

SEQ ID NO: 39 - protein, 125 aa, Artificial sequence
  Synthetic sequence
QVQLQQWGAGLLKPSETLSLTCAVYGGSFSGYYWSWIRQPPGKGLEWIGEINHIGSTNYNPSLKSRVTISVDTSNNQFSLKLSSVTAADTAVYYCARGRRLRYFDWAAYNWFDPWGQGTLVTVSS

SEQ ID NO: 40 - protein, 108 aa, Artificial sequence
  Synthetic sequence
QSALTQDPAVSVALGQTVRISCQGDSLRSHYASWYQQKPGQAPVVVVYDNSDRPSGIPDRFSGSNSGNTATLTISRVEVGDEADYYCQVWDSSSDHYVFGTGTKVTVL

SEQ ID NO: 41 - protein, 13 aa, Artificial sequence
  Synthetic sequence
WIGEINHIGSTNY

SEQ ID NO: 42 - protein, 11 aa, Artificial sequence
  Synthetic sequence
VVVYDNSDRPS

SEQ ID NO: 43 - protein, 11 aa, Artificial sequence
  Synthetic sequence
QVWDSSSDHYV

SEQ ID NO: 44 - protein, 121 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKGYGSGWRGDYWGQGTLVTVSS

SEQ ID NO: 45 - protein, 110 aa, Artificial sequence
  Synthetic sequence
QSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVL

SEQ ID NO: 46 - protein, 13 aa, Artificial sequence
  Synthetic sequence
RDKGYGSGWRGDY

SEQ ID NO: 47 - protein, 10 aa, Artificial sequence
  Synthetic sequence
YSNIGSNFVN

SEQ ID NO: 48 - protein, 11 aa, Artificial sequence
  Synthetic sequence
LLIHFNNQRPS

SEQ ID NO: 49 - protein, 10 aa, Artificial sequence
  Synthetic sequence
STWDDSLKGP

SEQ ID NO: 50 - protein, 119 aa, Artificial sequence
  Synthetic sequence
QVQLVQSGGGLVKPGGSLRLSCAASGFTFSSYSMNWVRQAPGKGLEWVSSISSSSSYIYYADSVKGRFTISRDNAKNSLYLQMNSLKAEDTAVYYCARDSLQWLALDYWGQGTLVTVSS

SEQ ID NO: 51 - protein, 108 aa, Artificial sequence
  Synthetic sequence
QPVLTQPPSVSVAPGETATITCGGNNIGRKSVHWYLQKPGQAPVLVIGHDTDRPSGIPARFSGSNSGNTATLTINRVEAGDEADYYCQVWDTSGHHYVFGTGTKVTVL

SEQ ID NO: 52 - protein, 9 aa, Artificial sequence
  Synthetic sequence
FTFSSYSMN

SEQ ID NO: 53 - protein, 14 aa, Artificial sequence
  Synthetic sequence
WVSSISSSSSYIYY

SEQ ID NO: 54 - protein, 11 aa, Artificial sequence
  Synthetic sequence
RDSLQWLALDY

SEQ ID NO: 55 - protein, 8 aa, Artificial sequence
  Synthetic sequence
NIGRKSVH

SEQ ID NO: 56 - protein, 11 aa, Artificial sequence
  Synthetic sequence
LVIGHDTDRPS

SEQ ID NO: 57 - protein, 10 aa, Artificial sequence
  Synthetic sequence
QVWDTSGHHY

SEQ ID NO: 58 - protein, 121 aa, Artificial sequence
  Synthetic sequence
QMQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKGYGSGWRGDYWGQGTLVTVSS

SEQ ID NO: 59 - protein, 110 aa, Artificial sequence
  Synthetic sequence
QSVLTQPPSASGTPGQRVTISCSGSYSNIGSNFVNWYQQLPGTAPKLLIHFDNQRPSGVPDRFSGSRSGTSASLAISGLQSEDEADYFCSTWDDSLKGPIFGGGTKLTVL

SEQ ID NO: 60 - protein, 11 aa, Artificial sequence
  Synthetic sequence
LLIHFDNQRPS

SEQ ID NO: 61 - protein, 125 aa, Artificial sequence
  Synthetic sequence
EVQLVESGGGLVKPGGSLRLSCAASGLTFSNAWMSWVRQAPGKGLEWVGRIKSKTDGGTTEYAAPVKGRFTISRDDSKNTLYLQMSSLKTEDTAVYYCTIVYYYDSSGPVFFQHWGQGTLVTVSS

SEQ ID NO: 62 - protein, 110 aa, Artificial sequence
  Synthetic sequence
QSVLTQPPSASATPGQRVTISCSGSDSNIATNYVYWYQQFPGTAPKLLIYRNHQRPSGVPDRFSGSRSGTSASLAISGLRSEDEADYYCATWDDSVNSWVFGGGTKLTVL

SEQ ID NO: 63 - protein, 9 aa, Artificial sequence
  Synthetic sequence
LTFSNAWMS

SEQ ID NO: 64 - protein, 16 aa, Artificial sequence
  Synthetic sequence
WVGRIKSKTDGGTTEY

SEQ ID NO: 65 - protein, 15 aa, Artificial sequence
  Synthetic sequence
IVYYYDSSGPVFFQH

SEQ ID NO: 66 - protein, 10 aa, Artificial sequence
  Synthetic sequence
DSNIATNYVY

SEQ ID NO: 67 - protein, 11 aa, Artificial sequence
  Synthetic sequence
LLIYRNHQRPS

SEQ ID NO: 68 - protein, 10 aa, Artificial sequence
  Synthetic sequence
ATWDDSVNSW

SEQ ID NO: 69 - protein, 121 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARNKGYVSGWRSDYWGQGTLVTVSS

SEQ ID NO: 70 - protein, 110 aa, Artificial sequence; identical to SEQ ID NO: 45
  Synthetic sequence

SEQ ID NO: 71 - protein, 13 aa, Artificial sequence
  Synthetic sequence
RNKGYVSGWRSDY

SEQ ID NO: 72 - protein, 121 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKGFGSGWRGDYWGQGTLVTVSS

SEQ ID NO: 73 - protein, 110 aa, Artificial sequence; identical to SEQ ID NO: 45
  Synthetic sequence

SEQ ID NO: 74 - protein, 13 aa, Artificial sequence
  Synthetic sequence
RDKGFGSGWRGDY

SEQ ID NO: 75 - protein, 121 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKGYGSGFRGDYWGQGTLVTVSS

SEQ ID NO: 76 - protein, 110 aa, Artificial sequence; identical to SEQ ID NO: 45
  Synthetic sequence

SEQ ID NO: 77 - protein, 13 aa, Artificial sequence
  Synthetic sequence
RDKGYGSGFRGDY

SEQ ID NO: 78 - protein, 121 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSGWRGDYWGQGTLVTVSS

SEQ ID NO: 79 - protein, 110 aa, Artificial sequence; identical to SEQ ID NO: 45
  Synthetic sequence

SEQ ID NO: 80 - protein, 13 aa, Artificial sequence
  Synthetic sequence
RDKWYGSGWRGDY

SEQ ID NO: 81 - protein, 121 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRIRIGTGWRGDYWGQGTLVTVSS

SEQ ID NO: 82 - protein, 109 aa, Artificial sequence
  Synthetic sequence
SVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVL

SEQ ID NO: 83 - protein, 13 aa, Artificial sequence
  Synthetic sequence
RRIRIGTGWRGDY

SEQ ID NO: 84 - protein, 121 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWFGSGWRGDYWGQGTLVTVSS

SEQ ID NO: 85 - protein, 110 aa, Artificial sequence; identical to SEQ ID NO: 45
  Synthetic sequence

SEQ ID NO: 86 - protein, 13 aa, Artificial sequence
  Synthetic sequence
RDKWFGSGWRGDY

SEQ ID NO: 87 - protein, 121 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSS

SEQ ID NO: 88 - protein, 110 aa, Artificial sequence; identical to SEQ ID NO: 45
  Synthetic sequence

SEQ ID NO: 89 - protein, 13 aa, Artificial sequence
  Synthetic sequence
RDKWYGSAWRGDY

SEQ ID NO: 90 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 227 aa, Artificial sequence
  Synthetic sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 92 - protein, 227 aa, Artificial sequence
  Synthetic sequence
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 93 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 94 - protein, 267 aa, Homo sapiens
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 95 - protein, 15 aa, Artificial sequence
  Synthetic sequence
  misc_feature 1: Xaa  = C  T  G  W  I  S  E  and K
  misc_feature 2: Xaa = Y  P  V  W  L  A  and G
  misc_feature 3: Xaa = S  T  Q  G  M  E  R  and K
  misc_feature 5: Xaa = A  V  S  and T
  misc_feature 6: Xaa = I  L  T  and V
  misc_feature 7: Xaa = S  T  E  D  and R
  misc_feature 9: Xaa = I  A  M  and V
  misc_feature 10: Xaa = S  T  N  Q  I  G  E  K  and R
  misc_feature 11: Xaa = V  L  and I
  misc_feature 12: Xaa = N  T  I  and L
  misc_feature 13: Xaa = V  A  and I
  misc_feature 14: Xaa = Q  L  G  K  and R
  misc_feature 15: Xaa = A  D  and E
XXXDXXXNXXXXXXX

SEQ ID NO: 96 - protein, 15 aa, Artificial sequence
  Synthetic sequence
GVTDSISNAIVLARE

SEQ ID NO: 97 - protein, 15 aa, Artificial sequence
  Synthetic sequence
KWGDAVSNARVLAGE

SEQ ID NO: 98 - protein, 15 aa, Artificial sequence
  Synthetic sequence
KWGDAVSNARVLAGA

SEQ ID NO: 99 - protein, 15 aa, Artificial sequence
  Synthetic sequence
TLMDTTDNIGVLVRE

SEQ ID NO: 100 - protein, 15 aa, Artificial sequence
  Synthetic sequence
EPSDVISNINVLVQE

SEQ ID NO: 101 - protein, 15 aa, Artificial sequence
  Synthetic sequence
SPQDSIENISVLVRE

SEQ ID NO: 102 - protein, 15 aa, Artificial sequence
  Synthetic sequence
WASDSIENITLLIQE

SEQ ID NO: 103 - protein, 15 aa, Artificial sequence
  Synthetic sequence
CPTDTIENITVLIQE

SEQ ID NO: 104 - protein, 15 aa, Artificial sequence
  Synthetic sequence
RYKDSLENMQIIIQE

SEQ ID NO: 105 - protein, 15 aa, Artificial sequence
  Synthetic sequence
TARDAVDNMRVIIQE

SEQ ID NO: 106 - protein, 15 aa, Artificial sequence
  Synthetic sequence
TPRDVVENMNVLVLE

SEQ ID NO: 107 - protein, 15 aa, Artificial sequence
  Synthetic sequence
TPSDVIENMEVLILD

SEQ ID NO: 108 - protein, 15 aa, Artificial sequence
  Synthetic sequence
TPSDAIENINVLIRE

SEQ ID NO: 109 - protein, 15 aa, Artificial sequence
  Synthetic sequence
TPSDVIENITVLVQE

SEQ ID NO: 110 - protein, 15 aa, Artificial sequence
  Synthetic sequence
GVGDTIDNINVLVKE

SEQ ID NO: 111 - protein, 15 aa, Artificial sequence
  Synthetic sequence
IGRDSIDNIKVIVQE

SEQ ID NO: 112 - protein, 15 aa, Artificial sequence
  Synthetic sequence
WATDTIRNVEVLVQE

SEQ ID NO: 113 - protein, 15 aa, Artificial sequence
  Synthetic sequence
TAEDVVTNITVLVQE

SEQ ID NO: 114 - protein, 15 aa, Artificial sequence
  Synthetic sequence
TAEDVISNIRVNVQE

SEQ ID NO: 115 - protein, 15 aa, Artificial sequence
  Synthetic sequence
TPSDVIDNVSITVQE

SEQ ID NO: 116 - protein, 15 aa, Artificial sequence
  Synthetic sequence
TARDAISNIRVIVQE

SEQ ID NO: 117 - protein, 15 aa, Artificial sequence
  Synthetic sequence
RARDAIDNIRVIVQE

SEQ ID NO: 118 - protein, 15 aa, Artificial sequence
  Synthetic sequence
TPRDAIDNINVIIQE

SEQ ID NO: 119 - protein, 15 aa, Artificial sequence
  Synthetic sequence
TPRDAIDNIRVIVQE

SEQ ID NO: 120 - protein, 15 aa, Artificial sequence
  Synthetic sequence
TPRDAIDNIRVIILE

SEQ ID NO: 121 - protein, 15 aa, Artificial sequence
  Synthetic sequence
TARDAISNINVIIQE

SEQ ID NO: 122 - protein, 15 aa, Artificial sequence
  Synthetic sequence
TARDAIDNINVIVQE

SEQ ID NO: 123 - protein, 15 aa, Artificial sequence
  Synthetic sequence
TARDAIDNIRVIVLE

SEQ ID NO: 124 - protein, 12 aa, Artificial sequence
  Synthetic sequence
PVLTRMLTIKFY

SEQ ID NO: 125 - protein, 12 aa, Artificial sequence
  Synthetic sequence
PKLTRMLTLKFP

SEQ ID NO: 126 - protein, 12 aa, Artificial sequence
  Synthetic sequence
PDLTSMLAFKFY

SEQ ID NO: 127 - protein, 12 aa, Artificial sequence
  Synthetic sequence
PGLTEMLTFKFY

SEQ ID NO: 128 - protein, 12 aa, Artificial sequence
  Synthetic sequence
PSLTRMLTGKFY

SEQ ID NO: 129 - protein, 12 aa, Artificial sequence
  Synthetic sequence
PELTIMLTPKFY

SEQ ID NO: 130 - protein, 12 aa, Artificial sequence
  Synthetic sequence
PCLTAMLTLKFA

SEQ ID NO: 131 - protein, 12 aa, Artificial sequence
  Synthetic sequence
PKLTRMLTHKFV

SEQ ID NO: 132 - protein, 12 aa, Artificial sequence
  Synthetic sequence
PCLTDMLTFKFY

SEQ ID NO: 133 - protein, 12 aa, Artificial sequence
  Synthetic sequence
PLLTDMLTRKFY

SEQ ID NO: 134 - protein, 12 aa, Artificial sequence
  Synthetic sequence
PLLTDMLTFKFY

SEQ ID NO: 135 - protein, 12 aa, Artificial sequence
  Synthetic sequence
PKLTDMLTFKFS

SEQ ID NO: 136 - protein, 12 aa, Artificial sequence
  Synthetic sequence
PKLTYMLTRKFY

SEQ ID NO: 137 - protein, 12 aa, Artificial sequence
  Synthetic sequence
PKLTRMLTFKFC

SEQ ID NO: 138 - protein, 12 aa, Artificial sequence
  Synthetic sequence
PKLTSMLTFKFS

SEQ ID NO: 139 - protein, 12 aa, Artificial sequence
  Synthetic sequence
PKLTYMLTFKFS

SEQ ID NO: 140 - protein, 12 aa, Artificial sequence
  Synthetic sequence
PKLTGMLTFKFS

SEQ ID NO: 141 - protein, 12 aa, Artificial sequence
  Synthetic sequence
PKLTVMLTFKFT

SEQ ID NO: 142 - protein, 12 aa, Artificial sequence
  Synthetic sequence
PKLTVMLTFKFS

SEQ ID NO: 143 - protein, 12 aa, Artificial sequence
  Synthetic sequence
PKLTVMLTFKFP

SEQ ID NO: 144 - protein, 12 aa, Artificial sequence
  Synthetic sequence
PKLTVMLTFKFF

SEQ ID NO: 145 - protein, 12 aa, Artificial sequence
  Synthetic sequence
PKLTCMLTFKFA

SEQ ID NO: 146 - protein, 12 aa, Artificial sequence
  Synthetic sequence
PKLTNMLTFKFA

SEQ ID NO: 147 - protein, 12 aa, Artificial sequence
  Synthetic sequence
PKLTNMLTFKFS

SEQ ID NO: 148 - protein, 451 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 216 aa, Artificial sequence
  Synthetic sequence
QSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 150 - protein, 455 aa, Artificial sequence
  Synthetic sequence
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGRRLRYFDWAAYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 214 aa, Artificial sequence
  Synthetic sequence
QSALTQDPAVSVALGQTVRITCQGDSLRSHYASWYQQKPGQAPVVVVYDNSDRPSGIPDRFSGSNSGNTATLTISRVEVGDEADYYCQVWDSSSDHYVFGTGTKVEIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 152 - protein, 450 aa, Artificial sequence
  Synthetic sequence
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKASGWYPWYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 213 aa, Artificial sequence
  Synthetic sequence
VIWMTQSPSSLSASLGDRVTITCRASQSVSTYINWYQQKSGKGPKLLIFNASTLQSGVPSRFSGSGSGTDFTLTITSLQPDDSATYYCQQSYRVPLSFGGGTKVEIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 154 - protein, 450 aa, Artificial sequence
  Synthetic sequence
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKAGSWYPYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 213 aa, Artificial sequence
  Synthetic sequence
DIVMTQSPSSLSASVGDRVTITCRASQTVSTYINWYQQRSGKGPKLLIFDASRLQSGAPSRFSGSGSGTEFTLTITSLQPDDSATYYCQQSYRIPLSFGGGTKVEIRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 156 - protein, 455 aa, Artificial sequence
  Synthetic sequence
QVQLQQWGAGLLKPSETLSLTCAVYGGSFSGYYWSWIRQPPGKGLEWIGEINHIGSTNYNPSLKSRVTISVDTSNNQFSLKLSSVTAADTAVYYCARGRRLRYFDWAAYNWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 214 aa, Artificial sequence
  Synthetic sequence
QSALTQDPAVSVALGQTVRISCQGDSLRSHYASWYQQKPGQAPVVVVYDNSDRPSGIPDRFSGSNSGNTATLTISRVEVGDEADYYCQVWDSSSDHYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 158 - protein, 451 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKGYGSGWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 216 aa, Artificial sequence; identical to SEQ ID NO: 149
  Synthetic sequence

SEQ ID NO: 160 - protein, 399 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 161 - protein, 399 aa, Artificial sequence
  Synthetic sequence
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLTGGGGSGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVL

SEQ ID NO: 162 - protein, 399 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGGGGSGGGGSGGGGSGGGGSAPTSSSTKKTQLQLEHLLLDLQMILNGINNYKNPKLTEMLTFKFYMPKKATELKHLQCLEEELKPLEEVLNLAQSKNFHLTARDAVDNMRVIIQELKGSETTFMCEYADETATIVEFLNRWITFAQSIISTLT

SEQ ID NO: 163 - protein, 399 aa, Artificial sequence
  Synthetic sequence
APTSSSTKKTQLQLEHLLLDLQMILNGINNYKNPKLTEMLTFKFYMPKKATELKHLQCLEEELKPLEEVLNLAQSKNFHLTARDAVDNMRVIIQELKGSETTFMCEYADETATIVEFLNRWITFAQSIISTLTGGGGSGGGGSGGGGSGGGGSQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVL

SEQ ID NO: 164 - protein, 400 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLGGGGSGGGGSGGGGSGGGGSAPTSSSTKKTQLQLEHLLLDLQMILNGINNYKNPLLTDMLTRFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 165 - protein, 360 aa, Artificial sequence
  Synthetic sequence
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 166 - protein, 360 aa, Artificial sequence
  Synthetic sequence
APASSSTKKTQLQLEHLLLDLQMILNGINNYKNPKLTRMLTFKFYMPKKATELKHLQCLEEELKPLEEVLNLAQSKNFHLGVTDSISNAIVLARELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 167 - protein, 360 aa, Artificial sequence
  Synthetic sequence
APASSSTKKTQLQLEHLLLDLQMILNGINNYKNPKLTEMLTFKFYMPKKATELKHLQCLEEELKPLEEVLNLAQSKNFHLTARDAVDNMRVIIQELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 168 - protein, 360 aa, Artificial sequence
  Synthetic sequence
APASSSTKKTQLQLEHLLLDLQMILNGINNYKNPLLTDMLTRKFYMPKKATELKHLQCLEEELKPLEEVLNLAQSKNFHLRPRDLISNINVIVLELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 169 - protein, 360 aa, Artificial sequence
  Synthetic sequence
APASSSTKKTQLQLEHLLLDLQMILNGINNYKNPLLTDMLTRKFYMPKKATELKHLQCLEEELKPLEEVLNLAQSKNFHLTARDAVDNMRVIIQELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 170 - protein, 360 aa, Artificial sequence
  Synthetic sequence
APASSSTKKTQLQLEHLLLDLQMILNGINNYKNPLLTDMLTRKFYMPKKATELKHLQCLEEELKPLEEVLNLAQSKNFHLGVTDSISNAIVLARELKGSETTFMCEYADETATIVEFLNRWITFSQSIISTLTDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 171 - protein, 365 aa, Artificial sequence
  Synthetic sequence
APASSSTKKTQLQLEHLLLDLQMILNGINNYKNPLLTDMLTRKFYMPKKATELKHLQCLEEELKPLEEVLNLAQSKNFHLTARDAIDNINVIVQELKGSETTFMCEYADETATIVEFLNRWITFSQSIISTLTEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 172 - protein, 365 aa, Artificial sequence
  Synthetic sequence
APASSSTKKTQLQLEHLLLDLQMILNGINNYKNPLLTDMLTRKFYMPKKATELKHLQCLEEELKPLEEVLNLAQSKNFHLTARDAISNINVIIQELKGSETTFMCEYADETATIVEFLNRWITFSQSIISTLTEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 173 - protein, 365 aa, Artificial sequence
  Synthetic sequence
APASSSTKKTQLQLEHLLLDLQMILNGINNYKNPLLTDMLTRKFYMPKKATELKHLQCLEEELKPLEEVLNLAQSKNFHLTPRDAIDNIRVIVQELKGSETTFMCEYADETATIVEFLNRWITFSQSIISTLTEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 174 - protein, 365 aa, Artificial sequence
  Synthetic sequence
APASSSTKKTQLQLEHLLLDLQMILNGINNYKNPLLTDMLTRKFYMPKKATELKHLQCLEEELKPLEEVLNLAQSKNFHLTPRDAIDNIRVIILELKGSETTFMCEYADETATIVEFLNRWITFSQSIISTLTEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 175 - protein, 365 aa, Artificial sequence
  Synthetic sequence
APASSSTKKTQLQLEHLLLDLQMILNGINNYKNPKLTDMLTFKFSMPKKATELKHLQCLEEELKPLEEVLNLAQSKNFHLTPRDAIDNIRVIVQELKGSETTFMCEYADETATIVEFLNRWITFSQSIISTLTEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 176 - protein, 473 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 177 - protein, 451 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 178 - protein, 451 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKGYGSGWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 179 - protein, 451 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKGFGSGWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 180 - protein, 451 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSGWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGK

SEQ ID NO: 181 - protein, 216 aa, Artificial sequence; identical to SEQ ID NO: 149
  Synthetic sequence

SEQ ID NO: 182 - protein, 451 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGK

SEQ ID NO: 183 - protein, 604 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 184 - protein, 604 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTRMLTFKFYMPKKATELKHLQCLEEELKPLEEVLNLAQSKNFHLTARDAVDNMRVIIQELKGSETTFMCEYADETATIVEFLNRWITFSQSIISTLT

SEQ ID NO: 185 - protein, 604 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSAPASSSTKKTQLQLEHLLLDLQMILNGINNYKNPKLTVMLTFKFSMPKKATELKHLQCLEEELKPLEEVLNLAQSKNFHLTPRDAISNIRVIIQELKGSETTFMCEYADETATIVEFLNRWITFSQSIISTLT

SEQ ID NO: 186 - protein, 584 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGKGGGGSGGGGSGGGGSGGGGSWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 187 - protein, 538 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSITCPPPMSVEHADIWVKSYSLYSRERYICNSGFKRKAGTSSLTECVLNKATNVAHWTTPSLKCIRDP

SEQ ID NO: 188 - protein, 606 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSALTVDKSRWQQGNVFSCSVMHEALHNHYTQKSLSLSPGKGGGGSGGGGSGGGGSGGGGSWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 189 - protein, 560 aa, Artificial sequence
  Synthetic sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARDKWYGSAWRGDYWGQGTLVTVSSGGGGSGGGGSGGGGSQSVLTQPPSASGTPGQRVTISCSGSYSNIGSNFVNWYQQLPGTAPKLLIHFNNQRPSGVPDRFSGSRSGTSASLAISGLQSEDEADYFCSTWDDSLKGPIFGGGTKLTVLDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFKLVSKLTVDKSRWQQGNVFSCSVMHEALHNHYTQKSLSLSPGKGGGGSGGGGSGGGGSGGGGSITCPPPMSVEHADIWVKSYSLYSRERYICNSGFKRKAGTSSLTECVLNKATNVAHWTTPSLKCIRDP

SEQ ID NO: 190 - DNA, 66 bp, Artificial sequence
  Synthetic sequence
gtgtattactgtgcgagagataaagggtatggcagtggctggaggggtgactactggggccaggga

SEQ ID NO: 191 - protein, 133 aa, Homo sapiens [sequence omitted]

SEQ ID NO: 192 - protein, 133 aa, Homo sapiens
  misc_feature 81: Xaa  = C  T  G  W  I  S  E  and K
  misc_feature 82: Xaa = Y  P  V  W  L  A  and G
  misc_feature 83: Xaa = S  T  Q  G  M  E  R  and K
  misc_feature 85: Xaa = A  V  S  and T
  misc_feature 86: Xaa = I  L  T  and V
  misc_feature 87: Xaa = S  T  E  D  and R
  misc_feature 89: Xaa = I  A  M  and V
  misc_feature 90: Xaa = S  T  N  Q  I  G  E  K  and R
  misc_feature 91: Xaa = V  L  and I
  misc_feature 92: Xaa = N  T  I  and L
  misc_feature 93: Xaa = V  A  and I
  misc_feature 94: Xaa = Q  L  G  K  and R
  misc_feature 95: Xaa = A  D  and E
APTSSSTKKTQLQLEHLLLDLQMILNGINNYKNPKLTRMLTFKFYMPKKATELKHLQCLEEELKPLEEVLNLAQSKNFHLXXXDXXXNXXXXXXXLKGSETTFMCEYADETATIVEFLNRWITFCQSIISTLT